SEQUENCE LISTING (224 sequences)

SEQ ID NO: 1 - protein, 19 aa, Artificial Sequence
  Description of Artificial Sequence Synthetic peptide
FVKDLLVHLKKLFREGQFN

SEQ ID NO: 2 - protein, 19 aa, Artificial Sequence
  Description of Artificial Sequence Synthetic peptide
FVKDLLVHLKKLFREGRFN

SEQ ID NO: 3 - protein, 19 aa, Artificial Sequence
  Description of Artificial Sequence Synthetic peptide
FVKDLLLHLKKLFREGQFN

SEQ ID NO: 4 - protein, 19 aa, Artificial Sequence
  Description of Artificial Sequence Synthetic peptide
FVKDLLLHLKKLFREGRFN

SEQ ID NO: 5 - protein, 16 aa, Artificial Sequence
  Description of Artificial Sequence Synthetic peptide
FVKDLLVHLKKLFREG

SEQ ID NO: 6 - protein, 16 aa, Artificial Sequence
  Description of Artificial Sequence Synthetic peptide
FVKDLLLHLKKLFREG

SEQ ID NO: 7 - protein, 17 aa, Artificial Sequence
  Description of Artificial Sequence Synthetic peptide
KDLLVHLKKLFREGQFN

SEQ ID NO: 8 - protein, 17 aa, Artificial Sequence
  Description of Artificial Sequence Synthetic peptide
KDLLVHLKKLFREGRFN

SEQ ID NO: 9 - protein, 17 aa, Artificial Sequence
  Description of Artificial Sequence Synthetic peptide
KDLLLHLKKLFREGQFN

SEQ ID NO: 10 - protein, 17 aa, Artificial Sequence
  Description of Artificial Sequence Synthetic peptide
KDLLLHLKKLFREGRFN

SEQ ID NO: 11 - protein, 13 aa, Artificial Sequence
  Description of Artificial Sequence Synthetic peptide
KDLLVHLKKLFRE

SEQ ID NO: 12 - protein, 13 aa, Artificial Sequence
  Description of Artificial Sequence Synthetic peptide
KDLLLHLKKLFRE

SEQ ID NO: 13 - protein, 8 aa, Artificial Sequence
  Description of Artificial Sequence Synthetic peptide
HLKKLFRE

SEQ ID NO: 14 - protein, 112 aa, Artificial Sequence
  Description of Artificial Sequence Synthetic polypeptide
SPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 15 - protein, 10 aa, Artificial Sequence
  Description of Artificial Sequence Synthetic peptide
GFNIKDTYIH

SEQ ID NO: 16 - protein, 17 aa, Artificial Sequence
  Description of Artificial Sequence Synthetic peptide
RIDPANDNIKYDPKFQG

SEQ ID NO: 17 - protein, 11 aa, Artificial Sequence
  Description of Artificial Sequence Synthetic peptide
SEENWYDFFDY

SEQ ID NO: 18 - protein, 16 aa, Artificial Sequence
  Description of Artificial Sequence Synthetic peptide
RSSQSIVHSNGNTYLE

SEQ ID NO: 19 - protein, 7 aa, Artificial Sequence
  Description of Artificial Sequence Synthetic peptide
KVSNRFS

SEQ ID NO: 20 - protein, 9 aa, Artificial Sequence
  Description of Artificial Sequence Synthetic peptide
FQGSHIPYT

SEQ ID NO: 21 - protein, 16 aa, Artificial Sequence
  Description of Artificial Sequence Synthetic peptide
XSSQSXXHSNGNTYLX

SEQ ID NO: 22 - protein, 7 aa, Artificial Sequence
  Description of Artificial Sequence Synthetic peptide
KXSXRFS

SEQ ID NO: 23 - protein, 7 aa, Artificial Sequence
  Description of Artificial Sequence Synthetic peptide
FQXXXXP

SEQ ID NO: 24 - protein, 132 aa, Macaca fascicularis
MALLLTMVIALTCLGGFASPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 25 - protein, 16 aa, Artificial Sequence
  Description of Artificial Sequence Synthetic peptide
XSSQSXXHSXGNXYLX

SEQ ID NO: 26 - protein, 16 aa, Artificial Sequence
  Description of Artificial Sequence Synthetic peptide
XSSQSXXHSXGNXYLE

SEQ ID NO: 27 - protein, 7 aa, Artificial Sequence
  Description of Artificial Sequence Synthetic peptide
KXSXXXS

SEQ ID NO: 28 - protein, 6 aa, Artificial Sequence
  Description of Artificial Sequence Synthetic peptide
QXXXIP

SEQ ID NO: 29 - protein, 9 aa, Artificial Sequence
  Description of Artificial Sequence Synthetic peptide
FQXXXXPYT

SEQ ID NO: 30 - protein, 102 aa, Artificial Sequence
  Description of Artificial Sequence Synthetic polypeptide
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHIPYT

SEQ ID NO: 31 - protein, 102 aa, Artificial Sequence
  Description of Artificial Sequence Synthetic polypeptide
DVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHIPYT

SEQ ID NO: 32 - protein, 102 aa, Artificial Sequence
  Description of Artificial Sequence Synthetic polypeptide
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHIPYT

SEQ ID NO: 33 - protein, 102 aa, Artificial Sequence
  Description of Artificial Sequence Synthetic polypeptide
DIVMTQTPLSLSVTPGQPASISCRSSQSIVHSNGNTYLEWYLQKPGQPPQLLIYKVSNRFSGVPDRFSGSGSGTDFTLKISRVEAEDVGVYYCFQGSHIPYT

SEQ ID NO: 34 - protein, 102 aa, Artificial Sequence
  Description of Artificial Sequence Synthetic polypeptide
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHIPYT

SEQ ID NO: 35 - protein, 102 aa, Artificial Sequence
  Description of Artificial Sequence Synthetic polypeptide
DIVMTQTPLSSPVTLGQPASISCRSSQSIVHSNGNTYLEWLQQRPGQPPRLLIYKVSNRFSGVPDRFSGSGAGTDFTLKISRVEAEDVGVYYCFQGSHIPYT

SEQ ID NO: 36 - protein, 102 aa, Artificial Sequence
  Description of Artificial Sequence Synthetic polypeptide
DIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYT

SEQ ID NO: 37 - protein, 102 aa, Artificial Sequence
  Description of Artificial Sequence Synthetic polypeptide
DVVMTQSPLSLPVTLGQPASISCRSSQSLVYSDGNTYLNWFQQRPGQSPRRLIYKVSNRFSGVPDRFSGSGSGTDFTLKISRVEAEDVGVYYCFQGSHIPYT

SEQ ID NO: 38 - protein, 102 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYT

SEQ ID NO: 39 - protein, 100 aa, Artificial Sequence
  Description of Artificial Sequence Synthetic polypeptide
DIVMTQTPLSLPVTPGEPASISCXSSQSXXHSXGNXYLXWYLQKPGQSPQLLIYKXSXXXSGVPDRFSGSGSGTDFTLKISRVEAEDVGVYYCFQXXXXP

SEQ ID NO: 40 - protein, 100 aa, Artificial Sequence
  Description of Artificial Sequence Synthetic polypeptide
DVVMTQSPLSLPVTLGQPASISCXSSQSXXHSXGNXYLXWFQQRPGQSPRRLIYKXSXXXSGVPDRFSGSGSGTDFTLKISRVEAEDVGVYYCFQXXXXP

SEQ ID NO: 41 - protein, 100 aa, Artificial Sequence
  Description of Artificial Sequence Synthetic polypeptide
DIVMTQTPLSLSVTPGQPASISCXSSQSXXHSXGNXYLXWYLQKPGQSPQLLIYKXSXXXSGVPDRFSGSGSGTDFTLKISRVEAEDVGVYYCFQXXXXP

SEQ ID NO: 42 - protein, 100 aa, Artificial Sequence
  Description of Artificial Sequence Synthetic polypeptide
DIVMTQTPLSLSVTPGQPASISCXSSQSXXHSXGNXYLXWYLQKPGQPPQLLIYKXSXXXSGVPDRFSGSGSGTDFTLKISRVEAEDVGVYYCFQXXXXP

SEQ ID NO: 43 - protein, 100 aa, Artificial Sequence
  Description of Artificial Sequence Synthetic polypeptide
DIVMTQSPLSLPVTPGEPASISCXSSQSXXHSXGNXYLXWYLQKPGQSPQLLIYKXSXXXSGVPDRFSGSGSGTDFTLKISRVEAEDVGVYYCFQXXXXP

SEQ ID NO: 44 - protein, 100 aa, Artificial Sequence
  Description of Artificial Sequence Synthetic polypeptide
DIVMTQTPLSSPVTLGQPASISCXSSQSXXHSXGNXYLXWLQQRPGQPPRLLIYKXSXXXSGVPDRFSGSGAGTDFTLKISRVEAEDVGVYYCFQXXXXP

SEQ ID NO: 45 - protein, 100 aa, Artificial Sequence
  Description of Artificial Sequence Synthetic polypeptide
DIQMTQSPSSLSASVGDRVTITCXSSQSXXHSXGNXYLXWYQQKPGKAPKLLIYKXSXXXSGVPSRFSGSGSGTDFTLTISSLQPEDFATYYCFQXXXXP

SEQ ID NO: 46 - protein, 100 aa, Artificial Sequence
  Description of Artificial Sequence Synthetic polypeptide
DVLMTQTPLSLPVSLGDQASISCXSSQSXXHSXGNXYLXWYLQKPGQSPKLLIYKXSXXXSGVPDRFSGSGSGTDFTLKISRVEAEDLGVYYCFQXXXXP

SEQ ID NO: 47 - protein, 11 aa, Artificial Sequence
  Description of Artificial Sequence Synthetic peptide
FGGGTKVEIKR

SEQ ID NO: 48 - protein, 10 aa, Artificial Sequence
  Description of Artificial Sequence Synthetic peptide
GXXIKDTYXH

SEQ ID NO: 49 - protein, 17 aa, Artificial Sequence
  Description of Artificial Sequence Synthetic peptide
XIDPXNDNIKYXXKFQG

SEQ ID NO: 50 - protein, 109 aa, Artificial Sequence
  Description of Artificial Sequence Synthetic polypeptide
QVQLVQSGAEVKKPGASVKVSCKASGFNIKDTYIHWVRQAPGQGLEWMGRIDPANDNIKYDPKFQGRVTMTRDTSISTAYMELSRLRSDDTAVYYCARSEENWYDFFDY

SEQ ID NO: 51 - protein, 109 aa, Artificial Sequence
  Description of Artificial Sequence Synthetic polypeptide
QVQLVQSGAEVKKPGASVKVSCKASGFNIKDTYIHWVRQAPGQRLEWMGRIDPANDNIKYDPKFQGRVTITRDTSASTAYMELSSLRSEDTAVYYCARSEENWYDFFDY

SEQ ID NO: 52 - protein, 109 aa, Artificial Sequence
  Description of Artificial Sequence Synthetic polypeptide
QVQLVQSGAEVKKPGASVKVSCKASGFNIKDTYIHWVRQATGQGLEWMGRIDPANDNIKYDPKFQGRVTMTRNTSISTAYMELSSLRSEDTAVYYCARSEENWYDFFDY

SEQ ID NO: 53 - protein, 109 aa, Artificial Sequence
  Description of Artificial Sequence Synthetic polypeptide
QVQLVQSGAEVKKPGASVKVSCKASGFNIKDTYIHWVRQAPGQGLEWMGRIDPANDNIKYDPKFQGRVTMTTDTSTSTAYMELRSLRSDDTAVYYCARSEENWYDFFDY

SEQ ID NO: 54 - protein, 109 aa, Artificial Sequence
  Description of Artificial Sequence Synthetic polypeptide
QVQLVQSGAEVKKPGASVKVSCKVSGFNIKDTYIHWVRQAPGKGLEWMGRIDPANDNIKYDPKFQGRVTMTEDTSTDTAYMELSSLRSEDTAVYYCATSEENWYDFFDY

SEQ ID NO: 55 - protein, 109 aa, Artificial Sequence
  Description of Artificial Sequence Synthetic polypeptide
QMQLVQSGAEVKKTGSSVKVSCKASGFNIKDTYIHWVRQAPGQALEWMGRIDPANDNIKYDPKFQGRVTITRDRSMSTAYMELSSLRSEDTAMYYCARSEENWYDFFDY

SEQ ID NO: 56 - protein, 109 aa, Artificial Sequence
  Description of Artificial Sequence Synthetic polypeptide
QVQLVQSGAEVKKPGASVKVSCKASGFNIKDTYIHWVRQAPGQGLEWMGRIDPANDNIKYDPKFQGRVTMTRDTSTSTVYMELSSLRSEDTAVYYCARSEENWYDFFDY

SEQ ID NO: 57 - protein, 107 aa, Artificial Sequence
  Description of Artificial Sequence Synthetic polypeptide
QMQLVQSGPEVKKPGTSVKVSCKASGFNIKDTYIHWVRQRQRLEWIGRIDPANDNIKYDPKFQGRVTITRDMSTSTAYMELSSLRSEDTAVYYCAASEENWYDFFDY

SEQ ID NO: 58 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISRDNAKNSLYLQMNSLRAEDTAVYYCARSEENWYDFFDY

SEQ ID NO: 59 - protein, 110 aa, Artificial Sequence
  Description of Artificial Sequence Synthetic polypeptide
EVQLVESGGGLVQPGRSLRLSCAASGFNIKDTYIHWVRQAPGKGLEWVSRIDPANDNIKYDPKFQGRFTISRDNAKNSLYLQMNSLRAEDTALYYCAKDSEENWYDFFDY

SEQ ID NO: 60 - protein, 109 aa, Artificial Sequence
  Description of Artificial Sequence Synthetic polypeptide
QVQLVESGGGLVKPGGSLRLSCAASGFNIKDTYIHWIRQAPGKGLEWVSRIDPANDNIKYDPKFQGRFTISRDNAKNSLYLQMNSLRAEDTAVYYCARSEENWYDFFDY

SEQ ID NO: 61 - protein, 109 aa, Artificial Sequence
  Description of Artificial Sequence Synthetic polypeptide
EVQLVESGGGLVKPGGSLRLSCAASGFNIKDTYIHWVRQAPGKGLEWVGRIDPANDNIKYDPKFQGRFTISRDDSKNTLYLQMNSLKTEDTAVYYCTTSEENWYDFFDY

SEQ ID NO: 62 - protein, 109 aa, Artificial Sequence
  Description of Artificial Sequence Synthetic polypeptide
EVQLVESGGGVVRPGGSLRLSCAASGFNIKDTYIHWVRQAPGKGLEWVSRIDPANDNIKYDPKFQGRFTISRDNAKNSLYLQMNSLRAEDTALYHCARSEENWYDFFDY

SEQ ID NO: 63 - protein, 109 aa, Artificial Sequence
  Description of Artificial Sequence Synthetic polypeptide
EVQLVESGGGLVKPGGSLRLSCAASGFNIKDTYIHWVRQAPGKGLEWVSRIDPANDNIKYDPKFQGRFTISRDNAKNSLYLQMNSLRAEDTAVYYCARSEENWYDFFDY

SEQ ID NO: 64 - protein, 109 aa, Artificial Sequence
  Description of Artificial Sequence Synthetic polypeptide
EVQLLESGGGLVQPGGSLRLSCAASGFNIKDTYIHWVRQAPGKGLEWVSRIDPANDNIKYDPKFQGRFTISRDNSKNTLYLQMNSLRAEDTAVYYCAKSEENWYDFFDY

SEQ ID NO: 65 - protein, 109 aa, Artificial Sequence
  Description of Artificial Sequence Synthetic polypeptide
QVQLVESGGGVVQPGRSLRLSCAASGFNIKDTYIHWVRQAPGKGLEWVARIDPANDNIKYDPKFQGRFTISRDNSKNTLYLQMNSLRAEDTAVYYCAKSEENWYDFFDY

SEQ ID NO: 66 - protein, 109 aa, Artificial Sequence
  Description of Artificial Sequence Synthetic polypeptide
QVQLVESGGGVVQPGRSLRLSCAASGFNIKDTYIHWVRQAPGKGLEWVARIDPANDNIKYDPKFQGRFTISRDNSKNTLYLQMNSLRAEDTAVYYCARSEENWYDFFDY

SEQ ID NO: 67 - protein, 110 aa, Artificial Sequence
  Description of Artificial Sequence Synthetic polypeptide
EVQLVESGGVVVQPGGSLRLSCAASGFNIKDTYIHWVRQAPGKGLEWVSRIDPANDNIKYDPKFQGRFTISRDNSKNSLYLQMNSLRTEDTALYYCAKDSEENWYDFFDY

SEQ ID NO: 68 - protein, 109 aa, Artificial Sequence
  Description of Artificial Sequence Synthetic polypeptide
EVQLVESGGGLVQPGGSLRLSCAASGFNIKDTYIHWVRQAPGKGLEWVSRIDPANDNIKYDPKFQGRFTISRDNAKNSLYLQMNSLRDEDTAVYYCARSEENWYDFFDY

SEQ ID NO: 69 - protein, 109 aa, Artificial Sequence
  Description of Artificial Sequence Synthetic polypeptide
EVQLVESGGGLVQPGRSLRLSCTASGFNIKDTYIHWFRQAPGKGLEWVGRIDPANDNIKYDPKFQGRFTISRDGSKSIAYLQMNSLKTEDTAVYYCTRSEENWYDFFDY

SEQ ID NO: 70 - protein, 109 aa, Artificial Sequence
  Description of Artificial Sequence Synthetic polypeptide
EVQLVESGGGLVQPGGSLRLSCAASGFNIKDTYIHWVRQAPGKGLEYVSRIDPANDNIKYDPKFQGRFTISRDNSKNTLYLQMGSLRAEDMAVYYCARSEENWYDFFDY

SEQ ID NO: 71 - protein, 109 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISRDNAKNSLYLQMNSLRAEDTAVYYCARSEENWYDFFDY

SEQ ID NO: 72 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISRDNAKNSLYLQMNSLRAEDTAVYYCARSEENWYDFFDY

SEQ ID NO: 73 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISADNAKNSLYLQMNSLRAEDTAVYYCARSEENWYDFFDY

SEQ ID NO: 74 - protein, 109 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISRDNAKNSLYLQMNSLRAEDTAVYYCARSEENWYDFFDY

SEQ ID NO: 75 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISADNAKNSLYLQMNSLRAEDTAVYYCARSEENWYDFFDY

SEQ ID NO: 76 - protein, 109 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSLYLQMNSLRAEDTAVYYCARSEENWYDFFDY

SEQ ID NO: 77 - protein, 109 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSLYLQMNSLRAEDTAVYYCARSEENWYDFFDY

SEQ ID NO: 78 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISRDNAKNSAYLQMNSLRAEDTAVYYCARSEENWYDFFDY

SEQ ID NO: 79 - protein, 109 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSAYLQMNSLRAEDTAVYYCARSEENWYDFFDY

SEQ ID NO: 80 - protein, 109 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDY

SEQ ID NO: 81 - protein, 109 aa, Artificial Sequence
  Description of Artificial Sequence Synthetic polypeptide
EVQLVESGGGLVQPGGSLRLSCTGSGFNIKDTYIHWVRQAPGKGLEWIGRIDPANDNIKYDPKFQGRFTISADNAKNSLYLQMNSLRAEDTAVYYCARSEENWYDFFDY

SEQ ID NO: 82 - protein, 109 aa, Artificial Sequence
  Description of Artificial Sequence Synthetic polypeptide
EVQLQQSGAELVKPGASVKLSCTGSGFNIKDTYIHWVKQRPEQGLEWIGRIDPANDNIKYDPKFQGKATITADTSSNTAYLQLNSLTSEDTAVYYCARSEENWYDFFDY

SEQ ID NO: 83 - protein, 109 aa, Artificial Sequence
  Description of Artificial Sequence Synthetic polypeptide
QVQLVQSGAEVKKPGASVKVSCKASGXXIKDTYXHWVRQAPGQGLEWMGXIDPXNDNIKYXXKFQGRVTMTRDTSISTAYMELSRLRSDDTAVYYCARSEENWYDFFDY

SEQ ID NO: 84 - protein, 109 aa, Artificial Sequence
  Description of Artificial Sequence Synthetic polypeptide
QVQLVQSGAEVKKPGASVKVSCKASGXXIKDTYXHWVRQAPGQRLEWMGXIDPXNDNIKYXXKFQGRVTITRDTSASTAYMELSSLRSEDTAVYYCARSEENWYDFFDY

SEQ ID NO: 85 - protein, 109 aa, Artificial Sequence
  Description of Artificial Sequence Synthetic polypeptide
QVQLVQSGAEVKKPGASVKVSCKASGXXIKDTYXHWVRQATGQGLEWMGXIDPXNDNIKYXXKFQGRVTMTRNTSISTAYMELSSLRSEDTAVYYCARSEENWYDFFDY

SEQ ID NO: 86 - protein, 109 aa, Artificial Sequence
  Description of Artificial Sequence Synthetic polypeptide
QVQLVQSGAEVKKPGASVKVSCKASGXXIKDTYXHWVRQAPGQGLEWMGXIDPXNDNIKYXXKFQGRVTMTTDTSTSTAYMELRSLRSDDTAVYYCARSEENWYDFFDY

SEQ ID NO: 87 - protein, 109 aa, Artificial Sequence
  Description of Artificial Sequence Synthetic polypeptide
QVQLVQSGAEVKKPGASVKVSCKVSGXXIKDTYXHWVRQAPGKGLEWMGXIDPXNDNIKYXXKFQGRVTMTEDTSTDTAYMELSSLRSEDTAVYYCATSEENWYDFFDY

SEQ ID NO: 88 - protein, 109 aa, Artificial Sequence
  Description of Artificial Sequence Synthetic polypeptide
QMQLVQSGAEVKKTGSSVKVSCKASGXXIKDTYXHWVRQAPGQALEWMGXIDPXNDNIKYXXKFQGRVTITRDRSMSTAYMELSSLRSEDTAMYYCARSEENWYDFFDY

SEQ ID NO: 89 - protein, 109 aa, Artificial Sequence
  Description of Artificial Sequence Synthetic polypeptide
QVQLVQSGAEVKKPGASVKVSCKASGXXIKDTYXHWVRQAPGQGLEWMGXIDPXNDNIKYXXKFQGRVTMTRDTSTSTVYMELSSLRSEDTAVYYCARSEENWYDFFDY

SEQ ID NO: 90 - protein, 107 aa, Artificial Sequence
  Description of Artificial Sequence Synthetic polypeptide
QMQLVQSGPEVKKPGTSVKVSCKASGXXIKDTYXHWVRQRQRLEWIGXIDPXNDNIKYXXKFQGRVTITRDMSTSTAYMELSSLRSEDTAVYYCAASEENWYDFFDY

SEQ ID NO: 91 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGRFTISRDNAKNSLYLQMNSLRAEDTAVYYCARSEENWYDFFDY

SEQ ID NO: 92 - protein, 110 aa, Artificial Sequence
  Description of Artificial Sequence Synthetic polypeptide
EVQLVESGGGLVQPGRSLRLSCAASGXXIKDTYXHWVRQAPGKGLEWVSXIDPXNDNIKYXXKFQGRFTISRDNAKNSLYLQMNSLRAEDTALYYCAKDSEENWYDFFDY

SEQ ID NO: 93 - protein, 109 aa, Artificial Sequence
  Description of Artificial Sequence Synthetic polypeptide
QVQLVESGGGLVKPGGSLRLSCAASGXXIKDTYXHWIRQAPGKGLEWVSXIDPXNDNIKYXXKFQGRFTISRDNAKNSLYLQMNSLRAEDTAVYYCARSEENWYDFFDY

SEQ ID NO: 94 - protein, 109 aa, Artificial Sequence
  Description of Artificial Sequence Synthetic polypeptide
EVQLVESGGGLVKPGGSLRLSCAASGXXIKDTYXHWVRQAPGKGLEWVGXIDPXNDNIKYXXKFQGRFTISRDDSKNTLYLQMNSLKTEDTAVYYCTTSEENWYDFFDY

SEQ ID NO: 95 - protein, 109 aa, Artificial Sequence
  Description of Artificial Sequence Synthetic polypeptide
EVQLVESGGGVVRPGGSLRLSCAASGXXIKDTYXHWVRQAPGKGLEWVSXIDPXNDNIKYXXKFQGRFTISRDNAKNSLYLQMNSLRAEDTALYHCARSEENWYDFFDY

SEQ ID NO: 96 - protein, 109 aa, Artificial Sequence
  Description of Artificial Sequence Synthetic polypeptide
EVQLVESGGGLVKPGGSLRLSCAASGXXIKDTYXHWVRQAPGKGLEWVSXIDPXNDNIKYXXKFQGRFTISRDNAKNSLYLQMNSLRAEDTAVYYCARSEENWYDFFDY

SEQ ID NO: 97 - protein, 109 aa, Artificial Sequence
  Description of Artificial Sequence Synthetic polypeptide
EVQLLESGGGLVQPGGSLRLSCAASGXXIKDTYXHWVRQAPGKGLEWVSXIDPXNDNIKYXXKFQGRFTISRDNSKNTLYLQMNSLRAEDTAVYYCAKSEENWYDFFDY

SEQ ID NO: 98 - protein, 109 aa, Artificial Sequence
  Description of Artificial Sequence Synthetic polypeptide
QVQLVESGGGVVQPGRSLRLSCAASGXXIKDTYXHWVRQAPGKGLEWVAXIDPXNDNIKYXXKFQGRFTISRDNSKNTLYLQMNSLRAEDTAVYYCAKSEENWYDFFDY

SEQ ID NO: 99 - protein, 109 aa, Artificial Sequence
  Description of Artificial Sequence Synthetic polypeptide
QVQLVESGGGVVQPGRSLRLSCAASGXXIKDTYXHWVRQAPGKGLEWVAXIDPXNDNIKYXXKFQGRFTISRDNSKNTLYLQMNSLRAEDTAVYYCARSEENWYDFFDY

SEQ ID NO: 100 - protein, 110 aa, Artificial Sequence
  Description of Artificial Sequence Synthetic polypeptide
EVQLVESGGVVVQPGGSLRLSCAASGXXIKDTYXHWVRQAPGKGLEWVSXIDPXNDNIKYXXKFQGRFTISRDNSKNSLYLQMNSLRTEDTALYYCAKDSEENWYDFFDY

SEQ ID NO: 101 - protein, 109 aa, Artificial Sequence
  Description of Artificial Sequence Synthetic polypeptide
EVQLVESGGGLVQPGGSLRLSCAASGXXIKDTYXHWVRQAPGKGLEWVSXIDPXNDNIKYXXKFQGRFTISRDNAKNSLYLQMNSLRDEDTAVYYCARSEENWYDFFDY

SEQ ID NO: 102 - protein, 109 aa, Artificial Sequence
  Description of Artificial Sequence Synthetic polypeptide
EVQLVESGGGLVQPGRSLRLSCTASGXXIKDTYXHWFRQAPGKGLEWVGXIDPXNDNIKYXXKFQGRFTISRDGSKSIAYLQMNSLKTEDTAVYYCTRSEENWYDFFDY

SEQ ID NO: 103 - protein, 109 aa, Artificial Sequence
  Description of Artificial Sequence Synthetic polypeptide
EVQLVESGGGLVQPGGSLRLSCAASGXXIKDTYXHWVRQAPGKGLEYVSXIDPXNDNIKYXXKFQGRFTISRDNSKNTLYLQMGSLRAEDMAVYYCARSEENWYDFFDY

SEQ ID NO: 104 - protein, 109 aa, Artificial Sequence
  Description of Artificial Sequence Synthetic polypeptide
EVQLVESGGGLVQPGGSLRLSCAASGXXIKDTYXHWVRQAPGKGLEWIGXIDPXNDNIKYXXKFQGRFTISRDNAKNSLYLQMNSLRAEDTAVYYCARSEENWYDFFDY

SEQ ID NO: 105 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGKATISRDNAKNSLYLQMNSLRAEDTAVYYCARSEENWYDFFDY

SEQ ID NO: 106 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGRFTISADNAKNSLYLQMNSLRAEDTAVYYCARSEENWYDFFDY

SEQ ID NO: 107 - protein, 109 aa, Artificial Sequence
  Description of Artificial Sequence Synthetic polypeptide
EVQLVESGGGLVQPGGSLRLSCAASGXXIKDTYXHWVRQAPGKGLEWVGXIDPXNDNIKYXXKFQGRFTISRDNAKNSLYLQMNSLRAEDTAVYYCARSEENWYDFFDY

SEQ ID NO: 108 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGKATISADNAKNSLYLQMNSLRAEDTAVYYCARSEENWYDFFDY

SEQ ID NO: 109 - protein, 109 aa, Artificial Sequence
  Description of Artificial Sequence Synthetic polypeptide
EVQLVESGGGLVQPGGSLRLSCAASGXXIKDTYXHWVRQAPGKGLEWIGXIDPXNDNIKYXXKFQGRFTISADNAKNSLYLQMNSLRAEDTAVYYCARSEENWYDFFDY

SEQ ID NO: 110 - protein, 109 aa, Artificial Sequence
  Description of Artificial Sequence Synthetic polypeptide
EVQLVESGGGLVQPGGSLRLSCAASGXXIKDTYXHWVRQAPGKGLEWVGXIDPXNDNIKYXXKFQGRFTISADNAKNSLYLQMNSLRAEDTAVYYCARSEENWYDFFDY

SEQ ID NO: 111 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGRFTISRDNAKNSAYLQMNSLRAEDTAVYYCARSEENWYDFFDY

SEQ ID NO: 112 - protein, 109 aa, Artificial Sequence
  Description of Artificial Sequence Synthetic polypeptide
EVQLVESGGGLVQPGGSLRLSCAASGXXIKDTYXHWVRQAPGKGLEWVGXIDPXNDNIKYXXKFQGRFTISADNAKNSAYLQMNSLRAEDTAVYYCARSEENWYDFFDY

SEQ ID NO: 113 - protein, 109 aa, Artificial Sequence
  Description of Artificial Sequence Synthetic polypeptide
EVQLVESGGGLVQPGGSLRLSCAASGXXIKDTYXHWVRQAPGKGLEWIGXIDPXNDNIKYXXKFQGRFTISADNAKNSAYLQMNSLRAEDTAVYYCARSEENWYDFFDY

SEQ ID NO: 114 - protein, 109 aa, Artificial Sequence
  Description of Artificial Sequence Synthetic polypeptide
EVQLVESGGGLVQPGGSLRLSCTGSGXXIKDTYXHWVRQAPGKGLEWIGXIDPXNDNIKYXXKFQGRFTISADNAKNSLYLQMNSLRAEDTAVYYCARSEENWYDFFDY

SEQ ID NO: 115 - protein, 109 aa, Artificial Sequence
  Description of Artificial Sequence Synthetic polypeptide
EVQLQQSGAELVKPGASVKLSCTGSGXXIKDTYXHWVKQRPEQGLEWIGXIDPXNDNIKYXXKFQGKATITADTSSNTAYLQLNSLTSEDTAVYYCARSEENWYDFFDY

SEQ ID NO: 116 - protein, 11 aa, Artificial Sequence
  Description of Artificial Sequence Synthetic peptide
WGQGTTLTVSS

SEQ ID NO: 117 - protein, 11 aa, Artificial Sequence
  Description of Artificial Sequence Synthetic peptide
WGQGTLVTVSS

SEQ ID NO: 118 - protein, 11 aa, Artificial Sequence
  Description of Artificial Sequence Synthetic peptide
QASQGTSINLN

SEQ ID NO: 119 - protein, 7 aa, Artificial Sequence
  Description of Artificial Sequence Synthetic peptide
GASNLED

SEQ ID NO: 120 - protein, 9 aa, Artificial Sequence
  Description of Artificial Sequence Synthetic peptide
LQHSYLPWT

SEQ ID NO: 121 - protein, 10 aa, Artificial Sequence
  Description of Artificial Sequence Synthetic peptide
GFSLTGYGVN

SEQ ID NO: 122 - protein, 14 aa, Artificial Sequence
  Description of Artificial Sequence Synthetic peptide
IIWGDGSTDYNSAL

SEQ ID NO: 123 - protein, 16 aa, Artificial Sequence
  Description of Artificial Sequence Synthetic peptide
DKTFYYDGFYRGRMDY

SEQ ID NO: 124 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 125 - protein, 427 aa, Homo sapiens [sequence omitted]

SEQ ID NO: 126 - protein, 101 aa, Homo sapiens
DVVMTQSPLSLPVTLGQPASISCRSSQSLVYSDGNTYLNWFQQRPGQSPRRLIYKVSNRFSGVPDRFSGSGSGTDFTLKISRVEAEDVGVYYCMQGTHWPP

SEQ ID NO: 127 - protein, 20 aa, Macaca fascicularis
MALLLTMVIALTCLGGFASP

SEQ ID NO: 128 - protein, 329 aa, Artificial Sequence
  Description of Artificial Sequence Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - DNA, 360 bp, Mus musculus
gaggttcagctgcagcagtctggggcagagcttgtgaagccaggggcctcagtcaagttgtcctgcacaggttctggcttcaacattaaagacacctatatacactgggtgaagcagaggcctgaacagggcctggagtggattggaaggattgatcctgcgaatgataatattaaatatgacccgaagttccagggcaaggccactataacagcagacacatcctccaacacagcctacctacagctcaacagcctgacatctgaggacactgccgtctattactgtgctagatctgaggaaaattggtacgacttttttgactactggggccaaggcaccactctcacagtctcctca

SEQ ID NO: 130 - protein, 120 aa, Mus musculus
EVQLQQSGAELVKPGASVKLSCTGSGFNIKDTYIHWVKQRPEQGLEWIGRIDPANDNIKYDPKFQGKATITADTSSNTAYLQLNSLTSEDTAVYYCARSEENWYDFFDYWGQGTTLTVSS

SEQ ID NO: 131 - protein, 19 aa, Artificial Sequence
  Description of Artificial Sequence Synthetic peptide
MKCSWVIFFLMAVVTGVNS

SEQ ID NO: 132 - DNA, 336 bp, Mus musculus
gatgttttgatgacccaaactccactctccctgcctgtcagtcttggagatcaagcctccatctcttgcaggtctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagattagcagagtggaggctgaggatctgggagtttattactgctttcaaggttcacatattccgtacacgttcggaggggggaccaagctggaaataaaa

SEQ ID NO: 133 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYTFGGGTKLEIK

SEQ ID NO: 134 - protein, 19 aa, Artificial Sequence
  Description of Artificial Sequence Synthetic peptide
MKLPVRLLVLMFWIPASSS

SEQ ID NO: 135 - DNA, 429 bp, Mus musculus
atggctgtcctggcattactcttctgcctggtaacattcccaagctgtatcctttcccaggtgcagctgaaggagtcaggacctggcctggtggcgccctcacagagcctgtccatcacatgcaccgtctcagggttctcattaaccggctatggtgtaaactgggttcgccagcctccaggaaagggtctggagtggctgggaataatttggggtgatggaagcacagactataattcagctctcaaatccagactgatcatcaacaaggacaactccaagagccaagttttcttaaaaatgaacagtctgcaaactgatgacacagccaggtacttctgtgccagagataagactttttactacgatggtttctacaggggcaggatggactactggggtcaaggaacctcagtcaccgtctcctca

SEQ ID NO: 136 - protein, 124 aa, Mus musculus
QVQLKESGPGLVAPSQSLSITCTVSGFSLTGYGVNWVRQPPGKGLEWLGIIWGDGSTDYNSALKSRLIINKDNSKSQVFLKMNSLQTDDTARYFCARDKTFYYDGFYRGRMDYWGQGTSVTVSS

SEQ ID NO: 137 - protein, 124 aa, Artificial Sequence; identical to SEQ ID NO: 136
  Description of Artificial Sequence Synthetic polypeptide

SEQ ID NO: 138 - DNA, 387 bp, Mus musculus
atgaacacgagggcccctgctgagttccttgggttcctgttgctctggtttttaggtgccagatgtgatgtccagatgattcagtctccatcctccctgtctgcatctttgggagacattgtcaccatgacttgccaggcaagtcagggcactagcattaatttaaactggtttcagcaaaaaccagggaaagctcctaagctcctgatctttggtgcaagcaacttggaagatggggtcccatcaaggttcagtggcagtagatatgggacaaatttcactctcaccatcagcagcctggaggatgaagatatggcaacttatttctgtctacagcatagttatctcccgtggacgttcggtggcggcaccaaactggaaatcaaa

SEQ ID NO: 139 - protein, 107 aa, Mus musculus
DVQMIQSPSSLSASLGDIVTMTCQASQGTSINLNWFQQKPGKAPKLLIFGASNLEDGVPSRFSGSRYGTNFTLTISSLEDEDMATYFCLQHSYLPWTFGGGTKLEIK

SEQ ID NO: 140 - protein, 22 aa, Artificial Sequence
  Description of Artificial Sequence Synthetic peptide
MNTRAPAEFLGFLLLWFLGARC

SEQ ID NO: 141 - protein, 329 aa, Homo sapiens; identical to SEQ ID NO: 128

SEQ ID NO: 142 - DNA, 417 bp, Mus musculus
atgaaatgcagctgggttatcttcttcctgatggcagtggttacaggggtcaattcagaggttcagctgcagcagtctggggcagagcttgtgaagccaggggcctcagtcaagttgtcctgcacaggttctggcttcaacattaaagacacctatatacactgggtgaagcagaggcctgaacagggcctggagtggattggaaggattgatcctgcgaatgataatattaaatatgacccgaagttccagggcaaggccactataacagcagacacatcctccaacacagcctacctacagctcaacagcctgacatctgaggacactgccgtctattactgtgctagatctgaggaaaattggtacgacttttttgactactggggccaaggcaccactctcacagtctcctca

SEQ ID NO: 143 - protein, 139 aa, Mus musculus
MKCSWVIFFLMAVVTGVNSEVQLQQSGAELVKPGASVKLSCTGSGFNIKDTYIHWVKQRPEQGLEWIGRIDPANDNIKYDPKFQGKATITADTSSNTAYLQLNSLTSEDTAVYYCARSEENWYDFFDYWGQGTTLTVSS

SEQ ID NO: 144 - DNA, 393 bp, Mus musculus
atgaagttgcctgttaggctgttggtgctgatgttctggattcctgcttccagcagtgatgttttgatgacccaaactccactctccctgcctgtcagtcttggagatcaagcctccatctcttgcaggtctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagattagcagagtggaggctgaggatctgggagtttattactgctttcaaggttcacatattccgtacacgttcggaggggggaccaagctggaaataaaa

SEQ ID NO: 145 - protein, 131 aa, Mus musculus
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYTFGGGTKLEIK

SEQ ID NO: 146 - DNA, 417 bp, Artificial Sequence
  Description of Artificial Sequence Synthetic polynucleotide
atggattggacctggcgcatcctgttcctggtggccgctgccaccggcgctcactctcaggtgcagctggtgcagtctggcgccgaggtgaagaagcctggcgcttccgtgaaggtgtcctgtaaggcctccggcttcaacatcaaggacacctacatccactgggtgcggcaggctcccggccagcggctggagtggatgggccggatcgatcctgccaacgacaacatcaagtacgaccccaagtttcagggccgcgtgaccatcacccgcgatacctccgcttctaccgcctacatggagctgtctagcctgcggagcgaggataccgccgtgtactactgcgcccgctccgaggagaactggtacgacttcttcgactactggggccagggcaccctggtgaccgtgtcctct

SEQ ID NO: 147 - protein, 144 aa, Artificial Sequence
  Description of Artificial Sequence Synthetic polypeptide
MDWTWRILFLVAAATGAHSQVQLVQSGAEVKKPGASVKVSCKASGFNIKDTYIHWVRQAPGQRLEWMGRIDPANDNIKYDPKFQGRVTITRDTSASTAYMELSSLRSEDTAVYYCARSEENWYDFFDYWGQGTLVTVSSGESCR

SEQ ID NO: 148 - DNA, 396 bp, Artificial Sequence
  Description of Artificial Sequence Synthetic polynucleotide
atgcggctgcccgctcagctgctgggcctgctgatgctgtgggtgcccggctcttccggcgacgtggtgatgacccagtcccctctgtctctgcccgtgaccctgggccagcccgcttctatctcttgccggtcctcccagtccatcgtgcactccaacggcaacacctacctggagtggtttcagcagagacccggccagtctcctcggcggctgatctacaaggtgtccaaccgcttttccggcgtgcccgatcggttctccggcagcggctccggcaccgatttcaccctgaagatcagccgcgtggaggccgaggatgtgggcgtgtactactgcttccagggctcccacatcccttacacctttggcggcggaaccaaggtggagatcaag

SEQ ID NO: 149 - protein, 132 aa, Artificial Sequence
  Description of Artificial Sequence Synthetic polypeptide
MRLPAQLLGLLMLWVPGSSGDVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHIPYTFGGGTKVEIK

SEQ ID NO: 150 - DNA, 417 bp, Artificial Sequence
  Description of Artificial Sequence Synthetic polynucleotide
atggagctgggcctgtcttgggtgttcctggtggctatcctggagggcgtgcagtgcgaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggccggttcaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 151 - protein, 139 aa, Artificial Sequence
  Description of Artificial Sequence Synthetic polypeptide
MELGLSWVFLVAILEGVQCEVQLVESGGGLVQPGGSLRLSCAASGFNIKDTYIHWVRQAPGKGLEWVARIDPANDNIKYDPKFQGRFTISRDNAKNSLYLQMNSLRAEDTAVYYCARSEENWYDFFDYWGQGTLVTVSS

SEQ ID NO: 152 - DNA, 405 bp, Artificial Sequence
  Description of Artificial Sequence Synthetic polynucleotide
atggatatgcgcgtgcccgctcagctgctgggcctgctgctgctgtggctgcgcggagcccgctgcgatatccagatgacccagtccccttcttctctgtccgcctctgtgggcgatcgcgtgaccatcacctgtcggtcctcccagtccatcgtgcactccaacggcaacacctacctggagtggtatcagcagaagcccggcaaggcccctaagctgctgatctacaaggtgtccaaccgcttttccggcgtgccttctcggttctccggctccggctccggcaccgatttcaccctgaccatctcctccctccagcccgaggatttcgccacctactactgcttccagggctcccacatcccttacacctttggcggcggaaccaaggtggagatcaagcgt

SEQ ID NO: 153 - protein, 135 aa, Artificial Sequence
  Description of Artificial Sequence Synthetic polypeptide
MDMRVPAQLLGLLLLWLRGARCDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYTFGGGTKVEIKR

SEQ ID NO: 154 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtggatcggccggatcgatcctgccaacgacaacatcaagtacgaccccaagttccagggccggttcaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 155 - protein, 120 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISRDNAKNSLYLQMNSLRAEDTAVYYCARSEENWYDFFDYWGQGTLVTVSS

SEQ ID NO: 156 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggcaaggccaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 157 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISRDNAKNSLYLQMNSLRAEDTAVYYCARSEENWYDFFDYWGQGTLVTVSS

SEQ ID NO: 158 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 159 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISADNAKNSLYLQMNSLRAEDTAVYYCARSEENWYDFFDYWGQGTLVTVSS

SEQ ID NO: 160 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtgggccggatcgatcctgccaacgacaacatcaagtacgaccccaagttccagggccggttcaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 161 - protein, 120 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISRDNAKNSLYLQMNSLRAEDTAVYYCARSEENWYDFFDYWGQGTLVTVSS

SEQ ID NO: 162 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggcaaggccaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 163 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISADNAKNSLYLQMNSLRAEDTAVYYCARSEENWYDFFDYWGQGTLVTVSS

SEQ ID NO: 164 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtggatcggccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 165 - protein, 120 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSLYLQMNSLRAEDTAVYYCARSEENWYDFFDYWGQGTLVTVSS

SEQ ID NO: 166 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtgggccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 167 - protein, 120 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSLYLQMNSLRAEDTAVYYCARSEENWYDFFDYWGQGTLVTVSS

SEQ ID NO: 168 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggccggttcaccatctctcgcgacaacgccaagaactccgcctacctccagatgaactctctgcgcgccgaggataccgccgtgtactactgcgcccggagcgaggagaactggtacgacttcttcgactactggggccagggcaccctggtgaccgtgtcctct

SEQ ID NO: 169 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISRDNAKNSAYLQMNSLRAEDTAVYYCARSEENWYDFFDYWGQGTLVTVSS

SEQ ID NO: 170 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtgggccggatcgatcctgccaacgacaacatcaagtacgaccccaagttccagggccggttcaccatctctgccgacaacgccaagaactccgcctacctccagatgaactctctgcgcgccgaggataccgccgtgtactactgcgcccggagcgaggagaactggtacgacttcttcgactactggggccagggcaccctggtgaccgtgtcctct

SEQ ID NO: 171 - protein, 120 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSAYLQMNSLRAEDTAVYYCARSEENWYDFFDYWGQGTLVTVSS

SEQ ID NO: 172 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtggatcggccggatcgatcctgccaacgacaacatcaagtacgaccccaagttccagggccggttcaccatctctgccgacaacgccaagaactccgcctacctccagatgaactctctgcgcgccgaggataccgccgtgtactactgcgcccggagcgaggagaactggtacgacttcttcgactactggggccagggcaccctggtgaccgtgtcctct

SEQ ID NO: 173 - protein, 120 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDYWGQGTLVTVSS

SEQ ID NO: 174 - DNA, 360 bp, Artificial Sequence
  Description of Artificial Sequence Synthetic polynucleotide
gaggtgcagctggtggagtctggcggcggactggtgcagcctggcggctctctgcggctgtcttgcaccggctccggcttcaacatcaaggacacctacatccactgggtgcggcaggctcccggcaagggcctggagtggatcggccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 175 - protein, 120 aa, Artificial Sequence
  Description of Artificial Sequence Synthetic polypeptide
EVQLVESGGGLVQPGGSLRLSCTGSGFNIKDTYIHWVRQAPGKGLEWIGRIDPANDNIKYDPKFQGRFTISADNAKNSLYLQMNSLRAEDTAVYYCARSEENWYDFFDYWGQGTLVTVSS

SEQ ID NO: 176 - protein, 450 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 219 aa, Artificial Sequence
  Description of Artificial Sequence Synthetic polypeptide
DIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 179 - protein, 13 aa, Macaca fascicularis
MALLLTMVIALTC

SEQ ID NO: 180 - protein, 12 aa, Macaca fascicularis
LGGFASPSPVPP

SEQ ID NO: 181 - protein, 17 aa, Macaca fascicularis
SPSPVPPSTALKELIEE

SEQ ID NO: 182 - protein, 19 aa, Macaca fascicularis
TALKELIEELVNITQNQKA

SEQ ID NO: 183 - protein, 22 aa, Macaca fascicularis
NQKAPLCNGSMVWSINLTAGVY

SEQ ID NO: 184 - protein, 21 aa, Macaca fascicularis
INLTAGVYCAALESLINVSGC

SEQ ID NO: 185 - protein, 21 aa, Macaca fascicularis
SLINVSGCSAIEKTQRMINGF

SEQ ID NO: 186 - protein, 18 aa, Macaca fascicularis
GFCPHKVSAGQFSSLRVR

SEQ ID NO: 187 - protein, 20 aa, Macaca fascicularis
VRDTKIEVAQFVKDLLVHLK

SEQ ID NO: 188 - protein, 19 aa, Macaca fascicularis
FVKDLLVHLKKLFREGQFN

SEQ ID NO: 189 - DNA, 396 bp, Artificial Sequence
  Description of Artificial Sequence Synthetic polynucleotide
atgcggctgcccgctcagctgctgggcctgctgatgctgtgggtgcccggctcttccggcgacgtggtgatgacccagtcccctctgtctctgcccgtgaccctgggccagcccgcttctatctcttgccggtcctcccagtccctggtgtactccgacggcaacacctacctgaactggttccagcagagacccggccagtctcctcggcggctgatctacaaggtgtccaaccgcttttccggcgtgcccgatcggttctccggctccggcagcggcaccgatttcaccctgaagatcagccgcgtggaggccgaggatgtgggcgtgtactactgcttccagggctcccacatcccttacacctttggcggcggaaccaaggtggagatcaag

SEQ ID NO: 190 - protein, 132 aa, Artificial Sequence
  Description of Artificial Sequence Synthetic polypeptide
MRLPAQLLGLLMLWVPGSSGDVVMTQSPLSLPVTLGQPASISCRSSQSLVYSDGNTYLNWFQQRPGQSPRRLIYKVSNRFSGVPDRFSGSGSGTDFTLKISRVEAEDVGVYYCFQGSHIPYTFGGGTKVEIK

SEQ ID NO: 191 - DNA, 336 bp, Artificial Sequence
  Description of Artificial Sequence Synthetic polynucleotide
gatgttgtgatgacccaatctccactctccctgcctgtcactcctggagagccagcctccatctcttgcagatctagtcagagcattgtgcatagtaatggaaacacctacctggaatggtacctgcagaaaccaggccagtctccacagctcctgatctacaaagtttccaaccgattttctggggtcccagacaggttcagtggcagtggatcagggacagatttcacactcaagatcagcagagtggaggctgaggatgtgggagtttattactgctttcaaagttcacatgttcctctcaccttcggtcaggggaccaagctggagatcaaa

SEQ ID NO: 192 - protein, 112 aa, Artificial Sequence
  Description of Artificial Sequence Synthetic polypeptide
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQSSHVPLTFGQGTKLEIK

SEQ ID NO: 193 - protein, 329 aa, Homo sapiens; identical to SEQ ID NO: 128

SEQ ID NO: 194 - protein, 132 aa, Homo sapiens; identical to SEQ ID NO: 178

SEQ ID NO: 195 - protein, 113 aa, Homo sapiens; identical to SEQ ID NO: 124

SEQ ID NO: 196 - protein, 10 aa, Mus musculus
LDGYYFGFAY

SEQ ID NO: 197 - protein, 15 aa, Mus musculus
KASESVDNYGKSLMH

SEQ ID NO: 198 - protein, 118 aa, Mus musculus
EVKLVESGGGLVKPGGSLKLSCAASGFTFISYAMSWVRQTPEKRLEWVASISSGGNTYYPDSVKGRFTISRDNARNILYLQMSSLRSEDTAMYYCARLDGYYFGFAYWGQGTTVTVSS

SEQ ID NO: 199 - protein, 111 aa, Mus musculus
DIVLTQSPASLAVSLGQRATISCKASESVDNYGKSLMHWYQQKPGQSPKLLIYRASNLESGIPARFSGSGSRTDFTLTINPVEADDVATYYCQQSNEDPWTFGGGTKLEIK

SEQ ID NO: 200 - protein, 7 aa, Mus musculus
RASNLES

SEQ ID NO: 201 - protein, 9 aa, Mus musculus
QQSNEDPWT

SEQ ID NO: 202 - protein, 6 aa, Mus musculus
ISYAMS

SEQ ID NO: 203 - protein, 16 aa, Mus musculus
SISSGGNTYYPDSVKG

SEQ ID NO: 204 - DNA, 351 bp, Mus musculus
gaagtgaagctggtggagtctgggggaggcttagtgaaacctggagggtccctgaaactctcctgtgcagcctctggattcactttcattagctatgccatgtcttgggttcgtcagactccagagaagaggctggagtgggtcgcatccattagtagtggtggtaacacctactatccagacagtgtgaagggccgattcaccatctccagagataatgccaggaacatcctatacctgcaaatgagcagtctgaggtctgaggacacggccatgtattactgtgcacgacttgatggttactactttggatttgcttactggggccaagggactctggtcgctgtctct

SEQ ID NO: 205 - DNA, 354 bp, Artificial Sequence
  Description of Artificial Sequence Synthetic partially humanized antibody heavy chain sequence
gaggtcaagctggtggagtcagggggaggcttagtgcaacctggagggtccctgagactctcctgtgcagcctctggattcactttcattagctatgccatgtcttgggttcgtcaggctccagggaaggggctggagtgggtcgcatccattagtagtggtggtaacacctactatccagacagcgtgaagggccgattcaccatctccagagataatgccaagaacagcctatacctgcaaatgaacagtctgagggctgaggacacggccgtgtattactgtgcacgacttgatggttactactttggatttgcttactggggccaagggaccctggtcaccgtctcctca

SEQ ID NO: 206 - DNA, 354 bp, Artificial Sequence
  Description of Artificial Sequence Synthetic fully humanized antibody heavy chain sequence
gaggtccagctggtggagtcagggggaggcttagtgcaacctggagggtccctgagactctcctgtgcagcctctggattcactttcattagctatgccatgtcttgggttcgtcaggctccagggaaggggctggagtgggtcgcatccattagtagtggtggtaacacctactatccagacagcgtgaagggccgattcaccatctccagagataatgccaagaacagcctatacctgcaaatgaacagtctgagggctgaggacacggccgtgtattactgtgcacgacttgatggttactactttggatttgcttactggggccaagggaccctggtcaccgtctcctca

SEQ ID NO: 207 - DNA, 354 bp, Artificial Sequence
  Description of Artificial Sequence Synthetic fully humanized antibody heavy chain sequence
gaggtccagctggtggagtcagggggaggcttagtgaaacctggagggtccctgagactctcctgtgcagcctctggattcactttcattagctatgccatgtcttgggttcgtcaggctccagggaaggggctggagtgggtctcatccattagtagtggtggtaacacctactatccagacagtgtgaagggccgattcaccatctccagagataatgccaagaacagcctatacctgcaaatgaacagtctgagggctgaggacacggccgtgtattactgtgcacgacttgatggttactactttggatttgcttactggggccaagggaccacggtcaccgtctcctca

SEQ ID NO: 208 - protein, 118 aa, Mus musculus; identical to SEQ ID NO: 198

SEQ ID NO: 209 - protein, 118 aa, Artificial Sequence
  Description of Artificial Sequence Synthetic partially humanized antibody heavy chain sequence
EVKLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSS

SEQ ID NO: 210 - protein, 118 aa, Artificial Sequence
  Description of Artificial Sequence Synthetic fully humanized antibody heavy chain sequence
EVQLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSS

SEQ ID NO: 211 - protein, 118 aa, Artificial Sequence
  Description of Artificial Sequence Synthetic fully humanized antibody heavy chain sequence
EVQLVESGGGLVKPGGSLRLSCAASGFTFISYAMSWVRQAPGKGLEWVSSISSGGNTYYPDSVKGRFTISRDNAKNSLYLQMNSLRAEDTAVYYCARLDGYYFGFAYWGQGTTVTVSS

SEQ ID NO: 212 - protein, 111 aa, Artificial Sequence
  Description of Artificial Sequence Synthetic fully humanized antibody light chain sequence
DIQMTQSPSSLSASVGDRVTITCKASESVDNYGKSLMHWYQQKPGKAPKLLIYRASNLESGVPSRFSGSGSGTDFTLTISSLQPEDFATYYCQQSNEDPWTFGGGTKVEIK

SEQ ID NO: 213 - protein, 111 aa, Mus musculus; identical to SEQ ID NO: 199

SEQ ID NO: 214 - protein, 111 aa, Artificial Sequence
  Description of Artificial Sequence Synthetic partially humanized antibody light chain sequence
DIQLTQSPSSLSASVGDRVTITCKASESVDNYGKSLMHWYQQKPGKAPKLLIYRASNLESGVPSRFSGSGSRTDFTLTISSLQPEDFATYYCQQSNEDPWTFGGGTKVEIK

SEQ ID NO: 215 - protein, 111 aa, Artificial Sequence
  Description of Artificial Sequence Synthetic fully humanized antibody light chain sequence
DIVLTQSPASLAVSPGQRATITCKASESVDNYGKSLMHWYQQKPGQPPKLLIYRASNLESGVPARFSGSGSGTDFTLTINPVEANDTANYYCQQSNEDPWTFGGGTKVEIK

SEQ ID NO: 216 - protein, 107 aa, Homo sapiens [sequence omitted]

SEQ ID NO: 217 - DNA, 351 bp, Mus musculus; identical to SEQ ID NO: 204

SEQ ID NO: 218 - DNA, 333 bp, Mus musculus
gacattgtgctgacccaatctccagcttctttggctgtgtctctagggcagagggccaccatatcctgcaaagccagtgaaagtgttgataattatggcaaaagtttaatgcactggtaccagcagaaaccaggacagtcacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtaatgaggatccgtggacgttcggtggaggcaccaagctggaaatcaaa

SEQ ID NO: 219 - DNA, 333 bp, Mus musculus; identical to SEQ ID NO: 218

SEQ ID NO: 220 - DNA, 333 bp, Artificial Sequence
  Description of Artificial Sequence Synthetic partially humanized antibody light chain sequence
gacatccagctgacccagtctccatcctccctgtctgcatctgtgggagacagagtcaccatcacttgcaaagccagtgaaagtgttgataattatggcaaaagtctgatgcactggtatcagcagaaaccagggaaagctcctaagctcctgatctatcgtgcatccaacctggaatctggcgtcccatcaaggttcagtggcagtggatctcgcacagatttcactctcaccatcagcagtctgcaacctgaagattttgcaacttactactgtcagcaaagtaatgaggatccctggaccttcggcggagggaccaaggtagagatcaaa

SEQ ID NO: 221 - DNA, 333 bp, Artificial Sequence
  Description of Artificial Sequence Synthetic fully humanized antibody light chain sequence
gacatccagatgacccagtctccatcctccctgtctgcatctgtgggagacagagtcaccatcacttgcaaagccagtgaaagtgttgataattatggcaaaagtctgatgcactggtatcagcagaaaccagggaaagctcctaagctcctgatctatcgtgcatccaacctggaatctggcgtcccatcaaggttcagtggcagtggatctggcacagatttcactctcaccatcagcagtctgcaacctgaagattttgcaacttactactgtcagcaaagtaatgaggatccctggaccttcggcggagggaccaaggtagagatcaaa

SEQ ID NO: 222 - DNA, 351 bp, Mus musculus; identical to SEQ ID NO: 204

SEQ ID NO: 223 - DNA, 333 bp, Artificial Sequence
  Description of Artificial Sequence Synthetic fully humanized antibody light chain sequence
gacatcgtgctcactcagtctccagcttctttggctgtgtctccagggcagagggccaccataacctgcaaagccagtgaaagtgttgataattatggcaaaagtttaatgcactggtaccagcagaaaccaggacagccacccaaactcctcatctatcgtgcatccaacctagaatctggggtccctgccaggttcagtggcagtgggtctgggacagacttcaccctcaccattaatcctgtggaggctaatgatactgcaaactattactgtcagcaaagtaatgaggatccgtggacgttcggtggagggaccaaggtggaaataaaa

SEQ ID NO: 224 - protein, 227 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSNIC

(19) United States
(12) Patent Application Publication
(10) Pub. No.: US 2009/0068195 A1
(43) Pub. Date: Mar. 12, 2009

(54) METHODS AND COMPOSITIONS FOR TREATING AND MONITORING TREATMENT OF IL-13-ASSOCIATED DISORDERS

(75) Inventors: Yulia Vugmeyster, North Reading, MA (US); Xin Xu, Andover, MA (US); Xianbin Tian, Andover, MA (US); Donald G. Raible, Devon, PA (US); Yuji Simon Zhou, Malvern, MA (US); Billie Durn, Spring City, PA (US)

Correspondence Address: LOWRIE, LANDO & ANASTASI, LLP;W2023, ONE MAIN STREET, SUITE 1100, CAMBRIDGE, MA, 02142, US

(73) Assignee: Wyeth, Cambridge, MA (US)

(21) Appl. No.: 12/107,456

(22) Filed: Apr. 22, 2008

Related U.S. Application Data
(60) Provisional application No. 60/926,078, filed on Apr. 23, 2007.
(60) Provisional application No. 60/925,932, filed on Apr. 23, 2007.

(51) Int. Cl. A61K 39/395; G06F 19/00; A61P 11/00; A61P 29/00; A61P 35/00

(52) U.S. Cl. 424/158.1; 702/19

(57) ABSTRACT

Methods and compositions for reducing or inhibiting, or preventing or delaying the onset of, one or more symptoms associated with an early and/or a late phase of an IL-13-associated disorder or condition using IL-13 binding agents are disclosed. Methods for evaluating the kinetics and/or efficacy of an IL-13 binding agent in treating or preventing an IL-13-associated disorder or condition in a subject, e.g., a human subject, are also disclosed.

### CROSS REFERENCE TO RELATED APPLICATIONS

This application claims priority to U.S. Ser. No. 60/926,078 and U.S. Ser. No. 60/925,932, both of which were filed on Apr. 23, 2007. The contents of the aforementioned applications are hereby incorporated by reference in their entirety. This application also incorporates by reference the International Application filed with the U.S. Receiving Office on Apr. 22, 2008, entitled “Methods and Compositions for Treating and Monitoring Treatment of IL-13-Associated Disorders” and bearing attorney docket number W2023-7007WO.

### SEQUENCE LISTING

An electronic copy of the Sequence Listing in both pdf and txt formats is being submitted herewith.

### BACKGROUND

Interleukin-13 (IL-13) is a cytokine secreted by T lymphocytes and mast cells (McKenzie et al. (1993) Proc. Natl. Acad. Sci. USA 90:3735-39; Bost et al. (1996) Immunology 87:663-41). IL-13 shares several biological activities with IL-4. For example, either IL-4 or IL-13 can cause IgE isotype switching in B cells (Tomkinson et al. (2001) J. Immunol. 166:5792-5800). Additionally, increased levels of cell surface CD23 and serum CD23 (sCD23) have been reported in asthmatic patients (Sanchez-Guererro et al. (1994) Allergy 49:587-92; DiLorenzo et al. (1999) Allergy Asthma Proc. 20:119-25). In addition, either IL-4 or IL-13 can upregulate the expression of MHC class II and the low-affinity IgE receptor (CD23) on B cells and monocytes, which results in enhanced antigen presentation and regulated macrophage function (Tomkinson et al., supra). Importantly, either IL-4 or IL-13 can increase the expression of VCAM-1 on endothelial cells, which facilitates preferential recruitment of eosinophils (and T cells) to the airway tissues (Tomkinson et al., supra). Either IL-4 or IL-13 can also increase airway mucus secretion, which can exacerbate airway responsiveness (Tomkinson et al., supra). These observations suggest that although IL-13 is not necessary for, or even capable of, inducing Th2 development, IL-13 may be a key player in the development of airway eosinophilia and AHR (Tomkinson et al., supra; Wills-Karp et al. (1998) Science 282:2258-61).

### SUMMARY

Methods and compositions for treating and/or monitoring treatment of IL-13-associated disorders or conditions are disclosed. In one embodiment, Applicants have discovered that administration of an IL-13 antagonist, e.g., an IL-13 antibody molecule, reduces at least one symptom of an allergen-induced early and/or a late asthmatic response in a subject, e.g., a human subject, relative to an untreated subject. The reduction in one or more asthmatic symptoms is detected within minutes following exposure of the subject to the allergen, and during an early asthmatic response (e.g., up to about 3 hours after exposure to the allergen). The reduction in symptoms is maintained during a late asthmatic response (e.g., for a period of about 3 to 24 hours after allergen exposure). In other embodiments, methods of evaluating an anti-IL13 antibody molecule and/or treatment modalities associated with said antibody molecule are disclosed. The evaluation methods include detecting at least one pharmacokinetic/pharmacodynamic (PK/PD) parameter of the anti-IL13 antibody molecule in the subject. Thus, uses of IL-13 binding agents or antagonists for reducing or inhibiting, and/or preventing or delaying the onset of, in a subject, one or more symptoms associated with an early and/or a late phase of an IL-13-associated disorder or condition are disclosed. In other embodiments, methods for evaluating the kinetics and/or efficacy of an IL-13 binding agent or antagonist in treating or preventing the IL-13-associated disorder or condition in a subject are also disclosed.

Accordingly, in one aspect, the invention features a method of treating or preventing an early and/or a late phase of an IL-13-associated disorder or condition in a subject. The method includes administering an IL-13 binding agent or an antagonist to the subject, in an amount effective to reduce one or more symptoms of the disorder or condition (e.g., in an amount effective to reduce one or more of: a respiratory symptom (e.g., bronchoconstriction), IgE levels, release or levels of histamine or leukotriene, or eotaxin levels in the subject). In the case of prophylactic use (e.g., to prevent, reduce or delay onset or recurrence of one or more symptoms of the disorder or condition), the subject may or may not have one or more symptoms of the disorder or condition. For example, the IL-13 binding agent or antagonist can be administered prior to exposure to an insult, or prior to the onset of any detectable manifestation of the symptoms, or after at least some, but not all the symptoms are detected. In the case of therapeutic use, the treatment may improve, cure, maintain, or decrease duration of, the disorder or condition in the subject. In therapeutic uses, the subject may have a partial or full manifestation of the symptoms. In a typical case, treatment improves the disorder or condition of the subject to an extent detectable by a physician, or prevents worsening of the disorder or condition.

In one embodiment, the IL-13 binding agent or antagonist inhibits or reduces one or more symptoms associated with an early phase of the IL-13 associated disorder, e.g., an “early asthmatic response” or “EAR”. For example, the IL-13 binding agent or antagonist reduces one or more symptoms associated with an EAR, e.g., about 0.25, about 0.5, about 1, about 1.5, about 2, about 2.5, or about 3 hours after an insult (e.g., allergen exposure) until about 3 hours after insult (e.g., allergen exposure). The IL-13 binding agent or antagonist can decrease or prevent one or more symptoms of the EAR including, but not limited to, one or more of: a release of at least one allergic mediator such as a leukotriene (e.g., LTA₄, LTB₄, LTC₄, LTD₄, LTE₄, and/or LTF₄) and/or histamine, e.g., from airway mast or basophil cells; an increase in the levels of at least one allergic mediator such as a leukotriene and/or histamine; bronchoconstriction; and/or airway edema. The IL-13 binding agent or antagonist can cause a decrease in one or more of these EAR symptoms in the subject, e.g., as compared to the level or degree of the symptom in the subject in the absence of the IL-13 binding agent or antagonist. Alternatively, the IL-13 binding agent or antagonist can prevent as large of an increase in the symptom, e.g., as compared to the level or degree of the symptom in the subject in the absence of the IL-13 binding agent or antagonist).

In other embodiments, the IL-13 binding agent or antagonist inhibits or reduces one or more symptoms associated with a late phase of an IL-13 associated disorder, e.g., a “late asthmatic response” or “LAR”. For example, the IL-13 binding agent or antagonist reduces one or more symptoms associated with an LAR, e.g., at least about 3, about 3.5, about 4, about 4.5, about 5, about 5.5, about 6, about 6.5, about 7, about 8, about 9, about 10, about 11, about 12, or about 13 hours after an insult (e.g., allergen exposure) up to about 24 hours after an insult (e.g., allergen exposure). For example, the IL-13 binding agent or antagonist can decrease or prevent one or more symptoms of the LAR, e.g., one or more of: airway reactivity and/or an influx and/or activation of inflammatory cells, such as lymphocytes, eosinophils and/or macrophages, e.g., in the airways and/or bronchial mucosa. The IL-13 binding agent or antagonist can cause a decrease in one or more of these symptoms of an LAR in a subject, e.g., as compared to the level or degree of the symptom in the subject in the absence of the IL-13 binding agent or antagonist. Alternatively, the IL-13 binding agent or antagonist can prevent as large of an increase in the symptom, e.g., as compared to the level or degree of the symptom in the subject in the absence of the IL-13 binding agent or antagonist).

The IL-13 binding agent or antagonist can be administered prior to the onset or recurrence of one or more symptoms associated with the IL-13-disorder or condition, but before a full manifestation of the symptoms associated with the disorder or condition. In certain embodiments, the IL-13 binding agent or antagonist is administered to the subject prior to exposure to an agent that triggers or exacerbates an IL-13-associated disorder or condition, e.g., an allergen, a pollutant, a toxic agent, an infection and/or stress. In some embodiments, the IL-13 binding agent or antagonist is administered prior to, during, or shortly after exposure to the agent that triggers and/or exacerbates the IL-13-associated disorder or condition. For example, the IL-13 binding agent or antagonist can be administered 1, 5, 10, 25, or 24 hours; 2, 3, 4, 5, 10, 15, 20, or 30 days; or 4, 5, 6, 7 or 8 weeks, or more before or after exposure to the triggering or exacerbating agent. Typically, the IL-13 binding agent or antagonist can be administered anywhere between 24 hours and 2 days before or after exposure to the triggering or exacerbating agent. In those embodiments where administration occurs after exposure to the agent, the subject may not be experiencing symptoms or may be experiencing a partial manifestation of the symptoms. For example, the subject may have symptoms of an early stage of the disorder or condition. Each dose can be administered by inhalation or by injection, e.g., subcutaneously, in an amount of about 0.5-10 mg/kg (e.g., about 0.7-5 mg/kg, about 0.9-4 mg/kg, about 1-3 mg/kg, about 1.5-2.5 mg/kg, or about 2 mg/kg). In one embodiment, the single treatment interval includes two subcutaneous doses of about 1-3 mg/kg, about 1.5-2.5 mg/kg, or about 2 mg/kg of an anti-IL13 antibody molecule at least 4, 7, 9 or 14 days apart. For example, the single treatment interval can include two subcutaneous doses of about 2 mg/kg of an anti-IL13 antibody molecule 7 days apart. In some embodiments, a flat dose of an anti-IL13 antibody molecule is administered to the subject, e.g., a flat dose of between about 50 mg and 500 mg, about 60 mg and 490 mg, about 70 mg to 480 mg, about 75 mg to 460 mg, about 80 mg to 450, about 100 mg and about 450 mg, about 150 mg to about 400 mg, about 200 mg to about 300 mg, about 200 mg to about 250 mg; or about 60 mg, 65 mg, 70 mg, 75 mg, 80 mg, 90 mg, 100 mg, 125 mg, 150 mg, 175 mg, 200 mg, 225 mg, or 250 mg. The flat dose (e.g., about 75 mg, 100 mg, 200 mg or 225 of the anti-IL13 antibody molecule) (or any combination of the flat dose) can be administered as a schedule of about once a week, every two weeks, every three weeks, four weeks, or month, or any combination thereof, or as determined by a clinician. An exemplary schedule of a flat dose of the anti-IL13 antibody is as follows: initial dose at day 1, followed by doses at about days 8, 28, 42, 56, 70 and 84.

In one embodiment, the IL-13 binding agent or antagonist is administered at a single treatment interval, e.g., as a single dose, or as a repeated dose of no more than two or three doses during a single treatment interval, e.g., the repeated dose is administered within one week or less from the initial dose.

The IL-13 antagonist or binding agent can be administered to a subject having, or at risk of having, an IL-13-associated disorder or condition. Typically, the subject is a mammal, e.g., a human (e.g., a child, an adolescent or an adult) suffering from or at risk of having an IL-13-associated disorder or condition. Examples of IL-13-associated disorders or conditions include, but are not limited to, disorders chosen from one or more of: IgE-related disorders, including but not limited to, atopic disorders, e.g., resulting from an increased sensitivity to IL-13 (e.g., atopic dermatitis, urticaria, eczema, and allergic conditions such as allergic rhinitis and allergic enterogastritis); respiratory disorders, e.g., asthma (e.g., allergic and nonallergic asthma (e.g., asthma due to infection with, e.g., respiratory syncytial virus (RSV), e.g., in younger children)), chronic obstructive pulmonary disease (COPD), and other conditions involving airway inflammation, eosinophilia, fibrosis and excess mucus production, e.g., cystic fibrosis and pulmonary fibrosis; inflammatory and/or autoimmune disorders or conditions, e.g., skin inflammatory disorders or conditions (e.g., atopic dermatitis), gastrointestinal disorders or conditions (e.g., inflammatory bowel diseases (IBD), ulcerative colitis and/or Crohn's disease), liver disorders or conditions (e.g., cirrhosis, hepatocellular carcinoma), and scleroderma; tumors or cancers (e.g., soft tissue or solid tumors), such as leukemia, glioblastoma, and lymphoma, e.g., Hodgkin's lymphoma; viral infections (e.g., from HTLV-1); fibrosis of other organs, e.g., fibrosis of the liver (e.g., fibrosis caused by a hepatitis B and/or C virus); and suppression of expression of protective type 1 immune responses, (e.g., during vaccination).

In certain embodiments, the subject is a human having mild, moderate or severe asthma, e.g., atopic asthma. The therapeutic and prophylactic methods disclosed herein can be practiced prior to, during or after allergen exposure. For example, the subject can be a human allergic to a seasonal allergen, e.g., ragweed, or an asthmatic patient after exposure to a cold or flu virus or during the cold or flu season. Prior to the onset of the symptoms (e.g., allergic or asthmatic symptoms, or prior to or during an allergy, or cold or flu season), a single dose interval of the anti-IL-13 binding agent or antagonist can be administered to the subject, such that the symptoms are reduced and/or the onset of the disorder or condition is delayed. Similarly, administration of the IL-13 binding agent or antagonist can be effected prior to the manifestation of one or more symptoms (e.g., before a full manifestations of the symptoms) associated with the disorder or condition when treating chronic conditions that are characterized by recurring flares or episodes of the disorder or condition. An exemplary method for treating allergic rhinitis or other allergic disorders can include initiating therapy with an IL-13 binding agent or antagonist prior to exposure to an allergen, e.g., prior to seasonal exposure to an allergen, e.g., prior to allergen blooms. Such therapy can include a single treatment interval, e.g., a single dose, of the IL-13 binding agent or antagonist. In other embodiments, the IL-13 binding agent or antagonist is administered in combination with allergy immunotherapy. For example the IL-13 binding agent or antagonist is administered in combination with an allergy immunization, e.g., a vaccine containing one or more allergens, such as ragweed, dust mite, and ryegrass. The administration of the Il-13 binding agent or antagonist can be repeated until a predetermined level of immunity is obtained in the subject.

In other embodiments, the IL-13 binding agent or antagonist is administered in an amount effective to reduce or inhibit, or prevent or delay the onset of, one or more of the symptoms of the IL-13-associated disorder or condition. For example the IL-13 binding agent or antagonist can be administered in an amount that decreases one or more of: (i) the levels of IL-13 (e.g., free IL-13) in the subject; (ii) the levels of eotaxin in the subject; (iii) the levels of histamine or leukotrienes in the subject; (iv) the amount of histamine or leukotrienes released by mast cells or basophils (e.g., blood basophils); (v) the IgE-titers in the subject; and/or (vi) one or more changes in the respiratory symptoms of the subject (e.g., bronchoconstriction, e.g., difficulty breathing, wheezing, coughing, shortness of breath and/or difficulty performing normal daily activities).

In other embodiments, the IL-13 binding agent or antagonist inhibits or reduces one or more biological activities of IL-13 or an IL-13 receptor (e.g., an IL-13 receptor α1 or an IL-13 receptor α2). Exemplary biological activities that can be reduced using the IL-13 binding agent or antagonist disclosed herein include, but is not limited to, one or more of: induction of CD23 expression; production of IgE by human B cells; phosphorylation of a transcription factor, e.g., STAT protein (e.g., STAT6 protein); antigen-induced eosinophilia in vivo; antigen-induced bronchoconstriction in vivo; and/or drug-induced airway hyperreactivity in vivo. Antagonism using an antagonist of IL-13/IL-13R does not necessarily indicate a total elimination of the biological activity of the IL-13/IL-13R polypeptide.

In one embodiment, the anti-IL-13 antibody molecule used in the therapeutic and prophylactic methods is described herein. In other embodiments, the anti-IL13 antibody molecule used in the methods is described in WO 05/123126, published on Dec. 29, 2005 or its U.S. equivalent U.S. 06/0063228 (the entire contents of both applications are incorporated herein by reference). For example, the antibody molecule is an antibody that interferes with (e.g., inhibits, blocks or otherwise reduces) binding of IL-13 to an epitope in either IL-13Rα1 or IL-13Rα2. In other embodiments, the antibody molecule binds to a complex that includes IL-13 and IL-13Rα1. In embodiments, the antibody molecule binds to IL-13 and interferes with (e.g., inhibits blocks or otherwise reduces) binding between a complex of IL-13 and IL-13Rα1 with IL-4Rα. In other embodiments, the antibody molecule can, e.g., confer a post-injection protective effect against exposure to Ascaris antigen in a sheep model at least 6 weeks after injection.

In one embodiment, the IL-13 binding agent or antagonist is administered in combination with another therapeutic agent. The combination therapy can include an IL-13 binding agent, e.g., an anti-IL-13 antibody molecule, co-formulated with and/or co-administered with one or more additional therapeutic agents, e.g., one or more cytokine and growth factor inhibitors, immunosuppressants, anti-inflammatory agents (e.g., systemic anti-inflammatory agents), metabolic inhibitors, enzyme inhibitors, and/or cytotoxic or cytostatic agents, as described in more herein. The IL-13 binding agent and the other therapeutic can also be administered separately.

Examples of preferred additional therapeutic agents that can be coadministered and/or coformulated with an IL-13 binding agent include: inhaled steroids; beta-agonists, e.g., short-acting or long-acting beta-agonists; antagonists of leukotrienes or leukotriene receptors; combination drugs such as ADVAIR®; IgE inhibitors, e.g., anti-IgE antibodies (e.g., XOLAIR®); phosphodiesterase inhibitors (e.g., PDE4 inhibitors); xanthines; anticholinergic drugs; mast cell-stabilizing agents such as cromolyn; IL-4 inhibitors (e.g., an IL-4 inhibitor antibody, IL-4 receptor fusion or an IL-4 mutein); IL-5 inhibitors; eotaxin/CCR3 inhibitors; and antihistamines. Such combinations can be used to treat asthma and other respiratory disorders. Additional examples of therapeutic agents that can be co-administered and/or co-formulated with an IL-13 binding agent include one or more of: TNF antagonists (e.g., a soluble fragment of a TNF receptor, e.g., p55 or p75 human TNF receptor or derivatives thereof, e.g., 75 kd TNFR-IgG (75 kD TNF receptor-IgG fusion protein, ENBREL®)); TNF enzyme antagonists, e.g., TNFα converting enzyme (TACE) inhibitors; muscarinic receptor antagonists; TGF-θ antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, Mk-2 and NFPB inhibitors, among others.

In another aspect, this application provides compositions, e.g., pharmaceutical compositions, that include a pharmaceutically acceptable carrier and at least one IL-13 binding agent, e.g., an anti-IL-13 antibody molecule. In one embodiment, the compositions, e.g., pharmaceutical compositions, comprise a combination of two or more IL-13 binding agents, e.g., two or more anti-IL-13 antibody molecules. A combinations of the IL-13 binding agent, e.g., the anti-IL-13 antibody molecule, and a drug, e.g., a therapeutic agent (e.g., one or more of an anti-histamine, an anti-leukotriene, a cytokine or a growth factor inhibitor, an immunosuppressant, an anti-inflammatory agent (e.g., systemic anti-inflammatory agent), a metabolic inhibitor, an enzyme inhibitor, and/or a cytotoxic or cytostatic agent, as described herein, can also be used.

In yet another embodiment, the methods disclosed herein further include: evaluating the efficacy of an IL-13 binding agent (e.g., an anti-IL13 antibody molecule as described herein or in WO 05/123126), in a subject, e.g., a human or non-human subject. The method of evaluating the efficacy of the IL-13 binding agent can be practiced alone, or in addition to the therapeutic and/or diagnostic methods described herein. In embodiments, the efficacy of the IL-13 binding agent in reducing pulmonary symptoms (e.g., eosinophilia, mucus production, bronchoconstriction, bronchospasm) is evaluated by assessing one or more of the following parameters: (i) detecting the levels of IL-13 in a sample (e.g., detecting the levels of IL-13 unbound and/or bound to an anti-IL13 antibody as described herein); (ii) measuring eotaxin levels in a sample; (iii) detecting the levels or release of histamine and/or leukotrienes; (iv) detecting IgE-titers (total and/or allergen-specific IgE); (v) detecting any changes to cysteinyl leukotriene receptor 1 or 2 protein or mRNA levels; (vi) evaluating changes in the symptoms of the subject (e.g., bronchoconstriction, e.g., difficulty breathing, wheezing, coughing, shortness of breath and/or difficulty performing normal daily activities); (vii) evaluating lung function in a subject (e.g., forced expiratory volume in 1 second (FEV1); (viii) evaluating a change in the level of one or more cytokines (e.g., MCP-1, TNFα and/or interleukin-6 (IL-6); (ix) evaluating a change in an inflammatory cell and/or marker in a sample from a subject; and/or (x) evaluating at least one pharmacokinetic/pharmacodynamic (PK/PD) parameter of the IL-13 binding agent, e.g., a PK/PD parameter as described herein. The evaluation of parameters (i)-(x) can be carried out before and/or after administration of the IL-13 binding agent (after single or multiple administrations) to the subject (e.g., at selected intervals after initiating therapy). The evaluation can be performed by a clinician or support staff. The sample can be a biological sample, such as serum, plasma, blood, or sputum or tissue sample. A change, e.g., a reduction, in one or more of (i)-(x) relative to a predetermined level (e.g., comparison before and after treatment) indicates that the IL-13 binding agent is effectively reducing lung inflammation in the subject. In embodiments, the subject is a human patient, e.g., an adult or a child.

In embodiments, the efficacy value, or an indication of whether the preselected efficacy standard is met, is recorded or memorialized, e.g., in a computer readable medium. Such values or indications of meeting pre-selected efficacy standard can be listed on the product insert, a compendium (e.g., the U.S. Pharmacopeia), or any other materials, e.g., labeling that may be distributed, e.g., for commercial use, or for submission to a U.S. or foreign regulatory agency.

In another aspect, the invention features a method of evaluating or selecting an IL-13 binding agent or antagonist, e.g., an anti-IL13 antibody molecule (e.g., an IL-13 antibody as described herein or in WO 05/123126). The method includes:

providing a test value, e.g., a mean test value, for at least one pharmacokinetic/pharmacodynamic (PK/PD) parameter of the IL-13 binding agent in a subject, e.g., a human or animal subject; and

comparing the test value, e.g., mean test value, provided with at least one reference value, to thereby evaluate or select the IL-13 binding agent.

The PK/PD parameter can be estimated using non-compartmental methods, compartmental methods (e.g., two-compartmental model methods), and/or a PK-PD model. The PK/PD parameter can be chosen from one or more of: an in vivo concentration of the anti-IL13 antibody molecule (e.g., a concentration in blood, serum, plasma and/or tissue); clearance of the anti-IL-13 antibody molecule (CL); steady-volume distribution of the anti-IL-13 antibody molecule (V_{dss}); half-life of the anti-IL-13 antibody molecule (t_{1/2}); bioavailability of the anti-IL-13 antibody molecule; dose normalized maximum blood, serum or plasma concentration of the anti-IL-13 antibody molecule; dose normalized exposure of the anti-IL-13 antibody molecule; or tissue-to-serum ratio of the anti-IL-13 antibody molecule.

In a related embodiment, the PK/PD parameter can be estimated from the two-compartmental or the PK-PD model. The PK/PD parameter can be chosen from one or more of: clearance from the central compartment (CL_{Ab}); a distribution clearance between the central compartment and the peripheral compartment (CL_{d,Ab}); an association rate constant (Kₒₙ); a dissociation rate constant (K_{off}); a serum clearance of the Ab-IL-13 complex (CL_{complex}); an endogenous rate constant for IL-13 production divided by a serum clearance of IL-13 (Ksyn/CL_{IL-13}); an in vivo concentration of anti-IL-13 antibody-IL-13 complex (C_{Ab-IL-13} and C(_{Ab-IL-13)2}) in blood, serum, plasma, or tissue; or an in vivo concentration of free IL-13 (C_{IL-13}) in blood, serum, plasma, or tissue.

The comparison can include determining if the test value has a pre-selected relationship with the reference value, e.g., determining if it falls within the range of the reference value (either inclusive or exclusive of the endpoints of the range); is equal to or greater than the reference value. In embodiments, if the test value meets a preselected relationship, e.g., falls within the reference value, the IL-13 binding agent is selected.

In embodiments where the IL-13 binding agent includes a full-length antibody, the reference value, e.g., the mean reference value, includes one or more of: a clearance (CL) mean value in the range of about 0.05 to 0.9, 0.06 to 0.5, 0.065 to 0.3, or 0.067 to 0.2 mL/hr/kg after intravenous administration of the IL-13 binding agent to the subject (e.g., a mean CL value is in the range of about 0.05 to 0.5, 0.06 to 0.1, or 0.065 to 0.15 mL/hr/kg after intravenous administration to a human); a mean steady state volume of distribution (V_{dss}) value of less than about 150, 130, 120, 110, 100, 90, 80, or 70 mL/kg after intravenous administration to the subject (e.g., a control or diseased subject); a mean half-life (t_{1/2}) of about 50-800, 70-750, 100 to 600, 400-800, 500-700, 550 to 750, 552 to 696, 576 to 720, 600 to 800, 650 to 750, 670 to 725, or 670 to 710 hours after administration, e.g., intravenous, subcutaneous, intraperitoneal administration, to the subject (e.g., a mean t_{1/2} of about 400-800, 480-780, or 500-700 after intravenous or subcutaneous administration to a human); a mean bioavailability of about 50 to 100, 60 to 90, or 70 to 85% after administration, e.g., subcutaneous or intraperitoneal administration, to the subject; a dose normalized (a parameter value divided by the dosage) mean maximum serum or plasma concentration of about 2 to 40, 4 to 25, 5 to 22, to 20, 20 to 40, or 11 to 15 μg/ml after intravenous administration to the subject, or about 0.1 to 30, 0.5 to 15, 0.75 to 12, 1 to 10, or 3 to 8 μg/ml after subcutaneous administration to the subject; a mean Tₘₐₓ of about or 6-200, 6-40, 20-50, or 40-120 hours after subcutaneous administration to the subject; a mean dose normalized exposure (i.e., mean value for area under the concentration-time profile curve from time zero to infinity divided by the dosage) of about 800 to 18,000, 600 to 15,000, 500 to 12,000, 300 to 10,000, 150 to 5,000 (μghr/mL)/(mg/kg) after intravenous administration to the subject, or 400 to 18000, 500 to 15,000, 600 to 12,000, 800 to 10,000, 1,000 to 5,000 (μghr/mL)/(mg/kg) after subcutaneous administration to the subject; a mean tissue-to-serum ratio of less than about 0.8, 0.6. 0.5. 0.4; or a mean preferential exposure of antibody molecule in a tissue selected from the group consisting of lung, kidney, liver, heart and spleen (e.g., an exposure or tissue concentration at a given time-point of greater than 50%, 60%, 70% or greater than other organs).

In embodiments where the IL-13 binding agent includes an antigen-binding site of the antibody molecule (e.g., a single chan antibody, a Fab fragment, a (Fab)′2, a V_{H}, a V_{HH}), an Fv, a single chain Fv fragment, or a fusion protein containing an antigen-binding site of the antibody molecule), the reference value, e.g., the mean reference value, includes one or more of: a mean half-life (t_{1/2}) of about 0.1 to 100, 0.2 to 75, 0.3 to 50, 0.4 to 45, 0.5 to 30, 0.5 to 15, 0.5 to 10, or 0.5 to 5 hours after administration, e.g., subcutaneous, intravenous, intraperitoneal administration, to the subject.

In embodiments where the IL-13 binding agent is complexed to IL-13, the reference value, e.g., the mean reference value, includes a mean clearance of less 0.02 mL/hr/kg, 0.009 ml/hr/kg, 0.004 mL/hr/kg, 0.003 mL/hr/kg, or 0.002 mL/hr/kg after administration e.g., subcutaneous, intravenous, intraperitoneal administration, to the non-human primate or human subject. In other embodiments, the IL-13 binding agent is evaluated using a two-compartmental integrated PK-PD model (e.g., “sequential binding”) as described herein. The model includes a central compartment (C_{Ab}, V) and a peripheral compartment (C_{2,Ab}, V₂). In those embodiments, one or more of the following PK/PD parameters are evaluated: an in vivo concentration of the anti-IL13 antibody molecule (e.g., a concentration in serum, plasma, blood, and/or tissue) (C_{Ab}); a clearance from the central compartment (CL_{Ab}); a distribution clearance between the central compartment and the peripheral compartment (CL_{d,Ab}); an association rate constant (Kₒₙ); a dissociation rate constant (K_{off}); a clearance of the Ab-IL-13 complex (CL_{complex}); or an endogenous rate constant for IL-13 production divided by a clearance (e.g., serum clearance) of IL-13 (Ksyn/CL_{IL-13}).

Exemplary reference values, e.g., mean reference values, of IL-13 binding agents evaluated using a two-compartmental model where the IL-13 binding agent is a full-length antibody includes one or more of: a clearance from the central compartment (CL_{Ab}) mean value in the range of about 0.05 to 0.9, 0.06 to 0.5, 0.065 to 0.3, or 0.67 to 0.2 mL/hr/kg after intravenous administration of the IL-13 binding agent to the subject (e.g., a mean CL_{Ab} value is in the range of about 0.05 to 0.5, 0.06 to 0.1, or 0.065 to 0.15 mL/hr/kg after intravenous administration to a human); a volume of distribution in the central compartment of less than about 150, 130, 120, 110, 90, 80, or 70 mL/kg after intravenous administration to the subject (e.g., less than about 120, 90, 80, or 70 mL/kg after intravenous administration to a human); a distribution clearance between the central compartment and the peripheral compartment (CL_{d,Ab}) mean value in the range of about 0.0001-6.0, 0.0005 to 5.0, 0.00067 to 4.5, 0.001 to 4.0 mL/hr/kg after intravenous administration to the subject (e.g., 0.0002 to 5.7, or 0.0005 to 4.6 mL/hr/kg after intravenous administration to a human); a volume distribution of the peripheral compartment (V₂) mean value of less than 150, 130, 120, 110, 90, 80, or 70 mL/kg after intravenous administration to the subject (e.g., less than about 120, 90, 80, or 70 mL/kg after intravenous administration to a human); an association rate constant (Kₒₙ) mean value in the range of about 0.9 to 0.001, 0.5 to 0.01, 0.3 to 0.02, or 0.026 to 0.06 nM⁻¹ day⁻¹, a dissociation rate constant (K_{off}) mean value in the range of about 0.4 to 0.00001, 0.3 to 0.0001, 0.2 to 0.001, or 0.19 to 0.01; a serum clearance of the Ab-IL-13 complex (CL_{complex}) mean value of about 0.40 to 0.00083, 0.25 to 0.0042, 0.17 to 0.0083, 0.15 to 0.0125 mL/hr/kg, or an endogenous rate constant for IL-13 production divided by a serum clearance of IL-13 (Ksyn/CL_{IL-13}) mean value of about 0.09 to 0.0001, 0.06 to 0.001, 0.05 to 0.003, 0.045 to 0.005 nM.

In embodiments, the test value, or an indication of whether the preselected relationship is met, is recorded or memorialized, e.g., in a computer readable medium. Such test values or indications of meeting pre-selected relationship can be listed on the product insert, a compendium (e.g., the U.S. Pharmacopeia), or any other materials, e.g., labeling that may be distributed, e.g., for commercial use, or for submission to a U.S. or foreign regulatory agency.

In embodiments, the step of providing a test value includes obtaining a sample of the antibody molecule, e.g., a sample batch of an antibody culture, and testing for at least one of the pharmacokinetic parameters described herein. Methods disclosed herein can be useful from a process standpoint, e.g., to monitor or ensure batch-to-batch consistency or quality.

In embodiments, a decision or step is taken depending on whether the test value meets the pre-selected relationship (e.g., falls within the range provided for the reference value). For example, the IL-13 binding agent, e.g., the anti-IL13 antibody molecule, can be classified, selected, accepted, released (e.g., released into commerce) or withheld, processed into a drug product, shipped, moved to a new location, formulated, labeled, packaged, sold, or offered for sale.

In other embodiments, the test value provided is obtained after single or multiple administrations of the antibody molecule at a dose of about 1 to 100 mg/kg, 1 to 10 mg/kg, or 1 to 2 mg/kg.

In other embodiments, the subject is a human or non-human animal, e.g., a rodent or a primate. For example, the subject can be chosen from one or more of, e.g., rodent (e.g., a mouse, rat), a primate (e.g., a monkey or a human, e.g., a patient). The human can have a body weight of about 45-130 kg, or about 50-80 kg, typically 60 kg.

In another aspect, the invention provides a method of determining a treatment modality (e.g., a dosage, timing, or mode of administration) of an IL-13 binding agent (e.g., an anti-IL13 antibody molecule (e.g., an IL-13 antibody as described herein or in WO 05/123126) for an IL-13-mediated disorder, in a subject. The method includes:

providing a test value, e.g., a mean test value, for at least one pharmacokinetic/pharmacodynamic (PK/PD) parameter of the IL-13 binding agent in a subject, e.g., a human or animal subject;

comparing the test value, e.g., mean test value, provided with at least one reference value, e.g., mean reference value; and

selecting one or more of dosage, timing, or mode of administration based on the comparison of at least one test value to the reference value.

The PK/PD parameter can be estimated using non-compartmental methods, compartmental methods (e.g., two-compartmental model methods), and/or a PK-PD model. The PK/PD parameter can be chosen from one or more of: an in vivo concentration of the anti-IL13 antibody molecule (e.g., a concentration in blood, serum, plasma and/or tissue); clearance of the anti-IL-13 antibody molecule (CL); steady-volume distribution of the anti-IL-13 antibody molecule (V_{dss}); half-life of the anti-IL-13 antibody molecule (t_{1/2}); bioavailability of the anti-IL-13 antibody molecule; dose normalized maximum blood, serum or plasma concentration of the anti-IL-13 antibody molecule; dose normalized exposure of the anti-IL-13 antibody molecule; or tissue-to-serum ratio of the anti-IL-13 antibody molecule.

In a related embodiment, the PK/PD parameter can be estimated from the two-compartmental or the PK-PD model. The PK/PD parameter can be chosen from one or more of: clearance from the central compartment (CL_{Ab}); a distribution clearance between the central compartment and the peripheral compartment (CL_{d,Ab}); an association rate constant (Kₒₙ); a dissociation rate constant (K_{off}); a serum clearance of the Ab-IL-13 complex (CL_{complex}); an endogenous rate constant for IL-13 production divided by a serum clearance of IL-13 (Ksyn/CL_{IL-13}); an in vivo concentration of anti-IL-13 antibody-IL-13 complex (C_{Ab-IL-13} and C(_{Ab-IL-13)2}) in blood, serum, plasma, or tissue; or an in vivo concentration of free IL-13 (C_{IL-13}) in blood, serum, plasma, or tissue.

The comparison can include determining if the test value has a pre-selected relationship with the reference value, e.g., determining if it falls within the range of the reference value (either inclusive or exclusive of the endpoints of the range); is equal to or greater than the reference value. In embodiments, if the test value meets a preselected relationship, e.g., falls within the reference value, the IL-13 binding agent is selected.

In embodiments where the IL-13 binding agent includes a full-length antibody, the reference value, e.g., the mean reference value, includes one or more of: a clearance (CL) mean value in the range of about 0.05 to 0.9, 0.06 to 0.5, 0.065 to 0.3, or 0.067 to 0.2 mL/hr/kg after intravenous administration of the IL-13 binding agent to the subject (e.g., a mean CL value is in the range of about 0.05 to 0.5, 0.06 to 0.1, or 0.065 to 0.15 mL/hr/kg after intravenous administration to a human); a mean steady state volume of distribution (V_{dss}) value of less than about 150, 130, 120, 110, 100, 90, 80, or 70 mL/kg after intravenous administration to the subject (e.g., a control or diseased subject); a mean half-life (t_{1/2}) of about 50-800, 70-750, 100 to 600, 400-800, 500-700, 550 to 750, 552 to 696, 576 to 720, 600 to 800, 650 to 750, 670 to 725, or 670 to 710 hours after administration, e.g., intravenous, subcutaneous, intraperitoneal administration, to the subject (e.g., a mean t_{1/2} of about 400-800, 480-780, or 500-700 after intravenous or subcutaneous administration to a human); a mean bioavailability of about 50 to 100, 60 to 90, or 70 to 85% after administration, e.g., subcutaneous or intraperitoneal administration, to the subject; a dose normalized (a parameter value divided by the dosage) mean maximum serum or plasma concentration of about 2 to 40, 4 to 25, 5 to 22, to 20, 20 to 40, or 11 to 15 μg/ml after intravenous administration to the subject, or about 0.1 to 30, 0.5 to 15, 0.75 to 12, 1 to 10, or 3 to 8 μg/ml after subcutaneous administration to the subject; a mean Tₘₐₓ of about or 6-200, 6-40, 20-50, or 40-120 hours after subcutaneous administration to the subject; a mean dose normalized exposure (i.e., mean value for area under the concentration-time profile curve from time zero to infinity divided by the dosage) of about 800 to 18,000, 600 to 15,000, 500 to 12,000, 300 to 10,000, 150 to 5,000 (μghr/mL)/(mg/kg) after intravenous administration to the subject, or 400 to 18000, 500 to 15,000, 600 to 12,000, 800 to 10,000, 1,000 to 5,000 (μghr/mL)/(mg/kg) after subcutaneous administration to the subject; a mean tissue-to-serum ratio of less than about 0.8, 0.6. 0.5. 0.4; or a mean preferential exposure of antibody molecule in a tissue selected from the group consisting of lung, kidney, liver, heart and spleen (e.g., an exposure or tissue concentration at a given time-point of greater than 50%, 60%, 70% or greater than other organs).

In embodiments where the IL-13 binding agent includes an antigen-binding site of the antibody molecule (e.g., a single chan antibody, a Fab fragment, a (Fab)′2, a V_{H}, a V_{HH}), an Fv, a single chain Fv fragment, or a fusion protein containing an antigen-binding site of the antibody molecule), the reference value, e.g., the mean reference value, includes one or more of: a mean half-life (t_{1/2}) of about 0.1 to 100, 0.2 to 75, 0.3 to 50, 0.4 to 45, 0.5 to 30, 0.5 to 15, 0.5 to 10, or 0.5 to 5 hours after administration, e.g., subcutaneous, intravenous, intraperitoneal administration, to the subject.

In embodiments where the IL-13 binding agent is complexed to IL-13, the reference value, e.g., the mean reference value, includes a mean clearance of less 0.02 mL/hr/kg, 0.009 ml/hr/kg, 0.004 mL/hr/kg, 0.003 mL/hr/kg, or 0.002 mL/hr/kg after administration e.g., subcutaneous, intravenous, intraperitoneal administration, to the non-human primate or human subject. In other embodiments, the IL-13 binding agent is evaluated using a two-compartmental integrated PK-PD model (e.g., “sequential binding”) as described herein. The model includes a central compartment (C_{Ab}, V) and a peripheral compartment (C_{2,Ab}, V₂). In those embodiments, one or more of the following PK/PD parameters are evaluated: an in vivo concentration of the anti-IL13 antibody molecule (e.g., a concentration in serum, plasma, blood, and/or tissue) (C_{Ab}); a clearance from the central compartment (CL_{Ab}); a distribution clearance between the central compartment and the peripheral compartment (CL_{d,Ab}); an association rate constant (Kₒₙ); a dissociation rate constant (K_{off}); a clearance of the Ab-IL-13 complex (CL_{complex}); or an endogenous rate constant for IL-13 production divided by a clearance (e.g., serum clearance) of IL-13 (Ksyn/CL_{IL-13}).

Exemplary reference values, e.g., mean reference values, of IL-13 binding agents evaluated using a two-compartmental model where the IL-13 binding agent is a full-length antibody includes one or more of: a clearance from the central compartment (CL_{Ab}) mean value in the range of about 0.05 to 0.9, 0.06 to 0.5, 0.065 to 0.3, or 0.67 to 0.2 mL/hr/kg after intravenous administration of the IL-13 binding agent to the subject (e.g., a mean CL_{Ab} value is in the range of about 0.05 to 0.5, 0.06 to 0.1, or 0.065 to 0.15 mL/hr/kg after intravenous administration to a human); a volume of distribution in the central compartment of less than about 150, 130, 120, 110, 90, 80, or 70 mL/kg after intravenous administration to the subject (e.g., less than about 120, 90, 80, or 70 mL/kg after intravenous administration to a human); a distribution clearance between the central compartment and the peripheral compartment (CL_{d,Ab}) mean value in the range of about 0.0001-6.0, 0.0005 to 5.0, 0.00067 to 4.5, 0.001 to 4.0 mL/hr/kg after intravenous administration to the subject (e.g., 0.0002 to 5.7, or 0.0005 to 4.6 mL/hr/kg after intravenous administration to a human); a volume distribution of the peripheral compartment (V₂) mean value of less than 150, 130, 120, 110, 90, 80, or 70 mL/kg after intravenous administration to the subject (e.g., less than about 120, 90, 80, or 70 mL/kg after intravenous administration to a human); an association rate constant (Kₒₙ) mean value in the range of about 0.9 to 0.001, 0.5 to 0.01, 0.3 to 0.02, or 0.026 to 0.06 nM⁻¹ day⁻¹, a dissociation rate constant (K_{off}) mean value in the range of about 0.4 to 0.00001, 0.3 to 0.0001, 0.2 to 0.001, or 0.19 to 0.01; a serum clearance of the Ab-IL-13 complex (CL_{complex}) mean value of about 0.40 to 0.00083, 0.25 to 0.0042, 0.17 to 0.0083, 0.15 to 0.0125 mL/hr/kg, or an endogenous rate constant for IL-13 production divided by a serum clearance of IL-13 (Ksyn/CL_{IL-13}) mean value of about 0.09 to 0.0001, 0.06 to 0.001, 0.05 to 0.003, 0.045 to 0.005 nM.

The selection of treatment modality (e.g., a dosage, timing, or mode of administration) can be based, in part, on the comparison of the test value and the reference value. The comparison can include determining if the test value has a pre-selected relationship with the reference value, e.g., determining if it falls within the range of the reference value (either inclusive or exclusive of the endpoints of the range); is equal to or greater than the reference value. For example, if the half-life of the binding agent falls within the range specified in the reference value, a practitioner may determine that the frequency of administration can be reduced to, e.g., once or twice per month. In combination or independently, a low dose of the binding agent can be administered, e.g., less than one of 5, 4, 3, 2, 1 mg/kg. Treatment modalities chosen based on the comparison can vary depending on the IL-13-associated disorder at issue. For respiratory disorders, e.g., asthma, the IL-13 binding agent can be delivered by inhalation, subcutaneously or intravenously.

In embodiments, the subject is a human or non-human animal, e.g., a rodent or a primate. For example, the subject can be chosen from one or more of, e.g., rodent (e.g., a mouse, rat), a primate (e.g., a monkey or a human, e.g., a patient). The human can have a body weight of about 45-130 kg, or about 50-80 kg, typically 60 kg. The human may be a control or diseased subject.

In another aspect, the invention features a method of treating an IL-13-associated disorder (e.g., an IL-13 disorder as described herein) in a subject, e.g., a subject as described herein, that includes administering, to a subject having, or being at risk of having, the IL-13-associated disorder, an effective amount of the IL-13 binding agent, e.g., the anti-IL-13 antibody molecule evaluated or selected using one or more of the PK/PD parameters described herein.

In another aspect, the invention features a method of instructing, or transferring information to, a recipient (e.g., a patient, a pharmacist, a caregiver, a clinician, a member of a medical staff, a manufacturer, or a distributor) on the use of an IL-13 binding agent, e.g., an anti-IL13 antibody molecule, to treat an IL-13-associated disorder. The method includes instructing, or sending information to, the recipient that said IL-13 binding agent has at least one test value, e.g., mean test value, for a PK/PD parameter selected from the group consisting of:

a clearance (CL) mean value in the range of about 0.05 to 0.9, 0.06 to 0.5, 0.065 to 0.3, or 0.067 to 0.2 mL/hr/kg after intravenous administration of the IL-13 binding agent to a subject (e.g., a mean CL value is in the range of about 0.05 to 0.5, 0.06 to 0.1, or 0.065 to 0.15 mL/hr/kg after intravenous administration to a human), wherein the IL-13 binding agent includes a full-length antibody; a mean steady state volume of distribution (V_{dss}) value of less than about 150, 130, 120, 110, 100, 90, 80, or 70 mL/kg after intravenous administration to the subject (e.g., a control or diseased subject), wherein the IL-13 binding agent includes a full-length antibody; a mean half-life (t_{1/2}) of about 50-800, 70-750, 100 to 600, 400-800, 500-700, 550 to 750, 552 to 696, 576 to 720, 600 to 800, 650 to 750, 670 to 725, or 670 to 710 hours after administration, e.g., intravenous, subcutaneous, intraperitoneal administration, to the subject (e.g., a mean t_{1/2} of about 400-800, 480-780, or 500-700 after intravenous or subcutaneous administration to a human); a mean bioavailability of about 50 to 100, 60 to 90, or 70 to 85% after administration, e.g., subcutaneous or intraperitoneal administration, to the subject; a dose normalized (a parameter value divided by the dosage) mean maximum serum or plasma concentration of about 2 to 40, 4 to 25, 5 to 22, 10 to 20, 20 to 40, or 11 to 15 μg/ml after intravenous administration to the subject, or about 0.1 to 30, 0.5 to 15, 0.75 to 12, 1 to 10, or 3 to 8 μg/ml after subcutaneous administration to the subject; a mean Tₘₐₓ of about or 6-200, 6-40, 20-50, or 40-120 hours after subcutaneous administration to the subject; a mean dose normalized exposure (i.e., mean value for area under the concentration-time profile curve from time zero to infinity divided by the dosage) of about 800 to 18,000, 600 to 15,000, 500 to 12,000, 300 to 10,000, 150 to 5,000 (μghr/mL)/(mg/kg) after intravenous administration to the subject, or 400 to 18000, 500 to 15,000, 600 to 12,000, 800 to 10,000, 1,000 to 5,000 (μghr/mL)/(mg/kg) after subcutaneous administration to the subject; a mean tissue-to-serum ratio of less than about 0.8, 0.6. 0.5. 0.4; or a mean preferential exposure of antibody molecule in a tissue selected from the group consisting of lung, kidney, liver, heart and spleen (e.g., an exposure or tissue concentration at a given time-point of greater than 50%, 60%, 70% or greater than other organs), wherein the IL-13 binding agent includes a full-length antibody; a mean half-life (t_{1/2}) of about 0.1 to 100, 0.2 to 75, 0.3 to 50, 0.4 to 45, 0.5 to 30, 0.5 to 15, 0.5 to 10, 0.5 to 5 hours after administration, e.g., subcutaneous, intravenous, intraperitoneal administration, to the subject, wherein the IL-13 binding agent includes an antigen-binding site of the antibody molecule (e.g., a single chan antibody, a Fab fragment, a (Fab)′2, a V_{H}, a V_{HH}), an Fv, a single chain Fv fragment, or a fusion protein containing an antigen-binding site of the antibody molecule); and a mean clearance rate of less than 0.004 mL/hr/kg, 0.003 mL/hr/kg, or 0.002 mL/hr/kg after administration to the subject, wherein the IL-13 binding agent is complexed to IL-13.

In other embodiments, the PK/PD parameter of the IL-13 binding agent is evaluated using a two-compartmental (e.g., “sequential binding”) model as described herein. The two-compartmental model includes a central compartment (C_{Ab}, V) and a peripheral compartment (C_{2,Ab}, V₂). In those embodiments, one or more of the following PK/PD parameters are evaluated: an in vivo concentration of the anti-IL13 antibody molecule (e.g., a concentration in serum, plasma, and/or tissue) (CL_{Ab}), a distribution clearance between the central compartment and the peripheral compartment (CL_{d,Ab}), an association rate constant (Kₒₙ), a dissociation rate constant (K_{off}), a serum clearance of the Ab-IL-13 complex (CL_{complex}), or an endogenous rate constant for IL-13 production divided by a serum clearance of IL-13 (Ksyn/CL_{IL-13}).

Exemplary reference values, e.g., mean reference values, of IL-13 binding agents evaluated using a two-compartmental model where the IL-13 binding agent is a full-length antibody includes one or more of: a clearance from the central compartment (CL_{Ab}) mean value in the range of about 0.05 to 0.9, 0.06 to 0.5, 0.065 to 0.3, or 0.67 to 0.2 mL/hr/kg after intravenous administration of the IL-13 binding agent to the subject (e.g., a mean CL_{Ab} value is in the range of about 0.05 to 0.5, 0.06 to 0.1, or 0.065 to 0.15 mL/hr/kg after intravenous administration to a human); a volume of distribution in the central compartment of less than about 150, 130, 120, 110, 90, 80, or 70 mL/kg after intravenous administration to the subject (e.g., less than about 120, 90, 80, or 70 mL/kg after intravenous administration to a human); a distribution clearance between the central compartment and the peripheral compartment (CL_{d,Ab}) mean value in the range of about 0.0001-6.0, 0.0005 to 5.0, 0.00067 to 4.5, 0.001 to 4.0 mL/hr/kg after intravenous administration to the subject (e.g., 0.0002 to 5.7, or 0.0005 to 4.6 mL/hr/kg after intravenous administration to a human); a volume distribution of the peripheral compartment (V₂) mean value of less than 150, 130, 120, 110, 90, 80, or 70 mL/kg after intravenous administration to the subject (e.g., less than about 120, 90, 80, or 70 mL/kg after intravenous administration to a human); an association rate constant (Kₒₙ) mean value in the range of about 0.9 to 0.001, 0.5 to 0.01, 0.3 to 0.02, or 0.026 to 0.06 nM⁻¹ day⁻¹, a dissociation rate constant (K_{off}) mean value in the range of about 0.4 to 0.00001, 0.3 to 0.0001, 0.2 to 0.001, or 0.19 to 0.01; a serum clearance of the Ab-IL-13 complex (CL_{complex}) mean value of about 0.40 to 0.00083, 0.25 to 0.0042, 0.17 to 0.0083, 0.15 to 0.0125 mL/hr/kg, or an endogenous rate constant for IL-13 production divided by a serum clearance of IL-13 (Ksyn/CL_{IL-13}) mean value of about 0.09 to 0.0001, 0.06 to 0.001, 0.05 to 0.003, 0.045 to 0.005 nM.

In embodiments, the method includes recording or memorializing, e.g., in a computer readable medium, one of more of the test values. Such test values can be listed on the product insert, a compendium (e.g., the U.S. Pharmacopeia), or any other materials, e.g., labeling that may be distributed, e.g., for commercial use, or for submission to a U.S. or foreign regulatory agency.

In embodiments, the method further includes administering the IL-13 binding agent to the patient. In embodiments, one or more of dosage, timing, or mode of administration of the binding agent, e.g., antibody molecule, is based, at least in part, on the comparison of the test value at least one PK/PD parameter of the antibody molecule with a reference value, e.g., a reference value as described herein.

In another aspect, the invention features method of treating an IL-13-associated disorder in a subject having, or being at risk of having, the IL-13-associated disorder. The method includes:

instructing a caregiver or a patient that an IL-13 binding agent, e.g., an anti-IL13 antibody has at least one test value, e.g., mean test value, for a PK/PD parameter selected from the group consisting of:

a clearance (CL) mean value in the range of about 0.05 to 0.9, 0.06 to 0.5, 0.065 to 0.3, or 0.067 to 0.2 mL/hr/kg after intravenous administration of the IL-13 binding agent to a subject (e.g., a mean CL value is in the range of about 0.05 to 0.5, 0.06 to 0.1, or 0.065 to 0.15 mL/hr/kg after intravenous administration to a human), wherein the IL-13 binding agent includes a full-length antibody; a mean steady state volume of distribution (V_{dss}) value of less than about 150, 130, 120, 110, 100, 90, 80, or 70 mL/kg after intravenous administration to the subject (e.g., a control or diseased subject), wherein the IL-13 binding agent includes a full-length antibody; a mean half-life (t_{1/2}) of about 50-800, 70-750, 100 to 600, 400-800, 500-700, 550 to 750, 552 to 696, 576 to 720, 600 to 800, 650 to 750, 670 to 725, or 670 to 710 hours after administration, e.g., intravenous, subcutaneous, intraperitoneal administration, to the subject (e.g., a mean t_{1/2} of about 400-800, 480-780, or 500-700 after intravenous or subcutaneous administration to a human); a mean bioavailability of about 50 to 100, 60 to 90, or 70 to 85% after administration, e.g., subcutaneous or intraperitoneal administration, to the subject; a dose normalized (a parameter value divided by the dosage) mean maximum serum or plasma concentration of about 2 to 40, 4 to 25, 5 to 22, 10 to 20, 20 to 40, or 11 to 15 μg/ml after intravenous administration to the subject, or about 0.1 to 30, 0.5 to 15, 0.75 to 12, 1 to 10, or 3 to 8 μg/ml after subcutaneous administration to the subject; a mean Tₘₐₓ of about or 6-200, 6-40, 20-50, or 40-120 hours after subcutaneous administration to the subject; a mean dose normalized exposure (i.e., mean value for area under the concentration-time profile curve from time zero to infinity divided by the dosage) of about 800 to 18,000, 600 to 15,000, 500 to 12,000, 300 to 10,000, 150 to 5,000 (μghr/mL)/(mg/kg) after intravenous administration to the subject, or 400 to 18000, 500 to 15,000, 600 to 12,000, 800 to 10,000, 1,000 to 5,000 (μghr/mL)/(mg/kg) after subcutaneous administration to the subject; a mean tissue-to-serum ratio of less than about 0.8, 0.6. 0.5. 0.4; or a mean preferential exposure of antibody molecule in a tissue selected from the group consisting of lung, kidney, liver, heart and spleen (e.g., an exposure or tissue concentration at a given time-point of greater than 50%, 60%, 70% or greater than other organs), wherein the IL-13 binding agent includes a full-length antibody; a mean half-life (t_{1/2}) of about 0.1 to 100, 0.2 to 75, 0.3 to 50, 0.4 to 45, 0.5 to 30, 0.5 to 15, 0.5 to 10, 0.5 to 5 hours after administration, e.g., subcutaneous, intravenous, intraperitoneal administration, to the subject, wherein the IL-13 binding agent includes an antigen-binding site of the antibody molecule (e.g., a single chan antibody, a Fab fragment, a (Fab)′2, a V_{H}, a V_{HH}), an Fv, a single chain Fv fragment, or a fusion protein containing an antigen-binding site of the antibody molecule); and a mean clearance rate of less than 0.004 mL/hr/kg, 0.003 mL/hr/kg, or 0.002 mL/hr/kg after administration to the subject, wherein the IL-13 binding agent is complexed to IL-13; and

administering the IL-13 binding agent, e.g., the anti-IL13 antibody molecule, to the patient. The administration step can be performed by the patient directly, e.g., self-administration, or by another party, e.g., a caregiver.

In other embodiments, the PK/PD parameter of the IL-13 binding agent is evaluated using a two-compartmental model as described herein. The two-compartmental model includes a central compartment (C_{Ab}, V) and a peripheral compartment (C_{2,Ab}, V₂). In those embodiments, one or more of the following PK/PD parameters are evaluated: an in vivo concentration of the anti-IL13 antibody molecule (e.g., a concentration in serum, plasma, and/or tissue) (CL_{Ab}), a distribution clearance between the central compartment and the peripheral compartment (CL_{d,Ab}), an association rate constant (Kₒₙ), a dissociation rate constant (K_{off}), a serum clearance of the Ab-IL-13 complex (CL_{complex}), or an endogenous rate constant for IL-13 production divided by a serum clearance of IL-13 (Ksyn/CL_{IL-13}).

Exemplary reference values, e.g., mean reference values, of IL-13 binding agents evaluated using a two-compartmental model where the IL-13 binding agent is a full-length antibody include one or more of: a clearance from the central compartment (CL_{Ab}) mean value in the range of about 0.05 to 0.9, 0.06 to 0.5, 0.065 to 0.3, or 0.67 to 0.2 mL/hr/kg after intravenous administration of the IL-13 binding agent to the subject (e.g., a mean CL_{Ab} value is in the range of about 0.05 to 0.5, 0.06 to 0.1, or 0.065 to 0.15 mL/hr/kg after intravenous administration to a human); a volume of distribution in the central compartment of less than about 150, 130, 120, 110, 90, 80, or 70 mL/kg after intravenous administration to the subject (e.g., less than about 120, 90, 80, or 70 mL/kg after intravenous administration to a human); a distribution clearance between the central compartment and the peripheral compartment (CL_{d,Ab}) mean value in the range of about 0.0001-6.0, 0.0005 to 5.0, 0.00067 to 4.5, 0.001 to 4.0 mL/hr/kg after intravenous administration to the subject (e.g., 0.0002 to 5.7, or 0.0005 to 4.6 mL/hr/kg after intravenous administration to a human); a volume distribution of the peripheral compartment (V₂) mean value of less than 150, 130, 120, 110, 90, 80, or 70 mL/kg after intravenous administration to the subject (e.g., less than about 120, 90, 80, or 70 mL/kg after intravenous administration to a human); an association rate constant (Kₒₙ) mean value in the range of about 0.9 to 0.001, 0.5 to 0.01, 0.3 to 0.02, or 0.026 to 0.06 nM⁻¹ day⁻¹, a dissociation rate constant (K_{off}) mean value in the range of about 0.4 to 0.00001, 0.3 to 0.0001, 0.2 to 0.001, or 0.19 to 0.01; a serum clearance of the Ab-IL-13 complex (CL_{complex}) mean value of about 0.40 to 0.00083, 0.25 to 0.0042, 0.17 to 0.0083, 0.15 to 0.0125 mL/hr/kg, or an endogenous rate constant for IL-13 production divided by a serum clearance of IL-13 (Ksyn/CL_{IL-13}) mean value of about 0.09 to 0.0001, 0.06 to 0.001, 0.05 to 0.003, 0.045 to 0.005 nM.

In embodiments, one or more of dosage, timing, or mode of administration of the binding agent, e.g., antibody molecule, is based, at least in part, on a comparison of the test value at least one PK/PD parameter of the antibody molecule with a reference value, e.g., a reference value as described herein.

In another aspect, the invention features a kit or package that includes an IL-13 binding agent, e.g., an anti-IL13 antibody molecule as described herein or in WO 05/123126), or a pharmaceutical composition thereof, and instructions for use. In embodiments, the IL-13 binding agent included in the kit is or has been evaluated or selected based, at least in part, on a comparison of a test value with a reference value, as described herein. In other embodiments, the IL-13 binding agent has at least one test value for a PK/PD parameter as described herein. In embodiments, the kit includes an IL-13 antibody molecule packaged to be administered as a flat dose, e.g., a flat dose as described herein, and instruction for administration as a flat dose.

In yet another aspect, the invention features an IL-13 binding agent, e.g., an anti-IL13 molecule, selected or evaluated by comparing a test value for a pharmacokinetic parameter with a reference value, as described herein. In embodiments, the binding agent is other than 13.2, MJ2-7 and C65 (or humanized versions thereof).

In another aspect, the invention features a method of evaluating the amount of a drug-ligand complex in a subject using a two-compartmental PK-PD model that includes a central compartment (C_{Ab}, V) and a peripheral compartment (C_{2,Ab}, V₂). The method includes:

providing at least one pharmacokinetic or pharmacodynamic parameter value of the drug-ligand concentration in the subject at a predetermined time interval, said value chosen from one or more of the following PK/PD parameters: an in vivo concentration of the drug, e.g., anti-IL13 antibody molecule (e.g., a concentration in blood, serum, plasma, and/or tissue) (CL_{Ab}); an in vivo concentration of unbound Il-13, anti-IL-13-bound IL-13 or total IL-13 ((e.g., a concentration in blood, serum, plasma, and/or tissue)) a distribution clearance between the central compartment and the peripheral compartment (CL_{d,Ab}); an association rate constant (Kₒₙ); a dissociation rate constant (K_{off}); a serum clearance of the drug-ligand (e.g., Ab-IL-13) complex (CL_{complex}); or an endogenous rate constant for ligand, e.g., IL-13, production divided by a serum clearance of the ligand, e.g., IL-13, (Ksyn/CL_{IL-13});

evaluating the at least one pharmacokinetic parameter in the subject using the two-comparmental PK-PD model as represented in FIG. 33.

In embodiments, the two-compartmental model is represented as follows:

dC_{Ab}/dt=[In(t)+CL_{d,Ab}·C_{2,Ab}−(CL_{d,Ab}+CL_{Ab})·C_{Ab}]/V−Kₒₙ·C_{Ab}*(C_{IL-13}−C_{Ab-(IL-13)}−C_{Ab-(IL-13)}_{₂})+K_{off}·C_{Ab-(IL-13)} when t=0,C_{Ab}⁰=In(0)/V (1)

dC_{2,Ab}/dt=(CL_{d,Ab}·C_{Ab}−CL_{d,Ab}·C_{2,Ab})/V₂ when t=0,C_{2,Ab}⁰=0 (2)

dC_{Ab-(IL-13)}/dt=Kₒₙ·C_{Ab}·(C_{IL-13}−C_{Ab-(IL-13)}−C_{Ab-(IL-13)}_{₂})−CL_{complex}·C_{Ab-(IL-13)}−K_{off}·C_{Ab-(IL-13)}+K_{off}·C_{Ab-(IL-13)}_{₂}−Kₒₙ·C_{Ab-(IL-13)}·(C_{IL-13}−C_{Ab-(IL-13)}−C_{Ab-(IL-13)}_{₂}) when t=0,C_{Ab-(IL-13)}⁰=0 (3)

dC_{Ab-(IL-13)}_{₂}/dt=Kₒₙ·C_{Ab-(IL-13)}·(C_{IL-13}−C_{Ab-(IL-13)}−C_{Ab-(IL-13)}_{₂})−CL_{complex}·C_{Ab-(IL-13})_{₂}−K_{off}·C_{Ab-(IL-3)}_{₂} when t=0,C_{Ab-(IL-13)}_{₂}⁰=0 (4)

dC_{IL-13}/dt=[K_{syn}−CL_{IL-13}·(C_{IL-13}−C_{Ab-(IL-13)}−C_{Ab-(IL-13)}_{₂})]/V−Kₒₙ·C_{Ab}·(C_{IL-13}−C_{Ab-(IL-13)}−C_{Ab-(IL-13)}_{₂})−Kₒₙ·C_{Ab-(IL-13)}·(C_{IL-13}−C_{Ab-(IL-13)}−C_{Ab-(IL-13)}_{₂})+k_{off}·C_{A-(IL-13)}+K_{off}·C_{Ab-(IL-13)}_{₂} when t=0,C_{Il-13}⁰=K_{syn}/CL_{IL-13} (5)

For iv bolus dose:

In(t)=Dose (6)

For sc dose:

In(t)=Kₐ·F·Dose (7)

wherein,

C_{Ab} is a concentration of antibody (binding agent);
In(t) is a dose administered (for a bolus dose), and In(t) is Kₐ*F*Dose for a subcutaneous does, wherein Kₐ is a first order rate constant and F is an estimate of bioavailability;
CL_{d,Ab} is a distribution clearance between the central compartment and the peripheral compartment;
C_{2,Ab} is a concentration of the ligand binding agent in the peripheral compartment;
V is a volume distribution in a central component;
Kₒₙ is a second order rate constant;
C_{ligand} (or C_{IL-13}) is a concentration of ligand;
C_{Ab-(ligand)} (or C_{Ab-(IL-13)}) is a concentration of ligand binding agent/ligand complex;
K_{off} is a first order disassociation rate constant, V₂ is a volume of distribution in a peripheral compartment;
CL_{complex} is the serum clearance of the ligand binding agent/ligand complex; and
K_{syn} is a zero order rate constant for endogenous ligand.

In certain embodiments, the method evaluates the amount of a drug-ligand complex selected from the group consisting of a ligand-antibody complex and a ligand-soluble receptor complex. For example, the ligand can be a cytokine, e.g., IL-5, IL-6, IL-12, IL-13, IL-21, IL-22; or a growth factor, e.g., VEGF, TNFα; and the drug can be an antibody against the ligand, or a soluble receptor.

In certain embodiments, the method evaluates the amount of IL-13-antibody complex in the subject. For example, the two compartmental model used in the methods includes pharmacokinetic/pharmacodynamic values for one the following:

the Ligand is IL-13 and the ligand binding agent (Ab) is a drug (e.g., is an antibody molecule (e.g., hMJ2-7v.2-11 HMJ2-7v.2-11));

Complex is a drug-ligand complex (e.g., hMJ2-7v.2-11 HMJ2-7v.2-11/IL-13 complex);

CL_{d,Ab} and CL_{Ab} are distribution clearance and serum clearance of the drug (e.g., an antibody molecule (e.g., hMJ2-7v.2-1 HMJ2-7v.2-11)), respectively;

CL_{complex} and CL_{Ligand} (or CL_{IL-13}) are serum clearance of the complex and the ligand, e.g., IL-13, respectively;

K_{syn} is a zero-order ligand, e.g., IL-13, synthesis rate constant;

Kₒₙ is a second-order association rate constant;

K_{off} is a first-order dissociation rate constant; and V and V₂ are volumes of distribution of the drug (e.g., hMJ2-7v.2-11 HMJ2-7v.2-11) in the serum (central) and the second compartment, respectively.

In some aspects, the invention features a method of treating an IL-13-associated disorder in a subject, e.g., using a flat dose of anti-IL-13 antibody. The method includes administering, to a subject having, or being at risk of having, the IL-13-associated disorder, a flat dose of an anti-IL-13 antibody molecule (e.g., hMJ2-7v.2-11 HMJ2-7v.2-11 or 13.2v2).

In some embodiments, the flat dose is a dose of between about 50 mg and 500 mg, about 60 mg and 490 mg, about 70 mg to 480 mg, about 75 mg to 460 mg, about 80 mg to 450, about 100 mg and about 450 mg, about 150 mg to about 400 mg, about 200 mg to about 300 mg, about 200 mg to about 250 mg; or about 60 mg, 65 mg, 70 mg, 75 mg, 80 mg, 90 mg, 100 mg, 125 mg, 150 mg, 175 mg, 200 mg, 225 mg, or 250 mg of an anti-IL-13 antibody molecule (e.g., hMJ2-7v.2-11 HMJ2-7v.2-11 or 13.2v2).

In some embodiments, the flat dose is about 75, 200 or 225 mg of an anti-IL-13 antibody molecule (e.g., hMJ2-7v.2-11 HMJ2-7v.2-11 or 13.2v2).

In some embodiments, the flat dose is administered to the subject approximately every week, approximately every 2 weeks, approximately every 3 weeks, or approximately every 4 weeks.

For purposes of clarity, the term “IL-13 antagonist” as used herein collectively refers to a compound such as a protein (e.g., a multi-chain polypeptide, a polypeptide), a peptide, small molecule, or inhibitory nucleic acid that reduces, inhibits or otherwise blocks one or more biological activities of IL-13 and an IL-13R. In one embodiment, the IL-13 antagonist interacts with, e.g., binds to, an IL-13 or IL-13R polypeptide (also referred to herein as an “antagonistic IL-13 binding agent.” For example, the IL-13 antagonist can interact with, e.g., can bind to, IL-13 or IL-13 receptor, preferably, mammalian, e.g., human IL-13 or IL-13R (also individually referred to herein as an “IL-13 antagonist” and “IL-13R antagonist,” respectively), and reduce or inhibit one or more IL-13- and/or IL-13R-associated biological activities. Antagonists bind to IL-13 or IL-13R with high affinity, e.g., with an affinity constant of at least about 10⁷ M⁻¹, preferably about 10⁸ M⁻¹, and more preferably, about 10⁹ M⁻¹ to 10¹⁰ M⁻¹ or stronger. It is noted that the term “IL-13 antagonist” includes agents that inhibit or reduce one or more of the biological activities disclosed herein, but may not bind to IL-13 directly.

The terms “anti-IL13 binding agent” and “IL-13 binding agent” are used interchangeably herein. These terms as used herein refers to any compound, such as a protein (e.g., a multi-chain polypeptide, a polypeptide) or a peptide, that includes an interface that binds to an IL-13 protein, e.g., a mammalian IL-13, particularly, a human IL-13. The binding agent generally binds with a Kd of less than 5×10⁻⁷ M. An exemplary IL-13 binding agent is a protein that includes an antigen binding site, e.g., an antibody molecule. The anti-IL13 binding agent or IL-13 binding agent can be an IL-13 antagonist that binds to IL13, or can also include IL-13 binding agents that simply bind to IL-13, but do not elicit an activity, or may in fact agonize an IL-13 activity. For example, certain IL-13 binding agents, e.g., anti-IL-13 antibody molecules, that bind to and inhibit one or more IL-13 biological activities, e.g., antibodies 13.2, MJ2-7 and C65, are also referred to herein as antagonistic IL-13 binding agents. Examples of IL-13 antagonists that are not IL-13 binding agents as defined herein include, e.g., inhibitors of upstream or downstream IL-13 signalling (e.g., STAT6 inhibitors).

Additional embodiments of the methods disclosed herein may include one or more of the following features:

In some embodiments, the IL-13 antagonist can be an antibody molecule that binds to IL-13 or an IL-13R. The IL-13 can also be a soluble form of the IL-13R (e.g., soluble IL-13Rα2 or IL-13Rα1), alone or fused to another moiety (e.g., an immunoglobulin Fc region) or as a heterodimer of subunits (e.g., a soluble IL-13R-IL-4R). In other embodiments, the antagonist is a cytokine mutein (e.g., an IL-13 mutein that binds to the corresponding receptor, but does not substantially activate the receptor).

In one embodiment, the IL-13 antagonist or binding agent (e.g., the antibody molecule, soluble receptor, cytokine mutein, or peptide inhibitor) binds to IL-13 or an IL13R and inhibits or reduces an interaction (e.g., binding) between IL-13 and an IL-13 receptor, e.g., IL-13Rα1, IL-13Rα2, and/or IL-4RI, thereby reducing or inhibiting signal transduction. For example, the IL-13 antagonist can bind to one or more components of a complex chosen from, e.g., IL-13 and IL-13Rα1 (“IL-13/IL-13αR1”); IL-13 and IL-4Rα (“IL-13/IL-4Rα”); IL-13, IL-13Rα1, and IL-4Rα (“IL-13/IL-13Rα1/IL-4Rα”); and IL-13 and IL-13Rα2 (“IL-13/IL13Rα2”). In embodiments, the IL-13 antagonist binds to IL-13 or an IL-13R and interferes with (e.g., inhibits, blocks or otherwise reduces) an interaction, e.g., binding, between IL-13 and an IL-13 receptor complex, e.g., a complex comprising IL-13Rα1 and IL-4R α. In other embodiments, the IL-13 antagonist binds to IL-13 and interferes with (e.g., inhibits, blocks or otherwise reduces) an interaction, e.g., binding, between IL-13 and a subunit of the IL-13 receptor complex, e.g., IL-13Rα1 or IL-4Rα, individually. In yet another embodiment, the IL-13 antagonist, e.g., the anti-IL-13 antibody or fragment thereof, binds to IL-13, and interferes with (e.g., inhibits, blocks or otherwise reduces) an interaction, e.g., binding, between IL-13/IL-13Rα1 and IL-4Rα. In another embodiment, the IL-13 antagonist, binds to IL-13 and interferes with (e.g., inhibits, blocks or otherwise reduces) an interaction, e.g., binding, between IL-13/IL-4Rα and IL-13Rα1. Typically, the IL-13 antagonist interferes with (e.g., inhibits, blocks or otherwise reduces) an interaction, e.g., binding, of IL-13/IL-13Rα1 with IL-4Rα. Exemplary antibodies inhibit or prevent formation of the ternary complex, IL-13/IL-13Rα1/IL-4Rα.

In one embodiment, the IL-13/IL-13R antagonist or binding agent is an antibody molecule (e.g., an antibody, or an antigen-binding fragment thereof) that binds to IL-13/IL-13R. For example, the antibody molecule can be a full length monoclonal or single specificity antibody that binds to IL-13 or an IL-13 receptor (e.g., an antibody molecule that includes at least one, and typically two, complete heavy chains, and at least one, and typically two, complete light chains); or an antigen-binding fragment thereof (e.g., a heavy or light chain variable domain monomer or dimer (e.g., V_{H}, V_{HH}), an Fab, F(ab′)₂, Fv, or a single chain Fv fragment). Typically, the antibody molecule is a human, camelid, shark, humanized, chimeric, or in vitro-generated antibody to human IL-13 or a human IL-13 receptor. In certain embodiments, the antibody molecule includes a heavy chain constant region chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant regions IgG1 (e.g., human IgG1 or a modified form thereof). In another embodiment, the antibody molecule has a light chain constant region chosen from, e.g., the light chain constant regions of kappa or lambda, preferably kappa (e.g., human kappa). In one embodiment, the constant region is altered, e.g., mutated, to modify the properties of the antibody molecule (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237, as described in Example 5, to decrease one or more of: Fc receptor binding, antibody glycosylation, the number of cysteine residues, effector cell function, or complement function. In embodiments, the antibody molecule includes a human IgG1 constant region mutated at one or more residues of SEQ ID NO:193, e.g., mutated at positions 116 and 119 of SEQ ID NO:193.

In one embodiment, the antibody molecule is an inhibitory or neutralizing antibody molecule. For example, the anti-IL13 antibody molecule can have a functional activity comparable to IL-13Rα2 (e.g., the anti-IL13 antibody molecule reduces or inhibits IL-13 interaction with IL-13Rα1). The anti-IL13 antibody molecule may prevent formation of a complex between IL-13 and IL-13Rα1, or disrupt or destabilize a complex between IL-13 and IL-13Rα1. In one embodiment, the anti-IL13 antibody molecule inhibits ternary complex formation, e.g., formation of a complex between IL 13, IL-13Rα1 and IL4-R. In one embodiment, the antibody molecule confers a post-injection protective effect against exposure to an antigen, e.g., an Ascaris antigen in a sheep model, at least 6 weeks after injection. In other embodiments, the anti-IL13 antibody molecule can inhibit one or more IL-13-associated biological activities with an IC₅₀ of about 50 nM to 5 pM, typically about 100 to 250 pM or less, e.g., better inhibition. In one embodiment, the anti-IL13 antibody molecule can associate with IL-13 with kinetics in the range of 10³ to 10⁸ M⁻¹ s⁻¹, typically 10⁴ to 10⁷ M⁻¹ s⁻¹. In one embodiment, the anti-IL13 antibody molecule binds to human IL-13 with a kₒₙ of between 5×10⁴ and 8×10⁵ M⁻¹ s⁻¹. In yet another embodiment, the anti-IL13 antibody molecule has dissociation kinetics in the range of 10⁻² to 10⁻⁶ s⁻¹, typically 10⁻² to 10⁻⁵ s⁻¹. In one embodiment, the anti-IL13 antibody molecule binds to IL-13, e.g., human IL-13, with an affinity and/or kinetics similar (e.g., within a factor 20, 10, or 5) to monoclonal antibody 13.2, MJ 2-7 or C65, or modified forms thereof, e.g., chimeric forms or humanized forms thereof. The affinity and binding kinetics of an IL-13 binding agent can be tested using, e.g., biosensor technology (BIACORE™).

In still another embodiment, the anti-IL13 antibody molecule specifically binds to an epitope, e.g., a linear or a conformational epitope, of IL-13, e.g., mammalian, e.g., human IL-13. For example, the antibody molecule binds to at least one amino acid in an epitope defined by IL-13Rα1 binding to human IL-13 or an epitope defined by IL-13Rα2 binding to human IL-13, or an epitope that overlaps with such epitopes. The anti-IL13 antibody molecule may compete with IL-13Rα1 and/or IL-13Rα2 for binding to IL-13, e.g., to human IL-13. The anti-IL13 antibody molecule may competitively inhibit binding of IL-13Rα1 and/or IL-13Rα2 to IL-13. The anti-IL13 antibody molecule may interact with an epitope on IL-13 which, when bound, sterically prevents interaction with IL-13Rα1 and/or IL-13Rα2. In embodiments, the anti-IL13 antibody molecule binds specifically to human IL-13 and competitively inhibits the binding of a second antibody to said human IL-13, wherein said second antibody is chosen from 13.2, MJ 2-7 and/or C65 (or any other anti-IL13 antibody disclosed herein) for binding to IL-13, e.g., to human IL-13. The anti-IL13 antibody molecule may competitively inhibit binding of 13.2, MJ 2-7 and/or C65 to IL-13. The anti-IL13 antibody molecule may specifically bind at least one amino acid in an epitope defined by 13.2, MJ 2-7 binding to human IL-13 or an epitope defined by C65 binding to human IL-13. In one embodiment, the anti-IL13 antibody molecule may bind to an epitope that overlaps with that of 13.2, MJ 2-7 or C65, e.g., includes at least one, two, three, or four amino acids in common, or an epitope that, when bound, sterically prevents interaction with 13.2, MJ 2-7 or C65. For example, the antibody molecule may contact one or more residues from IL-13 chosen from one or more of residues 81-93 and/or 114-132 of human IL-13 (SEQ ID NO: 194), or chosen from one or more of: Glutamate at position 68 [49], Asparagine at position 72 [53], Glycine at position 88 [69], Proline at position 91 [72], Histidine at position 92 [73], Lysine at position 93 [74], and/or Arginine at position 105 [86] of SEQ ID NO:194 [position in mature sequence; SEQ ID NO:195]. In other embodiments, the antibody molecule contacts one or more amino acid residues from IL-13 chosen from one or more of residues 116, 117, 118, 122, 123, 124, 125, 126, 127, and/or 128 of SEQ ID NO:24 or SEQ ID NO:178. In one embodiment, the antibody molecule binds to IL-13 irrespective of the polymorphism present at position 130 in SEQ ID NO:24.

In one embodiment, the antibody molecule includes one, two, three, four, five or all six CDR's from mAb13.2, MJ2-7, C65, or other antibodies disclosed herein, or closely related CDRs, e.g., CDRs which are identical or which have at least one amino acid alteration, but not more than two, three or four alterations (e.g., substitutions (e.g., conservative substitutions), deletions, or insertions). Optionally, the antibody molecule may include any CDR described herein. In embodiments, the heavy chain immunoglobulin variable domain comprises a heavy chain CDR3 that differs by fewer than 3 amino acid substitutions from a heavy chain CDR3 of monoclonal antibody MJ2-7 (SEQ ID NO:17), mAb 13.2 (SEQ ID NO:196) or C65 (SEQ ID NO:123). In other embodiments, the light chain immunoglobulin variable domain comprises a light chain CDR1 that differs by fewer than 3 amino acid substitutions from a corresponding light chain CDR of monoclonal antibody MJ2-7 (SEQ ID NO:18), mAb 13.2 (SEQ ID NO:197) or C65 (SEQ ID NO:118). The amino acid sequence of the heavy chan variable domain of MJ2-7 has the amino acid sequence shown as SEQ ID NO:130. The amino acid sequence of the light chan variable domain of MJ2-7 has the amino acid sequence shown as SEQ ID NO:133. The amino acid sequence of the heavy chan variable domain of monoclonal antibody 13.2 has the amino acid sequence shown as SEQ ID NO:198. The amino acid sequence of the light chan variable domain of monoclonal antibody 13.2 has the amino acid sequence shown as SEQ ID NO:199.

In certain embodiments, the heavy chain variable domain of the antibody molecule includes one or more of:

(SEQ ID NO:48)

G-(YF)-(NT)-I-K-D-T-Y-(MI)-H, in CDR1,

(SEQ ID NO:49)

(WR)-I-D-P-(GA)-N-D-N-I-K-Y-(SD)-(PQ)-K-F-Q-G,
in CDR2,
and/or

(SEQ ID NO:17)

SEENWYDFFDY, in CDR3;
or

(SEQ ID NO:15)

GFNIKDTYIH, in CDR1,

(SEQ ID NO:16)

RIDPANDNIKYDPKFQG, in CDR2,
and/or

(SEQ ID NO:17)

SEENWYDFFDY, in CDR3

In other embodiments, the light chain variable domain of the antibody molecule includes one or more of:

(SEQ ID NO:25)

(RK)-S-S-Q-S-(LI)-(KV)-H-S-(ND)-G-N-(TN)-Y-L-
(EDNQYAS), in CDR1,

(SEQ ID NO:27)

K-(LVI)-S-(NY)-(RW)-(FD)-S, in CDR2,
and/or

(SEQ ID NO:28)

Q-(GSA)-(ST)-(HEQ)-I-P, in CDR3;
or

(SEQ ID NO:18)

RSSQSIVHSNGNTYLE, in CDR1

(SEQ ID NO:19)

KVSNRFS, in CDR2,
and

(SEQ ID NO:20)

FQGSHIPYT, in CDR3.

In other embodiments, the antibody molecule includes one or more CDRs including an amino acid sequence selected from the group consisting of the amino acid sequence of SEQ ID NO:197, SEQ ID NO:200, SEQ ID NO:201, SEQ ID NO:202, SEQ ID NO:203, and SEQ ID NO:196.

In yet another embodiment, the antibody molecule includes at least one, two, or three Chothia hypervariable loops from a heavy chain variable region of an antibody chosen from, e.g., mAb13.2, MJ2-7, C65, or any other antibody disclosed herein, or at least particularly the amino acids from those hypervariable loops that contact IL-13. In yet another embodiment, the antibody or fragment thereof includes at least one, two, or three hypervariable loops from a light chain variable region of an antibody chosen from, e.g., mAb13.2, MJ2-7, C65, or other antibodies disclosed herein, or at least includes the amino acids from those hypervariable loops that contact IL-13. In yet another embodiment, the antibody or fragment thereof includes at least one, two, three, four, five, or six hypervariable loops from the heavy and light chain variable regions of an antibody chosen from, e.g., mAb13.2, MJ2-7, C65, or other antibodies disclosed herein.

In one embodiment, the protein includes all six hypervariable loops from mAb13.2, MJ2-7, C65, or other antibodies disclosed herein or closely related hypervariable loops, e.g., hypervariable loops which are identical or which have at least one amino acid alteration, but not more than two, three or four alterations, from the sequences disclosed herein. Optionally, the protein may include any hypervariable loop described herein.

In still another example, the protein includes at least one, two, or three hypervariable loops that have the same canonical structures as the corresponding hypervariable loop of mAb13.2, MJ2-7, C65, or other antibodies disclosed herein, e.g., the same canonical structures as at least loop 1 and/or loop 2 of the heavy and/or light chain variable domains of mAb13.2, MJ2-7, C65, or other antibodies disclosed herein. See, e.g., Chothia et al. (1992) J. Mol. Biol. 227:799-817; Tomlinson et al. (1992) J. Mol. Biol. 227:776-798 for descriptions of hypervariable loop canonical structures. These structures can be determined by inspection of the tables described in these references.

In one embodiment, the heavy chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the heavy chain framework of one of the following germline V segment sequences: DP-25, DP-1, DP-12, DP-9, DP-7, DP-31, DP-32, DP-33, DP-58, or DP-54, or another V gene which is compatible with the canonical structure class 1-3 (see, e.g., Chothia et al. (1992) J. Mol. Biol. 227:799-817; Tomlinson et al. (1992) J. Mol. Biol. 227:776-798). Other frameworks compatible with the canonical structure class 1-3 include frameworks with the one or more of the following residues according to Kabat numbering: Ala, Gly, Thr, or Val at position 26; Gly at position 26; Tyr, Phe, or Gly at position 27; Phe, Val, Ile, or Leu at position 29; Met, Ile, Leu, Val, Thr, Trp, or Ile at position 34; Arg, Thr, Ala, Lys at position 94; Gly, Ser, Asn, or Asp at position 54; and Arg at position 71.

In one embodiment, the light chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a Vκ II subgroup germline sequence or one of the following germline V segment sequences: A17, A1, A18, A2, A19/A3, or A23 or another V gene which is compatible with the canonical structure class 4-1 (see, e.g., Tomlinson et al. (1995) EMBO J. 14:4628). Other frameworks compatible with the canonical structure class 4-1 include frameworks with the one or more of the following residues according to Kabat numbering: Val or Leu or Ile at position 2; Ser or Pro at position 25; Ile or Leu at position 29; Gly at position 31d; Phe or Leu at position 33; and Phe at position 71.

In another embodiment, the light chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a Vκ I subgroup germline sequence, e.g., a DPK9 sequence.

In another embodiment, the heavy chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a VH I subgroup germline sequence, e.g., a DP-25 sequence or a VH III subgroup germline sequence, e.g., a DP-54 sequence.

In certain embodiments, the heavy chain immunoglobulin variable domain of the antibody molecule includes an amino acid sequence encoded by a nucleotide sequence that hybridizes under high stringency conditions to the complement of the nucleotide sequence encoding a heavy chain variable domain of V2.1 (SEQ ID NO:71), V2.3 (SEQ ID NO:73), V2.4 (SEQ ID NO:74), V2.5 (SEQ ID NO:75), V2.6 (SEQ ID NO:76), V2.7 (SEQ ID NO:77), V2.11 (SEQ ID NO:80), ch13.2 (SEQ ID NO:204), h13.2v1 (SEQ ID NO:205), h13.2v2 (SEQ ID NO:206) or h13.2v3 (SEQ ID NO:207); or includes an amino acid sequence that is at least 80%, 85%, 90%, 95%, 97%, 98%, 99% or higher identical identical to the amino acid sequence of the heavy chain variable domain of V2.1 (SEQ ID NO:71), V2.3 (SEQ ID NO:73), V2.4 (SEQ ID NO:74), V2.5 (SEQ ID NO:75), V2.6 (SEQ ID NO:76), V2.7 (SEQ ID NO:77), V2.11 (SEQ ID NO:80); ch13.2 (SEQ ID NO:208), h13.2v1 (SEQ ID NO:209), h13.2v2 (SEQ ID NO:210) or h13.2v3 (SEQ ID NO:211). In embodiments, the heavy chain immunoglobulin variable domain includes the amino acid sequence of SEQ ID NO:80, which may in turn further include a heavy chain variable domain framework region 4 (FR4) that includes the amino acid sequence of SEQ ID NO:116 or SEQ ID NO:117.

In other embodiments, the light chain immunoglobulin variable domain of the antibody molecule includes an amino acid sequence encoded by a nucleotide sequence that hybridizes under high stringency conditions to the complement of the nucleotide sequence encoding a light chain variable domain of V2.11 (SEQ ID NO:36) or h13.2v2 (SEQ ID NO:212); or includes an amino acid sequence that is at least 80%, 85%, 90%, 95%, 97%, 98%, 99% or higher identical to a light chain variable domain of V2.11 (SEQ ID NO:36) or h13.2v2 (SEQ ID NO:212). In embodiments, the light chain immunoglobulin variable domain includes the amino acid sequence of SEQ ID NO:36, which may in turn further include a light chain variable domain framework region 4 (FR4) that includes the amino acid sequence of SEQ ID NO:47.

In yet another embodiment, the antibody molecule includes a framework of the heavy chain variable domain sequence comprising:
(i) at a position corresponding to 49, Gly;
(ii) at a position corresponding to 72, Ala;
(iii) at positions corresponding to 48, Ile, and to 49, Gly;
(iv) at positions corresponding to 48, Ile, to 49, Gly, and to 72, Ala;
(v) at positions corresponding to 67, Lys, to 68, Ala, and to 72, Ala; and/or
(vi) at positions corresponding to 48, Ile, to 49, Gly, to 72, Ala, to 79, Ala.

In one embodiment, the anti-IL13 antibody molecule includes at least one light chain that comprises the amino acid sequence of SEQ ID NO:177 (or an amino acid sequence at least 80%, 85%, 90%, 95%, 97%, 98%, 99% or higher identical to SEQ ID NO:177) and at least one heavy chain that comprises the amino acid sequence of SEQ ID NO:176 (or an amino acid sequence at least 80%, 85%, 90%, 95%, 97%, 98%, 99% or higher identical to SEQ ID NO:176).

In one embodiment, the anti-IL13 antibody molecule includes two immunoglobulin chains: a light chain that includes SEQ ID NO:199, 213, 214, 212, or 215 and a heavy chain that includes SEQ ID NO:198, 208, 209, 210, or 211 (or an amino acid sequence at least 80%, 85%, 90%, 95%, 97%, 98%, 99% or higher identical to SEQ ID NO:199, 213, 214, 212, or 215, or SEQ ID NO:198, 208, 209, 210, or 211). The antibody molecule may further include in the heavy chain the amino acid sequence of SEQ ID NO:193 and in the light chain the amino acid sequence of SEQ ID NO:216 (or an amino acid sequence at least 80%, 85%, 90%, 95%, 97%, 98%, 99% or higher identical to SEQ ID NO:193 or SEQ ID NO:216).

In another embodiment, the IL-13 binding agent, e.g., anti-IL-13 antibody molecule, interferes with the interaction of IL-13 with the receptor IL-13RI1. In one embodiment, the IL-13 binding agent can interfere with the interaction of Phe107 of IL-13 (SEQ ID NO:124; FIG. 13A) with a hydrophobic pocket of IL-13Rα1 formed by the side chains of residues Leu319, Cys257, Arg256, and Cys320 (SEQ ID NO:125; FIG. 13B), e.g., by direct binding to these residues or steric hindrance. In another embodiment, the IL-13 binding agent can interfere with van der Waals interactions between amino acid residues Ile254, Ser255, Arg256, Lys318, Cys320, and Tyr321 of IL-13Rα1 (SEQ ID NO:125) and amino acid residues Arg11, Glu12, Leu13, Ile14, Glu15, Lys104, Lys105, Leu106, Phe107, and Arg108 of IL-13 (SEQ ID NO:124), e.g., by direct binding to these residues or steric hindrance.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The contents of all publications, pending patent applications, published patent applications (inclusive of US 06/0073148 and US 06/0063228), and published patents cited throughout this application are hereby incorporated by reference in their entirety.

Others features, objects and advantages of the invention will be apparent from the description and drawings, and from the claims.

### BRIEF DESCRIPTION OF THE DRAWINGS

FIG. 1A is an alignment of full-length human and cynomolgus monkey IL-13, SEQ ID NO:178 and SEQ ID NO:24, respectively. Amino acid differences are indicated by the shaded boxed residues. The location of the R to Q substitution (which corresponds to the polymorphism detected in allergic patients) is boxed at position 130. The location of the cleavage site is shown by the arrow.

FIG. 1B is a list of exemplary peptides from cynomolgus monkey IL-13 (SEQ ID NO:179-188, respectively) that can be used for generating anti-IL13 antibodies.

FIG. 2 is a graph depicting the neutralization of NHP IL-13 activity by various IL-13 binding agents, as measured by percentage of CD23⁺ monocytes (y-axis). Concentration of MJ2-7 (Δ), C65 ( [symbol] ), and sIL-13RI2-Fc () are indicated on the x-axis.

FIG. 3 is a graph depicting the neutralization of NHP IL-13 activity by MJ2-7 (murine; ) or humanized MJ2-7 v.211 (∘) (referred to interchangeably herein as “hMJ2-7v.2-11 or “MJ2-7v.2-11”). NHP IL-13 activity was measured by phosphorylation of STAT6 (y-axis) as a function of antibody concentration (x-axis).

FIG. 4 is a graph depicting the neutralization of NHP IL-13 activity by MJ2-7 v.211 (∘) or sIL-13RI2-Fc [symbol] (NHP IL-13 activity was measured by phosphorylation of STAT6 (y-axis) as a function of antagonist concentration (x-axis).

FIG. 5 is a graph depicting the neutralization of NHP IL-13 activity by MJ2-7 (Δ), C65 ( [symbol] ), or sIL-13RI2-Fc (). NHP IL-13 activity was measured by phosphorylation of STAT6 (y-axis) as a function of antagonist concentration (x-axis).

FIG. 6A is a graph depicting induction of tenascin production (y-axis) by native human IL-13 (x-axis).

FIG. 6B is a graph depicting the neutralization of NHP IL-13 activity by MJ2-7, as measured by inhibition of induction of tenascin production (y-axis) as a function of antibody concentration (x-axis).

FIG. 7 is a graph depicting binding of MJ2-7 or control antibodies to NHP-IL-13 bound to sIL-13RI2-Fc coupled to a SPR chip.

FIG. 8 is a graph depicting binding of varying concentrations (0.09-600 nM) of NHP IL-13 to captured hMJ2-7 v.2-11 antibody.

FIG. 9 is a graph depicting the neutralization of NHP IL-13 activity by mouse MJ2-7 () or humanized Version 1 (∘), Version 2 ( [symbol] ), or Version 3 (Δ) antibodies. NHP IL-13 activity was measured by phosphorylation of STAT6 (y-axis) as a function of antibody concentration (x-axis).

FIG. 10 is a graph depicting the neutralization of NHP IL-13 activity by antibodies including mouse MJ2-7 VH and VL (), mouse VH and humanized Version 2 VL (Δ), or Version 2 VH and VL ( [symbol] ). NHP IL-13 activity was measured by phosphorylation of STAT6 (y-axis) as a function of antibody concentration (x-axis).

FIGS. 11A and 11B are graphs depicting inhibition of binding of IL-13 to immobilized IL-13 receptor by MJ2-7 antibody, as measured by ELISA. Binding is depicted as absorbance at 450 nm (y-axis). Concentration of MJ2-7 antibody is depicted on the x-axis. FIG. 11A depicts binding to IL-13Rα1. FIG. 11B depicts binding to IL-13Rα2.

FIG. 12 is an alignment of DPK18 germline amino acid sequence (SEQ ID NO:126) and humanized MJ2-7 Version 3 VL (SEQ ID NO:190).

FIG. 13A is an amino acid sequence (SEQ ID NO:124) of mature, processed human IL-13.

FIG. 13B shows an amino acid sequence (SEQ ID NO:125) of human IL-13Rα1.

FIGS. 14A-14D show an increase in the total number of cells/ml and percentage of inflammatory cells present in BAL fluid post-Ascaris challenge compared to pre-(baseline) samples.

FIGS. 15A-15B show total of BAL cells/ml in BAL fluids in control and antibody-treated cynomolgus monkeys pre- and post-Ascaris challenge. Control (light circles (∘); MJ2-7v.2-11-treated samples (light triangles (light triangles)) and mAb 13.2v2-treated samples (dark triangles(▴)). (Humanized versions of MJ2-7 (MJ2-7v.2-11) and mAb 13.2v2 were used in this study).

FIGS. 16A-16B show changes in eotaxin levels in concentrated BAL fluid collected from antibody-treated cynomolgus monkeys post-Ascaris challenge relative to control. FIG. 16A depicts a bar graph showing an increase in eotaxin levels (pg/ml) post-Ascaris challenge relative to a baseline, pre-challenge values. FIG. 16B depicts a decrease in eotaxin levels in concentrated BAL fluids from cynomolgus monkeys treated with mAb 13.2-(gray circles) or MJ2-7-(gray triangles) antibodies compared to a control (dark circles). (Humanized versions of MJ2-7 (MJ2-7v.2-11) and mAb 13.2 v2 were used in this study).

FIGS. 17A-17B depict the changes in Ascaris-specific IgE-titers in control and antibody-treated samples 8-weeks post-challenge. FIG. 17A depicts representative examples showing no change in Ascaris-specific IgE titer in an individual monkey treated with irrelevant Ig (IVIG; animal 20-45; top panel), and decreased titer of Ascaris-specific IgE in an individual monkey treated with humanized MJ2-7v.2-11 (animal 120-434; bottom panel). FIG. 17B depicts a decrease in Ascaris-specific IgE-titers in mAb13.2 or hMJ2-7-11 (dark circles) relative to irrelevant Ig-treated cynomolgus monkeys (IVIG (gray circles)) 8-weeks post-Ascaris challenge.

FIGS. 18A-18B show the changes in Ascaris-specific basophil histamine release in control and antibody-treated samples 24-hours and 8-weeks post-challenge. FIG. 18A is a graph depicting the following samples in representative individual monkeys treated with saline (left) or humanized mAb13.2v.2 (right): pre-antibody or Ascaris challenged samples (circles); 48-hours post-antibody treatment, 24-hours post-Ascaris challenged samples (triangles); and 8 weeks post-Ascaris challenged samples (diamonds). FIG. 18B depicts a bar graph showing the changes in normalized histamine levels pre- and 8-week post-Ascaris challenge in control (solid black bar), humanized mAb13.2-(white bar) and humanized MJ2-7v.2-11-(hatched bar) treated cynomolgus monkeys.

FIG. 19 depicts the correlation between Ascaris-specific histamine release and Ascaris-specific IgE levels in control (light circles) and anti-IL13- or dexamethasone-treated samples (dark circles).

FIG. 20 is a series of bar graphs depicting the changes in serum IL-13 levels in individual cynomolgus monkeys treated with humanized MJ2-7 (hMJ2-7v.2-11). The label in each panel (e.g., 120-452) corresponds to the monkey identification number. The “pre” sample was collected prior to administration of the antibody. The time “0” was collected 24-hours post-antibody administration, but prior to Ascaris challenge. The remaining time points were post-Ascaris challenge.

FIG. 21 is a bar graph depicting the STAT6 phosphorylation activity of non-human primate IL-13 at 0, 1, or 10 ng/ml, either in the absence of serum (“no serum”); the presence of serum from saline or IVIG-treated animals (“control”); or in the presence of serum from anti-IL13 antibody-treated animals, either before antibody administration (“pre”), or 1-2 weeks post-administration of the indicated antibody. Serum was tested at 1:4 dilution. (Humanized versions of MJ2-7 (MJ2-7v.2-11) and mAb 13.2 v2 were used in this study).

FIGS. 22A-22C are linear graphs showing that levels of non-human primate IL-13 trapped by humanized MJ2-7 (hMJ2-7v.2-11) in cynomolgus monkey serum correlate with the level of inflammation measured in the BAL fluids post-Ascaris challenge.

FIGS. 23A-23B are line graphs showing altered lung function in mice in response to human recombinant R110Q IL-13 intratracheal administration; FIG. 23A shows the changes in airway resistance (RI) in response to increasing doses of nebulized metacholine; FIG. 23B shows the changes in dynamic lung compliance (Cdyn) in response to increasing doses of nebulized metacholine.

FIGS. 24A-24B are bar graphs showing increased lung inflammation and cytokine production in mice in response to human recombinant R100Q IL-13 intranasal administration. In FIG. 24A, the percentage of eosinophils and neutrophils in bronchoalveolar lavage (BAL) were determined by differential cell counts. In FIG. 24B, the levels of cytokines, MCP-1, TNF-I, and IL-6, in BAL were determined by cytometric bead array. Data is median±s.e.m. of 10 animals per group.

FIGS. 25A-25B are dot plots showing humanized MJ2-7-11 (hMJ2-7v.2-11) antibody levels in BAL and serum following intratracheal and intravenous administration. Animals were treated with human recombinant R110Q IL-13, or an equivalent volume (20 μL) of saline, intratracheally on days 1, 2, and 3. Humanized MJ2-7v.2-11 antibody was administered on day 0 and 2 hours before each dose of human recombinant R110Q IL-13. FIG. 25A depicts the results when the antibody is administered intravenously on day 0 and intraperitoneally on days 1, 2, and 3; or intranasally on days 0, 1, 2, and 3 (shown in FIG. 25B). Total human IgG levels in BAL and serum were assayed by ELISA.

FIGS. 26A-26C show the effect of humanized MJ2-7v.2-11 antibody after intranasal administration of human recombinant R110Q IL-13-induced altered lung function. (A) FIG. 26A shows the changes in lung resistance (RI; cm H₂O/ml/sec) expressed as change from baseline. FIG. 26B shows data expressed as methacholine dose required to elicit lung resistance (RI) corresponding to a change of 2.5 ml H₂O/cm/sec from baseline. Median values are shown for each treatment group. p-values were calculated by two-tailed t-test. FIG. 26C shows the median human IgG levels in BAL and sera.

FIGS. 27A-27D show the changes in BAL and serum levels of human recombinant R110Q IL-13 administered alone (FIGS. 27A-27B) or in complex with humanized MJ2-7v.2-11 antibody (FIGS. 26C-27D) following intratracheal administration of human recombinant R110Q IL-13 and intranasal administration of humanized MJ2-7v.2-11 antibody. Median values are indicated for each group. n.d. is not detectable.

FIGS. 28A-28B are dot plots showing eosinophil (FIG. 28A) and neutrophil (FIG. 28B) infiltration into BAL levels following intranasal administration of human recombinant R110Q IL-13 and intranasal administration of 500, 100, and 20 μg of humanized MJ2-7v.2-11 and humanized 13.2v.2, saline, or 500 μg of IVIG. Eosinophil and neutrophil percentages were determined by differential cell counts. Median values for each group are indicated. p-values were determined by two-tailed test and are indicated for each antibody-treated group as compared to IVIG.

FIGS. 29A-29C are dot plots showing changes in cytokine levels, MCP-1, TNF-I, and IL-6, respectively, following intranasal administration of human recombinant R110Q IL-13 and intranasal administration of 500 Tg of humanized MJ2-7v.2-11, humanized 13.2v.2, or IVIG, or saline. Dashed line indicates limit of assay sensitivity. Data represent median values for each group. p-value was ≦0.0001, according to a two-tailed t-test.

FIGS. 30A-30B are dot plots showing that human recombinant R110Q IL-13 levels are directly related to lung inflammation, as measured by eosinohilia; and inversely proportional to humanized MJ2-7v.2-11 BAL levels following intranasal administration of human recombinant R110Q IL-13 and intranasal administration of 500, 100, or 20 μg doses of humanized MJ2-7v.2-11 antibody. Humanized MJ2-7v.2-11 antibody BAL levels were measured by ELISA. Human recombinant R110Q IL-13 BAL levels were determined by cytometric bead assay. % eosinophil was determined by differential cell counting. Associations are shown between levels of: (FIG. 30A) % eosinophilic inflammation and human recombinant R110Q IL-13, including data from saline control animals, mice treated with human recombinant R110Q IL-13 alone, and mice treated with human recombinant R110Q IL-13 and 500, 100, and 20 Tg of humanized MJ2-7v.2-11 antibody or 500 μg IVIG; and (FIG. 30B) humanized MJ2-7v.2-11 and IL-6, including data from mice treated with 500, 100, and 20 Tg of humanized MJ2-7v.2-11. r² and p-values were determined by linear regression analysis.

FIGS. 31A-31B are line graphs showing concentrations of [¹²⁵I]-labeled humanized 13.2v.2 anti-IL-13 antibody and [²⁵¹I]-labeled humanized MJ2-7v.2-11 antibody in various mouse and rat tissue, respectively. Following IV administration of anti-IL-13 antibodies, tissue samples were collected at 1, 24, 168, and 336 hours (FIG. 31A) or 1, 48, 168, 336, and 840 hours (FIG. 31B).

FIGS. 32A-32B are line graphs showing observed and predicted IL-13 and anti-IL-13 antibody levels over time. In FIG. 32A, 1 mg/kg of humanized MJ2-7v.2-11 antibody was administered to naïve cynomolgus monkeys. Total IL-13 and humanized MJ2-7v.2-11 serum levels were quantified over a period of 0-45 days using a specific ELISA. Predicted IL-13 and humanized MJ2-7v.2-11 antibody levels based on model shown in FIG. 40 are shown for comparison. In FIG. 32B, humanized 13.2v.2 and humanized MJ2-7v.2-11 antibodies were administered to cynomolgus monkeys at day 0 and Ascaris challenge was performed at day 1. Total IL-13 serum levels were quantified over a period of up to 120 days using a specific ELISA.

FIG. 33 is a schematic representation of PK-PD model of humanized MJ2-7v.2-11. Ab is hMJ2-7v.2-11. Complex is hMJ2-7v.2-11/IL-13 complex. CL_{d,Ab} and CL_{Ab} are distribution clearance and serum clearance of hMJ2-7v.2-11, respectively. CL_{complex} and CL_{IL-13} are serum clearance of the complex and IL-13, respectively. K_{syn} is a zero-order IL-13 synthesis rate constant, Kₒₙ is a second-order association rate constant, and K_{off} is a first-order dissociation rate constant. V and V₂ are volumes of distribution of hMJ2-7v.2-11 in the serum (central) and the second compartment, respectively.

FIGS. 34A-34C show mean hMJ2-7v.2-11 and total IL-13 concentration time-profiles in cynomolgus monkeys. A single 1 mg/kg IV or 2 mg/kg SC dosage of hMJ2-7v.2-11 was administered to naive cynomolgus monkey and a single 10 mg/kg IV dosage of hMJ2-7v.2-11 was given to Ascaris-challenged cynomolgus monkeys. The challenge was performed that with 0.75 μg of Ascaris suum antigen 24 hours post administration of the hMJ2-7v.2-11. hMJ2-7v.2-11 (A, B) and total IL-13 (C) concentrations were determined using quantitative ELISAs. Data point show individual animal values (A) or mean values (B and C). For the mean values, N=3 for 1 mg/kg-IV group, N=2 for 2 mg/kg-SC group, and N=8 for 10 mg/kg-IV group, with Monkey #5 in the SC group being excluded from calculations of the mean values. Error bars indicated standard deviation from the mean values. M=monkey.

FIGS. 35A-35D are a series of goodness-of-fit plots showing hMJ2-7v.2-11 (closed circle) and total IL-13 (open circle) concentrations following a single dosage of hMJ2-7v.2-11 fitted using the integrated PK-PD model depicted in FIG. 33. Individual observed versus individual predicted concentrations (A) and individual weighted residuals versus individual predicted concentrations (B) following a single dosage of hMJ2-7v.2-11 are shown for five naive (N=3, 1 mg/kg IV and N=2, 2 mg/kg SC) and eight Ascaris-challenged cynomolgus monkeys (10 mg/kg, IV). One animal in the SC group was excluded from these analyses due to a sharp decline in hMJ2-7v.2-11 and total IL-13 levels in the terminal phase, compared to other naive monkeys in the study. Representative individual fits after IV administration of hMJ2-7v.2-11 are shown for a naive (C) and an Ascaris-challenged monkey (D), with predicted hMJ2-7v.2-11 and total IL-13 levels shown by solid line and dotted lines, respectively.

FIGS. 36A and 36B are graphs depicting simulated free IL-13 and total IL-13 concentration-time profiles after a single IV administration of hMJ2-7v.2-11 to cynomolgus monkeys. For naive monkeys (FIG. 36A), a 1 mg/kg dosage was assumed as in Study 1, while for Ascaris-challenged monkeys (FIG. 36B), a 10 mg/kg dosage and Ascaris challenge 24-hour post-hMJ2-7v.2-11 administration (Day 1) was assumed as in Study 2. Free IL-13 is shown by solid lines, while total IL-13 is shown by dotted lines.

FIGS. 37A and 37 B are graphs showing simulated free IL-13 concentration-time profiles after different dosing regimens of hMJ2-7v.2-11 to cynomolgus monkeys. A single 1, 5, 10, 20, or 50 mg/kg IV bolus dosage of hMJ2-7v.2-11 (as indicated) was assumed for both naïve (FIG. 37A) and Ascaris-challenged (FIG. 37B) monkeys. Ascaris challenge was assumed at pre-dose (Day 0) to mimic the “established airway inflammation” situation.

FIG. 38 is a line graph plotted from PK data showing concentration-time profiles of humanized MJ2-7v.2-11 in normal versus Ascaris-challenged cynomolgus monkeys.

FIG. 39 is a line graph plotted from PK data showing concentration-time profiles of humanized 13.2v.2 in normal versus Ascaris-challenged cynomolgus monkeys.

FIG. 40 is a stoichiometric PK-PD model of IL-13 and anti-IL-13 antibody disposition in cynomolgus monkeys, wherein; Ab is anti-IL-13 antibody; Complex is an Ab and IL-13 complex; Comp is compartment; CLd_{Ab} and CL_{Ab} are distribution clearance and serum clearance of Ab, respectively; CL_{complex} is serum clearance of the complex; K_{SYN} is the zero-order IL-13 synthesis rate constant; K_{DEG} is the first-order IL-13 degradation constant; Kon is the third-order association rate constant; Koff is the first-order dissociation rate constant; V_{Ab} and V2_{Ab} are apparent volumes of distribution in the serum and the second compartment, respectively; and the model is based on the assumptions that Kon is 3^{rd} order; anti-IL-13 and IL-13 have a 1:2 molar binding ratio; and V_{anti-IL-13}=V_{complex}=V_{IL-13}=V.

FIG. 41 is a line graph showing predicted serum concentrations of free and humanized MJ2-7v.2-11-bound IL-13 following 1 mg/kg IV administration of humanized MJ2-7v.2-11 to naïve cynomolgus monkeys. Data were predicted using the concentration-time profiles from studies described in Table 8 and depicted in FIG. 34, and the model presented in FIG. 40, and is represented for a period of up to 50 days.

FIG. 42 is a line graph showing predicted serum concentrations of free and humanized MJ2-7v.2-11-bound IL-13 following 1 mg/kg IV administration of humanized MJ2-7v.2-11 to Ascaris-challenged cynomolgus monkeys. Data were predicted using the concentration-time profile from studies described in Table 8 and depicted in FIG. 34, and the model presented in FIG. 40, and is represented for a period of up to 150 days.

FIG. 43 is a series of line graphs showing allometric scaling of humanized MJ2-7v.2-11 for three PK parameters, CL, V_{dss} and t_{1/2}. Solid line represents the fitted curve based on a linear regression using data from mice, rats and monkeys. The dotted lines represent the 95% confidence intervals.

FIG. 44 is a line graph showing the percent change in FEV1 (% Change in FEV1) at various time points after allergen challenge (Time after allergen challenge (h)) for human subjects that will be treated with anti-IL-13 antibody treated (open circles) or placebo treated (closed circles). The results shown are for allergen challenge on the screening day two weeks prior to the initial administration of anti-IL-13 antibody or placebo. (h): hours; EAR: early asthmatic response; LAR: late asthmatic response.

FIG. 45 is a line graph showing the percent change in FEV1 (% Change in FEV1) at various time points after allergen challenge (Time after allergen challenge (h)) for anti-IL-13 antibody treated (open circles) or placebo treated (closed circles) human subjects. The results shown are for allergen challenge on day 14 after initial administration of anti-IL-13 antibody or placebo. (h): hours; EAR: early asthmatic response; LAR: late asthmatic response.

FIG. 46 is a line graph showing the percent change in FEV1 (% Change in FEV1) at various time points after allergen challenge (Time after allergen challenge (h)) for anti-IL-13 antibody treated (open circles) or placebo treated (closed circles) human subjects. The results shown are for allergen challenge on day 35 after initial administration of anti-IL-13 antibody or placebo. (h): hours; EAR: early asthmatic response; LAR: late asthmatic response.

FIG. 47 is a graph showing serum concentration (ng/mL) of antibody at Day 14 and Day 35.

FIG. 48 is a table showing the maximum percent drop (max % drop) and area under the curve percent drop (AUC % drop) during the EAR (early phase) and LAR (late phase) on Day 14 and Day 35 after initial antibody (or placebo) administration. P values (P-val) are also indicated.

FIG. 49 is a line graph showing the 13.2v2 antibody serum concentration (ng/ml) in human subjects over time (days) after administration. The thin lines depict the PK profiles for 13.2v2 antibody administered in a single ascending dose of 4 mg/kg. The thicker lines depict the PK profiles for 13.2v2 antibody administered as two doses of 2 mg/kg. Administration of the two doses was separated by a week.

FIG. 50 is a graph showing individual AUC normalized by mg/kg dose against respective body weight in 81 subjects from both study A and study B.

FIG. 51 is a graph showing individual AUC normalized by total dose (body weight*mg/kg dose) against respective body weight in 81 subjects from both study A and study B.

FIG. 52 is a graph showing 13.2v2 AUC exposure normalized by actual dose (body weight*mg/kg dose).

### DETAILED DESCRIPTION

Methods and compositions for treating and/or monitoring treatment of IL-13-associated disorders or conditions are disclosed. In one embodiment, Applicants have discovered that administration of an IL-13 antagonist, e.g., an IL-13 antibody molecule, reduces at least one symptom of an allergen-induced early and/or a late asthmatic response in a subject, e.g., a human subject, relative to an untreated subject. The reduction in one or more asthmatic symptoms is detected within minutes following exposure of the subject to an insult, e.g., an allergen, and during an early asthmatic response (e.g., up to about 3 hours after exposure to the insult). The reduction in symptoms is maintained during a late asthmatic response (e.g., for a period of about 3 to 24 hours after insult exposure). In other embodiments, methods of evaluating an anti-IL13 antibody molecule and/or treatment modalities associated with said antibody molecule are disclosed. The evaluation methods include detecting at least one pharmacokinetic/pharmacodynamic (PK/PD) parameter of the anti-IL13 antibody molecule in the subject. Thus, uses of IL-13 binding agents or antagonists for reducing or inhibiting, and/or preventing or delaying the onset of, one or more symptoms associated with an early and/or a late phase of an IL-13-associated disorder or condition in a subject are disclosed. In other embodiments, methods for evaluating the kinetics and/or efficacy of an IL-13 binding agent or antagonist in treating or preventing the IL-13-associated disorder or condition in a subject are also disclosed.

### DEFINITIONS

For convenience, certain terms are defined herein. Additional definitions can be found throughout the specification.

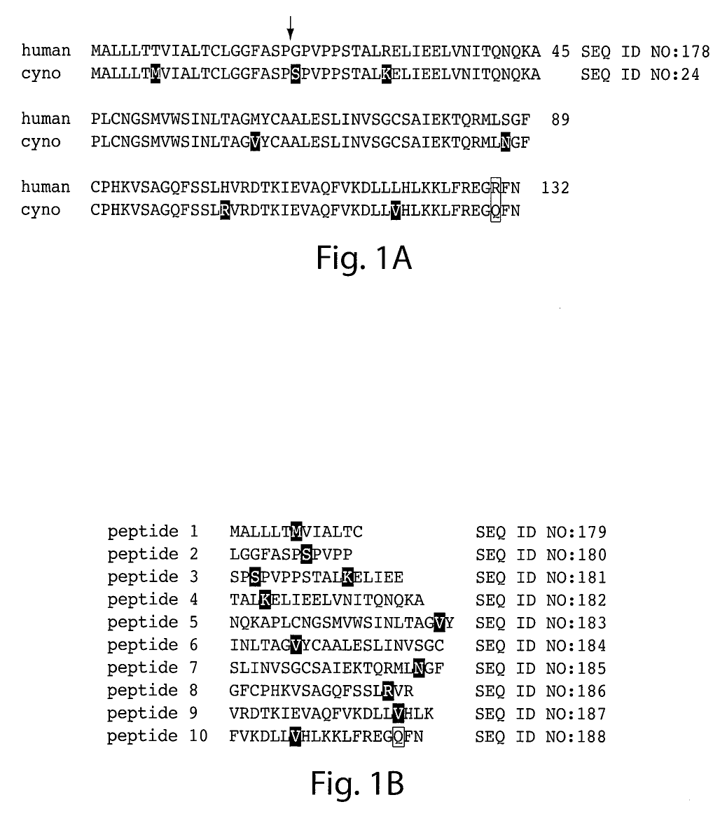

The term “IL-13” includes the full length unprocessed form of the cytokines known in the art as IL-13 (irrespective of species origin, and including mammalian, e.g., human and non-human primate IL-13) as well as mature, processed forms thereof, as well as any fragment (of at least 5 amino acids) or variant of such cytokines. Positions within the IL-13 sequence can be designated in accordance to the numbering for the full length, unprocessed human IL-13 sequence. For an exemplary full-length monkey IL-13, see SEQ ID NO:24; for mature, processed monkey IL-13, see SEQ ID NO:14; for full-length human IL-13, see SEQ ID NO:178, and for mature, processed human IL-13, see SEQ ID NO:124 (FIG. 1). An exemplary sequence is recited as follows:

(SEQ ID NO:178)

MALLLTTVIALTCLGGFASPGPVPPSTALRELIEELVNITQNQKAPLCNG
SMVWSINLTAGMYCAALESLINVSGCSAIEKTQRMLSGFCPHKVSAGQFS
SLHVRDTKIEVAQFVKDLLLHLKKLFREGRFN

There is about 94% amino acid sequence identity between the human and cyno monkey IL-13 sequences, due to 8 amino acid sequences. One of these differences, R130Q, represents a common human polymorphism typically expressed in asthmatic subjects (Heinzmann et al. (2000) Human Mol Genet. 9:549-559).

Exemplary sequences of IL-13 receptor proteins and soluble forms thereof (e.g., IL-13Rα1 and IL-13Rα2 or fusions thereof) are described, e.g., in Donaldson et al. (1998) J Immunol. 161:2317-24; U.S. Pat. No. 6,214,559; U.S. Pat. No. 6,248,714; and U.S. Pat. No. 6,268,480.

The phrase “a biological activity of” IL-13/IL-13R polypeptide refers to one or more of the biological activities of the corresponding mature IL-13 polypeptide, including, but not limited to, (1) interacting with, e.g., binding to, an IL-13R polypeptide (e.g., a human IL-13R polypeptide); (2) associating with signal transduction molecules, e.g., γ common; (3) stimulating phosphorylation and/or activation of stat proteins, e.g., STAT6; (4) induction of CD23 expression; (5) production of IgE by human B cells; (6) induction of antigen-induced eosinophilia in vivo; (7) induction of antigen-induced bronchoconstriction in vivo; (8) induction of drug-induced airway hyperreactivity in vivo; (9) induction of eotoxin levels in vivo; and/or (10) induction histamine release by basophils.

An “IL-13 associated disorder or condition” is one in which IL-13 contributes to a pathology or symptom of the disorder or condition. Accordingly, an IL-13 binding agent, e.g., an IL-13 binding agent that is an antagonist of one or more IL-13 associated activities, can be used to treat or prevent the disorder.

As used herein, a “therapeutically effective amount” of an IL-13/IL-13R antagonist refers to an amount of an agent which is effective, upon single or multiple dose administration to a subject, e.g., a human patient, at curing, reducing the severity of, ameliorating, or preventing one or more symptoms of a disorder, or in prolonging the survival of the subject beyond that expected in the absence of such treatment.

As used herein, a “prophylactically effective amount” of an IL-13/IL-13R antagonist refers to an amount of an IL-13/IL-13R antagonist which is effective, upon single or multiple dose administration to a subject, e.g., a human patient, in preventing, reducing the severity, or delaying the occurrence of the onset or recurrence of an IL-13-associated disorder or condition, e.g., a disorder or condition as described herein.

As used herein “a single treatment interval” referees to an amount and/or frequency of administration of an IL-13/IL-13R antagonist that when administered as a single dose, or as a repeated dose of limited frequency reduces the severity of, ameliorates, prevents, or delays the occurrence of the onset or recurrence of, one or more symptoms of an IL-13-associated disorder or condition, e.g., a disorder or condition as described herein. In embodiments, the frequency of administration is limited to no more than two or three doses during a single treatment interval, e.g., the repeated dose is administered within one week or less from the initial dose.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5 M sodium phosphate, 7% SDS at 65° C., followed by one or more washes at 0.2×SSC, 1% SDS at 65° C. Very high stringency conditions (4) are the preferred conditions and the ones that are used unless otherwise specified.

The methods and compositions of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to the sequence specified are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%. 91%, 92%, 93%, 94%, 95%, 96%, 97%, 98% or 99% identity to the sequence specified are termed substantially identical.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

The term “early asthmatic response” or “EAR” refers to the initial period of response after a subject's exposure to an allergen. For example, the response occurring in the first 3 hours (e.g., about 2.5, about 2.75, about 2.9, about 3, about 3.25, about 3.5 hours) following exposure to an allergen is considered to be the EAR. For example, the maximum airway construction can occur within about 15-30 minutes after exposure. Events that occur during the EAR can include the release of mediators such as leukotrienes (e.g., LTA₄, LTB₄, LTC₄, LTD₄, LTE₄, and/or LTF₄) and/or histamine from airway mast cells, e.g., leading to bronchoconstriction and/or airway edema, and/or increase in the levels of leukotrienes and/or histamine (e.g., an increase relative to the level of leukotrienes and/or histamine in the subject prior to exposure to allergen). Treatments for EAR include administration of an anti-IL-13 antibody (e.g., an antibody described herein), an anti-histamine (e.g., loratidine (e.g., CLARITIN®), cetirizine (e.g., ZYRTEC®), diphenhydramine), an anti-leukotriene (e.g., zafirlukast, montelukast (e.g., SINGULAIR®)), an IL-4 variant (e.g., pintrakinra), or a combination of two or more of these agents.

The term “late asthmatic response” or “LAR” refers to the period of response after a subject's exposure to an allergen that occurs after the EAR, or the response that begins about 3 hours after a subject's exposure to an allergen. As a further example, the LAR commences after about 3-5 hours, is maximal at about 6-12 hours, and can persist for up to about 24 hours. In contrast to the EAR, the LAR involves inflammatory cells and/or an increase in mucus. For example, the LAR can be associated with increases in airway reactivity and/or with an influx and activation of inflammatory cells, such as lymphocytes, eosinophils, and macrophages, e.g., in the airways and/or bronchial mucosa (e.g., an increase relative to the level of inflammatory cells, such as lymphocytes, eosinophils, and macrophages, e.g., in the airways and/or bronchial mucosa in the subject prior to exposure to allergen). Treatments for LAR include administration of an anti-IL-13 antibody (e.g., an antibody described herein), a steroid (e.g., inhaled steroid), a beta-agonist (e.g., albuterol (e.g., VENTOLIN®; PROVENTIL®, SALBUTAMOL®), metaproteronol (e.g., ALUPENT®, METAPREL®), terbutaline (e.g., BRETHINE®, BRICANYL®, or BRETHAIRE®) or a combination of two or more of these agents.

A “flat” dose of a therapeutic agent (e.g., anti-IL-13 antibody) refers to a dose that is administered to a subject without regard for the weight or body surface area of the subject. The flat dose is not provided as a mg/kg dose, but rather as an absolute amount of the therapeutic agent.

### Antibody Molecules

Examples of IL-13 antagonists and/or binding agents include antibody molecules. As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes one or two heavy (H) chain variable domain sequences and/or one of two light (L) chain variable domain sequence. Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a VH or VHH domain; (vi) a dAb fragment, which consists of a VH domain; (vii) a camelid or camelized variable domain; and (viii) a single chain Fv (scFv).

The VH and VL regions can be further subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an IL-13 binding agent that comprises determinants that form an interface that binds to the IL-13, e.g., a mammalian IL-13, e.g., human or non-human primate IL-13, or an epitope thereof. With respect to proteins (or protein mimetics), the antigen-binding site typically includes one or more loops (of at least four amino acids or amino acid mimics) that form an interface that binds to IL-13. Typically, the antigen-binding site of an antibody molecule includes at least one or two CDRs, or more typically at least three, four, five or six CDRs.

An “epitope” refers to the site on a target compound that is bound by a binding agent, e.g., an antibody molecule. An epitope can be a linear or conformational epitope, or a combination thereof. In the case where the target compound is a protein, for example, an epitope may refer to the amino acids that are bound by the binding agent. Overlapping epitopes include at least one common amino acid residue.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)). Numerous methods are available for obtaining antibody molecules.

One exemplary method of generating antibody molecules includes screening protein expression libraries, e.g., phage or ribosome display libraries. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809. In addition to the use of display libraries, other methods can be used to obtain an anti-IL-13 antibody molecule. For example, an IL-13 protein or a peptide thereof can be used as an antigen in a non-human animal, e.g., a rodent, e.g., a mouse, hamster, or rat.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

In another embodiment, a monoclonal antibody is obtained from the non-human animal, and then modified, e.g., humanized or deimmunized. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies can be generated by replacing sequences of the Fv variable domain that are not directly involved in antigen binding with equivalent sequences from human Fv variable domains. Exemplary methods for generating humanized antibody molecules are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

An antibody molecule may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used.

Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Additionally, chimeric, humanized, and single-chain antibody molecules (e.g., proteins that include both human and nonhuman portions), may be produced using standard recombinant DNA techniques. Humanized antibodies may also be produced, for example, using transgenic mice that express human heavy and light chain genes, but are incapable of expressing the endogenous mouse immunoglobulin heavy and light chain genes.

Additionally, the antibody molecules described herein also include those that bind to IL-13, interfere with the formation of a functional IL-13 signaling complex, and have mutations in the constant regions of the heavy chain. It is sometimes desirable to mutate and inactivate certain fragments of the constant region. For example, mutations in the heavy constant region can be made to produce antibodies with reduced binding to the Fc receptor (FcR) and/or complement; such mutations are well known in the art. An example of such a mutation to the amino sequence of the constant region of the heavy chain of IgG is provided in SEQ ID NO:128. Certain active fragments of the CL and CH subunits (e.g., CH1) are covalently link to each other. A further aspect provides a method for obtaining an antigen-binding site that is specific for a surface of IL-13 that participates in forming a functional IL-13 signaling complex.

Exemplary antibody molecules can include sequences of VL chains as set forth in SEQ ID NOs:30-46, and/or of VH chains as set forth in and SEQ ID NOs:50-115, but also can include variants of these sequences that retain IL-13 binding ability. Such variants may be derived from the provided sequences using techniques well known in the art. Amino acid substitutions, deletions, or additions, can be made in either the FRs or in the CDRs. Whereas changes in the framework regions are usually designed to improve stability and reduce immunogenicity of the antibody molecule, changes in the CDRs are usually designed to increase affinity of the antibody molecule for its target. Such affinity-increasing changes are typically determined empirically by altering the CDR region and testing the antibody molecule. Such alterations can be made according to the methods described in Antibody Engineering, 2nd. ed. (1995), ed. Borrebaeck, Oxford University Press.

An exemplary method for obtaining a heavy chain variable domain sequence that is a variant of a heavy chain variable domain sequence described herein, includes adding, deleting, substituting, or inserting one or more amino acids in a heavy chain variable domain sequence described herein, optionally combining the heavy chain variable domain sequence with one or more light chain variable domain sequences, and testing a protein that includes the modified heavy chain variable domain sequence for specific binding to IL-13, and (preferably) testing the ability of such antigen-binding domain to modulate one or more IL-13-associated activities. An analogous method may be employed using one or more sequence variants of a light chain variable domain sequence described herein.

Variants of antibody molecules can be prepared by creating libraries with one or more varied CDRs and screening the libraries to find members that bind to IL-13, e.g., with improved affinity. For example, Marks et al. (Bio/Technology (1992) 10:779-83) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. The repertoire may be combined with a CDR3 of a particular antibody. Further, the CDR3-derived sequences may be shuffled with repertoires of VH or VL domains lacking a CDR3, and the shuffled complete VH or VL domains combined with a cognate VL or VH domain to provide specific antigen-binding fragments. The repertoire may then be displayed in a suitable host system such as the phage display system of WO 92/01047, so that suitable antigen-binding fragments can be selected. Analogous shuffling or combinatorial techniques are also disclosed by Stemmer (Nature (1994) 370:389-91). A further alternative is to generate altered VH or VL regions using random mutagenesis of one or more selected VH and/or VL genes to generate mutations within the entire variable domain. See, e.g., Gram et al. Proc. Nat. Acad. Sci. USA (1992) 89:3576-80.

Another method that may be used is to direct mutagenesis to CDR regions of VH or VL genes. Such techniques are disclosed by, e.g., Barbas et al. (Proc. Nat. Acad. Sci. USA (1994) 91:3809-13) and Schier et al. (J. Mol. Biol. (1996) 263:551-67). Similarly, one or more, or all three CDRs may be grafted into a repertoire of VH or VL domains, or even some other scaffold (such as a fibronectin domain). The resulting protein is evaluated for ability to bind to IL-13.

In one embodiment, a binding agent that binds to a target is modified, e.g., by mutagenesis, to provide a pool of modified binding agents. The modified binding agents are then evaluated to identify one or more altered binding agents which have altered functional properties (e.g., improved binding, improved stability, lengthened stability in vivo). In one implementation, display library technology is used to select or screen the pool of modified binding agents. Higher affinity binding agents are then identified from the second library, e.g., by using higher stringency or more competitive binding and washing conditions. Other screening techniques can also be used.

In some embodiments, the mutagenesis is targeted to regions known or likely to be at the binding interface. If, for example, the identified binding agents are antibody molecules, then mutagenesis can be directed to the CDR regions of the heavy or light chains as described herein. Further, mutagenesis can be directed to framework regions near or adjacent to the CDRs, e.g., framework regions, particular within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable domain being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may including using two germline sequences that separately contribute to the amino-terminal portion and the carboxy-terminal portion. In other implementations more than one or two germline sequences are used, e.g., to form a consensus sequence.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem. 22:259-306. Removal of any carbohydrate moieties present on the antibodies may be accomplished chemically or enzymatically as described in the art (Hakimuddin et al. (1987) Arch. Biochem. Biophys. 259:52; Edge et al. (1981) Anal. Biochem. 118:131; and Thotakura et al. (1987) Meth. Enzymol. 138:350). See, e.g., U.S. Pat. No. 5,869,046 for a modification that increases in vivo half life by providing a salvage receptor binding epitope.

In one embodiment, the anti-IL-13 antibody molecule includes at least one, two and preferably three CDRs from the light or heavy chain variable domain of an antibody disclosed herein, e.g., MJ 2-7. For example, the protein includes one or more of the following sequences within a CDR region:

GFNIKDTYIH (SEQ ID NO:15),

RIDPANDNIKYDPKFQG (SEQ ID NO:16),

SEENWYDFFDY (SEQ ID NO:17),

RSSQSIVHSNGNTYLE (SEQ ID NO:18),

KVSNRFS (SEQ ID NO:19), and

FQGSHIPYT (SEQ ID NO:20), or a CDR having an amino acid sequence that differs by no more than 4, 3, 2.5, 2, 1.5, 1, or 0.5 alterations (e.g., substitutions, insertions or deletions) for every 10 amino acids (e.g., the number of differences being proportional to the CDR length) relative to a sequence listed above, e.g., at least one alteration but not more than two, three, or four per CDR.

For example, the anti-IL-13 antibody molecule can include, in the light chain variable domain sequence, at least one, two, or three of the following sequences within a CDR region:

RSSQSIVHSNGNTYLE (SEQ ID NO:18),

KVSNRFS (SEQ ID NO:19), and

FQGSHIPYT (SEQ ID NO:20), or an amino acid sequence that differs by no more than 4, 3, 2.5, 2, 1.5, 1, or 0.5 substitutions, insertions or deletions for every 10 amino acids relative to a sequence listed above.

The anti-IL-13 antibody molecule can include, in the heavy chain variable domain sequence, at least one, two, or three of the following sequences within a CDR region:

GFNIKDTYIH (SEQ ID NO:15),

RIDPANDNIKYDPKFQG (SEQ ID NO:16), and

SEENWYDFFDY (SEQ ID NO: 17), or an amino acid sequence that differs by no more than 4, 3, 2.5, 2, 1.5, 1, or 0.5 substitutions, insertions or deletions for every 10 amino acids relative to a sequence listed above. The heavy chain CDR3 region can be less than 13 or less than 12 amino acids in length, e.g., 11 amino acids in length (either using Chothia or Kabat definitions).

In another example, the anti-IL-13 antibody molecule can include, in the light chain variable domain sequence, at least one, two, or three of the following sequences within a CDR region (amino acids in parentheses represent alternatives for a particular position):

(i)

(SEQ ID NO:25)

(RK)-S-S-Q-S-(LI)-(KV)-H-S-(ND)-G-N-(TN)-Y-L-
(EDNQYAS)
or

(SEQ ID NO:26)

(RK)-S-S-Q-S-(LI)-(KV)-H-S-(ND)-G-N-(TN)-Y-L-E,
or

(SEQ ID NO:21)

(RK)-S-S-Q-S-(LI)-(KV)-H-S-N-G-N-T-Y-L-(EDNQYAS),
(ii)

(SEQ ID NO:27)

K-(LVI)-S-(NY)-(RW)-(FD)-S,
or

(SEQ ID NO:22)

K-(LV)-S-(NY)-R-F-S,
and
(iii)

(SEQ ID NO:28)

Q-(GSA)-(ST)-(HEQ)-I-P,

(SEQ ID NO:23)

F-Q-(GSA)-(SIT)-(HEQ)-(IL)-P,
or

(SEQ ID NO:194)

Q-(GSA)-(ST)-(HEQ)-I-P-Y-T,
or

(SEQ ID NO:29)

F-Q-(GSA)-(SIT)-(HEQ)-(IL)-P-Y-T.

In one preferred embodiment, the anti-IL-13 antibody molecule includes all six CDR's from MJ 2-7 or closely related CDRs, e.g., CDRs which are identical or which have at least one amino acid alteration, but not more than two, three or four alterations (e.g., substitutions, deletions, or insertions). The IL-13 binding agent can include at least two, three, four, five, six, or seven IL-13 contacting amino acid residues of MJ 2-7 In still another example, the anti-IL-13 antibody molecule includes at least one, two, or three CDR regions that have the same canonical structures and the corresponding CDR regions of MJ 2-7, e.g., at least CDR1 and CDR2 of the heavy and/or light chain variable domains of MJ 2-7.

In another example, the anti-IL-13 antibody molecule can include, in the heavy chain variable domain sequence, at least one, two, or three of the following sequences within a CDR region (amino acids in parentheses represent alternatives for a particular position):

(SEQ ID NO:48)

| | |
|---|---|
| (i) | G-(YF)-(NT)-I-K-D-T-Y-(MI)-H, |

(SEQ ID NO:49)

| | |
|---|---|
| (ii) | (WR)-I-D-P-(GA)-N-D-N-I-K-Y-(SD)-(PQ)-K-F- |
| Q-G, | |
| and | |

(SEQ ID NO:17)

| | |
|---|---|
| (iii) | SEENWYDFFDY. |

In one embodiment, the anti-IL-13 antibody molecule includes at least one, two and preferably three CDR's from the light or heavy chain variable domain of an antibody disclosed herein, e.g., C65. For example, the anti-IL-13 antibody molecule includes one or more of the following sequences within a CDR region:

QASQGTSINLN (SEQ ID NO:118),

GASNLED (SEQ ID NO:119), and

LQHSYLPWT (SEQ ID NO:120)

GFSLTGYGVN (SEQ ID NO:121),

IIWGDGSTDYNSAL (SEQ ID NO:122), and

DKTFYYDGFYRGRMDY (SEQ ID NO:123), or a CDR having an amino acid sequence that differs by no more than 4, 3, 2.5, 2, 1.5, 1, or 0.5 substitutions, insertions or deletions for every 10 amino acids (e.g., the number of differences being proportional to the CDR length) relative to a sequence listed above, e.g., at least one alteration but not more than two, three, or four per CDR. For example, the protein can include, in the light chain variable domain sequence, at least one, two, or three of the following sequences within a CDR region:

QASQGTSINLN (SEQ ID NO:118),

GASNLED (SEQ ID NO:119), and

LQHSYLPWT (SEQ ID NO:120), or an amino acid sequence that differs by no more than 4, 3, 2.5, 2, 1.5, 1, or 0.5 substitutions, insertions or deletions for every 10 amino acids relative to a sequence listed above.

The anti-IL-13 antibody molecule can include, in the heavy chain variable domain sequence, at least one, two, or three of the following sequences within a CDR region:

GFSLTGYGVN (SEQ ID NO:121),

IIWGDGSTDYNSAL (SEQ ID NO:122), and

DKTFYYDGFYRGRMDY (SEQ ID NO:123), or an amino acid sequence that differs by no more than 4, 3, 2.5, 2, 1.5, 1, or 0.5 substitutions, insertions or deletions for every 10 amino acids relative to a sequence listed above.

In embodiments, the IL-13 antibody molecule can include one of the following sequences:

(SEQ ID NO:30)

DIVMTQTPLSLPVTPGEPASISCRSSQSIVHSNGNTYLEWYLQKPGQSPQ
LLIYKVSNRFSGVPDRFSGSGSGTDFTLKISRVEAEDVGVYYCFQGSHIP
YT

(SEQ ID NO:31)

DVVMTQSPLSLPVTLGQPASISCRSSQSIVHSNGNTYLEWFQQRPGQSPR
RLIYKVSNRFSGVPDRFSGSGSGTDFTLKISRVEAEDVGVYYCFQGSHIP
YT

(SEQ ID NO:32)

DIVMTQTPLSLSVTPGQPASISCRSSQSIVHSNGNTYLEWYLQKPGQSPQ
LLIYKVSNRFSGVPDRFSGSGSGTDFTLKISRVEAEDVGVYYCFQGSHIP
YT

(SEQ ID NO:33)

DIVMTQTPLSLSVTPGQPASISCRSSQSIVHSNGNTYLEWYLQKPGQPPQ
LLIYKVSNRFSGVPDRFSGSGSGTDFTLKISRVEAEDVGVYYCFQGSHIP
YT

(SEQ ID NO:34)

DIVMTQSPLSLPVTPGEPASISCRSSQSIVHSNGNTYLEWYLQKPGQSPQ
LLIYKVSNRFSGVPDRFSGSGSGTDFTLKISRVEAEDVGVYYCFQGSHIP
YT

(SEQ ID NO:35)

DIVMTQTPLSSPVTLGQPASISCRSSQSIVHSNGNTYLEWLQQRPGQPPR
LLIYKVSNRFSGVPDRFSGSGAGTDFTLKISRVEAEDVGVYYCFQGSHIP
YT

(SEQ ID NO:36)

DIQMTQSPSSLSASVGDRVTITCRSSQSIVHSNGNTYLEWYQQKPGKAPK
LLIYKVSNRFSGVPSRFSGSGSGTDFTLTISSLQPEDFATYYCFQGSHIP
YT

(SEQ ID NO:37)

DVVMTQSPLSLPVTLGQPASISCRSSQSLVYSDGNTYLNWFQQRPGQSPR
RLIYKVSNRFSGVPDRFSGSGSGTDFTLKISRVEAEDVGVYYCFQGSHIP
YT

(SEQ ID NO:38)

DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHIP
YT

or a sequence that has fewer than eight, seven, six, five, four, three, or two alterations (e.g., substitutions, insertions or deletions, e.g., conservative substitutions or a substitution for an amino acid residue at a corresponding position in MJ 2-7). Exemplary substitutions are at one of the following Kabat positions: 2, 4, 6, 35, 36, 38, 44, 47, 49, 62, 64-69, 85, 87, 98, 99, 101, and 102. The substitutions can, for example, substitute an amino acid at a corresponding position from MJ 2-7 into a human framework region.

The IL-13 antibody molecule may also include one of the following sequences:

(SEQ ID NO:39)

DIVMTQTPLSLPVTPGEPASISC-(RK)-S-S-Q-S-(LI)-(KV)-H-
S-(ND)-G-N-(TN)-Y-L-(EDNQYAS) WYLQKPGQSPQLLIYK-
(LVI)-S-(NY)-(RW)-(FD)-SGVPDRFSGSGSGTDFTLKISRVEAED
VGVYYCF-Q-(GSA)-(SIT)(HEQ)(IL)P

(SEQ ID NO:40)

DVVMTQSPLSLPVTLGQPASISC-(RK)-S-S-Q-S-(LI)-(KV)-H-
S-(ND)-G-N-(TN)-Y-L-(EDNQYAS)WFQQRPGQSPRRLIYK-
(LVI)-S-(NY)-(RW)-(FD)-SGVPDRFSGSGSGTDFTLKISRVEAED
VGVYYCF-Q-(GSA)-(SIT)-(HEQ)(IL)P

(SEQ ID NO:41)

DIVMTQTPLSLSVTPGQPASISC-(RK)-S-S-Q-S-(LI)-(KV)-H-
S-(ND)-G-N-(TN)-Y-L-(EDNQYAS)WYLQKPGQSPQLLIYK-
(LVI)-S-(NY)-(RW)-(FD)-SGVPDRFSGSGSGTDFTLKISRVEAED
VGVYYCF-Q-(GSA)-(SIT)-(HEQ)(IL)P

(SEQ ID NO:42)

DIVMTQTPLSLSVTPGQPASISC (RK)-S-S-Q-S-(LI)-(KV)-H-
S-(ND)-G-N-(TN)-Y-L-(EDNQYAS)WYLQKPGQPPQLLIYK-
(LVI)-S-(NY)-(RW)-(FD)-SGVPDRFSGSGSGTDFTLKISRVEAED
VGVYYCF-Q-(GSA)-(SIT)-(HEQ)(IL)P

(SEQ ID NO:43)

DIVMTQSPLSLPVTPGEPASISC (RK)-S-S-Q-S-(LI)-(KV)-H-
S-(ND)-G-N-(TN)-Y-L-(EDNQYAS)WYLQKPGQSPQLLIYK-
(LVI)-S-(NY)-(RW)-(FD)-SGVPDRFSGSGSGTDFTLKISRVEAED
VGVYYCF-Q-(GSA)-(SIT)-(HEQ)(IL)P

(SEQ ID NO:44)

DIVMTQTPLSSPVTLGQPASISC (RK)-S-S-Q-S-(LI)-(KV)-H-
S-(ND)-G-N-(TN)-Y-L-(EDNQYAS)WLQQRPGQPPRLLIYK-
(LVI)-S-(NY)-(RW)-(FD)-SGVPDRFSGSGAGTDFTLKISRVEAED
VGVYYCF-Q-(GSA)-(SIT)-(HEQ)(IL)P

(SEQ ID NO:45)

DIQMTQSPSSLSASVGDRVTITC (RK)-S-S-Q-S-(LI)-(KV)-H-
S-(ND)-G-N-(TN)-Y-L-(EDNQYAS)WYQQKPGKAPKLLIYK-
(LVI)-S-(NY)-(RW)-(FD)-SGVPSRFSGSGSGTDFTLTISSLQPED
FATYYCF-Q-(GSA)-(SIT)-(HEQ)(IL)P

(SEQ ID NO:46)

DVLMTQTPLSLPVSLGDQASISC (RK)-S-S-Q-S-(LI)-(KV)-H-
S-(ND)-G-N-(TN)-Y-L-(EDNQYAS)WYLQKPGQSPKLLIYK-
(LVI)-S-(NY)-(RW)-(FD)-SGVPDRFSGSGSGTDFTLKISRVEAED
LGVYYCF-Q-(GSA)-(SIT)-(HEQ)(IL)P

or a sequence that has fewer than eight, seven, six, five, four, three, or two alterations (e.g., substitutions, insertions or deletions, e.g., conservative substitutions or a substitution for an amino acid residue at a corresponding position in MJ 2-7) in the framework region. Exemplary substitutions are at one or more of the following Kabat positions: 2, 4, 6, 35, 36, 38, 44, 47, 49, 62, 64-69, 85, 87, 98, 99, 101, and 102. The substitutions can, for example, substitute an amino acid at a corresponding position from MJ 2-7 into a human framework region. The sequences may also be followed by the dipeptide Tyr-Thr. The FR4 region can include, e.g., the sequence FGGGTKVEIKR (SEQ ID NO:47).

In other embodiments, the IL-13 antibody molecule can include one of the following sequences:

(SEQ ID NO:50)

QVQLVQSGAEVKKPGASVKVSCKASGFNIKDTYIHWVRQAPGQGLEWMGR
IDPANDNIKYDPKFQGRVTMTRDTSISTAYMELSRLRSDDTAVYYCARSE
ENWYDFFDY

(SEQ ID NO:51)

QVQLVQSGAEVKKPGASVKVSCKASGFNIKDTYIHWVRQAPGQRLEWMGR
IDPANDNIKYDPKFQGRVTITRDTSASTAYMELSSLRSEDTAVYYCARSE
ENWYDFFDY

(SEQ ID NO:52)

QVQLVQSGAEVKKPGASVKVSCKASGFNIKDTYIHWVRQATGQGLEWMGR
IDPANDNIKYDPKFQGRVTMTRNTSISTAYMELSSLRSEDTAVYYCARSE
ENWYDFFDY

(SEQ ID NO:53)

QVQLVQSGAEVKKPGASVKVSCKASGFNIKDTYIHWVRQAPGQGLEWMGR
IDPANDNIKYDPKFQGRVTMTTDTSTSTAYMELRSLRSDDTAVYYCARSE
ENWYDFFDY

(SEQ ID NO:54)

QVQLVQSGAEVKKPGASVKVSCKVSGFNIKDTYIHWVRQAPGKGLEWMGR
IDPANDNIKYDPKFQGRVTMTEDTSTDTAYMELSSLRSEDTAVYYCATSE
ENWYDFFDY

(SEQ ID NO:55)

QMQLVQSGAEVKKTGSSVKVSCKASGFNIKDTYIHWVRQAPGQALEWMGR
IDPANDNIKYDPKFQGRVTITRDRSMSTAYMELSSLRSEDTAMYYCARSE
ENWYDFFDY

(SEQ ID NO:56)

QVQLVQSGAEVKKPGASVKVSCKASGFNIKDTYIHWVRQAPGQGLEWMGR
IDPANDNIKYDPKFQGRVTMTRDTSTSTVYMELSSLRSEDTAVYYCARSE
ENWYDFFDY

(SEQ ID NO:57)

QMQLVQSGPEVKKPGTSVKVSCKASGFNIKDTYIHWVRQARGQRLEWIGR
IDPANDNIKYDPKFQGRVTITRDMSTSTAYMELSSLRSEDTAVYYCAASE
ENWYDFFDY

(SEQ ID NO:58)

EVQLVESGGGLVQPGGSLRLSCAASGFNIKDTYIHWVRQAPGKGLEWVAR
IDPANDNIKYDPKFQGRFTISRDNAKNSLYLQMNSLRAEDTAVYYCARSE
ENWYDFFDY

(SEQ ID NO:59)

EVQLVESGGGLVQPGRSLRLSCAASGFNIKDTYIHWVRQAPGKGLEWVSR
IDPANDNIKYDPKFQGRFTISRDNAKNSLYLQMNSLRAEDTALYYCAKSE
EENWYDFFDY

(SEQ ID NO:60)

QVQLVESGGGLVKPGGSLRLSCAASGFNIKDTYIHWIRQAPGKGLEWVSR
IDPANDNIKYDPKFQGRFTISRDNAKNSLYLQMNSLRAEDTAVYYCARSE
ENWYDFFDY

(SEQ ID NO:61)

EVQLVESGGGLVKPGGSLRLSCAASGFNIKDTYIHWVRQAPGKGLEWVGR
IDPANDNIKYDPKFQGRFTISRDDSKNTLYLQMNSLKTEDTAVYYCTTSE
ENWYDFFDY

(SEQ ID NO:62)

EVQLVESGGGVVRPGGSLRLSCAASGFNIKDTYIHWVRQAPGKGLEWVSR
IDPANDNIKYDPKFQGRFTISRDNAKNSLYLQMNSLRAEDTALYHCARSE
ENWYDFFDY

(SEQ ID NO:63)

EVQLVESGGGLVKPGGSLRLSCAASGFNIKDTYIHWVRQAPGKGLEWVSR
IDPANDNIKYDPKFQGRFTISRDNAKNSLYLQMNSLRAEDTAVYYCARSE
ENWYDFFDY

(SEQ ID NO:64)

EVQLLESGGGLVQPGGSLRLSCAASGFNIKDTYIHWVRQAPGKGLEWVSR
IDPANDNIKYDPKFQGRFTISRDNSKNTLYLQMNSLRAEDTAVYYCAKSE
ENWYDFFDY

(SEQ ID NO:65)

QVQLVESGGGVVQPGRSLRLSCAASGFNIKDTYIHWVRQAPGKGLEWVAR
IDPANDNIKYDPKFQGRFTISRDNSKNTLYLQMNSLRAEDTAVYYCAKSE
ENWYDFFDY

(SEQ ID NO:66)

QVQLVESGGGVVQPGRSLRLSCAASGFNIKDTYIHWVRQAPGKGLEWVAR
IDPANDNIKYDPKFQGRFTISRDNSKNTLYLQMNSLRAEDTAVYYCARSE
ENWYDFFDY

(SEQ ID NO:67)

EVQLVESGGVVVQPGGSLRLSCAASGFNIKDTYIHWVRQAPGKGLEWVSR
IDPANDNIKYDPKFQGRFTISRDNSKNSLYLQMNSLRTEDTALYYCAKDS
EENWYDFFDY

(SEQ ID NO:68)

EVQLVESGGGLVQPGGSLRLSCAASGFNIKDTYIHWVRQAPGKGLEWVSR
IDPANDNIKYDPKFQGRFTISRDNAKNSLYLQMNSLRDEDTAVYYCARSE
ENWYDFFDY

(SEQ ID NO:69)

EVQLVESGGGLVQPGRSLRLSCTASGFNIKDTYIHWFRQAPGKGLEWVGR
IDPANDNIKYDPKFQGRFTISRDGSKSIAYLQMNSLKTEDTAVYYCTRSE
ENWYDFFDY

(SEQ ID NO:70)

EVQLVESGGGLVQPGGSLRLSCAASGFNIKDTYIHWVRQAPGKGLEYVSR
IDPANDNIKYDPKFQGRFTISRDNSKNTLYLQMGSLRAEDMAVYYCARSE
ENWYDFFDY

(SEQ ID NO:71)

EVQLVESGGGLVQPGGSLRLSCAASGFNIKDTYIHWVRQAPGKGLEWIGR
IDPANDNIKYDPKFQGRFTISRDNAKNSLYLQMNSLRAEDTAVYYCARSE
ENWYDFFDY

(SEQ ID NO:72)

EVQLVESGGGLVQPGGSLRLSCAASGFNIKDTYIHWVRQAPGKGLEWVAR
IDPANDNIKYDPKFQGKATISRDNAKNSLYLQMNSLRAEDTAVYYCARSE
ENWYDFFDY

(SEQ ID NO:73)

EVQLVESGGGLVQPGGSLRLSCAASGFNIKDTYIHWVRQAPGKGLEWVAR
IDPANDNIKYDPKFQGRFTISADNAKNSLYLQMNSLRAEDTAVYYCARSE
ENWYDFFDY

(SEQ ID NO:74)

EVQLVESGGGLVQPGGSLRLSCAASGFNIKDTYIHWVRQAPGKGLEWVGR
IDPANDNIKYDPKFQGRFTISRDNAKNSLYLQMNSLRAEDTAVYYCARSE
ENWYDFFDY

(SEQ ID NO:75)

EVQLVESGGGLVQPGGSLRLSCAASGFNIKDTYIHWVRQAPGKGLEWVAR
IDPANDNIKYDPKFQGKATISADNAKNSLYLQMNSLRAEDTAVYYCARSE
ENWYDFFDY

(SEQ ID NO:76)

EVQLVESGGGLVQPGGSLRLSCAASGFNIKDTYIHWVRQAPGKGLEWIGR
IDPANDNIKYDPKFQGRFTISADNAKNSLYLQMNSLRAEDTAVYYCARSE
ENWYDFFDY

(SEQ ID NO:77)

EVQLVESGGGLVQPGGSLRLSCAASGFNIKDTYIHWVRQAPGKGLEWVGR
IDPANDNIKYDPKFQGRFTISADNAKNSLYLQMNSLRAEDTAVYYCARSE
ENWYDFFDY

(SEQ ID NO:78)

EVQLVESGGGLVQPGGSLRLSCAASGFNIKDTYIHWVRQAPGKGLEWVAR
IDPANDNIKYDPKFQGRFTISRDNAKNSAYLQMNSLRAEDTAVYYCARSE
ENWYDFFDY

(SEQ ID NO:79)

EVQLVESGGGLVQPGGSLRLSCAASGFNIKDTYIHWVRQAPGKGLEWVGR
IDPANDNIKYDPKFQGRFTISADNAKNSAYLQMNSLRAEDTAVYYCARSE
ENWYDFFDY

(SEQ ID NO:80)

EVQLVESGGGLVQPGGSLRLSCAASGFNIKDTYIHWVRQAPGKGLEWIGR
IDPANDNIKYDPKFQGRFTISADNAKNSAYLQMNSLRAEDTAVYYCARSE
ENWYDFFDY

(SEQ ID NO:81)

EVQLVESGGGLVQPGGSLRLSCTGSGFNIKDTYIHWVRQAPGKGLEWIGR
IDPANDNIKYDPKFQGRFTISADNAKNSLYLQMNSLRAEDTAVYYCARSE
ENWYDFFDY

(SEQ ID NO:82)

EVQLQQSGAELVKPGASVKLSCTGSGFNIKDTYIHWVKQRPEQGLEWIGR
IDPANDNIKYDPKFQGKATITADTSSNTAYLQLNSLTSEDTAVYYCARSE
ENWYDFFDY

or a sequence that has fewer than eight, seven, six, five, four, three, or two alterations

(e.g., substitutions, insertions or deletions, e.g., conservative substitutions or a substitution for an amino acid residue at a corresponding position in MJ 2-7). Exemplary substitutions are at one or more of the following Kabat positions: 2, 4, 6, 25, 36, 37, 39, 47, 48, 93, 94, 103, 104, 106, and 107. Exemplary substitutions can also be at one or more of the following positions (accordingly to sequential numbering): 48, 49, 67, 68, 72, and 79. The substitutions can, for example, substitute an amino acid at a corresponding position from MJ 2-7 into a human framework region. In one embodiment, the sequence includes (accordingly to sequential numbering) one or more of the following: Ile at 48, Gly at 49, Lys at 67, Ala at 68, Ala at 72, and Ala at 79; preferably, e.g., Ile at 48, Gly at 49, Ala at 72, and Ala at 79.

Further, the frameworks of the heavy chain variable domain sequence can include: (i) at a position corresponding to 49, Gly; (ii) at a position corresponding to 72, Ala; (iii) at positions corresponding to 48, Ile, and to 49, Gly; (iv) at positions corresponding to 48, Ile, to 49, Gly, and to 72, Ala; (v) at positions corresponding to 67, Lys, to 68, Ala, and to 72, Ala; and/or (vi) at positions corresponding to 48, Ile, to 49, Gly, to 72, Ala, to 79, Ala.

The IL-13 antibody molecule may also include one of the following sequences:

(SEQ ID NO:83)

QVQLVQSGAEVKKPGASVKVSCKASG-(YF)-(NT)-I-K-D-T-Y-
(MI)-H, WVRQAPGQGLEWMG (WR)-I-D-P-(GA)-N-D-N-I-K-
Y-(SD)-(PQ)-K-F-Q-GRVTMTRDTSISTAYMELSRLRSDDTAVYYCA
RSEENWYDFFDY

(SEQ ID NO:84)

QVQLVQSGAEVKKPGASVKVSCKASG-(YF)-(NT)-I-K-D-T-Y-
(MI)-H, WVRQAPGQRLEWMG (WR)-I-D-P-(GA)-N-D-N-I-K-
Y-(SD)-(PQ)-K-F-Q-GRVTITRDTSASTAYMELSSLRSEDTAVYYCA
RSEENWYDFFDY

(SEQ ID NO:85)

QVQLVQSGAEVKKPGASVKVSCKASG-(YF)-(NT)-I-K-D-T-Y-
0
(MI)-H, WVRQATGQGLEWMG (WR)-I-D-P-(GA)-N-D-N-I-K-
Y-(SD)-(PQ)-K-F-Q-GRVTMTRNTSISTAYMELSSLRSEDTAVYYCA
RSEENWYDFFDY

(SEQ ID NO:86)

QVQLVQSGAEVKKPGASVKVSCKASG-(YF)-(NT)-I-K-D-T-Y-
(MI)-H, WVRQAPGQGLEWMG (WR)-I-D-P-(GA)-N-D-N-I-K-
Y-(SD)-(PQ)-K-F-Q-GRVTMTTDTSTSTAYMELRSLRSDDTAVYYCA
RSEENWYDFFDY

(SEQ ID NO:87)

QVQLVQSGAEVKKPGASVKVSCKVSG-(YF)-(NT)-I-K-D-T-Y-
(MI)-H, WVRQAPGKGLEWMG (WR)-I-D-P-(GA)-N-D-N-I-K-
Y-(SD)-(PQ)-K-F-Q-GRVTMTEDTSTDTAYMELSSLRSEDTAVYYCA
TSEENWYDFFDY

(SEQ ID NO:88)

QMQLVQSGAEVKKTGSSVKVSCKASG-(YF)-(NT)-I-K-D-T-Y-
(MI)-H, WVRQAPGQALEWMG (WR)-I-D-P-(GA)-N-D-N-I-K-
Y-(SD)-(PQ)-K-F-Q-GRVTITRDRSMSTAYMELSSLRSEDTAMYYCA
RSEENWYDFFDY

(SEQ ID NO:89)

QVQLVQSGAEVKKPGASVKVSCKASG-(YF)-(NT)-I-K-D-T-Y-
(MI)-H, WVRQAPGQGLEWMG (WR)-I-D-P-(GA)-N-D-N-I-K-
Y-(SD)-(PQ)-K-F-Q-GRVTMTRDTSTSTVYMELSSLRSEDTAVYYCA
RSEENWYDFFDY

(SEQ ID NO:90)

QMQLVQSGPEVKKPGTSVKVSCKASG-(YF)-(NT)-I-K-D-T-Y-
(MI)-H, WVRQARGQRLEWIG (WR)-I-D-P-(GA)-N-D-N-I-K-
Y-(SD)-(PQ)-K-F-Q-GRVTITRDMSTSTAYMELSSLRSEDTAVYYCA
ASEENWYDFFDY

(SEQ ID NO:91)

EVQLVESGGGLVQPGGSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GRFTISRDNAKNSLYLQMNSLRAEDTAVYYCA
RSEENWYDFFDY

(SEQ ID NO:92)

EVQLVESGGGLVQPGRSLRLSCAASG-(YF)-(NT)-I-K-D-T-Y-
(MI)-H, WVRQAPGKGLEWVS (WR)-I-D-P-(GA)-N-D-N-I-K-
Y-(SD)-(PQ)-K-F-Q-GRFTISRDNAKNSLYLQMNSLRAEDTALYYCA
KDSEENWYDFFDY

(SEQ ID NO:93)

QVQLVESGGGLVKPGGSLRLSCAASG-(YF)-(NT)-I-K-D-T-Y-
(MI)-H, WIRQAPGKGLEWVS (WR)-I-D-P-(GA)-N-D-N-I-K-
Y-(SD)-(PQ)-K-F-Q-GRFTISRDNAKNSLYLQMNSLRAEDTAVYYCA
RSEENWYDFFDY

(SEQ ID NO:94)

EVQLVESGGGLVKPGGSLRLSCAASG-(YF)-(NT)-I-K-D-T-Y-
(MI)-H, WVRQAPGKGLEWVG (WR)-I-D-P-(GA)-N-D-N-I-K-
Y-(SD)-(PQ)-K-F-Q-GRFTISRDDSKNTLYLQMNSLKTEDTAVYYCT
TSEENWYDFFDY

(SEQ ID NO:95)

EVQLVESGGGVVRPGGSLRLSCAASG-(YF)-(NT)-I-K-D-T-Y-
(MI)-H, WVRQAPGKGLEWVS (WR)-I-D-P-(GA)-N-D-N-I-K-Y
(SD)-(PQ)-K-F-Q-GRFTISRDNAKNSLYLQMNSLRAEDTALYHCARS
EENWYDFFDY

(SEQ ID NO:96)

EVQLVESGGGLVKPGGSLRLSCAASG-(YF)-(NT)-I-K-D-T-Y-
(MI)-H, WVRQAPGKGLEWVS (WR)-I-D-P-(GA)-N-D-N-I-K-
Y-(SD)-(PQ)-K-F-Q-GRFTISRDNAKNSLYLQMNSLRAEDTAVYYCA
RSEENWYDFFDY

(SEQ ID NO:97)

EVQLLESGGGLVQPGGSLRLSCAASG-(YF)-(NT)-I-K-D-T-Y-
(MI)-H, WVRQAPGKGLEWVS (WR)-I-D-P-(GA)-N-D-N-I-K-
Y-(SD)-(PQ)-K-F-Q-GRFTISRDNSKNTLYLQMNSLRAEDTAVYYCA
KSEENWYDFFDY

(SEQ ID NO:98)

QVQLVESGGGVVQPGRSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GRFTISRDNSKNTLYLQMNSLRAEDTAVYYCA
KSEENWYDFFDY

(SEQ ID NO:99)

QVQLVESGGGVVQPGRSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GRFTISRDNSKNTLYLQMNSLRAEDTAVYYCA
RSEENWYDFFDY

(SEQ ID NO:100)

EVQLVESGGVVVQPGGSLRLSCAASG-(YF)-(NT)-I-K-D-T-Y-
(MI)-H, WVRQAPGKGLEWVS (WR)-I-D-P-(GA)-N-D-N-I-K-
Y-(SD)-(PQ)-K-F-Q-GRFTISRDNSKNSLYLQMNSLRTEDTALYYCA
KDSEENWYDFFDY

(SEQ ID NO:101)

EVQLVESGGGLVQPGGSLRLSCAASG-(YF)-(NT)-I-K-D-T-Y-
(MI)-H, WVRQAPGKGLEWVS (WR)-I-D-P-(GA)-N-D-N-I-K-
Y-(SD)-(PQ)-K-F-Q-GRFTISRDNAKNSLYLQMNSLRDEDTAVYYCA
RSEENWYDFFDY

(SEQ ID NO:102)

EVQLVESGGGLVQPGRSLRLSCTASG-(YF)-(NT)-I-K-D-T-Y-
(MI)-H, WFRQAPGKGLEWVG (WR)-I-D-P-(GA)-N-D-N-I-K-
Y-(SD)-(PQ)-K-F-Q-GRFTISRDGSKSIAYLQMNSLKTEDTAVYYCT
RSEENWYDFFDY

(SEQ ID NO:103)

EVQLVESGGGLVQPGGSLRLSCAASG-(YF)-(NT)-I-K-D-T-Y-
(MI)-H, WVRQAPGKGLEYVS (WR)-I-D-P-(GA)-N-D-N-I-K-
Y-(SD)-(PQ)-K-F-Q-GRFTISRDNSKNTLYLQMGSLRAEDMAVYYCA
RSEENWYDFFDY

(SEQ ID NO:104)

EVQLVESGGGLVQPGGSLRLSCAASG-(YF)-(NT)-I-K-D-T-Y-
(MI)-H, WVRQAPGKGLEWIG (WR)-I-D-P-(GA)-N-D-N-I-K-
Y-(SD)-(PQ)-K-F-Q-GRFTISRDNAKNSLYLQMNSLRAEDTAVYYCA
RSEENWYDFFDY

(SEQ ID NO:105)

EVQLVESGGGLVQPGGSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GKATISRDNAKNSLYLQMNSLRAEDTAVYYCA
RSEENWYDFFDY

(SEQ ID NO:106)

EVQLVESGGGLVQPGGSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GRFTISADNAKNSLYLQMNSLRAEDTAVYYCA
RSEENWYDFFDY

(SEQ ID NO:107)

EVQLVESGGGLVQPGGSLRLSCAASG-(YF)-(NT)-I-K-D-T-Y-
(MI)-H, WVRQAPGKGLEWVG (WR)-I-D-P-(GA)-N-D-N-I-K-
Y-(SD)-(PQ)-K-F-Q-GRFTISRDNAKNSLYLQMNSLRAEDTAVYYCA
RSEENWYDFFDY

(SEQ ID NO:108)

EVQLVESGGGLVQPGGSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GKATISADNAKNSLYLQMNSLRAEDTAVYYCA
RSEENWYDFFDY

(SEQ ID NO:109)

EVQLVESGGGLVQPGGSLRLSCAASG-(YF)-(NT)-I-K-D-T-Y-
(MI)-H, WVRQAPGKGLEWIG (WR)-I-D-P-(GA)-N-D-N-I-K-
Y-(SD)-(PQ)-K-F-Q-GRFTISADNAKNSLYLQMNSLRAEDTAVYYCA
RSEENWYDFFDY

(SEQ ID NO:110)

EVQLVESGGGLVQPGGSLRLSCAASG-(YF)-(NT)-I-K-D-T-Y-
(MI)-H, WVRQAPGKGLEWVG (WR)-I-D-P-(GA)-N-D-N-I-K-
Y-(SD)-(PQ)-K-F-Q-GRFTISADNAKNSLYLQMNSLRAEDTAVYYCA
RSEENWYDFFDY

(SEQ ID NO:111)

EVQLVESGGGLVQPGGSLRLSCAASG-(YF)-(NT)-I-K-D-T-Y-
(MI)-H, WVRQAPGKGLEWVA (WR)-I-D-P-(GA)-N-D-N-I-K-
Y-(SD)-(PQ)-K-F-Q-GRFTISRDNAKNSAYLQMNSLRAEDTAVYYCA
RSEENWYDFFDY

(SEQ ID NO:112)

EVQLVESGGGLVQPGGSLRLSCAASG-(YF)-(NT)-I-K-D-T-Y-
(MI)-H, WVRQAPGKGLEWVG (WR)-I-D-P-(GA)-N-D-N-I-K-
Y-(SD)-(PQ)-K-F-Q-GRFTISADNAKNSAYLQMNSLRAEDTAVYYCA
RSEENWYDFFDY

(SEQ ID NO:113)

EVQLVESGGGLVQPGGSLRLSCAASG-(YF)-(NT)-I-K-D-T-Y-
(MI)-H, WVRQAPGKGLEWIG (WR)-I-D-P-(GA)-N-D-N-I-K-
Y-(SD)-(PQ)-K-F-Q-GRFTISADNAKNSAYLQMNSLRAEDTAVYYCA
RSEENWYDFFDY

(SEQ ID NO:114)

EVQLVESGGGLVQPGGSLRLSCTGSG-(YF)-(NT)-I-K-D-T-Y-
(MI)-H, WVRQAPGKGLEWIG (WR)-I-D-P-(GA)-N-D-N-I-K-
Y-(SD)-(PQ)-K-F-Q-GRFTISADNAKNSLYLQMNSLRAEDTAVYYCA
RSEENWYDFFDY

(SEQ ID NO:115)

EVQLQQSGAELVKPGASVKLSCTGSG-(YF)-(NT)-I-K-D-T-Y-
(MI)-H, WVKQRPEQGLEWIG (WR)-I-D-P-(GA)-N-D-N-I-K-
Y-(SD)-(PQ)-K-F-Q-GKATITADTSSNTAYLQLNSLTSEDTAVYYCA
RSEENWYDFFDY

or a sequence that has fewer than eight, seven, six, five, four, three, or two alterations (e.g., substitutions, insertions or deletions, e.g., conservative substitutions or a substitution for an amino acid residue at a corresponding position in MJ 2-7) in the framework region. Exemplary substitutions are at one or more of the following Kabat positions: 2, 4, 6, 25, 36, 37, 39, 47, 48, 93, 94, 103, 104, 106, and 107. The substitutions can, for example, substitute an amino acid at a corresponding position from MJ 2-7 into a human framework region. The FR4 region can include, e.g., the sequence WGQGTTLTVSS (SEQ ID NO:116) or WGQGTLVTVSS (SEQ ID NO:117).

Additional examples of IL-13 antibodies, that interfere with IL-13 binding to IL-13R (e.g., an IL-13 receptor complex), or a subunit thereof, include “mAb13.2” and modified, e.g., chimeric or humanized forms thereof. The amino acid and nucleotide sequences for the heavy chain variable region of mAb13.2 are set forth herein as SEQ ID NO:198 and SEQ ID NO:217, respectively. The amino acid and nucleotide sequences for the light chain variable region of mAb13.2 are set forth herein as SEQ ID NO:199 and SEQ ID NO:218, respectively. An exemplary chimeric form (e.g., a form comprising the heavy and light chain variable region of mAb13.2) is referred to herein as “ch13.2.” The amino acid and nucleotide sequences for the heavy chain variable region of ch13.2 are set forth herein as SEQ ID NO:208 and SEQ ID NO:204, respectively. The amino acid and nucleotide sequences for the light chain variable region of ch13.2 are set forth herein as SEQ ID NO:213 and SEQ ID NO:219, respectively. A humanized form of mAb13.2, which is referred to herein as “h13.2v1,” has amino acid and nucleotide sequences for the heavy chain variable region set forth herein as SEQ ID NO:209 and SEQ ID NO:205, respectively. The amino acid and nucleotide sequences for the light chain variable region of h13.2v1 are set forth herein as SEQ ID NO:214 and SEQ ID NO:220, respectively. Another humanized form of mAb13.2, which is referred to herein as “h13.2v2,” has amino acid and nucleotide sequences for the heavy chain variable region set forth herein as SEQ ID NO:210 and SEQ ID NO:206, respectively. The amino acid and nucleotide sequences for the light chain variable region of h13.2v2 are set forth herein as SEQ ID NO:212 and SEQ ID NO:221, respectively. Another humanized form of mAb13.2, which is referred to herein as “h13.2v3,” has amino acid and nucleotide sequences for the heavy chain variable region set forth herein as SEQ ID NO:211 and SEQ ID NO:207, respectively. The amino acid and nucleotide sequences for the light chain variable region of h13.2v3 are set forth herein as SEQ ID NO:35 and SEQ ID NO:223, respectively.

In another embodiment, the anti-IL-13 antibody molecule comprises at least one, two, three, or four antigen-binding regions, e.g., variable regions, having an amino acid sequence as set forth in SEQ ID NOs:198, 208, 209, 210, or 211 for VH, and/or SEQ ID NOs:199, 213, 214, 212, or 215 for VL), or a sequence substantially identical thereto (e.g., a sequence at least about 85%, 90%, 95%, 99% or more identical thereto, or which differs by no more than 1, 2, 5, 10, or 15 amino acid residues from SEQ ID NOs:199, 213, 214, 212, 198, 208, 209, 210, 215, or 211). In another embodiment, the antibody includes a VH and/or VL domain encoded by a nucleic acid having a nucleotide sequence as set forth in SEQ ID NOs222, 204, 205, 208, or 207 for VH, and/or SEQ ID NOs:218, 219, 220, 221, or 223 for VL), or a sequence substantially identical thereto (e.g., a sequence at least about 85%, 90%, 95%, 99% or more identical thereto, or which differs by no more than 3, 6, 15, 30, or 45 nucleotides from SEQ ID NOs:218, 219, 220, 221, 222, 204, 205, 206, 223, or 207). In yet another embodiment, the antibody or fragment thereof comprises at least one, two, or three CDRs from a heavy chain variable region having an amino acid sequence as set forth in SEQ ID NOs:202, 203, or 196 for VH CDRs 1-3, respectively, or a sequence substantially homologous thereto (e.g., a sequence at least about 85%, 90%, 95%, 99% or more identical thereto, and/or having one or more substitutions, e.g., conserved substitutions). In yet another embodiment, the antibody or fragment thereof comprises at least one, two, or three CDRs from a light chain variable region having an amino acid sequence as set forth in SEQ ID NOs:197, 200, or 201 for VL CDRs 1-3, respectively, or a sequence substantially homologous thereto (e.g., a sequence at least about 85%, 90%, 95%, 99% or more identical thereto, and/or having one or more substitutions, e.g., conserved substitutions). In yet another embodiment, the antibody or fragment thereof comprises at least one, two, three, four, five or six CDRs from heavy and light chain variable regions having an amino acid sequence as set forth in SEQ ID NOs:202, 203, 196 for VH CDRs 1-3, respectively; and SEQ ID NO:197, 200, or 201 for VL CDRs 1-3, respectively, or a sequence substantially homologous thereto (e.g., a sequence at least about 85%, 90%, 95%, 99% or more identical thereto, and/or having one or more substitutions, e.g., conserved substitutions).

In one embodiment, the anti-IL-13 antibody molecule includes all six CDRs from C65 or closely related CDRs, e.g., CDRs which are identical or which have at least one amino acid alteration, but not more than two, three or four alterations (e.g., substitutions, deletions, or insertions).

In still another embodiment, the IL-13 binding agent includes at least one, two or three CDR regions that have the same canonical structures and the corresponding CDR regions of C65, e.g., at least CDR1 and CDR2 of the heavy and/or light chain variable domains of C65.

In one embodiment, the heavy chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the heavy chain framework of one of the following germline V segment sequences: DP-71 or DP-67 or another V gene which is compatible with the canonical structure class of C65 (see, e.g., Chothia et al. (1992) J. Mol. Biol. 227:799-817; Tomlinson et al. (1992) J. Mol. Biol. 227:776-798).

In one embodiment, the light chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of DPK-1 or DPK-9 germline sequence or another V gene which is compatible with the canonical structure class of C65 (see, e.g., Tomlinson et al. (1995) EMBO J. 14:4628).

In another embodiment, the light chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a Vκ I subgroup germline sequence, e.g., a DPK-9 or DPK-1 sequence.

In another embodiment, the heavy chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a VH IV subgroup germline sequence, e.g., a DP-71 or DP-67 sequence.

In one embodiment, the light or the heavy chain variable framework (e.g., the region encompassing at least FR1, FR2, FR3, and optionally FR4) can be chosen from: (a) a light or heavy chain variable framework including at least 80%, 85%, 90%, 95%, or 100% of the amino acid residues from a human light or heavy chain variable framework, e.g., a light or heavy chain variable framework residue from a human mature antibody, a human germline sequence, a human consensus sequence, or a human antibody described herein; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the heavy chain variable domain sequence includes human residues or human consensus sequence residues at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering).

In one embodiment, the anti-IL13 antibody molecules includes at least one non-human CDR, e.g., a murine CDR, e.g., a CDR from e.g., mAb13.2, MJ2-7, C65, and/or modified forms thereof (e.g., humanized or chimeric variansts thereof), and at least one framework which differs from a framework of e.g., mAb13.2, MJ2-7, C65, and/or modified forms thereof (e.g., humanized or chimeric variansts thereof) by at least one amino acid, e.g., at least 5, 8, 10, 12, 15, or 18 amino acids. For example, the proteins include one, two, three, four, five, or six such non-human CDRs and includes at least one amino acid difference in at least three of HC FR1, HC FR2, HC FR3, LC FR1, LC FR2, and LC FR3.

In one embodiment, the heavy or light chain variable domain sequence of the anti-IL-13 antibody molecule includes an amino acid sequence, which is at least 80%, 85%, 90%, 95%, 97%, 98%, 99% or higher identical to a variable domain sequence of an antibody described herein, e.g., mAb13.2, MJ2-7, C65, and/or modified forms thereof (e.g., humanized or chimeric variansts thereof); or which differs at least 1 or 5 residues, but less than 40, 30, 20, or 10 residues, from a variable domain sequence of an antibody described herein, e.g., mAb13.2, MJ2-7, C65, and/or modified forms thereof (e.g., humanized or chimeric variansts thereof). In one embodiment, the heavy or light chain variable domain sequence of the protein includes an amino acid sequence encoded by a nucleic acid sequence described herein or a nucleic acid that hybridizes to a nucleic acid sequence described herein or its complement, e.g., under low stringency, medium stringency, high stringency, or very high stringency conditions.

In one embodiment, one or both of the variable domain sequences include amino acid positions in the framework region that are variously derived from both a non-human antibody (e.g., a murine antibody such as mAb13.2) and a human antibody or germline sequence. For example, a variable domain sequence can include a number of positions at which the amino acid residue is identical to both the non-human antibody and the human antibody (or human germline sequence) because the two are identical at that position. Of the remaining framework positions where the non-human and human differ, at least 50, 60, 70, 80, or 90% of the positions of the variable domain are preferably identical to the human antibody (or human germline sequence) rather than the non-human. For example, none, or at least one, two, three, or four of such remaining framework position may be identical to the non-human antibody rather than to the human. For example, in HC FR1, one or two such positions can be non-human; in HC FR2, one or two such positions can be non-human; in FR3, one, two, three, or four such positions can be non-human; in LC FR1, one, two, three, or four such positions can be non-human; in LC FR2, one or two such positions can be non-human; in LC FR3, one or two such positions can be non-human. The frameworks can include additional non-human positions.

In one embodiment, an antibody molecule has CDR sequences that differ only insubstantially from those of MJ 2-7, C65, or 13.2. Insubstantial differences include minor amino acid changes, such as substitutions of 1 or 2 out of any of typically 5-7 amino acids in the sequence of a CDR, e.g., a Chothia or Kabat CDR. Typically, an amino acid is substituted by a related amino acid having similar charge, hydrophobic, or stereochemical characteristics. Such substitutions are within the ordinary skills of an artisan. Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al. (1991) J. Immunol. 147:2657-62; Morgan et al. (1995) Immunology 86:319-24), or changing the species from which the constant region is derived. Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. In the IgG1 or IgG2 heavy chain, for example, such mutations may be made to resemble the amino acid sequence set forth in SEQ ID NO:17. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08).

Additional examples of anti-IL13 antibody molecules are disclosed in U.S. Ser. No. 07/012,8192 or WO 05/007699 and in Blanchard, C. et al. (2005) Clinical and Experimental Allergy 35(8):1096-1103 disclosing CAT-354; WO 05/062967, WO 05/062972 and Clinical Trials Gov. Identifier: NCT00441818 disclosing TNX-650; Clinical Trials Gov. Identifier: NCT532233 disclosing QAX-576; US 06/0140948 or WO 06/055638, filed in the name of Abgenix; U.S. Pat. No. 6,468,528 assigned to AMGEN; WO 05/091856 naming Centocor, Inc. as the applicant; and in Yang et al. (2004) Cytokine 28(6):224-32 and Yang et al. (2005) J Pharmacol Exp Ther: 313(1):8-15; and anti-IL13 antibodies as disclosed in WO 07/080,174 filed in the name of Glaxo, and as disclosed in WO 07/045,477 in the name of Novartis.

The anti-IL-13 antibody molecule can be in the form of intact antibodies, antigen-binding fragments of antibodies, e.g., Fab, F(ab′)₂, Fd, dAb, and scFv fragments, and intact antibodies and fragments that have been mutated either in their constant and/or variable domain (e.g., mutations to produce chimeric, partially humanized, or fully humanized antibodies, as well as to produce antibodies with a desired trait, e.g., enhanced IL-13 binding and/or reduced FcR binding).

The anti-IL-13 antibody molecule can be derivatized or linked to another functional molecule, e.g., another peptide or protein (e.g., an Fab fragment). For example, the binding agent can be functionally linked (e.g., by chemical coupling, genetic fusion, noncovalent association or otherwise) to one or more other molecular entities, such as another antibody molecule (e.g., to form a bispecific or a multispecific antibody molecule), toxins, radioisotopes, cytotoxic or cytostatic agents, among others.

### Additional IL-13/IL-13R Binding Agents

Also provided are other binding agents, other than antibody molecules, that bind to IL-13 polypeptide or nucleic acid, or an IL-13R polypeptide or nucleic acid. In embodiments, the other binding agents described herein are antagonists and thus reduce, inhibit or otherwise diminish one or more biological activities of IL-13 (e.g., one or more biological activities of IL-13 as described herein).

Binding agents can be identified by a number of means, including modifying a variable domain described herein or grafting one or more CDRs of a variable domain described herein onto another scaffold domain. Binding agents can also be identified from diverse libraries, e.g., by screening. One method for screening protein libraries uses phage display. Particular regions of a protein are varied and proteins that interact with IL-13, or its receptors, are identified, e.g., by retention on a solid support or by other physical association. For example, to identify particular binding agents that bind to the same epitope or an overlapping epitope as MJ2-7, C65 or mAb 13.2 on IL-13, binding agents can be eluted by adding MJ2-7, C65 or mAb13.2 (or related antibody), or binding agents can be evaluated in competition experiments with MJ2-7, C65 or mAb13.2 (or related antibody). It is also possible to deplete the library of agents that bind to other epitopes by contacting the library to a complex that contains IL-13 and MJ2-7, C65 or mAb13.2 (or related antibody). The depleted library can then be contacted to IL-13 to obtain a binding agent that binds to IL-13 but not to IL-13 when it is bound by MJ 2-7, C65 or mAb13.2. It is also possible to use peptides from IL-13 that contain the MJ 2-7, C65 epitope, or mAb13.2 as a target.

Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; WO 94/05781; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; and Barbas et al. (1991) PNAS 88:7978-7982. Yeast surface display is described, e.g., in Boder and Wittrup (1997) Nat. Biotechnol. 15:553-557. Another form of display is ribosome display. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Binding agents that bind to IL-13 or IL-4, or its receptors, can have structural features of one scaffold proteins, e.g., a folded domain. An exemplary scaffold domain, based on an antibody, is a “minibody” scaffold has been designed by deleting three beta strands from a heavy chain variable domain of a monoclonal antibody (Tramontano et al., 1994, J. Mol. Recognit. 7:9; and Martin et al., 1994, EMBO J. 13:5303-5309). This domain includes 61 residues and can be used to present two hypervariable loops, e.g., one or more hypervariable loops of a variable domain described herein or a variant described herein. In another approach, the binding agent includes a scaffold domain that is a V-like domain (Coia et al. WO 99/45110). V-like domains refer to a domain that has similar structural features to the variable heavy (VH) or variable light (VL) domains of antibodies. Another scaffold domain is derived from tendamistatin, a 74 residue, six-strand beta sheet sandwich held together by two disulfide bonds (McConnell and Hoess, 1995, J. Mol. Biol. 250:460). This parent protein includes three loops. The loops can be modified (e.g., using CDRs or hypervariable loops described herein) or varied, e.g., to select domains that bind to IL-13 or IL-4, or its receptors. WO 00/60070 describes a β-sandwich structure derived from the naturally occurring extracellular domain of CTLA-4 that can be used as a scaffold domain.

Still another scaffold domain for an IL-13/13R binding agent is a domain based on the fibronectin type III domain or related fibronectin-like proteins. The overall fold of the fibronectin type III (Fn3) domain is closely related to that of the smallest functional antibody fragment, the variable domain of the antibody heavy chain. Fn3 is a β-sandwich similar to that of the antibody VH domain, except that Fn3 has seven β-strands instead of nine. There are three loops at the end of Fn3; the positions of BC, DE and FG loops approximately correspond to those of CDR1, 2 and 3 of the VH domain of an antibody. Fn3 is advantageous because it does not have disulfide bonds. Therefore, Fn3 is stable under reducing conditions, unlike antibodies and their fragments (see WO 98/56915; WO 01/64942; WO 00/34784). An Fn3 domain can be modified (e.g., using CDRs or hypervariable loops described herein) or varied, e.g., to select domains that bind to IL-13 or IL-4, or its receptors.

Still other exemplary scaffold domains include: T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). US 20040009530 describes examples of some alternative scaffolds.

Examples of small scaffold domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds). Examples of small intracellular scaffold domains include SH2, SH3, and EVH domains. Generally, any modular domain, intracellular or extracellular, can be used.

Exemplary criteria for evaluating a scaffold domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffold domain is a small, stable protein domains, e.g., a protein of less than 100, 70, 50, 40 or 30 amino acids. The domain may include one or more disulfide bonds or may chelate a metal, e.g., zinc.

Still other binding agents are based on peptides, e.g., proteins with an amino acid sequence that are less than 30, 25, 24, 20, 18, 15, or 12 amino acids. Peptides can be incorporated in a larger protein, but typically a region that can independently bind to IL-13, e.g., to an epitope described herein. Peptides can be identified by phage display. See, e.g., US 20040071705.

A binding agent may include non-peptide linkages and other chemical modification. For example, the binding agent may be synthesized as a peptidomimetic, e.g., a peptoid (see, e.g., Simon et al. (1992) Proc. Natl. Acad. Sci. USA 89:9367-71 and Horwell (1995) Trends Biotechnol. 13:132-4). A binding agent may include one or more (e.g., all) non-hydrolyzable bonds. Many non-hydrolyzable peptide bonds are known in the art, along with procedures for synthesis of peptides containing such bonds. Exemplary non-hydrolyzable bonds include —[CH₂NH]— reduced amide peptide bonds, —[COCH₂]— ketomethylene peptide bonds, —[CH(CN)NH]—(cyanomethylene)amino peptide bonds, —[CH₂CH(OH)]— hydroxyethylene peptide bonds, —[CH₂O]— peptide bonds, and —[CH₂S]— thiomethylene peptide bonds (see e.g., U.S. Pat. No. 6,172,043).

In another embodiment, the IL-13 antagonist is derived from a lipocalin, e.g., a human lipocalin scaffold.

### Variant IL-13 Binding Molecules

In yet another embodiment, the IL-13 binding agent, antagonist is a variant molecule or a small molecule. An example of a variant molecule typically includes a binding domain polypeptide that is fused or otherwise connected to a hinge or hinge-acting region polypeptide, which in turn is fused or otherwise connected to a region comprising one or more native or engineered constant regions from a heavy chain, other than CH1, for example, the CH2 and CH3 regions of IgG and IgA, or the CH3 and CH4 regions of IgE (see e.g., U.S. 05/0136049 by Ledbetter, J. et al. for a more complete description). The binding domain-fusion protein can further include a region that includes a native or engineered heavy chain CH2 constant region polypeptide (or CH3 in the case of a construct derived in whole or in part from IgE) that is fused or otherwise connected to the hinge region polypeptide and a native or engineered heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-fusion proteins are capable of at least one activity selected from the group consisting of fusion protein-dependent cell-mediated cytotoxicity, complement fixation, and/or binding to a target, for example, a IL-13.

Another example of an IL-13 binding variant is a soluble form of an IL-13 receptor or a fusion thereof. For example, a modified soluble receptor form can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. As used herein, a “fusion protein” refers to a protein containing two or more operably associated, e.g., linked, moieties, e.g., protein moieties. Typically, the moieties are covalently associated. The moieties can be directly associated, or connected via a spacer or linker. The fusion proteins may additionally include a linker sequence joining the first moiety to the second moiety. For example, the fusion protein can include a peptide linker, e.g., a peptide linker of about 4 to 20, more preferably, 5 to 10, amino acids in length; the peptide linker is 8 amino acids in length. Each of the amino acids in the peptide linker is selected from the group consisting of Gly, Ser, Asn, Thr and Ala; the peptide linker includes a Gly-Ser element. In other embodiments, the fusion protein includes a peptide linker and the peptide linker includes a sequence having the formula (Ser-Gly-Gly-Gly-Gly)_{y} wherein y is 1, 2, 3, 4, 5, 6, 7, or 8.

In other embodiments, additional amino acid sequences can be added to the N- or C-terminus of the fusion protein to facilitate expression, steric flexibility, detection and/or isolation or purification. The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second receptor polypeptide. In embodiments, the second polypeptide includes at least a region of an immunoglobulin polypeptide. Immunoglobulin fusion polypeptide are known in the art and are described in e.g., U.S. Pat. Nos. 5,516,964; 5,225,538; 5,428,130; 5,514,582; 5,714,147; and 5,455,165. For example, a soluble form of a receptor or a ligand binding fusion can be fused to a heavy chain constant region of the various isotypes, including: IgG1, IgG2, IgG3, IgG4, IgM, IgA1, IgA2, IgD, and IgE).

The Fc sequence can be mutated at one or more amino acids to reduce effector cell function, Fc receptor binding and/or complement activity. Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions. For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In embodiments, the second polypeptide has less effector function that the effector function of a Fc region of a wild-type immunoglobulin heavy chain. Fc effector function includes for example, Fc receptor binding, complement fixation and T cell depleting activity (see for example, U.S. Pat. No. 6,136,310). Methods for assaying T cell depleting activity, Fc effector function, and antibody stability are known in the art. In one embodiment, the second polypeptide has low or no detectable affinity for the Fc receptor. In an alternative embodiment, the second polypeptide has low or no detectable affinity for complement protein C1q.

It will be understood that the antibody molecules and soluble receptor or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

### Nucleic Acid Antagonists

In yet another embodiment, the antagonist inhibits the expression of nucleic acid encoding an IL-13 or IL-13R. Examples of such antagonists include nucleic acid molecules, for example, antisense molecules, ribozymes, RNAi, triple helix molecules that hybridize to a nucleic acid encoding an IL-13 or IL-13R, or a transcription regulatory region, and blocks or reduces mRNA expression of an IL-13 or IL-13R.

In embodiments, nucleic acid antagonists are used to decrease expression of an endogenous gene encoding an IL-13 or IL-13R. In one embodiment, the nucleic acid antagonist is an siRNA that targets mRNA encoding an IL-13 or IL-13R. Other types of antagonistic nucleic acids can also be used, e.g., a dsRNA, a ribozyme, a triple-helix former, or an antisense nucleic acid. Accordingly, isolated nucleic acid molecules that are nucleic acid inhibitors, e.g., antisense, RNAi, to an IL-13 or IL-13R-encoding nucleic acid molecule are provided.

The antisense nucleic acid molecules of the invention are typically administered to a subject (e.g., by direct injection at a tissue site), or generated in situ such that they hybridize with or bind to cellular mRNA and/or genomic DNA encoding a receptor protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

In still another embodiment, an antisense nucleic acid of the invention is a ribozyme. A ribozyme having specificity for an IL-13 or IL-13R, or an IL-4 or IL-4R-encoding nucleic acid can include one or more sequences complementary to the nucleotide sequence of an IL-13 or IL-13R, or an IL-4 or IL-4R cDNA disclosed herein, and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a receptor-encoding mRNA. See, e.g., Cech et al. U.S. Pat. No. 4,987,071; and Cech et al. U.S. Pat. No. 5,116,742. Alternatively, mRNA can be used to select a catalytic RNA having a specific ribonuclease activity from a pool of RNA molecules. See, e.g., Bartel, D. and Szostak, J. W. (1993) Science 261:1411-1418.

IL-13 or IL-13R gene expression can be inhibited by targeting nucleotide sequences complementary to the regulatory region of the IL-13 or IL-13R (e.g., the an IL-113 or IL-13R promoter and/or enhancers) to form triple helical structures that prevent transcription of an IL-13 or IL-13R gene in target cells. See generally, Helene, C. (1991) Anticancer Drug Des. 6:569-84; Helene, C. (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

An IL-13 or IL-13R nucleic acid molecule can be modified at the base moiety, sugar moiety or phosphate backbone to improve, e.g., the stability, hybridization, or solubility of the molecule. For non-limiting examples of synthetic oligonucleotides with modifications see Toulme (2001) Nature Biotech. 19:17 and Faria et al. (2001) Nature Biotech. 19:40-44. Such phosphoramidite oligonucleotides can be effective antisense agents. For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; WO88/09810) or the blood-brain barrier (see, e.g., WO 89/10134). In addition, oligonucleotides can be modified with hybridization-triggered cleavage agents (see, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### Binding Agent Production

Some antibody molecules, e.g., Fabs, or binding agents can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibody molecules can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibody molecules are produced in mammalian cells. Typical mammalian host cells for expressing the clone antibodies or antigen-binding fragments thereof include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells and SP2 cells, COS cells, and a cell from a transgenic animal, e.g., a transgenic mammal. For example, the cell is a mammary epithelial cell.

In addition to the nucleic acid sequences encoding the antibody molecule, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced.

In an exemplary system for recombinant expression of an antibody molecule, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells can be cultured to allow for expression of the antibody heavy and light chains and intact antibody is recovered from the culture medium. Standard molecular biology techniques can be used to prepare the recombinant expression vector, transfect the host cells, select for transformants, culture the host cells and recover the antibody molecule from the culture medium. For example, some antibody molecules can be isolated by affinity chromatography with a Protein A or Protein G coupled matrix.

For antibody molecules that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibody molecules can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody molecule and a signal sequence for secretion. The milk produced by females of such transgenic mammals includes, secreted-therein, the antibody of interest. The antibody molecule can be purified from the milk, or for some applications, used directly.

### Characterization of Binding Agents

The binding properties of a binding agent may be measured by any method, e.g., one of the following methods: BIACORE™ analysis, Enzyme Linked Immunosorbent Assay (ELISA), x-ray crystallography, sequence analysis and scanning mutagenesis. The ability of a protein to neutralize and/or inhibit one or more IL-13-associated activities may be measured by the following methods: assays for measuring the proliferation of an IL-13 dependent cell line, e.g. TFI; assays for measuring the expression of IL-13-mediated polypeptides, e.g., flow cytometric analysis of the expression of CD23; assays evaluating the activity of downstream signaling molecules, e.g., STAT6; assays evaluating production of tenascin; assays testing the efficiency of an antibody described herein to prevent asthma in a relevant animal model, e.g., the cynomolgus monkey, and other assays. An IL-13 binding agent, particularly an IL-13 antibody molecule, can have a statistically significant effect in one or more of these assays. Exemplary assays for binding properties include the following.

The binding interaction of a IL-13 or IL-4 binding agent and a target (e.g., IL-13) can be analyzed using surface plasmon resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a molecule to a target. Such data can be used to compare different molecules. Information from SPR can also be used to develop structure-activity relationships (SAR). For example, the kinetic and equilibrium binding parameters of different antibody molecule can be evaluated. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

### Respiratory Disorders

An IL-13 binding agent or antagonist can be used to treat or prevent respiratory disorders including, but are not limited to asthma (e.g., allergic and nonallergic asthma (e.g., due to infection, e.g., with respiratory syncytial virus (RSV), e.g., in younger children)); bronchitis (e.g., chronic bronchitis); chronic obstructive pulmonary disease (COPD) (e.g., emphysema (e.g., cigarette-induced emphysema); conditions involving airway inflammation, eosinophilia, fibrosis and excess mucus production, e.g., cystic fibrosis, pulmonary fibrosis, and allergic rhinitis. For example, an IL-13 binding agent (e.g., an anti-IL-13 antibody molecule) can be administered in an amount effective to treat or prevent the disorder or to ameliorate at least one symptom of the disorder.

Asthma can be triggered by myriad conditions, e.g., inhalation of an allergen, presence of an upper-respiratory or ear infection, etc. (Opperwall (2003) Nurs. Clin. North Am. 38:697-711). Allergic asthma is characterized by airway hyperresponsiveness (AHR) to a variety of specific and nonspecific stimuli, elevated serum immunoglobulin E (IgE), excessive airway mucus production, edema, and bronchial epithelial injury (Wills-Karp, supra). Allergic asthma begins when the allergen provokes an immediate early airway response, which is frequently followed several hours later by a delayed late-phase airway response (LAR) (Henderson et al. (2000) J. Immunol. 164:1086-95). During LAR, there is an influx of eosinophils, lymphocytes, and macrophages throughout the airway wall and the bronchial fluid. (Henderson et al., supra). Lung eosinophilia is a hallmark of allergic asthma and is responsible for much of the damage to the respiratory epithelium (Li et al. (1999) J. Immunol. 162:2477-87).

CD4⁺ T helper (Th) cells are important for the chronic inflammation associated with asthma (Henderson et al., supra). Several studies have shown that commitment of CD4+ cells to type 2 T helper (Th2) cells and the subsequent production of type 2 cytokines (e.g., IL-4, IL-5, IL-10, and IL-13) are important in the allergic inflammatory response leading to AHR (Tomkinson et al. (2001) J. Immunol. 166:5792-5800, and references cited therein). First, CD4⁺ T cells have been shown to be necessary for allergy-induced asthma in murine models. Second, CD4⁺ T cells producing type 2 cytokines undergo expansion not only in these animal models but also in patients with allergic asthma. Third, type 2 cytokine levels are increased in the airway tissues of animal models and asthmatics. Fourth, Th2 cytokines have been implicated as playing a central role in eosinophil recruitment in murine models of allergic asthma, and adoptively transferred Th2 cells have been correlated with increased levels of eotaxin (a potent eosinophil chemoattractant) in the lung as well as lung eosinophilia (Wills-Karp et al., supra; Li et al., supra).

The methods for treating or preventing asthma described herein include those for extrinsic asthma (also known as allergic asthma or atopic asthma), intrinsic asthma (also known as non-allergic asthma or non-atopic asthma) or combinations of both, which has been referred to as mixed asthma. Extrinsic or allergic asthma includes incidents caused by, or associated with, e.g., allergens, such as pollens, spores, grasses or weeds, pet danders, dust, mites, etc. As allergens and other irritants present themselves at varying points over the year, these types of incidents are also referred to as seasonal asthma. Also included in the group of extrinsic asthma is bronchial asthma and allergic bronchopulmonary aspergillosis.

Disorders that can be treated or alleviated by the agents described herein include those respiratory disorders and asthma caused by infectious agents, such as viruses (e.g., cold and flu viruses, respiratory syncytial virus (RSV), paramyxovirus, rhinovirus and influenza viruses. RSV, rhinovirus and influenza virus infections are common in children, and are one leading cause of respiratory tract illnesses in infants and young children. Children with viral bronchiolitis can develop chronic wheezing and asthma, which can be treated using the methods described herein. Also included are the asthma conditions which may be brought about in some asthmatics by exercise and/or cold air. The methods are useful for asthmas associated with smoke exposure (e.g., cigarette-induced and industrial smoke), as well as industrial and occupational exposures, such as smoke, ozone, noxious gases, sulfur dioxide, nitrous oxide, fumes, including isocyanates, from paint, plastics, polyurethanes, varnishes, etc., wood, plant or other organic dusts, etc. The methods are also useful for asthmatic incidents associated with food additives, preservatives or pharmacological agents. Also included are methods for treating, inhibiting or alleviating the types of asthma referred to as silent asthma or cough variant asthma.

The methods disclosed herein are also useful for treatment and alleviation of asthma associated with gastroesophageal reflux (GERD), which can stimulate bronchoconstriction. GERD, along with retained bodily secretions, suppressed cough, and exposure to allergens and irritants in the bedroom can contribute to asthmatic conditions and have been collectively referred to as nighttime asthma or nocturnal asthma. In methods of treatment, inhibition or alleviation of asthma associated with GERD, a pharmaceutically effective amount of the IL-13 antagonist can be used as described herein in combination with a pharmaceutically effective amount of an agent for treating GERD. These agents include, but are not limited to, proton pump inhibiting agents like PROTONIX® brand of delayed-release pantoprazole sodium tablets, PRILOSEC® brand omeprazole delayed release capsules, ACIPHEX® brand rebeprazole sodium delayed release tablets or PREVACID® brand delayed release lansoprazole capsules.

### Atopic Disorders and Symptoms Thereof

It has been observed that cells from atopic patients have enhanced sensitivity to IL-13. Accordingly, an IL-13 and/or IL-4 antagonist can be administered in an amount effective to treat or prevent an atopic disorder. “Atopic” refers to a group of diseases in which there is often an inherited tendency to develop an allergic reaction.

Examples of atopic disorders include allergy, allergic rhinitis, atopic dermatitis, asthma and hay fever. Asthma is a phenotypically heterogeneous disorder associated with intermittent respiratory symptoms such as, e.g., bronchial hyperresponsiveness and reversible airflow obstruction. Immunohistopathologic features of asthma include, e.g., denudation of airway epithelium, collagen deposition beneath the basement membrane; edema; mast cell activation; and inflammatory cell infiltration (e.g., by neutrophils, eosinophils, and lymphocytes). Airway inflammation can further contribute to airway hyperresponsiveness, airflow limitation, acute bronchoconstriction, mucus plug formation, airway wall remodeling, and other respiratory symptoms. An IL-13 binding agent (e.g., an IL-13 binding agent such as an antibody molecule described herein) can be administered in an amount effective to ameliorate one or more of these symptoms.

Symptoms of allergic rhinitis (hay fever) include itchy, runny, sneezing, or stuffy nose, and itchy eyes. An IL-13 antagonist can be administered to ameliorate one or more of these symptoms. Atopic dermatitis is a chronic (long-lasting) disease that affects the skin. Information about atopic dermatitis is available, e.g., from NIH Publication No. 03-4272. In atopic dermatitis, the skin can become extremely itchy, leading to redness, swelling, cracking, weeping clear fluid, and finally, crusting and scaling. In many cases, there are periods of time when the disease is worse (called exacerbations or flares) followed by periods when the skin improves or clears up entirely (called remissions). Atopic dermatitis is often referred to as “eczema,” which is a general term for the several types of inflammation of the skin. Atopic dermatitis is the most common of the many types of eczema. Examples of atopic dermatitis include: allergic contact eczema (dermatitis: a red, itchy, weepy reaction where the skin has come into contact with a substance that the immune system recognizes as foreign, such as poison ivy or certain preservatives in creams and lotions); contact eczema (a localized reaction that includes redness, itching, and burning where the skin has come into contact with an allergen (an allergy-causing substance) or with an irritant such as an acid, a cleaning agent, or other chemical); dyshidrotic eczema (irritation of the skin on the palms of hands and soles of the feet characterized by clear, deep blisters that itch and burn); neurodermatitis (scaly patches of the skin on the head, lower legs, wrists, or forearms caused by a localized itch (such as an insect bite) that become intensely irritated when scratched); nummular eczema (coin-shaped patches of irritated skin—most common on the arms, back, buttocks, and lower legs—that may be crusted, scaling, and extremely itchy); seborrheic eczema (yellowish, oily, scaly patches of skin on the scalp, face, and occasionally other parts of the body). Additional particular symptoms include stasis dermatitis, atopic pleat (Dennie-Morgan fold), cheilitis, hyperlinear palms, hyperpigmented eyelids (eyelids that have become darker in color from inflammation or hay fever), ichthyosis, keratosis pilaris, lichenification, papules, and urticaria. An IL-13 antagonist can be administered to ameliorate one or more of these symptoms.

An exemplary method for treating allergic rhinitis or other allergic disorder can include initiating therapy with an IL-13 antagonist prior to exposure to an allergen, e.g., prior to seasonal exposure to an allergen, e.g., prior to allergen blooms. Such therapy can include one or more doses, e.g., doses at regular intervals.

### Cancer

IL-13 and its receptors may be involved in the development of at least some types of cancer, e.g., a cancer derived from hematopoietic cells or a cancer derived from brain or neuronal cells (e.g., a glioblastoma). For example, blockade of the IL-13 signaling pathway, e.g., via use of a soluble IL-13 receptor or a STAT6−/− deficient mouse, leads to delayed tumor onset and/or growth of Hodgkins lymphoma cell lines or a metastatic mammary carcinoma, respectively (Trieu et al. (2004) Cancer Res. 64: 3271-75; Ostrand-Rosenberg et al. (2000) J. Immunol. 165: 6015-6019). Cancers that express IL-13R(2 (Husain and Puri (2003) J. Neurooncol. 65:37-48; Mintz et al. (2003) J. Neurooncol. 64:117-23) can be specifically targeted by anti-IL-13 antibodies described herein. IL-13 antagonists can be useful to inhibit cancer cell proliferation or other cancer cell activity. A cancer refers to one or more cells that has a loss of responsiveness to normal growth controls, and typically proliferates with reduced regulation relative to a corresponding normal cell.

Examples of cancers against which IL-13 antagonists (e.g., an IL-13 binding agent such as an antibody or antigen binding fragment described herein) can be used for treatment include leukemias, e.g., B-cell chronic lymphocytic leukemia, acute myelogenous leukemia, and human T-cell leukemia virus type 1 (HTLV-1) transformed T cells; lymphomas, e.g. T cell lymphoma, Hodgkin's lymphoma; glioblastomas; pancreatic cancers; renal cell carcinoma; ovarian carcinoma; AIDS-Kaposi's sarcoma, and breast cancer (as described in Aspord, C. et al. (2007) JEM 204:1037-1047). For example, an IL-13 binding agent (e.g., an anti-IL-13 antibody molecule) can be administered in an amount effective to treat or prevent the disorder, e.g., to reduce cell proliferation, or to ameliorate at least one symptom of the disorder.

### Fibrosis

IL-13 and/or IL-4 antagonists can also be useful in treating inflammation and fibrosis, e.g., fibrosis of the liver. IL-13 production has been correlated with the progression of liver inflammation (e.g., viral hepatitis) toward cirrhosis, and possibly, hepatocellular carcinoma (de Lalla et al. (2004) J. Immunol. 173:1417-1425). Fibrosis occurs, e.g., when normal tissue is replaced by scar tissue, often following inflammation. Hepatitis B and hepatitis C viruses both cause a fibrotic reaction in the liver, which can progress to cirrhosis. Cirrhosis, in turn, can evolve into severe complications such as liver failure or hepatocellular carcinoma. Blocking IL-13 activity using the IL-13 and/or IL-4 antagonists described herein can reduce inflammation and fibrosis, e.g., the inflammation, fibrosis, and cirrhosis associated with liver diseases, especially hepatitis B and C. For example, the antagonists(s) can be administered in an amount effective to treat or prevent the disorder or to ameliorate at least one symptom of the inflammatory and/or fibrotic disorder.

### Inflammatory Bowel Disease

Inflammatory bowel disease (IBD) is the general name for diseases that cause inflammation of the intestines. Two examples of inflammatory bowel disease are Crohn's disease and ulcerative colitis. IL-13/STAT6 signaling has been found to be involved in inflammation-induced hypercontractivity of mouse smooth muscle, a model of inflammatory bowel disease (Akiho et al. (2002) Am. J. Physiol. Gastrointest. Liver Physiol. 282:G226-232). For example, an IL-13 antagonist can be administered in an amount effective to treat or prevent the disorder or to ameliorate at least one symptom of the inflammatory bowel disorder.

### Pharmaceutical Compositions

The IL-13 antagonists (such as those described herein) can be used in vitro, ex vivo, or in vivo. They can be incorporated into a pharmaceutical composition, e.g., by combining the IL-13 binding agent with a pharmaceutically acceptable carrier. Such a composition may contain, in addition to the IL-13 binding agent and carrier, various diluents, fillers, salts, buffers, stabilizers, solubilizers, and other materials well known in the art. Pharmaceutically acceptable materials is generally a nontoxic material that does not interfere with the effectiveness of the biological activity of an IL-13 binding agent. The characteristics of the carrier can depend on the route of administration.

The pharmaceutical composition described herein may also contain other factors, such as, but not limited to, other anti-cytokine antibody molecules or other anti-inflammatory agents as described in more detail below. Such additional factors and/or agents may be included in the pharmaceutical composition to produce a synergistic effect with an IL-13 and/or IL-4 antagonist described herein. For example, in the treatment of allergic asthma, a pharmaceutical composition described herein may include anti-IL-4 antibody molecules or drugs known to reduce an allergic response.

The pharmaceutical composition described herein may be in the form of a liposome in which an IL-13 antagonist, such as one described herein is combined, in addition to other pharmaceutically acceptable carriers, with amphipathic agents such as lipids that exist in aggregated form as micelles, insoluble monolayers, liquid crystals, or lamellar layers while in aqueous solution. Suitable lipids for liposomal formulation include, without limitation, monoglycerides, diglycerides, sulfatides, lysolecithin, phospholipids, saponin, bile acids, and the like. Exemplary methods for preparing such liposomal formulations include methods described in U.S. Pat. Nos. 4,235,871; 4,501,728; 4,837,028; and 4,737,323.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

Administration of an IL-13 antagonist used in the pharmaceutical composition can be carried out in a variety of conventional ways, such as oral ingestion, inhalation, or cutaneous, subcutaneous, or intravenous injection. When a therapeutically effective amount of an IL-13 antagonist is administered by intravenous, cutaneous or subcutaneous injection, the binding agent can be prepared as a pyrogen-free, parenterally acceptable aqueous solution. The composition of such parenterally acceptable protein solutions can be adapted in view factors such as pH, isotonicity, stability, and the like, e.g., to optimize the composition for physiological conditions, binding agent stability, and so forth. A pharmaceutical composition for intravenous, cutaneous, or subcutaneous injection can contain, e.g., an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition may also contain stabilizers, preservatives, buffers, antioxidants, or other additive.

The amount of an IL-13 antagonist in the pharmaceutical composition can depend upon the nature and severity of the condition being treated, and on the nature of prior treatments that the patient has undergone. The pharmaceutical composition can be administered to normal patients or patients who do not show symptoms, e.g., in a prophylactic mode. An attending physician may decide the amount of IL-13 and/or IL-4 antagonist with which to treat each individual patient. For example, an attending physician can administer low doses of antagonist and observe the patient's response. Larger doses of antagonist may be administered until the optimal therapeutic effect is obtained for the patient, and at that point the dosage is not generally increased further. For example, a pharmaceutical may contain between about 0.1 mg to 50 mg antibody per kg body weight, e.g., between about 0.1 mg and 5 mg or between about 8 mg and 50 mg antibody per kg body weight. In one embodiment in which the antibody is delivered subcutaneously at a frequency of no more than twice per month, e.g., every other week or monthly, the composition includes an amount of about 0.7-3.3, e.g., 1.0-3.0 mg/kg, e.g., about 0.8-1.2, 1.2-2.8, or 2.8-3.3 mg/kg. In other embodiments, each dose can be administered by inhalation or by injection, e.g., subcutaneously, in an amount of about 0.5-10 mg/kg (e.g., about 0.7-5 mg/kg, 0.9-4 mg/kg, 1-3 mg/kg, 1.5-2.5 mg/kg, 2 mg/kg). In one embodiment, the single treatment interval includes two subcutaneous doses of about 1-3 mg/kg, 1.5-2.5 mg/kg, 2 mg/kg of an anti-IL13 antibody molecule at least 4, 7, 9 or 14 days apart. For example, the single treatment interval can include two subcutaneous doses of about 2 mg/kg of an anti-IL13 antibody molecule 7 days apart.

The duration of therapy using the pharmaceutical composition may vary, depending on the severity of the disease being treated and the condition and potential idiosyncratic response of each individual patient. In one embodiment, the IL-13 and/or IL-4 antagonist can also be administered via the subcutaneous route, e.g., in the range of once a week, once every 24, 48, 96 hours, or not more frequently than such intervals. Exemplary dosages can be in the range of 0.1-20 mg/kg, more preferably 1-10 mg/kg. The agent can be administered, e.g., by intravenous infusion at a rate of less than 20, 10, 5, or 1 mg/min to reach a dose of about 1 to 50 mg/m² or about 5 to 20 mg/m².

In one embodiment, an administration of an IL-13 antagonist to the patient includes varying the dosage of the protein, e.g., to reduce or minimize side effects. For example, the subject can be administered a first dosage, e.g., a dosage less than a therapeutically effective amount. In a subsequent interval, e.g., at least 6, 12, 24, or 48 hours later, the patient can be administered a second dosage, e.g., a dosage that is at least 25, 50, 75, or 100% greater than the first dosage. For example, the second and/or a comparable third, fourth and fifth dosage can be at least about 70, 80, 90, or 100% of a therapeutically effective amount.

### Inhalation

A composition that includes an IL-13 antagonist can be formulated for inhalation or other mode of pulmonary delivery. The term “pulmonary tissue” as used herein refers to any tissue of the respiratory tract and includes both the upper and lower respiratory tract, except where otherwise indicated. An IL-13 and/or IL-4 antagonist can be administered in combination with one or more of the existing modalities for treating pulmonary diseases.

In one example, the IL-13 antagonist is formulated for a nebulizer. In one embodiment, the IL-13 antagonist can be stored in a lyophilized form (e.g., at room temperature) and reconstituted in solution prior to inhalation. It is also possible to formulate the IL-13 antagonist for inhalation using a medical device, e.g., an inhaler. See, e.g., U.S. Pat. Nos. 6,102,035 (a powder inhaler) and 6,012,454 (a dry powder inhaler). The inhaler can include separate compartments for the IL-13 antagonist at a pH suitable for storage and another compartment for a neutralizing buffer and a mechanism for combining the IL-13 antagonist with a neutralizing buffer immediately prior to atomization. In one embodiment, the inhaler is a metered dose inhaler.

The three common systems used to deliver drugs locally to the pulmonary air passages include dry powder inhalers (DPIs), metered dose inhalers (MDIs) and nebulizers. MDIs, the most popular method of inhalation administration, may be used to deliver medicaments in a solubilized form or as a dispersion. Typically MDIs comprise a Freon or other relatively high vapor pressure propellant that forces aerosolized medication into the respiratory tract upon activation of the device. Unlike MDIs, DPIs generally rely entirely on the inspiratory efforts of the patient to introduce a medicament in a dry powder form to the lungs. Nebulizers form a medicament aerosol to be inhaled by imparting energy to a liquid solution. Direct pulmonary delivery of drugs during liquid ventilation or pulmonary lavage using a fluorochemical medium has also been explored. These and other methods can be used to deliver an IL-13 antagonist. In one embodiment, the IL-13 antagonist is associated with a polymer, e.g., a polymer that stabilizes or increases half-life of the compound.

For example, for administration by inhalation, an IL-13 antagonist is delivered in the form of an aerosol spray from pressured container or dispenser which contains a suitable propellant or a nebulizer. The IL-13 antagonist may be in the form of a dry particle or as a liquid. Particles that include the IL-13 antagonist can be prepared, e.g., by spray drying, by drying an aqueous solution of the IL-13 antagonist with a charge neutralizing agent and then creating particles from the dried powder or by drying an aqueous solution in an organic modifier and then creating particles from the dried powder.

The IL-13 antagonist may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges for use in an inhaler or insufflator may be formulated containing a powder mix of an IL-13 antagonist and a suitable powder base such as lactose or starch, if the particle is a formulated particle. In addition to the formulated or unformulated compound, other materials such as 100% DPPC or other surfactants can be mixed with the an IL-13 antagonist to promote the delivery and dispersion of formulated or unformulated compound. Methods of preparing dry particles are described, for example, in WO 02/32406.

An IL-13 antagonist can be formulated for aerosol delivery, e.g., as dry aerosol particles, such that when administered it can be rapidly absorbed and can produce a rapid local or systemic therapeutic result. Administration can be tailored to provide detectable activity within 2 minutes, 5 minutes, 1 hour, or 3 hours of administration. In some embodiments, the peak activity can be achieved even more quickly, e.g., within one half hour or even within ten minutes. An IL-13 antagonist can be formulated for longer biological half-life (e.g., by association with a polymer such as PEG) for use as an alternative to other modes of administration, e.g., such that the IL-13 antagonist enters circulation from the lung and is distributed to other organs or to a particular target organ.

In one embodiment, the IL-13 antagonist is delivered in an amount such that at least 5% of the mass of the polypeptide is delivered to the lower respiratory tract or the deep lung. Deep lung has an extremely rich capillary network. The respiratory membrane separating capillary lumen from the alveolar air space is very thin (≦6 Tm) and extremely permeable. In addition, the liquid layer lining the alveolar surface is rich in lung surfactants. In other embodiments, at least 2%, 3%, 5%, 10%, 20%, 30%, 40%, 50%, 60%, 70%, or 80% of the composition of an IL-13 antagonist is delivered to the lower respiratory tract or to the deep lung. Delivery to either or both of these tissues results in efficient absorption of the IL-13 antagonist and high bioavailability. In one embodiment, the IL-13 antagonist is provided in a metered dose using, e.g., an inhaler or nebulizer. For example, the IL-13 binding agent is delivered in a dosage unit form of at least about 0.02, 0.1, 0.5, 1, 1.5, 2, 5, 10, 20, 40, or 50 mg/puff or more. The percent bioavailability can be calculated as follows: the percent bioavailability=(AUCₙₒₙ₋ᵢₙᵥₐₛᵢᵥₑ/AUC_{i.v. or s.c.)}×(dose_{i.v. or s.c.}/doseₙₒₙ₋ᵢₙᵥₐₛᵢᵥₑ)×100.

Although not necessary, delivery enhancers such as surfactants can be used to further enhance pulmonary delivery. A “surfactant” as used herein refers to an IL-13 antagonist having a hydrophilic and lipophilic moiety, which promotes absorption of a drug by interacting with an interface between two immiscible phases. Surfactants are useful in the dry particles for several reasons, e.g., reduction of particle agglomeration, reduction of macrophage phagocytosis, etc. When coupled with lung surfactant, a more efficient absorption of the IL-13 antagonist can be achieved because surfactants, such as DPPC, will greatly facilitate diffusion of the compound. Surfactants are well known in the art and include but are not limited to phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9-; auryl ether; palmitic acid; oleic acid; sorbitan trioleate (Span 85); glycocholate; surfactin; poloxomer; sorbitan fatty acid ester; sorbitan trioleate; tyloxapol; and phospholipids.

### Stabilization

In one embodiment, an IL-13 antagonist is physically associated with a moiety that improves its stabilization and/or retention in circulation, e.g., in blood, serum, lymph, bronchopulmonary lavage, or other tissues, e.g., by at least 1.5, 2, 5, 10, or 50 fold.

For example, an IL-13 antagonist can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used.

For example, an IL-13 antagonist can be conjugated to a water soluble polymer, e.g., hydrophilic polyvinyl polymers, e.g. polyvinylalcohol and polyvinylpyrrolidone. A non-limiting list of such polymers includes polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g. hyaluronic acid; polymers of sugar alcohols such as polysorbitol and polymannitol; heparin or heparan.

The conjugates of an IL-13 antagonist and a polymer can be separated from the unreacted starting materials, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

### Other Uses of IL-13 Antagonists

In yet another aspect, the invention features a method for modulating (e.g., decreasing, neutralizing and/or inhibiting) one or more associated activities of IL-13 in vivo by administering an IL-13 antagonist described herein in an amount sufficient to inhibit its activity. An IL-13 antagonist can also be administered to subjects for whom inhibition of an IL-13-mediated inflammatory response is required. These conditions include, e.g., airway inflammation, asthma, fibrosis, eosinophilia and increased mucus production.

The efficacy of an IL-13 antagonist described herein can be evaluated, e.g., by evaluating ability of the antagonist to modulate airway inflammation in cynomolgus monkeys exposed to an Ascaris suum allergen. An IL-13 antagonist can be used to neutralize or inhibit one or more IL-13-associated activities, e.g., to reduce IL-13 mediated inflammation in vivo, e.g., for treating or preventing IL-13-associated pathologies, including asthma and/or its associated symptoms.

In one embodiment, an IL-13 antagonist, or a pharmaceutical compositions thereof, is administered in combination therapy, i.e., combined with other agents, e.g., therapeutic agents, that are useful for treating pathological conditions or disorders, such as allergic and inflammatory disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more IL-13 binding agents that bind to IL-13 and interfere with the formation of a functional IL-13 signaling complex, coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Furthermore, one or more IL-13 binding agents (e.g., the IL-13 antagonist alone or in combination with the IL-4 antagonist) may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL-13/IL-13-receptor pathway, and thus are expected to enhance and/or synergize with the effects of the IL-13 binding agents.

Therapeutic agents that interfere with different triggers of asthma or airway inflammation, e.g., therapeutic agents used in the treatment of allergy, upper respiratory infections, or ear infections, may be used in combination with an IL-13 binding agent. In one embodiment, one or more IL-13 binding agents (e.g., the IL-13 antagonist alone or in combination with the IL-4 antagonist) may be coformulated with, and/or coadministered with, one or more additional agents, such as other cytokine or growth factor antagonists (e.g., soluble receptors, peptide inhibitors, small molecules, adhesins), antibody molecules that bind to other targets (e.g., antibodies that bind to other cytokines or growth factors, their receptors, or other cell surface molecules), and anti-inflammatory cytokines or agonists thereof. Non-limiting examples of the agents that can be used in combination with IL-13 binding agents include, but are not limited to, inhaled steroids; beta-agonists, e.g., short-acting or long-acting beta-agonists; antagonists of leukotrienes or leukotriene receptors; combination drugs such as ADVAIR®; IgE inhibitors, e.g., anti-IgE antibodies (e.g., XOLAIR®); phosphodiesterase inhibitors (e.g., PDE4 inhibitors); xanthines; anticholinergic drugs; mast cell-stabilizing agents such as cromolyn; IL-5 inhibitors; eotaxin/CCR3 inhibitors; and antihistamines.

In other embodiments, the IL-13 binding agents can be administered in combination with an IL-4 antagonist. Examples of IL-4 antagonists include, but are not limited to, antibody molecules against IL-4 (e.g., pascolizumab and related antibodies disclosed in Hart, T. K. et al. (2002) Clin Exp Immunol. 130(1):93-100; Steinke, J. W. (2004) Immunol. Allergy Clin North Am 24(4):599-614; and in Ramanthan et al. U.S. Pat. No. 6,358,509), IL-4Rα (e.g., AMG-317 and related anti-IL4R antibodies disclosed in US 05/0118176, US 05/0112694 and in Clinical Trials Gov. Identifier: NCT00436670); IL-13Rα1 (e.g., anti-13Rα1 antibodies disclosed in WO 03/080675 which names AMRAD as the applicant); and mono- or bi-specific antibody molecules that bind to IL4 and/or IL-13 (disclosed, e.g., in WO 07/085,815).

In other embodiments, the IL-13 or IL-4 antagonist is an IL-13 or IL-4 mutein (e.g., a truncated or variant form of the cytokine that binds to the IL-13R or an IL-4 receptor, but does not significantly increase the activity of the receptor), or a cytokine-conjugated to a toxin. IL-4 muteins are disclosed by Weinzel et al. (2007) Lancet 370:1422-31. Additional examples of IL-13/IL-4 inhibiting peptides are disclosed in Andrews, A. L. et al. (2006) J. Allergy and Clin Immunol 118:858-865. An example of a cytokine-toxin conjugate is disclosed in WO 03/047632, in Kunwar, S. et al. (2007) J. Clin Oncol 25(7):837-44 and in Husain, S. R. et al. (2003) J. Neurooncol 65(1):37-48.

In yet other embodiments, the IL13 antagonist or the IL-4 antagonist is a full length, or a fragment or modified form of an IL-13 receptor polypeptide (e.g., IL-13Rα2 or IL13Rα1) or an IL-4 receptor polypeptide (e.g., IL-4Rα). For example, the antagonist can be a soluble form of an IL-13 receptor or an IL-14 receptor (e.g., a soluble form of mammalian (e.g., human) IL-13Rα2, IL13Rα1 or IL-4Rα comprising a cytokine-binding domain; e.g., a soluble form of an extracellular domain of mammalian (e.g., human) IL-13Rα2, IL13Rα1 or IL-4Rα). Exemplary receptor antagonists include, e.g., IL-4R-IL-13R binding fusions as described in WO 05/085284 and Economides, A. N. et al. (2003) Nat Med 9(1):47-52, as well as in Borish, L. C. et al. (1999) Am J Respir Crit Care Med 160(6):1816-23.

A soluble form of an IL-13 receptor or IL-4 receptor, or an IL-13 or IL-4 mutein can be used alone or functionally linked (e.g., by chemical coupling, genetic or polypeptide fusion, non-covalent association or otherwise) to a second moiety to facilitate expression, steric flexibility, detection and/or isolation or purification, e.g., an immunoglobulin Fc domain, serum albumin, pegylation, a GST, Lex-A or an MBP polypeptide sequence. The fusion proteins may additionally include a linker sequence joining the first moiety to the second moiety. For example, a soluble IL-13 receptor or IL-4 receptor, or an IL-13 or IL-4 mutein can be fused to a heavy chain constant region of the various isotypes, including: IgG1, IgG2, IgG3, IgG4, IgM, IgA1, IgA2, IgD, and IgE). Typically, the fusion protein can include the extracellular domain of a human soluble IL-13 receptor or IL-4 receptor, or an IL-13 or IL-4 mutein (or a sequence homologous thereto), and, e.g., fused to, a human immunoglobulin Fc chain, e.g., human IgG (e.g., human IgG1or human IgG2, or a mutated form thereof). The Fc sequence can be mutated at one or more amino acids to reduce effector cell function, Fc receptor binding and/or complement activity.

It will be understood that the antibody molecules and soluble or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents.

In another embodiment, the IL-13 or IL-4 antagonist inhibits the expression of nucleic acid encoding an IL-13 or IL-13R, or an IL-4 or IL-4R. Examples of such antagonists include nucleic acid molecules, for example, antisense molecules, ribozymes, RNAi, siRNA, triple helix molecules that hybridize to a nucleic acid encoding an IL-13 or IL-13R, or an IL-4 or IL-4R, or a transcription regulatory region, and blocks or reduces mRNA expression of IL-13 or IL-13R, or an IL-4 or IL-4R. ISIS-369645 provides an example of an antisense nucleic acid that inhibits expression of IL-4R developed by ISIS Pharmaceuticals and disclosed in, e.g., Karras, J. G. et al. (2007) Am J Respir Cell Mol Biol. 36(3):276-86). Exemplary short interference RNAs (siRNAs) that interfere with RNA encoding IL-4 or IL-13 are disclosed in WO 07/131,274.

In yet another embodiment, the IL-13 or IL-4 antagonist is an inhibitor, e.g., a small molecule inhibitor, of upstream or downstream IL-13 signalling (e.g., STAT6 inhibitors). Examples of STAT6 inhibitors are disclosed in WO 04/002964, in Canadian Patent Application: CA 2490888 and in Nagashima, S. et al. (2007) Bioorg Med Chem 15(2):1044-55; and in U.S. Pat. No. 6,207,391 and WO 01/083517.

In other embodiments, one or more IL-13 antagonists alone or in combination with one or more IL-4 antagonists can be co-formulated with, and/or coadministered with, one or more anti-inflammatory drugs, immunosuppressants, or metabolic or enzymatic inhibitors. Examples of the drugs or inhibitors that can be used in combination with the IL-13 binding agents include, but are not limited to, one or more of: TNF antagonists (e.g., a soluble fragment of a TNF receptor, e.g., p55 or p75 human TNF receptor or derivatives thereof, e.g., 75 kd TNFR-IgG (75 kD TNF receptor-IgG fusion protein, ENBREL™)); TNF enzyme antagonists, e.g., TNFα converting enzyme (TACE) inhibitors; muscarinic receptor antagonists; TGF-θ antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, Mk-2 and NFPB inhibitors.

### Vaccine Formulations

In another aspect, the invention features a method of modifying an immune response associated with immunization. An IL-13 antagonist, alone or in combination with an IL-4 antagonist, can be used to increase the efficacy of immunization by inhibiting IL-13 activity. Antagonists can be administered before, during, or after delivery of an immunogen, e.g., administration of a vaccine. In one embodiment, the immunity raised by the vaccination is a cellular immunity, e.g., an immunity against cancer cells or virus infected, e.g., retrovirus infected, e.g., HIV infected, cells. In one embodiment, the vaccine formulation contains one or more antagonists and an antigen, e.g., an immunogen. In one embodiment, the IL-13 and/or IL-4 antagonists are administered in combination with immunotherapy (e.g., in combination with an allergy immunization with one or more immunogens chosen from ragweed, ryegrass, dust mite and the like. In another embodiment, the antagonist and the immunogen are administered separately, e.g., within one hour, three hours, one day, or two days of each other.

Inhibition of IL-13 can improve the efficacy of, e.g., cellular vaccines, e.g., vaccines against diseases such as cancer and viral infection, e.g., retroviral infection, e.g., HIV infection. Induction of CD8⁺ cytotoxic T lymphocytes (CTL) by vaccines is down modulated by CD4⁺ T cells, likely through the cytokine IL-13. Inhibition of IL-13 has been shown to enhance vaccine induction of CTL response (Ahlers et al. (2002) Proc. Natl. Acad. Sci. USA 99:13020-10325). An IL-13 antagonist can be used in conjunction with a vaccine to increase vaccine efficacy. Cancer and viral infection (such as retroviral (e.g., HIV) infection) are exemplary disorders against which a cellular vaccine response can be effective. Vaccine efficacy is enhanced by blocking IL-13 signaling at the time of vaccination (Ahlers et al. (2002) Proc. Nat. Acad. Sci. USA 99:13020-25). A vaccine formulation may be administered to a subject in the form of a pharmaceutical or therapeutic composition.

Methods for Diagnosing, Prognosing, and/or Monitoring IL-1,3-Associated Disorders

The binding agents described herein can be used, e.g., in methods for diagnosing, prognosing, and monitoring the progress of IL-13-associated disorders, e.g., asthma, by measuring the level of IL-13 in a biological sample. In addition, this discovery enables the identification of new inhibitors of IL-13 signaling, which will also be useful in the treatment of IL-13-associated disorders, e.g., asthma. Such methods for diagnosing allergic and nonallergic asthma can include detecting an alteration (e.g., a decrease or increase) of IL-13 in a biological sample, e.g., serum, plasma, bronchoalveolar lavage fluid, sputum, etc. “Diagnostic” or “diagnosing” means identifying the presence or absence of a pathologic condition. Diagnostic methods involve detecting the presence of IL-13 by determining a test amount of IL-13 polypeptide in a biological sample, e.g., in bronchoalveolar lavage fluid, from a subject (human or nonhuman mammal), and comparing the test amount with a normal amount or range (i.e., an amount or range from an individual(s) known not to suffer from asthma) for the IL-13 polypeptide. While a particular diagnostic method may not provide a definitive diagnosis of asthma, it suffices if the method provides a positive indication that aids in diagnosis.

Methods for prognosing asthma and/or atopic disorders can include detecting upregulation of IL-13, at the mRNA or protein level. “Prognostic” or “prognosing” means predicting the probable development and/or severity of a pathologic condition. Prognostic methods involve determining the test amount of IL-13 in a biological sample from a subject, and comparing the test amount to a prognostic amount or range (i.e., an amount or range from individuals with varying severities of asthma) for IL-13. Various amounts of the IL-13 in a test sample are consistent with certain prognoses for asthma. The detection of an amount of IL-13 at a particular prognostic level provides a prognosis for the subject.

The present application also provides methods for monitoring the course of asthma by detecting the upregulation of IL-13. Monitoring methods involve determining the test amounts of IL-13 in biological samples taken from a subject at a first and second time, and comparing the amounts. A change in amount of IL-13 between the first and second time can indicate a change in the course of asthma and/or atopic disorder, with a decrease in amount indicating remission of asthma, and an increase in amount indicating progression of asthma and/or atopic disorder. Such monitoring assays are also useful for evaluating the efficacy of a particular therapeutic intervention (e.g., disease attenuation and/or reversal) in patients being treated for an IL-13 associated disorder.

Fluorophore- and chromophore-labeled binding agents can be prepared. The fluorescent moieties can be selected to have substantial absorption at wavelengths above 310 nm, and preferably above 400 nm. A variety of suitable fluorescers and chromophores are described by Stryer (1968) Science, 162:526 and Brand, L. et al. (1972) Annual Review of Biochemistry, 41:843-868. The binding agents can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the binding agent can be used to detect the presence or localization of the IL-13 in a sample, e.g., using fluorescent microscopy (such as confocal or deconvolution microscopy).

Histological Analysis. Immunohistochemistry can be performed using the binding agents described herein. For example, in the case of an antibody, the antibody can synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation. The antibody (or other polypeptide or peptide) can be unlabeled at the time of binding. After binding and washing, the antibody is labeled in order to render it detectable.

Protein Arrays. An IL-13 binding agent (e.g., a protein that is an IL-13 binding agent) can also be immobilized on a protein array. The protein array can be used as a diagnostic tool, e.g., to screen medical samples (such as isolated cells, blood, sera, biopsies, and the like). The protein array can also include other binding agents, e.g., ones that bind to IL-13 or to other target molecules.

Methods of producing protein arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer. For example, the array can be an array of antibodies, e.g., as described in De Wildt, supra. Cells that produce the protein can be grown on a filter in an arrayed format. proteins production is induced, and the expressed protein are immobilized to the filter at the location of the cell.

A protein array can be contacted with a sample to determine the extent of IL-13 in the sample. If the sample is unlabeled, a sandwich method can be used, e.g., using a labeled probe, to detect binding of the IL-13. Information about the extent of binding at each address of the array can be stored as a profile, e.g., in a computer database. The protein array can be produced in replicates and used to compare binding profiles, e.g., of different samples.

Flow Cytometry. The IL-13 binding agent can be used to label cells, e.g., cells in a sample (e.g., a patient sample). The binding agent can be attached (or attachable) to a fluorescent compound. The cells can then be analyzed by flow cytometry and/or sorted using fluorescent activated cell sorted (e.g., using a sorter available from Becton Dickinson Immunocytometry Systems, San Jose Calif.; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample. The sorter can also deflect the cell and separate cells bound by the binding agent from those cells not bound by the binding agent. The separated cells can be cultured and/or characterized.

In vivo Imaging. In still another embodiment, the invention provides a method for detecting the presence of a IL-13 within a subject in vivo. The method includes (i) administering to a subject (e.g., a patient having an IL-13 associated disorder) an anti-IL-13 antibody molecule, conjugated to a detectable marker; (ii) exposing the subject to a means for detecting the detectable marker. For example, the subject is imaged, e.g., by NMR or Other Tomographic Means.

Examples of labels useful for diagnostic imaging include radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The binding agent can be labeled with such reagents using known techniques. For example, see Wensel and Meares (1983) Radioimmunoimaging and Radioimmunotherapy, Elsevier, N.Y. for techniques relating to the radiolabeling of antibodies and Colcher et al. (1986) Meth. Enzymol. 121: 802-816. A radiolabeled binding agent can also be used for in vitro diagnostic tests. The specific activity of a isotopically-labeled binding agent depends upon the half-life, the isotopic purity of the radioactive label, and how the label is incorporated into the antibody. Procedures for labeling polypeptides with the radioactive isotopes (such as ¹⁴C, ³H, ³⁵S, ¹²⁵I, ^{99m}Tc, ³²P, ³³P, and ¹³¹I) are generally known. See, e.g., U.S. Pat. No. 4,302,438; Goding, J. W. (Monoclonal antibodies: principles and practice: production and application of monoclonal antibodies in cell biology, biochemistry, and immunology 2nd ed. London; Orlando: Academic Press, 1986. pp 124-126) and the references cited therein; and A. R. Bradwell et al., “Developments in Antibody Imaging”, Monoclonal Antibodies for Cancer Detection and Therapy, R. W. Baldwin et al., (eds.), pp 65-85 (Academic Press 1985).

IL-13 binding agents described herein can be conjugated to Magnetic Resonance Imaging (MRI) contrast agents. Some MRI techniques are summarized in EP-A-0 502 814. Generally, the differences in relaxation time constants T1 and T2 of water protons in different environments is used to generate an image. However, these differences can be insufficient to provide sharp high resolution images. The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe³⁺, Mn²⁺, Gd³⁺). Other agents can be in the form of particles, e.g., less than 10 μm to about 10 nm in diameter) and having ferromagnetic, antiferromagnetic, or superparamagnetic properties. The IL-13 binding agents can also be labeled with an indicating group containing the NMR active ¹⁹F atom, as described by Pykett (1982) Scientific American, 246:78-88 to locate and image IL-13 distribution.

Also within the scope described herein are kits comprising an IL-13 binding agent and instructions for diagnostic use, e.g., the use of the IL-13 binding agent (e.g., an antibody molecule or other polypeptide or peptide) to detect IL-13, in vitro, e.g., in a sample, e.g., a biopsy or cells from a patient having an IL-13 associated disorder, or in vivo, e.g., by imaging a subject. The kit can further contain a least one additional reagent, such as a label or additional diagnostic agent. For in vivo use the binding agent can be formulated as a pharmaceutical composition.

### Kits

An IL-13 binding agent, e.g., an anti-IL-13 antibody molecule, and/or the IL-4 antagonist can be provided in a kit, e.g., as a component of a kit. For example, the kit includes (a) an IL-13 binding agent, e.g., an anti-IL-13 antibody molecule, and/or the IL-4 antagonist and, optionally (b) informational material. The informational material can be descriptive, instructional, marketing or other material that relates to a method, e.g., a method described herein. The informational material of the kits is not limited in its form. In one embodiment, the informational material can include information about production of the compound, molecular weight of the compound, concentration, date of expiration, batch or production site information, and so forth. In one embodiment, the informational material relates to using the IL-13 binding agent to treat, prevent, diagnose, prognose, or monitor a disorder described herein. In one embodiment the informational material includes instructions for administration of the IL-13 binding as a single treatment interval.

In one embodiment, the informational material can include instructions to administer an IL-13 binding agent, e.g., an anti-IL-13 antibody molecule, in a suitable manner to perform the methods described herein, e.g., in a suitable dose, dosage form, or mode of administration (e.g., a dose, dosage form, mode of administration, pharmacokinetic/phamacodynamic properties described herein). In another embodiment, the informational material can include instructions to administer an IL-13 binding agent, e.g., an anti-IL-13 antibody molecule, to a suitable subject, e.g., a human, e.g., a human having, or at risk for, allergic asthma, non-allergic asthma, or an IL-13 mediated disorder, e.g., an allergic and/or inflammatory disorder, or HTLV-1 infection. IL-13 production has been correlated with HTLV-1 infection (Chung et al., (2003) Blood 102: 4130-36).

For example, the material can include instructions to administer an IL-13 binding agent, e.g., an anti-IL-13 antibody molecule, to a patient, a patient with or at risk for allergic asthma, non-allergic asthma, or an IL-13 mediated disorder, e.g., an allergic and/or inflammatory disorder, or HTLV-1 infection.

The kit can include one or more containers for the composition containing an IL-13 binding agent, e.g., an anti-IL-13 antibody molecule. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an IL-13 binding agent, e.g., anti-IL-13 antibody molecule. For example, the kit includes a plurality of syringes, ampules, foil packets, atomizers or inhalation devices, each containing a single unit dose of an IL-13 binding agent, e.g., an anti-IL-13 antibody molecule, or multiple unit doses.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is an implantable device that dispenses metered doses of the binding agent.

The Examples that follow are set forth to aid in the understanding of the inventions but are not intended to, and should not be construed to, limit its scope in any way.

### EXAMPLES

### Example 1

### MJ 2-7 Antibody

Total RNA was prepared from MJ 2-7 hybridoma cells using the QIAGEN RNEASY3 Mini Kit (Qiagen). RNA was reverse transcribed to cDNA using the SMART3 PCR Synthesis Kit (BD Biosciences Clontech). The variable region of MJ 2-7 heavy chain was extrapolated by PCR using SMART3 oligonucleotide as a forward primer and mIgG1 primer annealing to DNA encoding the N-terminal part of CH1 domain of mouse IgG1 constant region as a reverse primer. The DNA fragment encoding MJ 2-7 light chain variable region was generated using SMART3 and mouse kappa specific primers. The PCR reaction was performed using DEEP VENT3 DNA polymerase (New England Biolabs) and 25 nM of dNTPs for 24 cycles (94° C. for 1 minute, 60° C. for 1 minute, 72° C. for 1 minute). The PCR products were subcloned into the pED6 vector, and the sequence of the inserts was identified by DNA sequencing. N-terminal protein sequencing of the purified mouse MJ 2-7 antibody was used to confirm that the translated sequences corresponded to the observed protein sequence.

Exemplary nucleotide and amino acid sequences of mouse monoclonal antibody MJ 2-7 which interacts with NHP IL-13 and which has characteristics which suggest that it may interact with human IL-13 are as follows:

An exemplary nucleotide sequence encoding the heavy chain variable domain includes:

(SEQ ID NO:129)

GAG GTTCAGCTGC AGCAGTCTGG GGCAGAGCTT GTGAAGCCAG
GGGCCTCAGT CAAGTTGTCC TGCACAGGTT CTGGCTTCAA
CATTAAAGAC ACCTATATAC ACTGGGTGAA GCAGAGGCCT
GAACAGGGCC TGGAGTGGAT TGGAAGGATT GATCCTGCGA
ATGATAATAT TAAATATGAC CCGAAGTTCC AGGGCAAGGC
CACTATAACA GCAGACACAT CCTCCAACAC AGCCTACCTA
CAGCTCAACA GCCTGACATC TGAGGACACT GCCGTCTATT
ACTGTGCTAG ATCTGAGGAA AATTGGTACG ACTTTTTTGA
CTACTGGGGC CAAGGCACCA CTCTCACAGT CTCCTCA

An exemplary amino acid sequence for the heavy chain variable domain includes:

(SEQ ID NO:130)

EVQLQQSGAELVKPGASVKLSCTGSGFNIKDTYIHWVKQRPEQGLEWIGR
IDPANDNIKYDPKFQGKATITADTSSNTAYLQLNSLTSEDTAVYYCARSE
ENWYDFFDYWGQGTTLTVSS

CDRs are underlined. The variable domain optionally is preceded by a leader sequence. e.g., MKCSWVIFFLMAVVTGVNS (SEQ ID NO:131). An exemplary nucleotide sequence encoding the light chain variable domain includes:

(SEQ ID NO:132)

GAT GTTTTGATGA CCCAAACTCC ACTCTCCCTG CCTGTCAGTC
TTGGAGATCA AGCCTCCATC TCTTGCAGGT CTAGTCAGAG
CATTGTACAT AGTAATGGAA ACACCTATTT AGAATGGTAC
CTGCAGAAAC CAGGCCAGTC TCCAAAGCTC CTGATCTACA
AAGTTTCCAA CCGATTTTCT GGGGTCCCAG ACAGGTTCAG
TGGCAGTGGA TCAGGGACAG ATTTCACACT CAAGATTAGC
AGAGTGGAGG CTGAGGATCT GGGAGTTTAT TACTGCTTTC
AAGGTTCACA TATTCCGTAC ACGTTCGGAG GGGGGACCAA
GCTGGAAATA AAA

An exemplary amino acid sequence for the light chain variable domain includes:

(SEQ ID NO:133)

DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHIP
YTFGGGTKLEIK

CDRs are underlined. The amino acid sequence optionally is preceded by a leader sequence, e.g., MKLPVRLLVLMFWIPASSS (SEQ ID NO:134). The term “MJ 2-7” is used interchangeably with the term “mAb7.1. 1,” herein.

### Example 2

### C65 Antibody

Exemplary nucleotide and amino acid sequences of mouse monoclonal antibody C65, which interacts with NHP IL-13 and which has characteristics that suggest that it may interact with human IL-13 are as follows:

An exemplary nucleic acid sequence for the heavy chain variable domain includes:

```
(SEQ ID NO:135)
    1 ATGGCTGTCC TGGCATTACT CTTCTGCCTG GTAACATTCC CAAGCTGTAT
  51 CCTTTCCCAG GTGCAGCTGA AGGAGTCAGG ACCTGGCCTG GTGGCGCCCT
101 CACAGAGCCT GTCCATCACA TGCACCGTCT CAGGGTTCTC ATTAACCGGC
151 TATGGTGTAA ACTGGGTTCG CCAGCCTCCA GGAAAGGGTC TGGAGTGGCT
201 GGGAATAATT TGGGGTGATG GAAGCACAGA CTATAATTCA GCTCTCAAAT
251 CCAGACTGAT CATCAACAAG GACAACTCCA AGAGCCAAGT TTTCTTAAAA
301 ATGAACAGTC TGCAAACTGA TGACACAGCC AGGTACTTCT GTGCCAGAGA
351 TAAGACTTTT TACTACGATG GTTTCTACAG GGGCAGGATG GACTACTGGG
401 GTCAAGGAAC CTCAGTCACC GTCTCCTCA
```

An exemplary amino acid sequence for the heavy chain variable domain includes:

| | |
|---|---|
| QVQLKESGPGL VAPSQSLSIT CTVSGFSLTG YGVNWVRQPP GKGLEWLGII | (SEQ ID NO: 136) |
| WGDGSTDYNS ALKSRLIINK DNSKSQVFLK MNSLQTDDTA RYFCARDKTF | |
| YYDGFYRGRM DYWGQGTSVT VSS | |

CDRs are underlined. The amino acid sequence optionally is preceded by a leader sequence, e.g., MAVLALLFCL VTFPSCILS (SEQ ID NO:137).

An exemplary nucleotide sequence encoding the light chain variable domain includes:

| | | |
|---|---|---|
| 1 | ATGAACACGA GGGCCCCTGC TGAGTTCCTT GGGTTCCTGT TGCTCTGGTT | (SEQ ID NO: 138) |
| 51 | TTTAGGTGCC AGATGTGATG TCCAGATGAT TCAGTCTCCA TCCTCCCTGT | |
| 101 | CTGCATCTTT GGGAGACATT GTCACCATGA CTTGCCAGGC AAGTCAGGGC | |
| 151 | ACTAGCATTA ATTTAAACTG GTTTCAGCAA AAACCAGGGA AAGCTCCTAA | |
| 201 | GCTCCTGATC TTTGGTGCAA GCAACTTGGA AGATGGGGTC CCATCAAGGT | |
| 251 | TCAGTGGCAG TAGATATGGG ACAAATTTCA CTCTCACCAT CAGCAGCCTG | |
| 301 | GAGGATGAAG ATATGGCAAC TTATTTCTGT CTACAGCATA GTTATCTCCC | |
| 351 | GTGGACGTTC GGTGGCGGCA CCAAACTGGA AATCAAA | |

An exemplary amino acid sequence for the light chain variable domain includes:

| | |
|---|---|
| DVQMIQSP SSLSASLGDI VTMTCQASQG TSINLNWFQQ KPGKAPKLLI | (SEQ ID NO: 139) |
| FGASNLEDGV PSRFSGSRYG TNFTLTISSL EDEDMATYFC LQHSYLPWTF | |
| GGGTKLEIK | |

CDRs are underlined. The amino acid sequence optionally is preceded by a leader sequence, e.g., MNTRAPAEFLGFLLLWFLGARC (SEQ ID NO:140).

### Example 3

### Fc Sequences

The Ser at position #1 of SEQ ID NO:128 represents amino acid residue #119 in a first exemplary full length antibody numbering scheme in which the Ser is preceded by residue #118 of a heavy chain variable domain. In the first exemplary full length antibody numbering scheme, mutated amino acids are at numbered 234 and 237, and correspond to positions 116 and 119 of SEQ ID NO:128. Thus, the following sequence represents an Fc domain with two mutations: L234A and G237A, according to the first exemplary full length antibody numbering scheme.

### Mus Musculus (SEQ ID NO:128)

The following is another exemplary human Fc domain sequence:

| | |
|---|---|
| STKGPSVFPLAPSSKSTSGGTAALGCLVKDYFPEPVTVSWNSGALTSGVHTFPAV | (SEQ ID NO: 141) |
| LQSSGLYSLSSVVTVPSSSLGTQTYICNVNHKPSNTKVDKKVEPKSCDKTHTCPP | |
| CPAPELLGGPSVFLFPPKPKDTLMISRTPEVTCVVVDVSHEDPEVKFNWYVDGVE | |
| VHNAKTKPREEQYNSTYRVVSVLTVLHQDWLNGKEYKCKVSNKALPAPIEKTIS | |
| KAKGQPREPQVYTLPPSREEMTKNQVSLTCLVKGFYPSDIAVEWESNGQPENNY | |
| KTTPPVLDSDGSFFLYSKLTVDKSRWQQGNVFSCSVMHEALHNHYTQKSLSLSP | |
| GK | |

Other exemplary alterations that can be used to decrease effector function include L234A; L235A), (L235A; G237A), and N297A.

### Example 4

### IL-13 and Atopic Disorders

The ability of MJ2-7 to inhibit the bioactivity of native human IL-13 (at 1 ng/ml) was evaluated in an assay for STAT6 phosphorylation. MJ2-7 inhibited the activity of native human IL-13 with an IC50 of about 0.293 nM in this assay. An antibody with the murine heavy chain of MJ2-7 and a humanized light chain inhibited the activity of native human IL-13 with an IC50 of about 0.554 nM in this assay.

The ability of MJ2-7 to inhibit non-human primate IL-13 (at 1 ng/ml) was evaluated in an assay for CD23 expression. The MJ2-7 inhibited the activity of non-human primate IL-13 with an IC50 of about 0.242 nM in this assay. An antibody with the murine heavy chain of MJ2-7 and a humanized light chain inhibited the activity of non-human primate IL-13 with an IC50 of about 0.308 nM in this assay.

### Example 5

### Nucleotide and amino acid sequences of mouse MJ 2-7 Antibody

The nucleotide sequence encoding the heavy chain variable region (with an optional leader) is as follows:

| | | |
|---|---|---|
| 1 | ATGAAATGCA GCTGGGTTAT CTTCTTCCTG ATGGCAGTGG TTACAGGGGT | (SEQ ID NO: 142) |
| 51 | CAATTCAGAG GTTCAGCTGC AGCAGTCTGG GGCAGAGCTT GTGAAGCCAG | |
| 101 | GGGCCTCAGT CAAGTTGTCC TGCACAGGTT CTGGCTTCAA CATTAAAGAC | |
| 151 | ACCTATATAC ACTGGGTGAA GCAGAGGCCT GAACAGGGCC TGGAGTGGAT | |
| 201 | TGGAAGGATT GATCCTGCGA ATGATAATAT TAAATATGAC CCGAAGTTCC | |
| 251 | AGGGCAAGGC CACTATAACA GCAGACACAT CCTCCAACAC AGCCTACCTA | |
| 301 | CAGCTCAACA GCCTGACATC TGAGGACACT GCCGTCTATT ACTGTGCTAG | |
| 351 | ATCTGAGGAA AATTGGTACG ACTTTTTTGA CTACTGGGGC CAAGGCACCA | |
| 401 | CTCTCACAGT CTCCTCA | |

The amino acid sequence of the heavy chain variable region with an optional leader (underscored) is as follows:

| | | |
|---|---|---|
| 1 | MKCSWVIFFL MAVVTGVNSE VQLQQSGAEL VKPGASVKLS CTGSGFNIKD | (SEQ ID NO: 143) |
| 51 | TYIHWVKQRP EQGLEWIGRI DPANDNIKYD PKFQGKATIT ADTSSNTAYL | |
| 101 | QLNSLTSEDT AVYYCARSEE NWYDFFDYWG QGTTLTVSS | |

The nucleotide sequence encoding the light chain variable region is as follows:

| | | |
|---|---|---|
| 1 | ATGAAGTTGC CTGTTAGGCT GTTGGTGCTG ATGTTCTGGA TTCCTGCTTC | (SEQ ID NO: 144) |
| 51 | CAGCAGTGAT GTTTTGATGA CCCAAACTCC ACTCTCCCTG CCTGTCAGTC | |
| 101 | TTGGAGATCA AGCCTCCATC TCTTGCAGGT CTAGTCAGAG CATTGTACAT | |
| 151 | AGTAATGGAA ACACCTATTT AGAATGGTAC CTGCAGAAAC CAGGCCAGTC | |
| 201 | TCCAAAGCTC CTGATCTACA AAGTTTCCAA CCGATTTTCT GGGGTCCCAG | |
| 251 | ACAGGTTCAG TGGCAGTGGA TCAGGGACAG ATTTCACACT CAAGATTAGC | |
| 301 | AGAGTGGAGG CTGAGGATCT GGGAGTTTAT TACTGCTTTC AAGGTTCACA | |
| 351 | TATTCCGTAC ACGTTCGGAG GGGGGACCAA GCTGGAAATA AAA | |

The amino acid sequence of the light chain variable region with an optional leader (underscored) is as follows:

| | | |
|---|---|---|
| 1 | MKLPVRLLVL MFWIPASSSD VLMTQTPLSL PVSLGDQASI SCRSSQSIVH | (SEQ ID NO: 145) |
| 51 | SNGNTYLEWY LQKPGQSPKL LIYKVSNRFS GVPDRFSGSG SGTDFTLKIS | |
| 101 | RVEAEDLGVY YCFQGSHIPY TFGGGTKLEI K | |

### Example 6

### Nucleotide and Amino Acid Sequences of Exemplary First Humanized Variants of the MJ 2-7 Antibody

Humanized antibody Version 1 (V1) is based on the closest human germline clones. The nucleotide sequence of hMJ 2-7 V1 heavy chain variable region (hMJ 2-7 VH V1) (with a sequence encoding an optional leader sequence) is as follows:

| | | |
|---|---|---|
| 1 | ATGGATTGGA CCTGGCGCAT CCTGTTCCTG GTGGCCGCTG CCACCGGCGC | (SEQ ID NO: 146) |
| 51 | TCACTCTCAG GTGCAGCTGG TGCAGTCTGG CGCCGAGGTG AAGAAGCCTG | |
| 101 | GCGCTTCCGT GAAGGTGTCC TGTAAGGCCT CCGGCTTCAA CATCAAGGAC | |
| 151 | ACCTACATCC ACTGGGTGCG GCAGGCTCCC GGCCAGCGGC TGGAGTGGAT | |
| 201 | GGGCCGGATC GATCCTGCCA ACGACAACAT CAAGTACGAC CCCAAGTTTC | |
| 251 | AGGGCCGCGT GACCATCACC CGCGATACCT CCGCTTCTAC CGCCTACATG | |
| 301 | GAGCTGTCTA GCCTGCGGAG CGAGGATACC GCCGTGTACT ACTGCGCCCG | |
| 351 | CTCCGAGGAG AACTGGTACG ACTTCTTCGA CTACTGGGGC CAGGGCACCC | |
| 401 | TGGTGACCGT GTCCTCT | |

The amino acid sequence of the heavy chain variable region (hMJ 2-7 V1) is based on a CDR grafted to DP-25, VH-I, 1-03. The amino acid sequence with an optional leader (first underscored region; CDRs based on AbM definition shown in subsequent underscored regions) is as follows:

| | | |
|---|---|---|
| 1 | MDWTWRILFL VAAATGAHS - Q VQLVQSGAEV KKPGASVKVS CKASGFNIKD | (SEQ ID NO: 147) |
| 51 | TYIHWVRQAP GQRLEWMGRI DPANDNIKYD PKFQGRVTIT RDTSASTAYM | |
| 101 | ELSSLRSEDT AVYYCARSEE NWYDFFDYWG QGTLVTVSSG ESCR | |

The nucleotide sequence of the hMJ 2-7 V1 light chain variable region (hMJ 2-7 VL V1) (with a sequence encoding an optional leader sequence) is as follows:

| | | |
|---|---|---|
| 1 | ATGCGGCTGC CCGCTCAGCT GCTGGGCCTG CTGATGCTGT GGGTGCCCGG | (SEQ ID NO: 148) |
| 51 | CTCTTCCGGC GACGTGGTGA TGACCCAGTC CCCTCTGTCT CTGCCCGTGA | |
| 101 | CCCTGGGCCA GCCCGCTTCT ATCTCTTGCC GGTCCTCCCA GTCCATCGTG | |
| 151 | CACTCCAACG GCAACACCTA CCTGGAGTGG TTTCAGCAGA GACCCGGCCA | |
| 201 | GTCTCCTCGG CGGCTGATCT ACAAGGTGTC CAACCGCTTT TCCGGCGTGC | |
| 251 | CCGATCGGTT CTCCGGCAGC GGCTCCGGCA CCGATTTCAC CCTGAAGATC | |
| 301 | AGCCGCGTGG AGGCCGAGGA TGTGGGCGTG TACTACTGCT TCCAGGGCTC | |
| 351 | CCACATCCCT TACACCTTTG GCGGCGGAAC CAAGGTGGAG ATCAAG | |

This version is based on a CDR graft to DPK18, V kappaII. The amino acid sequence of hMJ 2-7 V1 light chain variable region (hMJ 2-7 VL V1) (with optional leader as first underscored region; CDRs based on AbM definition in subsequent underscored regions) is as follows:

| | | |
|---|---|---|
| 1 | MRLPAQLLGL LMLWVPGSSG -DVVMTQSPLS LPVTLGQPAS ISCRSSQSIV | (SEQ ID NO: 149) |
| 51 | HSNGNTYLEW FQQRPGQSPR RLIYKVSNRF SGVPDRFSGS GSGTDFTLKI | |
| 101 | SRVEAEDVGV YYCFQGSHIP YTFGGGTKVE IK | |

### Example 7

### Nucleotide and Amino Acid Sequences of Exemplary Second Humanized Variants of the MJ 2-7 Antibody

The following heavy chain variable region is based on a CDR graft to DP-54, VH-3, 3-07. The nucleotide sequence of hMJ 2-7 Version 2 (V2) heavy chain variable region (hMJ 2-7 VH V2) (with a sequence encoding an optional leader sequence) is as follows:

| | | |
|---|---|---|
| 1 | ATGGAGCTGG GCCTGTCTTG GGTGTTCCTG GTGGCTATCC TGGAGGGCGT | (SEQ ID NO: 150) |
| 51 | GCAGTGCGAG GTGCAGCTGG TGGAGTCTGG CGGCGGACTG GTGCAGCCTG | |
| 101 | GCGGCTCTCT GCGGCTGTCT TGCGCCGCTT CCGGCTTCAA CATCAAGGAC | |
| 151 | ACCTACATCC ACTGGGTGCG GCAGGCTCCC GGCAAGGGCC TGGAGTGGGT | |
| 201 | GGCCCGGATC GATCCTGCCA ACGACAACAT CAAGTACGAC CCCAAGTTCC | |
| 251 | AGGGCCGGTT CACCATCTCT CGCGACAACG CCAAGAACTC CCTGTACCTC | |
| 301 | CAGATGAACT CTCTGCGCGC CGAGGATACC GCCGTGTACT ACTGCGCCCG | |
| 351 | GAGCGAGGAG AACTGGTACG ACTTCTTCGA CTACTGGGGC CAGGGCACCC | |
| 401 | TGGTGACCGT GTCCTCT | |

The amino acid sequence of hMJ 2-7 V2 heavy chain variable region (hMJ 2-7 VH V2) with an optional leader (first underscored region; CDRs based on AbM definition shown in subsequent underscored regions) is as follows:

| | | |
|---|---|---|
| 1 | MELGLSWVFL VAILEGVQC- E VQLVESGGGL VQPGGSLRLS CAASGFNIKD | (SEQ ID NO: 151) |
| 51 | TYIHWVRQAP GKGLEWVARI DPANDNIKYD PKFQGRFTIS RDNAKNSLYL | |
| 101 | QMNSLRAEDT AVYYCARSEE NWYDFFDYWG QGTLVTVSS | |

The hMJ 2-7 V2 light chain variable region was based on a CDR graft to DPK9, V kappaI, 02. The nucleotide sequence of hMJ 2-7 V2 light chain variable region (hMJ 2-7 VL V2) (with a sequence encoding an optional leader sequence) is as follows:

| | | |
|---|---|---|
| 1 | ATGGATATGC GCGTGCCCGC TCAGCTGCTG GGCCTGCTGC TGCTGTGGCT | (SEQ ID NO: 152) |
| 51 | GCGCGGAGCC CGCTGCGATA TCCAGATGAC CCAGTCCCCT TCTTCTCTGT | |
| 101 | CCGCCTCTGT GGGCGATCGC GTGACCATCA CCTGTCGGTC CTCCCAGTCC | |
| 151 | ATCGTGCACT CCAACGGCAA CACCTACCTG GAGTGGTATC AGCAGAAGCC | |
| 201 | CGGCAAGGCC CCTAAGCTGC TGATCTACAA GGTGTCCAAC CGCTTTTCCG | |
| 251 | GCGTGCCTTC TCGGTTCTCC GGCTCCGGCT CCGGCACCGA TTTCACCCTG | |
| 301 | ACCATCTCCT CCCTCCAGCC CGAGGATTTC GCCACCTACT ACTGCTTCCA | |
| 351 | GGGCTCCCAC ATCCCTTACA CCTTTGGCGG CGGAACCAAG GTGGAGATCA | |
| 401 | AGCGT | |

The amino acid sequence of the light chain variable region of hMJ 2-7 V2 light chain variable region (hMJ 2-7 VL V2) (with optional leader peptide underscored and CDRs based on AbM definition shown in subsequent underscored regions) is as follows:

```
(SEQ ID NO:153)
    1 MDMRVPAQLL GLLLLWLRGA RC-DIQMTQSP SSLSASVGDR VTITCRSSQS
  51 IVHSNGNTYL EWYQQKPGKA PKLLIYKVSN RFSGVPSRFS GSGSGTDFTL
101 TISSLQPEDF ATYYCFQGSH IPYTFGGGTK VEIKR
```

Additional humanized versions of MJ 2-7 V2 heavy chain variable region were made. These versions included backmutations that have murine amino acids at selected framework positions.

The nucleotide sequence encoding the heavy chain variable region “Version 2.1” or V2.1 with the back mutations V48I, A29G is as follows:

```
(SEQ ID NO:154)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GATCGGCCGG
151 ATCGATCCTG CCAACGACAA CATCAAGTAC GACCCCAAGT TCCAGGGCCG
201 GTTCACCATC TCTCGCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.1 (CDRs based on AbM definition shown in subsequent underscored regions) is as follows:

```
(SEQ ID NO:155)
    1 EVQLVESGGG LVQPGGSLRL SCAASGFNIK DTYIHWVRQA PGKGLEWIGR
  51 IDPANDNIKY DPKFQGRFTI SRDNAKNSLY LQMNSLRAED TAVYYCARSE
101 ENWYDFFDYW GQGTLVTVSS
```

The nucleotide sequence encoding the heavy chain variable region V2.2 with the back mutations (R67K, F68A) is as follows:

```
(SEQ ID NO:156)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGCCCGG
151 ATCGATCCTG CCAACGACAA CATCAAGTAC GACCCCAAGT TCCAGGGCAA
201 GGCCACCATC TCTCGCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.2 (CDRs based on AbM definition shown in subsequent underscored regions) is as follows:

```
(SEQ ID NO:157)
    1 EVQLVESGGG LVQPGGSLRL SCAASGFNIK DTYIHWVRQA PGKGLEWVAR
  51 IDPANDNIKY DPKFQGKATI SRDNAKNSLY LQMNSLRAED TAVYYCARSE
102 ENWYDFFDYW GQGTLVTVSS
```

The nucleotide sequence encoding the heavy chain variable region V2.3 with the back mutations (R72A):

```
(SEQ ID NO:158)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGCCCGG
151 ATCGATCCTG CCAACGACAA CATCAAGTAC GACCCCAAGT TCCAGGGCCG
201 GTTCACCATC TCTGCCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.3 (CDRs based on AbM definition shown in subsequent underscored regions) is as follows:

```
(SEQ ID NO:159)
    1 EVQLVESGGG LVQPGGSLRL SCAASGFNIK DTYIHWVRQA PGKGLEWVAR
  51 IDPANDNIKY DPKFQGRFTI SADNAKNSLY LQMNSLRAED TAVYYCARSE
103 ENWYDFFDYW GQGTLVTVSS
```

The nucleotide sequence encoding the heavy chain variable region V2.4 with the back mutations (A49G) is as follows:

```
(SEQ ID NO:160)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGGCCGG
151 ATCGATCCTG CCAACGACAA CATCAAGTAC GACCCCAAGT TCCAGGGCCG
201 GTTCACCATC TCTCGCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.4 (CDRs based on AbM definition shown in subsequent underscored regions) is as follows:

```
(SEQ ID NO:161)
    1 EVQLVESGGG LVQPGGSLRL SCAASGFNIK DTYIHWVRQA PGKGLEWVGR
  51 IDPANDNIKY DPKFQGRFTI SRDNAKNSLY LQMNSLRAED TAVYYCARSE
104 ENWYDFFDYW GQGTLVTVSS
```

The nucleotide sequence encoding the heavy chain variable region V2.5 with the back mutations (R67K; F68A; R72A) is as follows:

```
(SEQ ID NO:162)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGCCCGG
151 ATCGATCCTG CCAACGACAA CATCAAGTAC GACCCCAAGT TCCAGGGCAA
201 GGCCACCATC TCTGCCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
352 CGTGTCCTCT
```

The amino acid sequence of the heavy chain variable region of V2.5 (CDRs based on AbM definition shown in subsequent underscored regions) is as follows:

```
(SEQ ID NO:163)
    1 EVQLVESGGG LVQPGGSLRL SCAASGFNIK DTYIHWVRQA PGKGLEWVAR
  51 IDPANDNIKY DPKFQGKATI SADNAKNSLY LQMNSLRAED TAVYYCARSE
105 ENWYDFFDYW GQGTLVTVSS
```

The nucleotide sequence encoding the heavy chain variable region V2.6 with the back mutations (V481; A49G; R72A) is as follows:

```
(SEQ ID NO:164)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GATCGGCCGG
151 ATCGATCCTG CCAACGACAA CATCAAGTAC GACCCCAAGT TCCAGGGCCG
201 GTTCACCATC TCTGCCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.6 (CDRs based on AbM definition shown in subsequent underscored regions) is as follows:

```
(SEQ ID NO:165)
    1 EVQLVESGGG LVQPGGSLRL SCAASGFNIK DTYIHWVRQA PGKGLEWIGR
  51 IDPANDNIKY DPKFQGRFTI SADNAKNSLY LQMNSLRAED TAVYYCARSE
106 ENWYDFFDYW GQGTLVTVSS
```

The nucleotide sequence encoding the heavy chain variable region V2.7 with the back mutations (A49G; R72A) is as follows:

```
(SEQ ID NO:166)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGGCCGG
151 ATCGATCCTG CCAACGACAA CATCAAGTAC GACCCCAAGT TCCAGGGCCG
201 GTTCACCATC TCTGCCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.7 (CDRs based on AbM definition shown in subsequent underscored regions) is as follows:

```
(SEQ ID NO:167)
    1 EVQLVESGGG LVQPGGSLRL SCAASGFNIK DTYIHWVRQA PGKGLEWVGR
  51 IDPANDNIKY DPKFQGRFTI SADNAKNSLY LQMNSLRAED TAVYYCARSE
107 ENWYDFFDYW GQGTLVTVSS
```

The nucleotide sequence encoding the heavy chain variable region V2.8 with the back mutations (L79A) is as follows:

```
(SEQ ID NO:168)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGCCCGG
151 ATCGATCCTG CCAACGACAA CATCAAGTAC GACCCCAAGT TCCAGGGCCG
201 GTTCACCATC TCTCGCGACA ACGCCAAGAA CTCCGCCTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.8 (CDRs based on AbM definition shown in subsequent underscored regions) is as follows:

```
(SEQ ID NO:169)
    1 EVQLVESGGG LVQPGGSLRL SCAASGFNIK DTYIHWVRQA PGKGLEWVAR
  51 IDPANDNIKY DPKFQGRFTI SRDNAKNSAY LQMNSLRAED TAVYYCARSE
108 ENWYDFFDYW GQGTLVTVSS
```

The nucleotide sequence encoding the heavy chain variable region V2.10 with the back mutations (A49G; R72A; L79A) is as follows:

```
(SEQ ID NO:170)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GGTGGGCCGG
151 ATCGATCCTG CCAACGACAA CATCAAGTAC GACCCCAAGT TCCAGGGCCG
201 GTTCACCATC TCTGCCGACA ACGCCAAGAA CTCCGCCTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.10 (CDRs based on AbM definition shown in subsequent underscored regions) is as follows:

```
(SEQ ID NO:171)
    1 EVQLVESGGG LVQPGGSLRL SCAASGFNIK DTYIHWVRQA PGKGLEWVGR
  51 IDPANDNIKY DPKFQGRFTI SADNAKNSAY LQMNSLRAED TAVYYCARSE
109 ENWYDFFDYW GQGTLVTVSS
```

The nucleotide sequence encoding the heavy chain variable region V2.11 with the back mutations (V48I; A49G; R72A; L79A) is as follows:

```
(SEQ ID NO:172)
    1 GAGGTGCAGC TGGTGGAGTC TGGCGGCGGA CTGGTGCAGC CTGGCGGCTC
  51 TCTGCGGCTG TCTTGCGCCG CTTCCGGCTT CAACATCAAG GACACCTACA
101 TCCACTGGGT GCGGCAGGCT CCCGGCAAGG GCCTGGAGTG GATCGGCCGG
151 ATCGATCCTG CCAACGACAA CATCAAGTAC GACCCCAAGT TCCAGGGCCG
201 GTTCACCATC TCTGCCGACA ACGCCAAGAA CTCCGCCTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.11 (CDRs based on AbM definition shown in subsequent underscored regions) is as follows:

```
(SEQ ID NO:173)
    1 EVQLVESGGG LVQPGGSLRL SCAASGFNIK DTYIHWVRQA PGKGLEWIGR
  51 IDPANDNIKY DPKFQGRFTI SADNAKNSAY LQMNSLRAED TAVYYCARSE
110 ENWYDFFDYW GQGTLVTVSS
```

The nucleotide sequence encoding the heavy chain variable region V2.16 with the back mutations (V48I; A49G; R72A) is as follows:

```
(SEQ ID NO:174)
    1 GAGGTGCAGC TGGTGGAGTC TGGCGGCGGA CTGGTGCAGC CTGGCGGCTC
  51 TCTGCGGCTG TCTTGCACCG GCTCCGGCTT CAACATCAAG GACACCTACA
101 TCCACTGGGT GCGGCAGGCT CCCGGCAAGG GCCTGGAGTG GATCGGCCGG
151 ATCGATCCTG CCAACGACAA CATCAAGTAC GACCCCAAGT TCCAGGGCCG
201 GTTCACCATC TCTGCCGACA ACGCCAAGAA CTCCCTGTAC CTCCAGATGA
251 ACTCTCTGCG CGCCGAGGAT ACCGCCGTGT ACTACTGCGC CCGGAGCGAG
301 GAGAACTGGT ACGACTTCTT CGACTACTGG GGCCAGGGCA CCCTGGTGAC
351 CGTGTCCTCT
```

The amino acid sequence of the heavy chain variable region of V2.16 (CDRs based on AbM definition shown in subsequent underscored regions) is as follows:

```
(SEQ ID NO:175)
    1 EVQLVESGGG LVQPGGSLRL SCTGSGFNIK DTYIHWVRQA PGKGLEWIGR
  51 IDPANDNIKY DPKFQGRFTI SADNAKNSLY LQMNSLRAED TAVYYCARSE
111 ENWYDFFDYW GQGTLVTVSS
```

The following is the amino acid sequence of a humanized MH 2-7 V2.11 IgG1 with a mutated CH2 domain:

(SEQ ID NO:176)

EVQLVESGGGLVQPGGSLRLSCAASGFNIKDTYIHWVRQAPGKGLEWIGR
IDPANDNIKYDPKFQGRFTISADNAKNSAYLQMNSLRAEDTAVYYCARSE
ENWYDFFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEALGAPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

The variable domain is at amino acids 1-120; CH1 at 121-218; hinge at 219-233; CH2 at 234-343; and CH3 at 344-450. The light chain includes the following sequence with variable domain at 1-133.

(SEQ ID NO:177)

DIQMTQSPSSLSASVGDRVTITCRSSQSIVHSNGNTYLEWYQQKPGKAPK
LLIYKVSNRFSGVPSRFSGSGSGTDFTLTISSLQPEDFATYYCFQGSHIP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

### Example 8

### Functional Assays of Exemplary Variants of MJ2-7

We evaluated the ability of the MJ2-7 antibody and humanized variants to inhibit human IL-13 in assays for IL-13 activity.

STAT6 Phosphorylation Assay.

HT-29 human colonic epithelial cells (ATCC) were grown as an adherent monolayer in McCoy's 5A medium containing 10% FBS, Pen-Strep, glutamine, and sodium bicarbonate. For assay, the cells were dislodged from the flask using trypsin, washed into fresh medium, and distributed into 12×75 mm polystyrene tubes. Recombinant human IL-13 (R&D Systems, Inc.) was added at concentrations ranging from 100-0.01 ng/ml. For assays testing the ability of antibody to inhibit the IL-13 response, 1 ng/ml recombinant human IL-13 was added along with dilutions of antibody ranging from 500-0.4 ng/ml. Cells were incubated in a 37° C. water bath for 30-60 minutes, then washed into ice-cold PBS containing 1% BSA. Cells were fixed by incubating in 1% paraformaldehyde in PBS for 15 minutes at 37° C., then washed into PBS containing 1% BSA. To permeabilize the nucleus, cells were incubated overnight at −20° C. in absolute methanol. They were washed into PBS containing 1% BSA, then stained with ALEXA3 Fluor 488-labeled antibody to STAT6 (BD Biosciences). Fluorescence was analyzed with a FACSCAN3 and CELLQUEST3 software (BD Biosciences).

CD23 Induction on Human Monocytes

Mononuclear cells were isolated from human peripheral blood by layering over HISTOPAQUE® (Sigma). Cells were washed into RPMI containing 10% heat-inactivated FCS, 50 U/ml penicillin, 50 mg/ml streptomycin, 2 mM L-glutamine, and plated in a 48-well tissue culture plate (Costar/Corning). Recombinant human IL-13 (R&D Systems, Inc.) was added at dilutions ranging from 100-0.01 ng/ml. For assays testing the ability of antibody to inhibit the IL-13 response, 1 ng/ml recombinant human IL-13 was added along with dilutions of antibody ranging from 500-0.4 ng/ml. Cells were incubated overnight at 37° C. in a 5% CO₂ incubator. The next day, cells were harvested from wells using non-enzymatic Cell Dissociation Solution (Sigma), then washed into ice-cold PBS containing 1% BSA. Cells were incubated with phycoerythrin (PE)-labeled antibody to human CD23 (BD Biosciences, San Diego, Calif.), and Cy-Chrome-labeled antibody to human CD11b (BD Biosciences). Monocytes were gated based on high forward and side light scatter, and expression of CD11b. CD23 expression on monocytes was determined by flow cytometry using a FACSCAN3 (BD Biosciences), and the percentage of CD23⁺ cells was analyzed with CELLQUEST3 software (BD Biosciences).

TF-1 Cell Proliferation

TF-1 cells are a factor-dependent human hemopoietic cell line requiring interleukin 3 (IL-3) or granulocyte/macrophage colony-stimulating factor (GM-CSF) for their long-term growth. TF-1 cells also respond to a variety of other cytokines, including interleukin 13 (IL-13). TF-1 cells (ATCC) were maintained in RPMI medium containing 10% heat-inactivated FCS, 50 U/ml penicillin, 50 mg/ml streptomycin, 2 mM L-glutamine, and 5 ng/ml recombinant human GM-CSF (R&D Systems). Prior to assay, cells were starved of GM-CSF overnight. For assay, TF-1 cells were plated in duplicate at 5000 cells/well in 96-well flat-bottom microtiter plates (Costar/Corning), and challenged with human IL-13 (R&D Systems), ranging from 100-0.01 ng/ml. After 72 hours in a 37° C. incubator with 5% CO₂, the cells were pulsed with 1 TCi/well ³H-thymidine (Perkin Elmer/New England Nuclear). They were incubated an additional 4.5 hours, then cells were harvested onto filter mats using a TOMTEK3 harvester. ³H-thymidine incorporation was assessed by liquid scintillation counting.

Tenascin Production Assay

BEAS-2B human bronchial epithelial cells (ATCC) were maintained BEGM media with supplements (Clonetics). Cells were plated at 20,000 per well in a 96-well flat-bottom culture plate overnight. Fresh media is added containing IL-13 in the presence or absence of the indicated antibody. After overnight incubation, the supernatants are harvested, and assayed for the presence of the extracellular matrix component, tenascin C, by ELISA. ELISA plates are coated overnight with 1 ug/ml of murine monoclonal antibody to human tenascin (IgG1, k; Chemicon International) in PBS. Plates are washed with PBS containing 0.05% TWEEN®-20 (PBS-Tween), and blocked with PBS containing 1% BSA. Fresh blocking solution was added every 6 minutes for a total of three changes. Plates were washed 3× with PBS-Tween. Cell supernatants or human tenascin standard (Chemicon International) were added and incubated for 60 minutes at 37° C. Plates were washed 3× with PBS-Tween. Tenascin was detected with murine monoclonal antibody to tenascin (IgG2a, k; Biohit). Binding was detected with HRP-labeled antibody to mouse IgG2a, followed by TMB substrate. The reaction was stopped with 0.01 N sulfuric acid. Absorbance was read at 450 nm.

The HT 29 human epithelial cell line can be used to assay STAT6 phosphorylation. HT 29 cells are incubated with 1 ng/ml native human IL-13 crude preparation in the presence of increasing concentrations of the test antibody for 30 minutes at 37° C. Western blot analysis of cell lysates with an antibody to phosphorylated STAT6 can be used to detect dose-dependent IL 13-mediated phosphorylation of STAT6. Similarly, flow cytometric analysis can detect phosphorylated STAT6 in HT 29 cells that were treated with a saturating concentration of IL-13 for 30 minutes at 37° C., fixed, permeabilized, and stained with an ALEXA™ Fluor 488-labeled mAb to phospho-STAT6. An exemplary set of results is set forth in the Table 1. The inhibitory activity of V2.11 was comparable to that of sIL-13Ra2-Fc.

TABLE 1

| | | | Expression | Native hIL-13 |
|---|---|---|---|---|
| Construct | | Backmutations | μg/ml/ | STAT6 assay |
| VH | VL | VH | COS; 48 h | IC 50, nM |
| V2.0 | V2 | None, CDR grafted | 8-10 | >100 |
| | CDR graft | | | |
| V 2.1 | V2 | V48I; A49G | 9-14 | 2.8 |
| V 2.2 | V2 | R67K; F68A | 5-6 | >100 |
| V 2.3 | V2 | R72A | 8-9 | 1.67-2.6 |
| V 2.4 | V2 | A49G | 10 | 17.5 |
| V 2.5 | V2 | R67K; F68A; R72A | 4-5 | 1.75 |
| V 2.6 | V2 | V48I; A49G: R72A | 11-12 | 1.074-3.37 |
| V 2.7 | V2 | A49G; R72A | 10-11 | 1.7 |
| V 2.11 | V2 | V48I; A49G: | 24 | 0.25-0.55 |
| | | R72A: L79A | | |

### Example 9

### Binding Interaction Site Between IL-13 and IL-13RI1

A complex of IL-13, the extracellular domain of IL-13RI1 (residues 27-342 of SEQ ID NO:125), and an antibody that binds human IL-13 was studied by x-ray crystallography. See, e.g., 16163-029001. Two points of substantial interaction were found between IL-13 and IL-13Rα1. The interaction between Ig domain 1 of IL-13Rα1 and IL-13 results in the formation of an extended beta sheet spanning the two molecules. Residues Thr88 [Thr107], Lys89 [Lys108], Ile90 [Ile109], and Glu91 [Glu110] of IL-13 (SEQ ID NO:124, mature sequence [full-length sequence (SEQ ID NO:178)]) form a beta strand that interacts with residues Lys76, Lys77, Ile78 and Ala79 of the receptor (SEQ ID NO:125). Additionally, the side chain of Met33 [Met52] of IL-13 (SEQ ID NO:124 [SEQ ID NO:178]) extends into a hydrophobic pocket that is created by the side chains of these adjoining strands.

The predominant feature of the interaction with Ig domain 3 is the insertion of a hydrophobic residue (Phe107 [Phe126]) of IL-13 (SEQ ID NO:124 [SEQ ID NO:178]) into a hydrophobic pocket in Ig domain 3 of the receptor IL-13Rα1. The hydrophobic pocket of IL-13Rα1 is formed by the side chains of residues Leu319, Cys257, Arg256, and Cys320 (SEQ ID NO:125). The interaction with Phe107 [Phe126] of IL-13 (SEQ ID NO:124 [SEQ ID NO:178]) results in an extensive set of van der Waals interactions between amino acid residues Ile254, Ser255, Arg256, Lys318, Cys320, and Tyr321 of IL-13Rα1 (SEQ ID NO:125) and amino acid residues Arg11 [Arg30], Glu12 [Glu31], Leu13 [Leu32], Ile14 [Ile33], Glu15 [Ile34], Lys104 [Lys123], Lys105 [Lys124], Leu106 [Leu125], Phe107 [Phe126], and Arg108 [Arg 127] of IL-13 (SEQ ID NO:124 [SEQ ID NO:178]). These results demonstrate that an IL-13 binding agent that binds to the regions of IL-13 involved in interaction with IL-13RI1 can be used to inhibit IL-13 signaling.

### Example 10

### Expression of Humanized MJ 2-7 Antibody in COS Cells

To evaluate the production of chimeric anti-NHP IL13 antibodies in the mammalian recombinant system, the variable regions of mouse MJ 2-7 antibody were subcloned into a pED6 expression vector containing human kappa and IgG1mut constant regions. Monkey kidney COS-1 cells were grown in DME media (Gibco) containing 10% heat-inactivated fetal bovine serum, 1 mM glutamine and 0.1 mg/ml Penicillin/Streptomycin. Transfection of COS cells was performed using TRANSITIT3-LT1 Transfection reagent (Mirus) according to the protocol suggested by the reagent supplier. Transfected COS cells were incubated for 24 hours at 37° C. in the presence of 10% CO₂, washed with sterile PBS, and then grown in serum-free media R1CD1 (Gibco) for 48 hours to allow antibody secretion and accumulation in the conditioned media. The expression of chMJ 2-7 antibody was quantified by total human IgG ELISA using purified human IgG1/kappa antibody as a standard.

The production of chimeric MJ 2-7 antibody in COS cells was significantly lower then the control chimeric antibody (Table 2). Therefore, optimization of Ab expression was included in the MJ 2-7 humanization process. The humanized MJ 2-7 V1 was constructed by CDR grafting of mouse MJ 2-7 heavy chain CDRs onto the most homologous human germline clone, DP 25, which is well expressed and represented in typical human antibody response. The CDRs of light chain were subcloned onto human germline clone DPK 18 in order to generate huMJ 2-7 V1 VL. The humanized MJ 2-7 V2 was made by CDR grafting of CDRs MJ 2-7 heavy chain variable region onto DP54 human germline gene framework and CDRs of MJ 2-7 light chain variable region onto DPK9 human germline gene framework. The DP 54 clone belongs to human VH III germline subgroup and DPK9 is from the V kappa I subgroup of human germline genes. Antibody molecules that include VH III and V kappa I frameworks have high expression level in E. coli system and possess high stability and solubility in aqueous solutions (see, e.g., Stefan Ewert et al., J. Mol. Biol. (2003), 325; 531-553, Adrian Auf et al., Methods (2004) 34:215-224). We have used the combination of DP54/DPK9 human frameworks in the production of several recombinant antibodies and have achieved a high expression of antibody (>20 Tg/ml) in the transient COS transfection experiments.

TABLE 2

| mAb | Expression, μg/ml |
|---|---|
| 3D6 | 10.166 |
| Ch MJ 2-7 pED6 (1) | 2.44 |
| Ch MJ 2-7pED6 (2) | 2.035 |
| h12A11 V2 | 1.639 |

The CDR grafted MJ 2-7 V1 and V2 VH and VL genes were subcloned into two mammalian expression vector systems (pED6kappa/pED6 IgG1mut and pSMEN2kappa/pSMED2IgG1mut), and the production of humanized MJ 2-7 antibodies was evaluated in transient COS transfection experiments as described above. In the first set of the experiments the effect of various combinations of huMJ 2-7 VL and VH on the antibody expression was evaluated (Table 3). Changing of MJ 2-7 VL framework regions to DKP9 increased the antibody production 8-10 fold, whereas VL V1 (CDR grafted onto DPK 18) showed only a moderate increase in antibody production. This effect was observed when humanized VL was combined with chimeric MJ 2-7 VH and humanized MJ 2-7 V1 and V2. The CDR grafted MJ 2-7 V2 had a 3-fold higher expression level then CDR grafted MJ 2-7 V1 in the same assay conditions.

TABLE 3

| mAb | Expression, μg/ml |
|---|---|
| ChMJ 2-7 | 1.83 |
| hVH V1/mVL | 3.04 |
| hVH V1/hVL V1 | 6.34 |
| hVH V1/hVL V2 | 15.4 |
| hVH-V2/mVL | 0.2 |
| mVH/hVL-V2 | 18.41 |
| hVH-V2/hVL-V1 | 5.13 |
| hVH-V2/hVL-V2 | 10.79 |

Similar experiments were performed with huMJ 2-7 V2 containing back mutations in the heavy chain variable regions (Table 4). The highest expression level was detected for huMJ 2-7 V2.11 that retained the antigen binding and neutralization properties of mouse MJ 2-7 antibody. Introduction of back mutations at the positions 48 and 49 (V481 and A49G) increased the production of huMJ 2-7 V2 antibody in COS cells, whereas the back mutations of amino acids at the positions 23, 24, 67 and 68 (A23T; A24G; R67K and F68A) had a negative impact on antibody expression.

TABLE 4

| mAb | Expression, μg/ml |
|---|---|
| V2 | 8.27 |
| V2.1 | 12.1 |
| V2.2 | 5.29 |
| V2.3 | 9.60 |
| V2.4 | 8.20 |
| V2.5 | 6.05 |
| V2.6 | 11.3 |
| V2.10 | 9.84 |
| V2.11 | 14.85 |
| V2.16 | 1.765 |

### Example 11

### Molecular Modeling of Humanized MJ2-7 V.2VH

Structure templates for modeling humanized MJ2-7 heavy chain version 2 (MJ2-7 v.2VH) were selected based on BLAST homology searches against Protein Data Bank (PDB). Besides the two structures selected from the BLAST search output, an additional template was selected from an in-house database of protein structures. Model of MJ2-7 v.2VH was built using the three template structures 1JPS (co-crystal structure of human tissue factor in complex with humanized Fab D3h44), 1N8Z (co-crystal structure of human Her2 in complex with Herceptin Fab) and F13.2 (IL-13 in complex with mouse antibody Fab fragment) as templates and the Homology module of InsightII (Accelrys, San Diego). The structurally conserved regions (SCRs) of 1JPS, 1N8Z and F13.2 (available from WO05/121177) were determined based on the Cα distance matrix for each molecule and the template structures were superimposed based on minimum RMS deviation of corresponding atoms in SCRs. The sequence of the target protein MJ2-7 v.2VH was aligned to the sequences of the superimposed templates proteins and coordinates of the SCRs were assigned to the corresponding residues of the target protein. Based on the degree of sequence similarity between the target and the templates in each of the SCRs, coordinates from different templates were used for different SCRs. Coordinates for loops and variable regions not included in the SCRs were generated by Search Loop or Generate Loop methods as implemented in Homology module. Briefly, Search Loop method scans protein structures that would fit properly between two SCRs by comparing the Cα distance matrix of flanking SCR residues with a pre-calculated matrix derived from protein structures that have the same number of flanking residues and an intervening peptide segment of a given length. Generate Loop method that generate atom coordinates de novo was used in those cases where Search Loops did not produce desired results. Conformation of amino acid side chains was kept the same as that in the template if the amino acid residue was identical in the template and the target. However, a conformational search of rotamers was done and the energetically most favorable conformation was retained for those residues that are not identical in the template and target. This was followed by Splice Repair that sets up a molecular mechanics simulation to derive proper bond lengths and bond angles at junctions between two SCRs or between SCR and a variable region. Finally the model was subjected to energy minimization using Steepest Descents algorithm until a maximum derivative of 5 kcal/(mol A) or 500 cycles and Conjugate Gradients algorithm until a maximum derivative of 5 kcal/(mol A) or 2000 cycles. Quality of the model was evaluated using ProStat/Struct_Check command.

Molecular model of mouse MJ2-7 VH was built by following the procedure described for humanized MJ2-7 v.2VH except the templates used were 1QBL and 1QBM, crystal structures for horse anti-cytochrome c antibody FabE8.

Potential differences in CDR-Framework H-bonds predicted by the models hMJ2-7 v.2VH:G26-hMJ2-7 v.2VH:A24 hMJ2-7 v.2VH:Y109-hMJ2-7 v.2VH:S25 mMJ2-7 VH:D61-mMJ2-7 VH:148 mMJ2-7 VH:K₆₃-mMJ2-7 VH:E46 mMJ2-7 VH:Y109-mMJ2-7 VH:R98 These differences suggested the following optional back mutations: A23T, A24G and V481.

Other optional back mutations suggested based on significant RMS deviation of individual amino acids and differences in amino acid residues adjacent to these are: G9A, L115T and R87T.

### Example 12

### IL-13 Neutralization Activity of MJ2-7 and C65

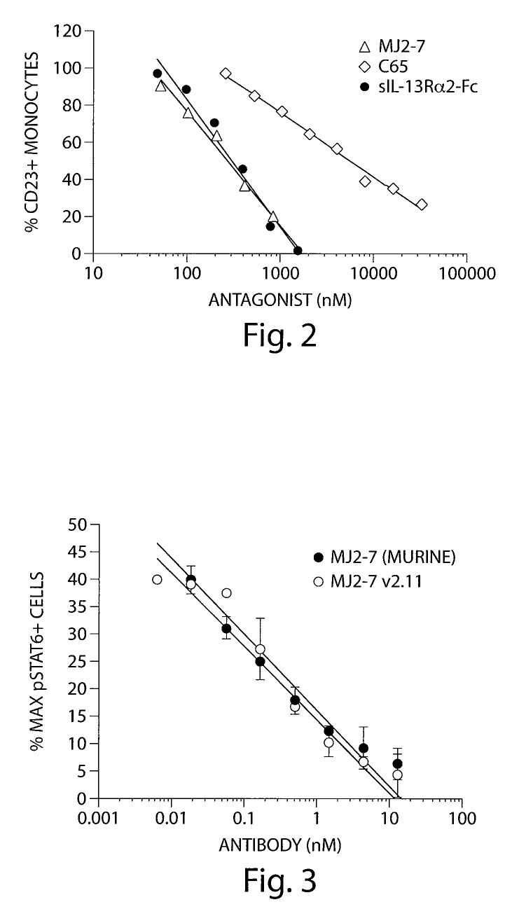

The IL-13 neutralization capacities of MJ2-7 and C65 were tested in a series of bioassays. First, the ability of these antibodies to neutralize the bioactivity of NHP IL-13 was tested in a monocyte CD23 expression assay. Freshly isolated human PBMC were incubated overnight with 3 ng/ml NHP IL-13 in the presence of increasing concentrations of MJ2-7, C65, or sIL-13RI2-Fc. Cells were harvested, stained with CYCHROME3-labeled antibody to the monocyte-specific marker, CD11b, and with PE-labeled antibody to CD23. In response to IL-13 treatment, CD23 expression is up-regulated on the surface of monocytes, which were gated based on expression of CD11b. MJ2-7, C65, and sIL13RI2-Fc all were able to neutralize the activity of NHP IL-13 in this assay. The potencies of MJ2-7 and sIL-13RI2-Fc were equivalent. C65 was approximately 20-fold less active (FIG. 2).

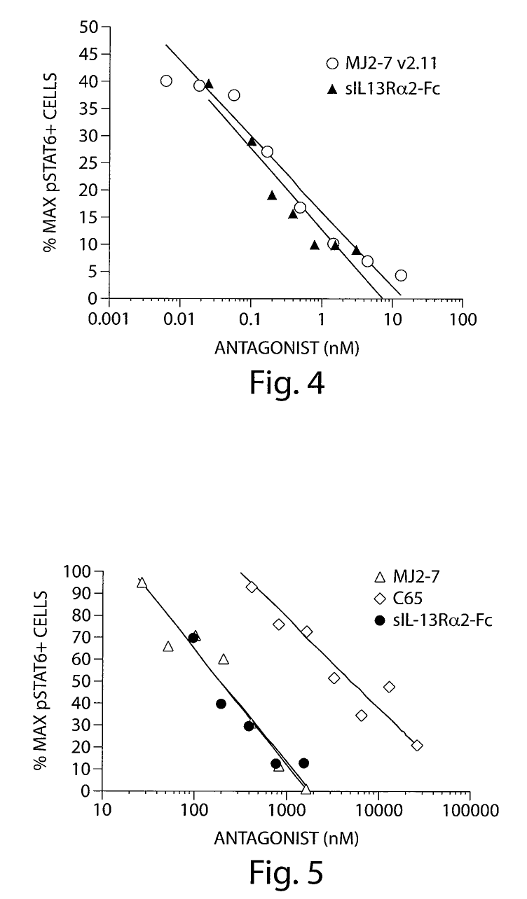

In a second bioassay, the neutralization capacities of MJ2-7 and C65 for native human IL-13 were tested in a STAT6 phosphorylation assay. The HT-29 epithelial cell line was incubated with 0.3 ng/ml native human IL-13 in the presence of increasing concentrations of MJ2-7, C65, or sIL-13RI2-Fc, for 30 minutes at 37° C. Cells were fixed, permeabilized, and stained with ALEXA3 Fluor 488-labeled antibody to phosphorylated STAT6. IL-13 treatment stimulated STAT6 phosphorylation. MJ2-7, C65, and sIL13Ra2-Fc all were able to neutralize the acitivity of native human IL-13 in this assay (FIG. 3). The IC50's for the murine MJ-27 antibody and the humanized form (V2.11) were 0.48 nM and 0.52 nM respectively. The potencies of MJ2-7 and sIL-13RI2-Fc were approximately equivalent. The IC50 for sIL-13Ra2-Fc was 0.33 nM (FIG. 4). C65 was approximately 20-fold less active (FIG. 5).

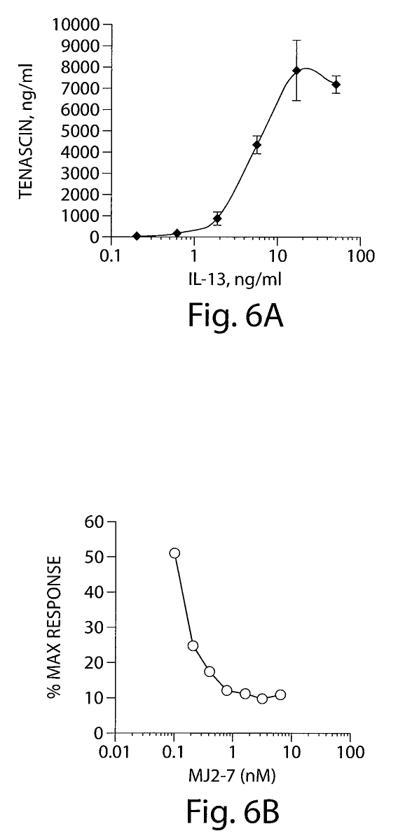

In a third bioassay, the ability of MJ2-7 to neutralize native human IL-13 was tested in a tenascin production assay. The human BEAS-2B lung epithelial cell line was incubated overnight with 3 ng/ml native human IL-13 in the presence of increasing concentrations of MJ2-7. Supernatants were harvested and tested for production of the extracellular matrix protein, tenascin C, by ELISA (FIG. 6A). MJ2-7 inhibited this response with IC50 of approximately 0.1 nM (FIG. 6B).

These results demonstrate that MJ2-7 is an effective neutralizer of both NHP IL-13 and native human IL-13. The IL-13 neutralization capacity of MJ2-7 is equivalent to that of sIL-13RI2-Fc. MJ1-65 also has IL-13 neutralization activity, but is approximately 20-fold less potent than MJ2-7.

### Example 13

### Epitope Mapping of MJ2-7 Antibody by SPR

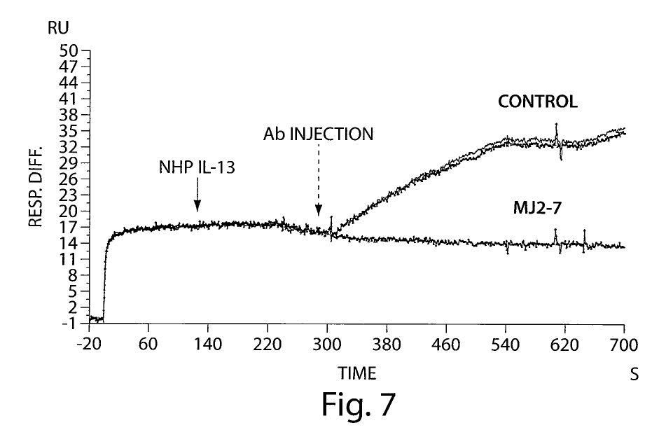

sIL-13RI2-Fc was directly coated onto a CM5 chip by standard amine coupling. NHP-IL-13 at 100 nM concentration was injected, and its binding to the immobilized IL-13RI2-Fc was detected by BIACORE3. An additional injection of 100 nM of anti IL-13 antibodies was added, and changes in binding were monitored. MJ2-7 antibody did not bind to NHP-IL-13 when it was in a complex with hu IL-13RI2, whereas a positive control anti-IL-13 antibody did (FIG. 7). These results indicate that hu IL-13RI2 and MJ2-7 bind to the same or overlapping epitopes of NHP IL-13.

### Example 14

### Measurement of Kinetic Rate Constants for the Interaction Between NHP-IL-13 and Humanized MJ2-7 v.2-11 Antibody

To prepare the biosensor surface, goat anti-human IgG Fc specific antibody was immobilized onto a research-grade carboxy methyl dextran chip (CM5) using amine coupling. The surface was activated with a mixture of 0.1 M 1-ethyl-3-(3-dimethylaminopropyl) carbodiimide (EDC) and 0.05 M N-Hydroxysuccinimide (NHS). The capturing antibody was injected at a concentration of 10 Tg/ml in sodium acetate buffer (pH 5.5). Remaining activated groups were blocked with 1.0 M ethanolamine (pH 8.0). As a control, the first flow cell was used as a reference surface to correct for bulk refractive index, matrix effect's and non-specific binding, the second, third and fourth flow cells were coated with the capturing molecule.

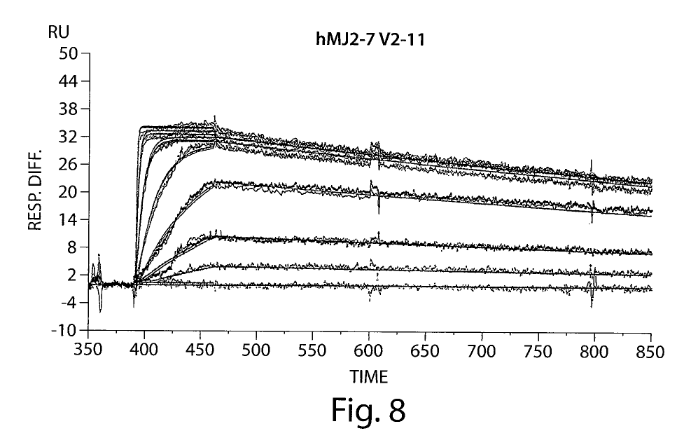

For kinetic analysis, the monoclonal antibody hMJ2-7 v.2-11 was captured onto the anti IgG antibody surface by injecting 40 Tl of a 1 Tg/ml solution. The net difference between the baseline and the point approximately 30 seconds after completion of injection was taken to represent the amount of target bound. Solutions of NHP-IL-13 at 600, 200, 66.6, 22.2, 7.4, 2.5, 0.8, 0.27, 0.09 and 0 nM concentrations were injected in triplicate at a flow rate of 100 Tl per min for 2 minutes, and the amount of bound material as a function of time was recorded (FIG. 8). The dissociation phase was monitored in HBS/EP buffer (10 mM HEPES, pH 7.4, containing 150 mM NaCl, 3 mM EDTA and 0.005% (v/v) Surfactant P20) for 5 minutes at the same flow rate followed by two 5 Tl injections of glycine, pH 1.5, to regenerate a fully active capturing surface. All kinetic experiments were done at 22.5° C. in HBS/EP buffer. Blank and buffer effects were subtracted for each sensorgram using double referencing.

The kinetic data were analyzed using BIAEVALUATION3 software 3.0.2 applied to a 1:1 model. The apparent dissociation (kd) and association (ka) rate constants were calculated from the appropriate regions of the sensorgrams using a global analysis. The affinity constant of the interaction between antibody and NHP IL-13 was calculated from the kinetic rate constants by the following formula: Kd=kd/ka. These results indicate that huMJ2-7 v.2-11 has on and off-rates of 2.05×10⁷ M⁻¹ s⁻¹ and 8.89×10⁻⁴ l/s, respectively, resulting in an antibody with 43 pM affinity for NHP-IL-13.

### Example 15

### Inhibitory Activity of MJ2-7 Humanization Intermediates in Bioassays

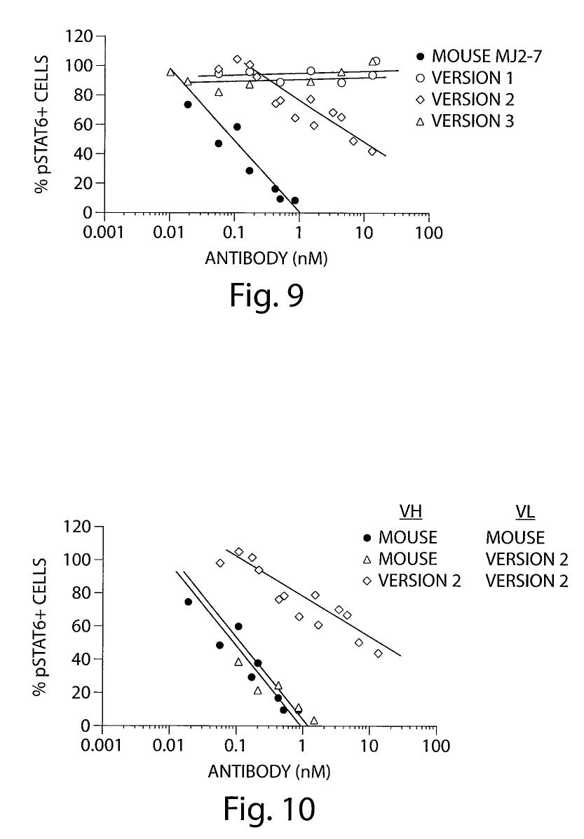

The inhibitory activity of various intermediates in the humanization process was tested by STAT6 phosphorylation and tenascin production bioassays. A sub-maximal level of NHP IL-13 or native human IL-13 crude preparation was used to elicit the biological response, and the concentration of the humanized version of MJ2-7 required for half-maximal inhibition of the response was determined. Analysis hMJ2-7 V1, hMJ2-7 V2 and hMJ2-7 V3, expressed with the human IgG1, and kappa constant regions, showed that Version 2 retained neutralization activity against native human IL-13. This concentration of the Version 2 humanized antibody required for half-maximal inhibition of native human IL-13 bioactivity was approximately 110-fold greater than that of murine MJ2-7 (FIG. 9). Analysis of a semi-humanized form, in which the V1 or V2 VL was combined with murine MJ2-7 VH, demonstrated that the reduction of native human IL-13 neutralization activity was not due to the humanized VL, but rather to the VH sequence (FIG. 10). Whereas the semi-humanized MJ2-7 antibody with VL V1 only partially retained the neutralization activity the version with humanized VL V2 was as active as parental mouse antibody. Therefore, a series of back-mutations were introduced into the V1 VH sequence to improve the native human IL-13 neutralization activity of murine MJ2-7.

### Example 16

### MJ2-7 Blocks IL-13 Interaction with IL-13RI1 and IL-13RI2

MJ2-7 is specific for the C-terminal 19-mer of NHP IL-13, corresponding to amino acid residues 114-132 of the immature protein (SEQ ID NO:24), and residues 95-113 of the mature protein (SEQ ID NO:14). For human IL-13, this region, which forms part of the D alpha-helix of the protein, has been reported to contain residues important for binding to both IL-13RI1 and IL-13RI2. Analysis of human IL-13 mutants identified the A, C, and D-helices as containing important contacts site for the IL-13RI1/IL-4RI signaling complex (Thompson and Debinski (1999) J. Biol. Chem. 274: 29944-50). Alanine scanning mutagenesis of the D-helix identified residues K123, K124, and R127 (SEQ ID NO:24) as responsible for interaction with IL-13RI2, and residues E110, E128, and L122 as important contacts for IL-13RI1 (Madhankmuar et al. (2002) J. Biol. Chem. 277: 43194-205). High resolution solution structures of human IL-13 determined by NMR have predicted the IL-13 binding interactions based on similarities to related ligand-receptor pairs of known structure. These NMR studies have supported a key role for the IL-13 A and D-helices in making important contacts with IL-13RI1 (Eisenmesser et al. (2001) J. Mol. Biol. 310:231-241; Moy et al. (2001) J. Mol. Biol. 310:219-230). Binding of MJ2-7 to this epitope located in the C-terminal, D-helix of IL-13 was predicted to disrupt interaction of IL-13 with IL-13RI1 and IL-13RI2.

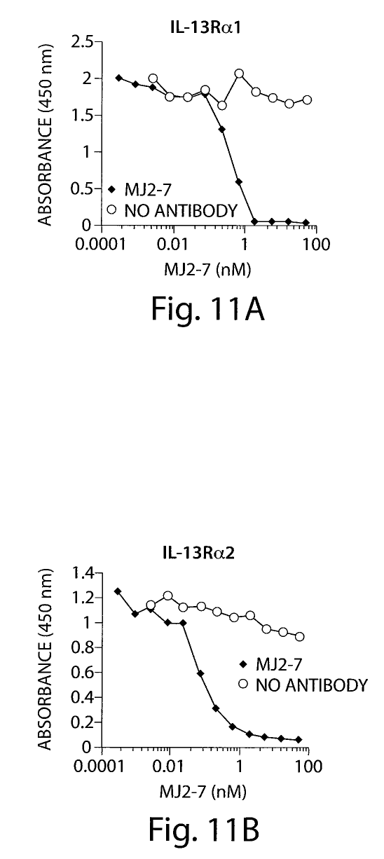

The ability of MJ2-7 to inhibit binding of NHP IL-13 to IL-13RI1 and IL-13RI2 was tested by ELISA. Recombinant soluble forms of human IL-13RI1-Fc and IL-13RI2-Fc were coated onto ELISA plates. FLAG-tagged NHP IL-13 was added in the presence of increasing concentrations of MJ2-7. Results showed that MJ2-7 competed with both soluble receptor forms for binding to NHP IL-13 (FIGS. 11A and 11B). This provides a basis for the neutralization of IL-13 bioactivity by MJ2-7.

### Example 17

### The MJ 2-7 Light Chain CDRs Contribute to Antigen Binding

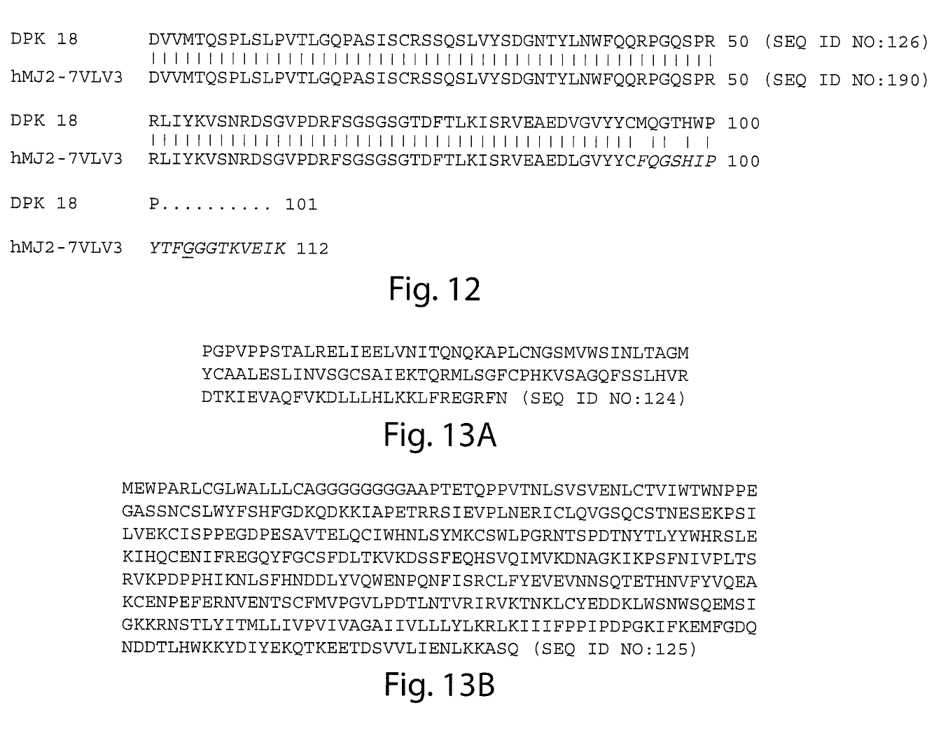

To evaluate if all three light chain CDR regions are required for the binding of MJ 2-7 antibody to NHP IL-13, two additional humanized versions of MJ 2-7 VL were constructed by CDR grafting. The VL version 3 was designed based on human germline clone DPK18, contained CDR1 and CDR2 of the human germline clone and CDR3 from mouse MJ2-7 antibody (FIG. 12). In the second construct (hMJ 2-7 V4), only CDR1 and CDR2 of MJ 2-7 antibody were grafted onto DPK 18 framework, and CDR3 was derived from irrelevant mouse monoclonal antibody.

The humanized MJ 2-7 V3 and V4 were produced in COS cells by combining hMJ 2-7 VH V1 with hMJ 2-7 VL V3 and V4. The antigen binding properties of the antibodies were examined by direct NHP IL-13 binding ELISA. The hMJ 2-7 V4 in which MJ 2-7 light chain CDR3 was absent retained the ability to bind NHP IL-13, whereas V3 that contained human germline CDR1 and CDR2 in the light chain did not bind to immobilized NHP IL-13. These results demonstrate that CDR1 and CDR2 of MJ 2-7 antibody light chain are most likely responsible for the antigen binding properties of this antibody.

```
Nucleotide sequence of hMJ 2-7 VL V3
(SEQ ID NO:189)
    1 ATGCGGCTGC CCGCTCAGCT GCTGGGCCTG CTGATGCTGT GGGTGCCCGG
  51 CTCTTCCGGC GACGTGGTGA TGACCCAGTC CCCTCTGTCT CTGCCCGTGA
101 CCCTGGGCCA GCCCGCTTCT ATCTCTTGCC GGTCCTCCCA GTCCCTGGTG
151 TACTCCGACG GCAACACCTA CCTGAACTGG TTCCAGCAGA GACCCGGCCA
201 GTCTCCTCGG CGGCTGATCT ACAAGGTGTC CAACCGCTTT TCCGGCGTGC
251 CCGATCGGTT CTCCGGCTCC GGCAGCGGCA CCGATTTCAC CCTGAAGATC
301 AGCCGCGTGG AGGCCGAGGA TGTGGGCGTG TACTACTGCT TCCAGGGCTC
351 CCACATCCCT TACACCTTTG GCGGCGGAAC CAAGGTGGAG ATCAAG
Amino acid sequence of hMJ 2-7 VL V3
(SEQ ID NO:190)
MRLPAQLLGLLMLWVPGSSG-DVVMTQSPLSLPVTLGQPASISCRSSQSLVYSDGNTYLN
WFQQRPGQSPRRLIYKVSNRFSGVPDRFSGSGSGTDFTLKISRVEAEDVGVYYCFQGSHI
PYTFGGGTKVEIK
Nucleotide sequence of hMJ 2-7 VL V4
(SEQ ID NO:191)
GATGTTGTGATGACCCAATCTCCACTCTCCCTGCCTGTCACTCCTGGAGAGCCAGCCTCC
ATCTCTTGCAGATCTAGTCAGAGCATTGTGCATAGTAATGGAAACACCTACCTGGAATGG
TACCTGCAGAAACCAGGCCAGTCTCCACAGCTCCTGATCTACAAAGTTTCCAACCGATTT
TCTGGGGTCCCAGACAGGTTCAGTGGCAGTGGATCAGGGACAGATTTCACACTCAAGATC
AGCAGAGTGGAGGCTGAGGATGTGGGAGTTTATTACTGCTTTCAAAGTTCACATGTTCCT
CTCACCTTCGGTCAGGGGACCAAGCTGGAGATCAAA
Amino acid sequence of hMJ 2-7 VL V4
(SEQ ID NO:192)
DVVMTQSPLS LPVTPGEPAS ISCRSSQSIV HSNGNTYLEW YLQKPGQSPQ
LLIYKVSNRF SGVPDRFSGS GSGTDFTLKISRVEAEDVGV YYCFQSSHVP
LTFGQGTKLE IK
```

### Example 18

### Neutralizing Activities of Anti-IL-13 Antibodies in Cynomolgus Monkey Model

The efficacy of an IL-13 binding agent (e.g., an anti-IL-13 antibody) in neutralizing one or more IL-13-associated activities in vivo can be tested using a model of antigen-induced airway inflammation in cynomolgus monkeys naturally allergic to Ascaris suum. These assays can be used to confirm that the binding agent effectively reduces airway eosinophilia in allergic animals challenged with an allergen. In this model, challenge of an allergic monkey with Ascaris suum antigen results in one or more of the following: (i) an influx of inflammatory cells, e.g., eosinophils into the airways; (ii) increased eotaxin levels; (iii) increase in Ascaris-specific basophil histamine release; and/or (iv) increase in Ascaris-specific IgE titers.

To test the ability of an anti-IL-13 antibody to prevent the influx of inflammatory cells, the antibody can be administered 24 hours prior to challenge with Ascaris suum antigen. On the day of challenge, a baseline bronchoalveolar lavage (BAL) sample can be obtained from the left lung. Ascaris suum antigen can be instilled intratracheally into the right lung. Twenty-four hours later, the right lung is lavaged, and the BAL fluid from animals treated intravenously with the antibody were compared to BAL fluid from untreated animals. If the antibody reduces airway inflammation, an increase in percent BAL eosinophils may be observed among the untreated group, but not for the antibody-treated group.

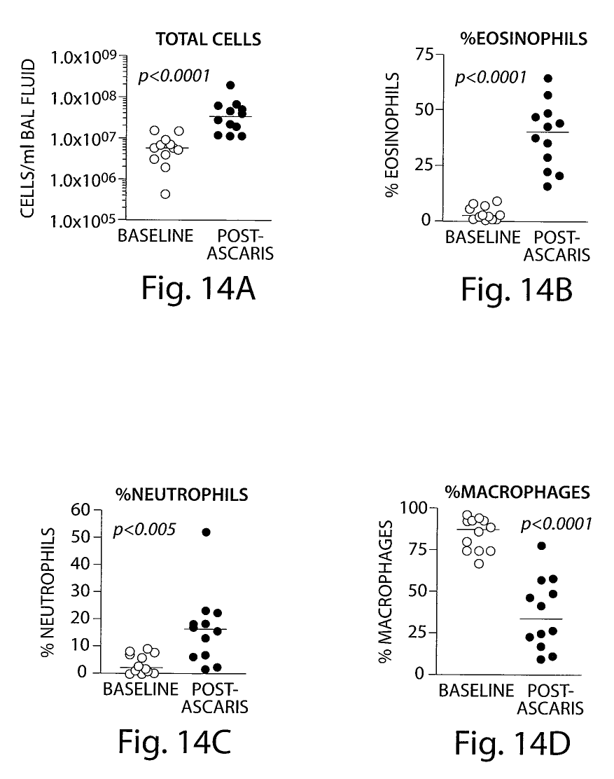

FIGS. 14A-14D depict an increase in the total number of cells and percentage of inflammatory cells, for example, eosinophils (FIG. 14B), neutrophils (FIG. 14C) and macrophages (FIG. 14D) 24-hours following airway challenge with Ascaris. A statistically significant increase in the percentage of inflammatory cells was observed 24 hours after challenge compared to the baseline values.

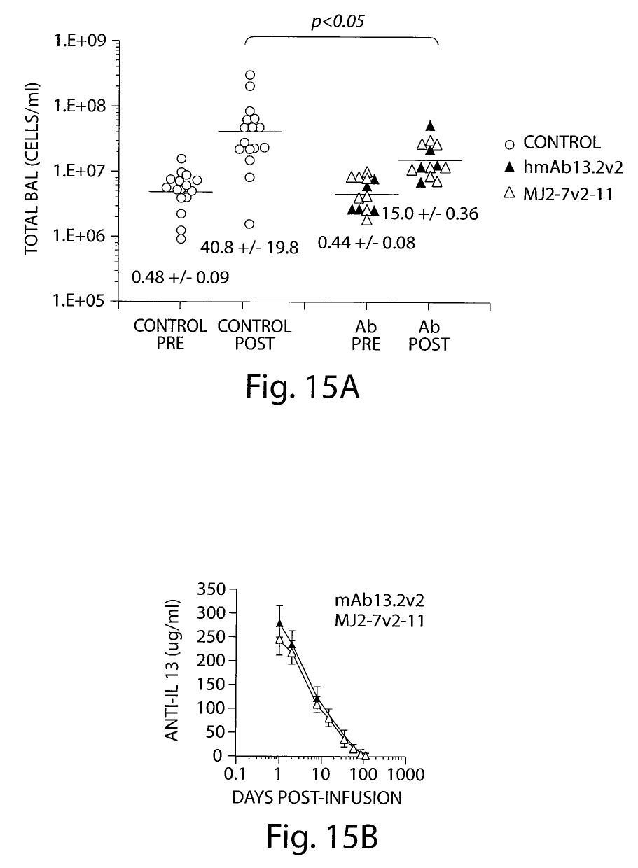

Anti-IL13 antibodies (humanized MJ2-7v.2-11 and humanized mAb13.2v.2) were administered to cynomolgus monkeys 24 hours prior to challenge with Ascaris suum antigen. (mAb 13.2 and its humanized form hmAb13.2v2 were described in commonly owned PCT application WO 05/123126, the contents of which are incorporated herein by reference in their entirety). Control monkeys were treated with saline. 10 mg/kg of hMJ2-7v.2-11, hmAb13.2v2, or irrelevant human Ig (IVIG) were administered intravenously. The following day, prechallenged BAL samples from control and treated monkeys (referred to in FIG. 15A as “control pre” and “Ab pre”) were collected from the left lung of the monkeys. The monkeys were treated with 0.75 micrograms of Ascaris suum antigen intratracheally into the right lung. Twenty-four hours post-challenge, BAL samples were collected from the right lung of control and treated monkeys, and assayed for cellular infiltrate (referred to in FIG. 15B as “control post” and “Ab post,” respectively). BAL samples collected from antibody-treated monkeys showed a statistically significant reduction in the total number of cell infiltrate compared to control animals (FIG. 15A). Control samples are represented in FIG. 15A as circles, hmAb13.2v2- and hMJ2-7v.2-11-treated samples are shown as dark and light triangles, respectively. hMJ2-7v.2-11 and hmAb13.2v2 showed comparable efficacy in this model. FIG. 15B shows a linear graph depicting the concentration of either hMJ2-7v.2-11 or hmAb13.2v2 with respect to days post-Ascaris infusion. A comparable decrease in kinetics is detected for both antibodies.

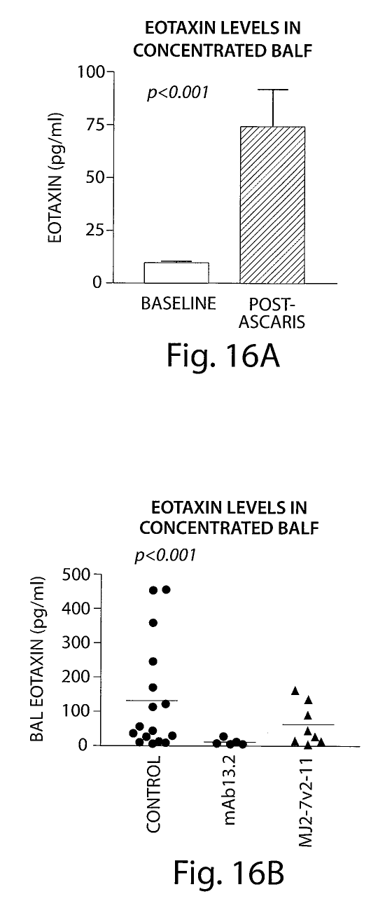

Eotaxin levels were significantly increased 24 hours following Ascaris challenge (FIG. 16A). Both hMJ2-7v.2-11 and hmAb13.2v2 reduced eotaxin levels detected in BAL fluids from cynomolgus monkeys 24 hours after to challenge with Ascaris suum antigen, compared to saline treated controls.

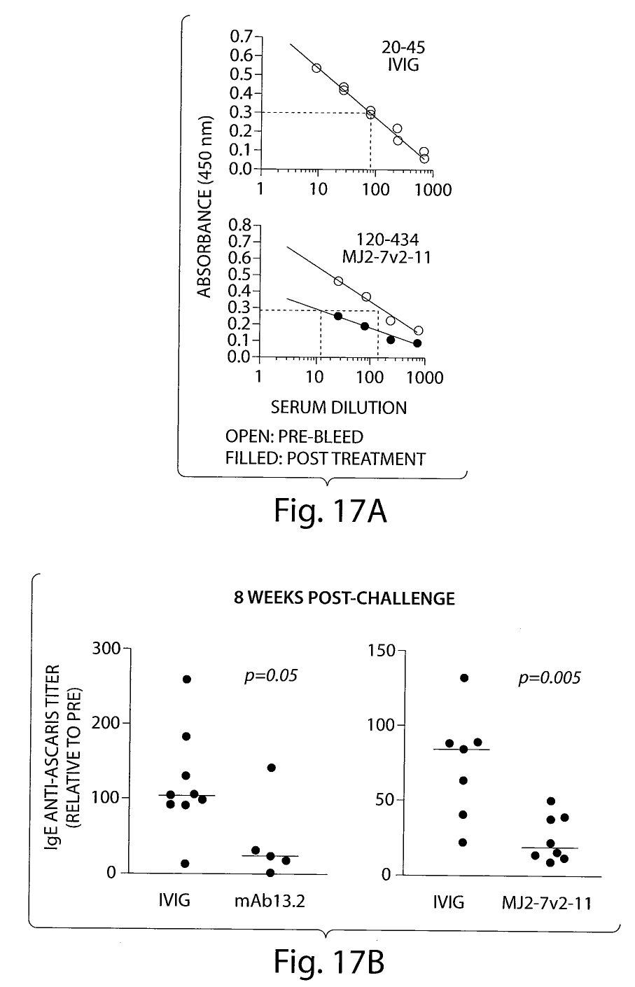

Cynomolgus monkeys sensitized to Ascaris suum develop IgE to Ascaris antigen. The IgE binds to FcεRI on circulating basophils, such that in vitro challenge of peripheral blood basophils with Ascaris antigen induces degranulation and release of histamine. Repeated antigen exposure boosts basophil sensitization, resulting in enhanced histamine release responses. To test the effects of hMJ2-7v.2-11 and hmAb 13.2v2 in IgE- and basophil levels, cynomolgus monkeys dosed with humanized hMJ2-7v.2, hmAb13.2v2, irrelevant Ig (IVIG), or saline, as described above, were bled 8 weeks post-Ascaris challenge, and levels of total and Ascaris-specific IgE in plasma were determined by ELISA. FIG. 17A shows a linear graph of the changes in absorbance with respect to dilution of samples obtained pre- and 8-weeks post-challenge from animals treated with IVIG or hMJ2-7v.2-11. Open-circles represent pre-bleed measurements; filled circles represent post-treatment measurements. A significant reduction in absorbance was detected in post-challenged samples treated with hMJ2-7v.2-11 relative to the pre-challenge values in all dilutions assayed FIG. 17A depicts representative examples showing no change in Ascaris-specific IgE titer in an individual monkey treated with irrelevant Ig (IVIG; animal 20-45; top panel), and decreased titer of Ascaris-specific IgE in an individual monkey treated with hMJ2-7v.2-11 (animal 120-434; bottom panel).

Animals treated with either humanized hMJ2-7v.2-11 or hmAb13.2v2 showed a significant reduction in levels of circulating IgE-specific for Ascaris in cynomolgus monkey sera (FIG. 17B). There was no significant change in total IgE titer for any of the treatment groups. FIG. 17A shows a linear graph of the changes in absorbance with respect to dilution of samples obtained pre- and 8-weeks post-challenge from animals treated with IVIG or hMJ2-7v.2-11. Open-circles represent pre-bleed measurements; filled circles represent post-treatment measurements. A significant reduction in absorbance was detected in post-challenged samples treated with hMJ2-7v.2-11 relative to the pre-challenge values in all dilutions assayed. The designations “20-45” and “120-434” refer to the cynomolgus monkey identification number.

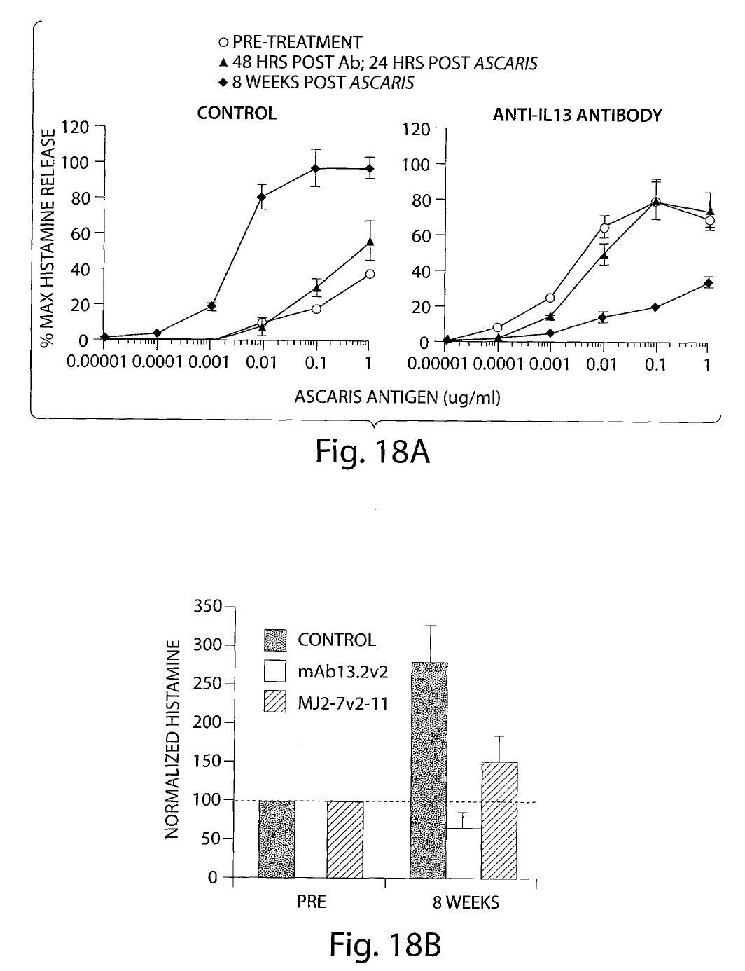

To evaluate the effects of anti-IL13 antibodies on basophil histamine release, the animals were bled at 24 hours and 8 weeks post-Ascaris challenge. Whole blood was challenged with Ascaris antigen for 30 minutes at 37° C., and histamine released into the supernatant was quantitated by ELISA (Beckman Coulter, Fullerton, Calif.). As shown in FIGS. 18A-18B, the control animals demonstrated increased levels of Ascaris-induced basophil histamine release particularly 8 weeks following antigen challenge (represented by the diamonds in FIG. 18A and left-hand bar in FIG. 18B). In contrast, the animals treated with either humanized hMJ2-7v.2-11 or hmAb13.2v2 did not show this increase in basophil sensitization in response to Ascaris 8 weeks after challenge (FIGS. 18A-18B). The majority of individual animals treated with humanized hMJ2-7v.2-11 or hmAb13.2v2 showed either a decrease (example in FIG. 18A) or no change in basophil histamine release 8 weeks post-challenge compared to pre- or 24 hour post-challenge. Thus, a single administration of the humanized anti-IL13 antibody had a lasting effect in modifying histamine release in this model.

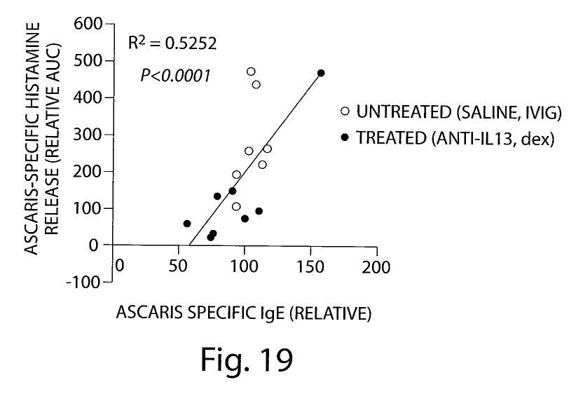

FIG. 19 depicts the correlation between Ascaris-specific histamine release and Ascaris-specific IgE levels. Higher values were detected in control samples (saline- or IVIG-treated samples) (light blue circles) compared to anti-IL13 antibody- or dexamethasone (dex)-treated (dark red circles). Humanized anti-IL13 antibody (humanized mAb13.2v.2) administered i.v. 24 hours prior to Ascaris challenge, or dexamethasone administered intramuscular in two injections each one at a concentration of 1 mg/kg 24 hours and 30 mins. prior to Ascaris challenge. Twenty four hours post-challenge, BAL lavage was collected from the right lung and assayed for histamine release and IgE levels.

The results shown herein demonstrated that pretreatment of cynomolgus monkeys with either MJ2-7 or mAb13.2 reduced airway inflammation induced by Ascaris suum antigen at comparable levels as detected by cytokine levels in BAL samples, serum levels of Ascaris-specific IgE's and basophil histamine release in response to Ascaris challenge in vitro.

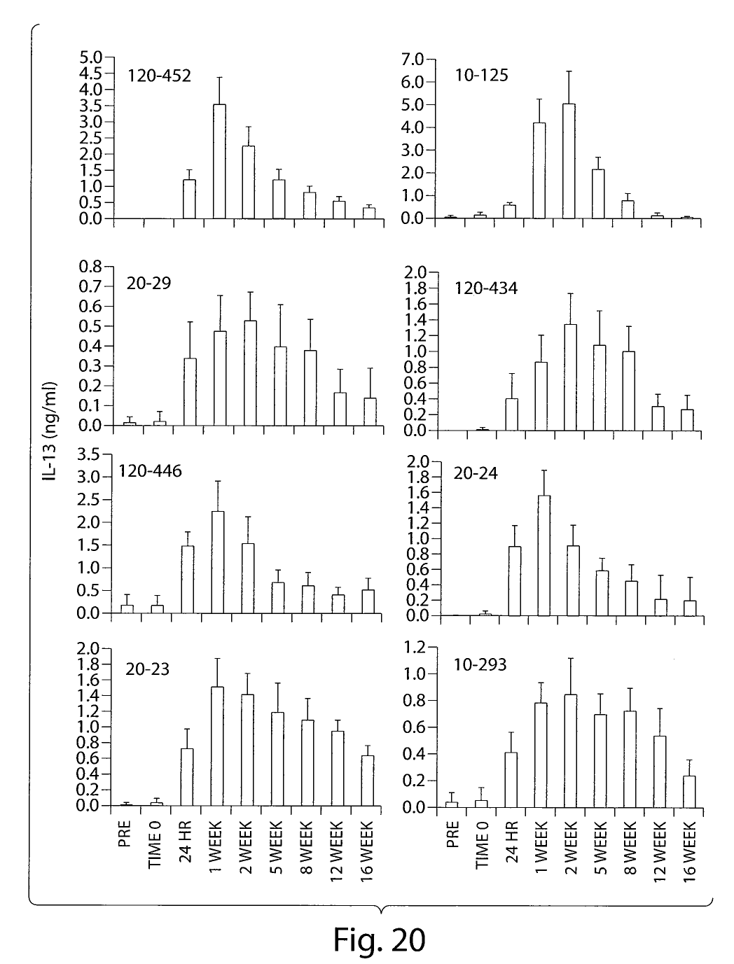

FIG. 20 is a series of bar graphs depicting the increases in serum IL-13 levels in individual cynomolgus monkeys treated with humanized MJ2-7 (hMJ2-7v.2-11). The label in each panel (e.g., 120-452) corresponds to the monkey identification number. The “pre” sample was collected prior to administration of the antibody. The time “0” was collected 24-hours post-antibody administration, but prior to Ascaris challenge. The remaining time points were post-Ascaris challenge. The assays used to detect IL-13 levels are able to detect IL-13 in the presence of hMJ2-7v.2-11 or hmAb13.2v2 antibodies. More specifically, ELISA plates (MaxiSorp; Nunc, Rochester, N.Y.), were coated overnight at 4° C. with 0.5 ug/ml mAb13.2 in PBS. Plates were washed in PBS containing 0.05% Tween-20 (PBS-Tween). NHP IL-13 standards, or serum dilutions from cynomolgus monkeys, were added and incubated for 2 hours at room temperature. Plates were washed, and 0.3 ug/ml biotinylated MJ1-64 (referred to herein as C65 antibody) was added in PBS-Tween. Plates were incubated 2 hours, room temperature, washed, and binding detected using HRP-streptavidin (Southern Biotechnology Associates) and Sure Blue substrate (Kirkegaard and Perry Labs). For detection of IL-13 in the presence of mAb13.2, the same protocol was followed, except that ELISA plates were coated with 0.5 ug/ml MJ2-7.

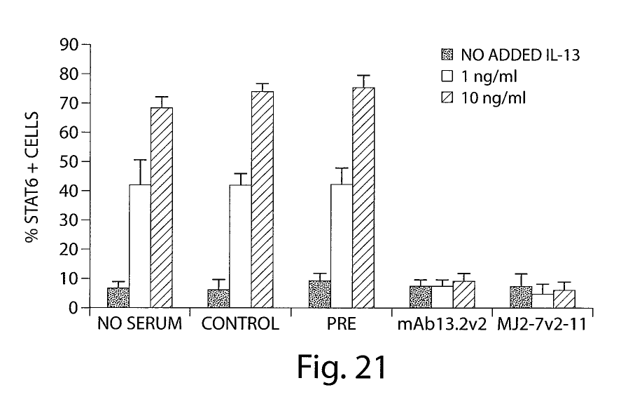

FIG. 21 shows data demonstrating that sera from cynomolgus monkeys treated with anti-IL13 antibodies have residual IL-13 neutralization capacity at the concentrations of non-human primate IL-13 tested. FIG. 21 is a bar graph depicting the STAT6 phosphorylation activity of non-human primate IL-13 at 0, 1, or 10 ng/ml, either in the absence of serum (“no serum”); the presence of serum from saline or IVIG-treated animals (“control”); or in the presence of serum from anti-IL13 antibody-treated animals, either before antibody administration (“pre”), or 1-2 weeks post-administration of the indicated antibody. Serum was tested at 1:4 dilution. A humanized version of MJ2-7 (MJ2-7v.2-11) was used in this study. Assays for measuring STAT6 phosphorylation are disclosed herein.

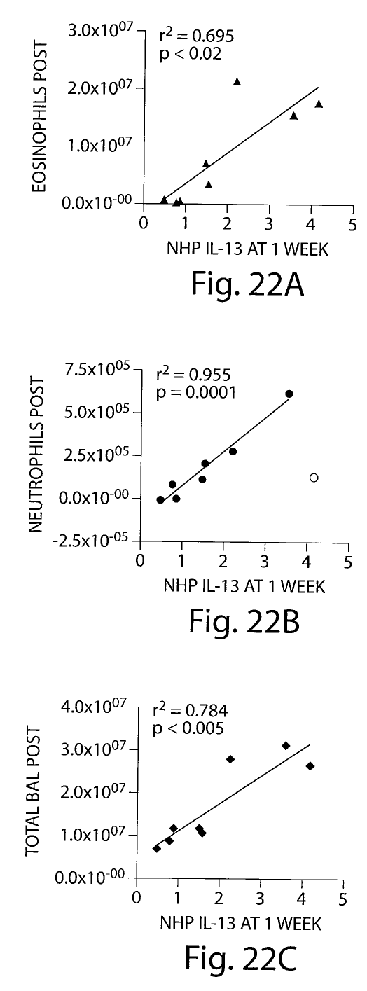

FIG. 22 are linear graphs showing that levels of non-human primate IL-13 trapped by humanized MJ2-7 (hMJ2-7v.2-11) at a 1-week time point in cynomolgus monkey serum correlate with the level of inflammation measured in the BAL fluids post-Ascaris challenge. Such correlation supports that detection of serum IL-13 (either unbound or bound to an anti-IL13 antibody) as a biomarker for detecting subjects having inflammation. Subjects having more severe inflammation showed higher levels of serum IL-13. Although levels of unbound IL-13 are typically difficult to quantitate, the assays disclosed herein above in FIG. 20 provides a reliable assay for measuring IL-13 bound to an anti-IL-13 antibody.

### Example 19

### Effects of Humanized Anti-IL-13 Antibodies on Airway Inflammation, Lung Resistance, and Dynamic Lung Compliance Induced by Administration of Human IL-13 to Mice

Murine models of asthma have proved invaluable tools for understanding the role of IL-13 in this disease. The use of this model to evaluate in vivo efficacies of the antibody series (humanized 13.2v.2 and humanized MJ2-7v.2-11) was initially hampered by the inability of these antibodies to cross react with rodent IL-13. This limitation was circumvented herein by administering human recombinant IL-13 to mice. Human IL-13 is capable of binding to the murine IL-13 receptor, and when administered exogenously induces airway inflammation, hyperresponsiveness, and other correlates of asthma.

In non-human primates, the IL-13 epitope recognized by humanized MJ2-7v.2-11 includes a GLN at position 110. In humans, however, position 110 is a polymorphic variant, typically with ARG replacing GLN (e.g., R110). The R110Q polymorphic variant is widely associated with increased prevalence of atopic disease.

In this example, recombinant human R110Q IL-13 was expressed in E. coli and refolded. Antibody 13.2 (IgG1, k) was cloned from BALB/c mice immunized with human IL-13, and the humanized version of this antibody is designated humanized 13.2v.2 (or h13.2v.2). Antibody MJ2-7 (IgG1, k) was cloned from BALB/c mice immunized with the N-terminal 19 amino acids of nonhuman primate IL-13, and the humanized version of this antibody is designated humanized MJ2-7v.2-11 (or hMJ2-7v.2-11). Both antibodies were formulated in 10 mM L-histidine, pH 6, containing 5% sucrose. Carimune NH immune globulin intravenous (human IVIG) (ZLB Bioplasma Inc., Switzerland) was purified by Protein A chromatography and formulated in 10 mM L-histidine, pH 6, containing 5% sucrose.

To analyze the mouse lung response to the presence of recombinant human R110Q IL-13, BABL/c female mice were treated with 5 μg of recombinant human R110Q IL-13 (e.g., approximately 250 Tg/kg), or an equivalent volume of saline (20 μL), administered intratracheally on days 1, 2, and 3. On day 4, animals were tested for signs of airway resistance (RI) and compliance (Cdyn) in response to increasing doses of nebulized methacholine. Briefly, anesthetized and tracheostomized mice were placed into whole body plethysmographs, each with a manifold built into the head plate of the chamber, with ports to connect to the trachea, to the inspiration and expiration ports of a ventilator, and to a pressure transducer, monitoring the tracheal pressure. A pneumotachograph in the wall of each plethysmograph monitored the airflow into and out of the chamber, due to the thoracic movement of the ventilated animal. Animals were ventilated at a rate of 150 breaths/min and a tidal volume of 150 ml. Resistance computations were derived from the tracheal pressure and airflow signals, using an algorithm of covariance.

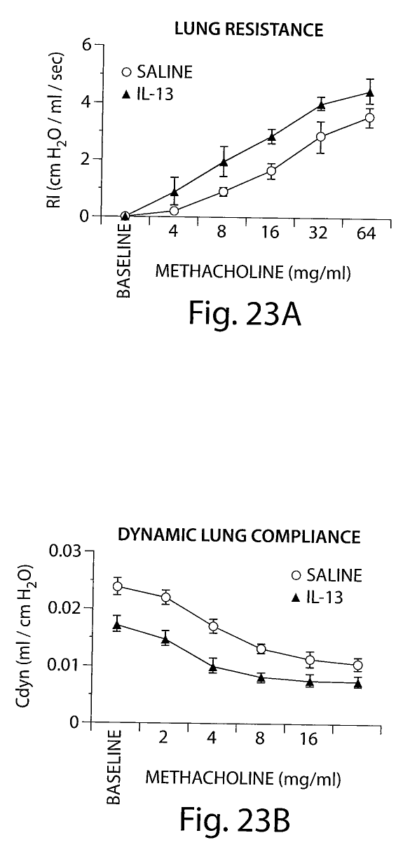

As shown in FIGS. 23A-23B, intratracheal administration of recombinant human R110Q IL-13 elicited increased lung resistance and decreased dynamic compliance in response to methacholine challenge. These observations were not, however, accompanied by strong lung inflammation.

To enhance the lung inflammatory response in mice, 5 μg of recombinant human R110Q IL-13, or an equivalent volume (50 μL) of saline, was administered to C57BL/6 mice intranasally on days 1, 2, and 3. Animals were sacrificed on day 4 and bronchoalveolar lavage (BAL) fluid collected. Pre-analysis, BAL was filtered through a 70 μm cell strainer and centrifuged at 2,000 rpm for 15 minutes to pellet cells. Cell fractions were analyzed for total leukocyte count, spun onto microscope slides (Cytospin; Pittsburgh, Pa.), and stained with Diff-Quick (Dade Behring, Inc. Newark Del.) for differential analysis. IL-6, TNFα, and MCP-1 levels were determined by cytometric bead array (CBA; BD Pharmingen, San Diego, Calif.). The limits of assay sensitivity were 1 pg/ml for IL-6, and 5 pg/ml for TNFα and MCP-1.

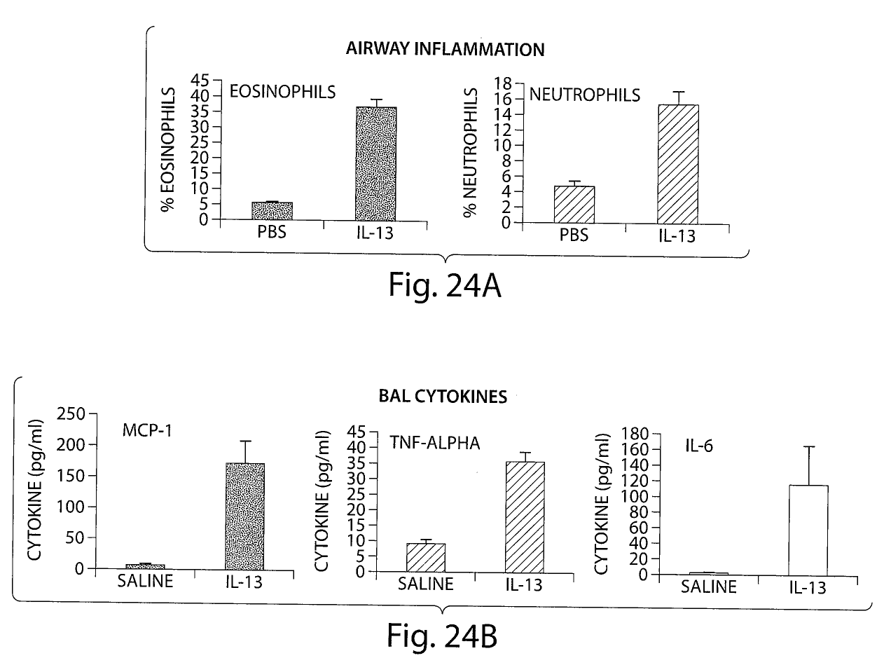

As shown in FIG. 24A, intranasal administration of recombinant human R110Q IL-13 induced a strong airway inflammatory response, as indicated by elevated eosinophil and neutrophil infiltration into BAL. Cell infiltrates consisted primarily of eosinophils (e.g., approximately 40%). As shown in FIG. 24B, intranasal administration of recombinant human R110Q IL-13 also significantly increased the levels of several cytokines in BAL including, for example, MCP-1, TNF-I, and IL-6.

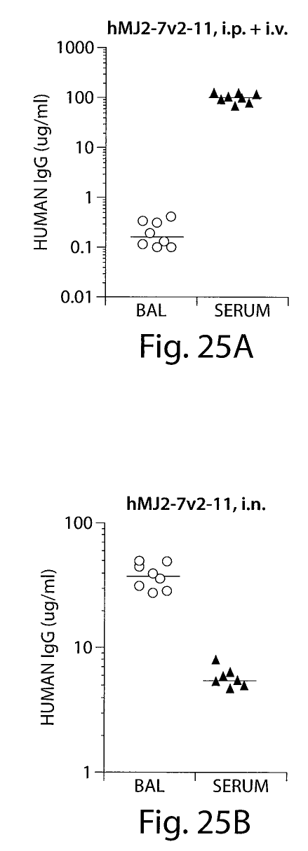

To determine the best delivery method for humanized MJ2-7v.2-11, antibody levels in BAL and serum were analyzed following intraperitoneal and intravenous, or intranasal administration following treatment with recombinant human R110Q IL-13 administered intranasally or intratracheally. Briefly, BALB/c female mice were administered 5 μg of recombinant human R110Q IL-13 or an equivalent volume of saline intratracheally on days 1, 2, and 3. On day 0, and 2 hours prior to administering each IL-13 dose, mice were treated with 500 μg humanized MJ2-7v.2 administered intravenously on day 0, and by IP on days 1, 2, and 3 (FIG. 25A). Alternatively, 500 μg of humanized MJ2-7v.2-11 were administered intranasally on days 0, 1, 2, and 3. Total human IgG was measured by ELISA, as follows: ELISA plates (MaxiSorp; Nunc, Rochester, N.Y.) were coated overnight at 4° C. with 1:1500 dilution of goat anti-human Ig (M+G+A) Fc (ICN-Cappel, Costa Mesa, Calif.) at 50 μl/well in 25 mM carbonate-bicarbonate buffer, pH 9.6. Plates were blocked for 1 hour at room temperature with 0.5% gelatin in PBS, washed in PBS containing 0.05% Tween-20 (PBS-Tween). Humanized MJ2-7v.2-11 standard or 6×1:2 dilutions of sheep serum starting at 1:500-1:50,000 were added and incubated for 2 hours at room temperature. Plates were washed with PBS-Tween, and a 1:5000 dilution of biotinylated mouse anti-human IgG (Southern Biotechnology Associates) was incubated for 2 hours at room temperature. Plates were washed with PBS-Tween, and binding was detected with peroxidase-linked streptavidin (Southern Biotechnology Associates) and Sure Blue substrate (KPL Inc.). Assay sensitive was 0.5 ng/ml human IgG.

FIG. 25A shows elevated levels of human IgG in serum compared to BAL following intraperitoneal and intravenous administraton of the humanized MJ2-7v.2-11 antibody. As shown in FIG. 25B, total IgG levels in μg/ml were significantly higher in BAL than serum levels following intranasal administration of humanized MJ2-7v.2-11 antibody.

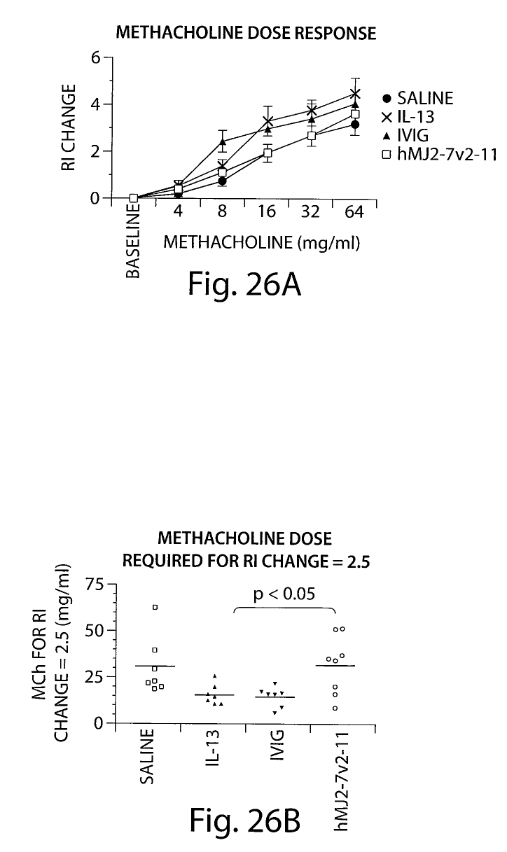

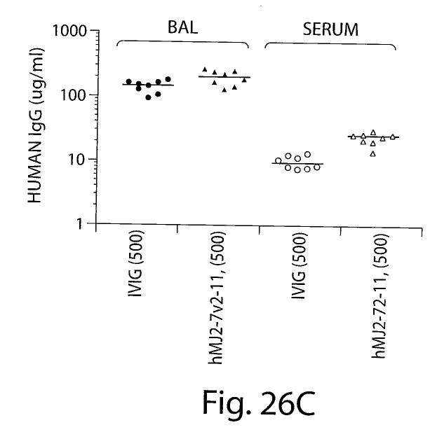

To determine if the humanized MJ2-7v.2-11 antibody was capable of modulating the above observed lung function and inflammatory response, airway hyperresponsiveness was induced by intratracheal administration of 5 μg recombinant human R110Q IL-13 or an equivalent volume (20 μL) of saline on days 1, 2, and 3. On day 0, and 2 hours before administering each dose of recombinant human R110Q IL-13, animals were treated with 500 μg of humanized MJ2-7v.2-11, 500 μg dose of IVIG, or an equivalent volume of saline, administered intranasally. Animals were tested on day 4 for airway resistance (RI) and compliance (Cdyn) in response to increasing doses of nebulized methacholine, as described above. Humanized MJ2-7v.2 and IVIG levels in BAL and serum were analyzed by ELISA, as described above. As shown in FIGS. 26A-26B, humanized MJ2-7v.2-11 effectively reduced the asthmatic response, resulting in a significant reduction in the dose of methacholine required to achieve half-maximal degree of lung resistance. In contrast, an equivalent dose of IVIG had no effect. Changes in dynamic lung compliance were not apparent under these conditions. As shown in FIG. 26C, BAL IgG antibody levels were approximately 10-20 times higher than serum levels.

To determine if humanized MJ2-7v.2-11 anti-IL-13 antibody administration promoted an increase in the circulating levels of IL-13, BAL and sera were assayed for IL-13 levels by ELISA, as follows: Briefly, BALB/c female mice were treated as described for FIG. 26A-B. ELISA plates (Nunc Maxi-Sorp) were coated overnight with 50 μl/well mouse anti-IL-13 antibody, mAb13.2, diluted to 0.5 mg/ml in PBS. Plates were washed 3 times with PBS containing 0.05% Tween-20 (PBS-Tween) and blocked for 2 hours at room temperature with 0.5% gelatin in PBS. Plates were then washed and human IL-13 standard (Wyeth, Cambridge, Mass.), or dilutions of mouse serum (serial 3× dilutions starting at 1:4) were added, in PBS-Tween containing 2% fetal calf serum (FCS). Plates were incubated for a further 4 hours at room temperature, and washed. Biotinylated mouse anti-human IL-13 antibody, MJ1-64, was added at 0.3 μg/ml in PBS-Tween. Plates were incubated for 1-2 hours at room temperature, washed, then incubated with HRP-streptavidin (Southern Biotechnology Associates, Birmingham, Ala.) for 1 hour at room temperature. Color was developed using Sure Blue peroxidase substrate (KPL, Gaithersburg, Md.), and the reaction stopped with 0.01M sulfuric acid. Absorbance was read at 450 nm in read in a SpectraMax plate reader (Molecular Devices Corp., Sunnyvale, Calif.). Serum IL-13 levels were determined by reference to a human IL-13 standard curve, which was independently established for each plate.

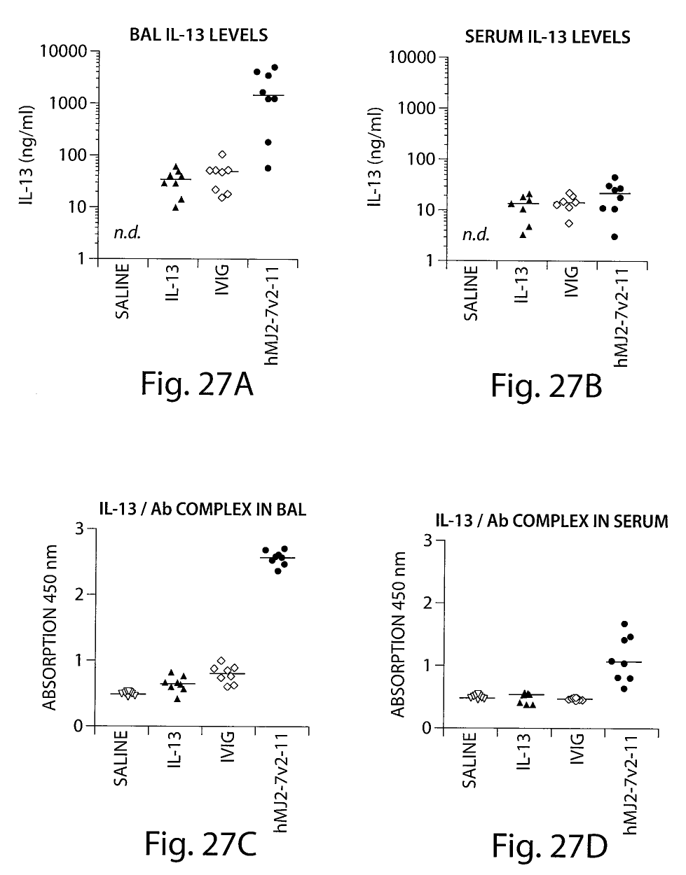

As shown in FIGS. 27A-27B, consistent with FIG. 26C, IL-13 levels were elevated in BAL of antibody-treated mice, but not serum. In addition, we observed that IL-13 isolated from these samples had no detectable biological activity (data not shown). To determine if this observed lack of IL-13 biological activity was due to IL-13 and humanized MJ2-7v.2-11 complex formation, an ELISA was developed to specifically detect IL-13 and humanized MJ2-7v.2-11 in complex. Briefly, ELISA plates (Nunc Maxi-Sorp) were coated overnight with 50 μl/well mouse anti-IL-13 antibody, mAb13.2, diluted to 0.5 mg/ml in PBS. Plates were washed 3 times with PBS containing 0.05% Tween-20 (PBS-Tween) and blocked for 2 hours at room temperature with 0.5% gelatin in PBS. Plates were then rewashed, and human IL-13 standard (Wyeth, Cambridge, Mass.), or dilutions of mouse serum (serial 3× dilutions starting at 1:4) were added, in PBS-Tween containing 2% fetal calf serum (FCS). Plates were subsequently incubated for 4 hours at room temperature. Biotinylated anti-human IgG (Fc specific) (Southern Biotechnology Associates, Birmingham, Ala.) diluted 1:5000 in PBS-Tween was then added. Plates were incubated for 1-2 hours at room temperature, washed, and finally incubated with HRP-streptavidin (Southern Biotechnology Associates, Birmingham, Ala.) for 1 hour at room temperature. Color was developed using Sure Blue peroxidase substrate (KPL, Gaithersburg, Md.), and the reaction stopped with 0.01M sulfuric acid. Absorbance was read at 450 nm in read in a SpectraMax plate reader (Molecular Devices Corp., Sunnyvale, Calif.).

As shown in FIGS. 27C-27D, IL-13 and humanized MJ2-7v.2-11 complexes were recovered from BAL and serum of mice in this model. This observation indicates that humanized MJ2-7v.2-11 is capable of binding IL-13 in vivo, and that this interaction may negate IL-13 biological activity.

The effects of humanized MJ2-7v.2-11 on human IL-13-mediated lung inflammation and cytokine production were tested in mice, and compared with a second antibody, humanized 13.2v.2, as follows. Briefly, C57BL/6 female mice (10/group) were treated with 5 μg of recombinant human R110Q IL-13 (e.g., approximately 250 μg/kg), or an equivalent volume (50 μl) of saline, on days 1, 2, and 3, administered intranasally. On day 0, and 2 hours before administering each dose of IL-13, mice were given intranasal doses of 500 μg, 100 μg, or 20 μg of humanized MJ2-7v.2-11 or humanized 13.2v.2. Control groups received 500 μg IVIG, or an equivalent volume of saline. Animals were sacrificed on day 4, and BAL collected. Eosinophil and neutrophil infiltration into BAL were determined by differential cell count and expressed as a percentage.

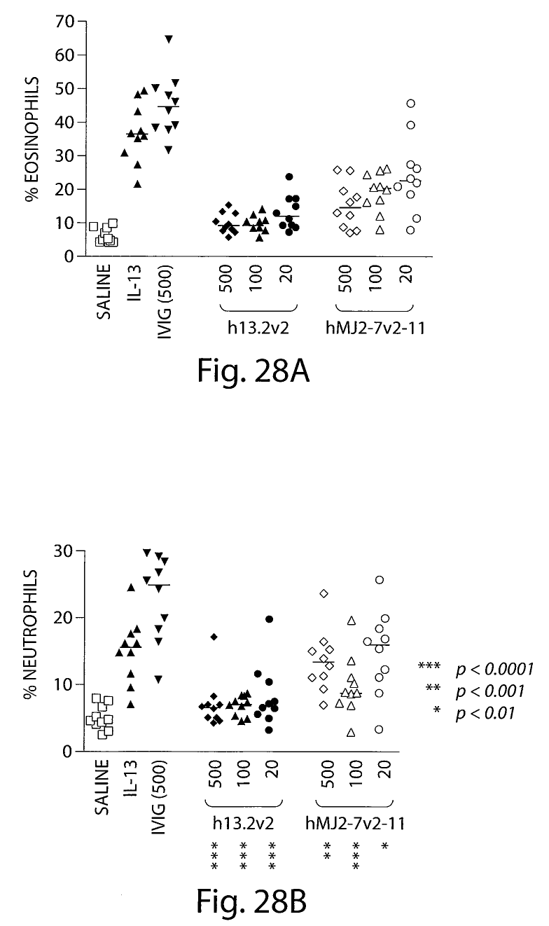

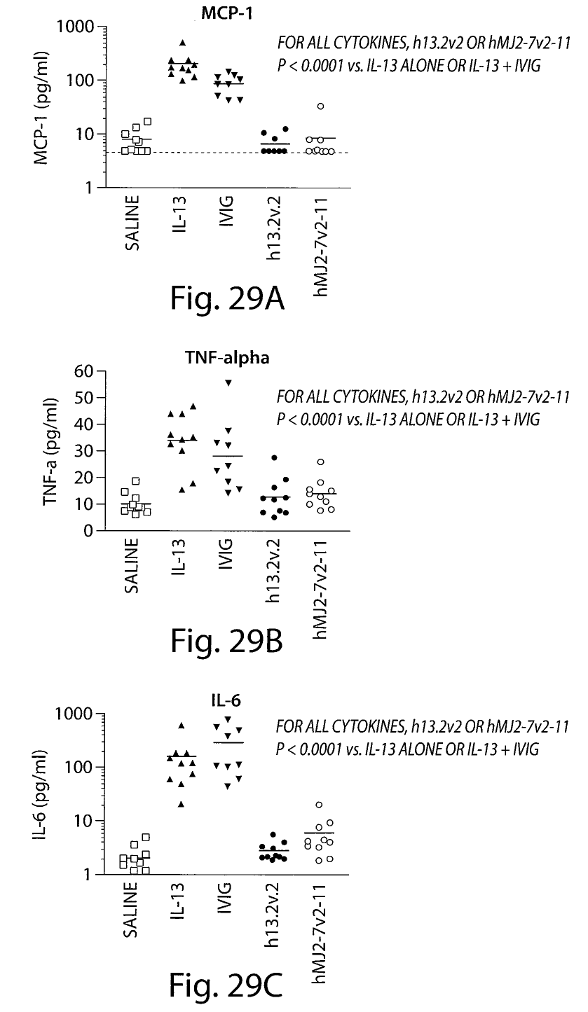

As shown in FIGS. 28A-28B, consistent with FIG. 24A, recombinant human R110Q IL-13 treatment evoked an increase in eosinophil and neutrophil infiltration levels. Interestingly, humanized MJ2-7v.2-11 and humanized 13.2v.2 significantly reduced eosinophil (FIG. 28A) and neutrophil (FIG. 28B) infiltration compared to controls (e.g., saline, IL-13, IVIG). As shown in FIG. 29A-29C, HMJ2-7V2-11 and humanized MJ2-7v.2-11 also abrogated increases in MCP-1, TNF-I, and IL-6 cytokine levels.

To confirmation that BAL cytokine levels accurately represent the degree of inflammation C57BL/6 female mice were treated with 5 μg of recombinant human R110Q IL-13 (e.g., approximately 250 μg/kg) or an equivalent volume (50 μl) of saline on days 1, 2, and 3, administered intranasally. On day 0, and 2 hours before administering each dose of IL-13, mice were given intranasal doses of 500, 100, or 20 μg of humanized MJ2-7v.2-11. On day 4, animals were sacrificed and BAL collected. Humanized MJ2-7v.2-11 antibody levels in BAL were determined by ELISA, as described above. BAL IL-6 levels were determined by cytometric bead array. Eosinophil percentages were determined by differential cell counting.

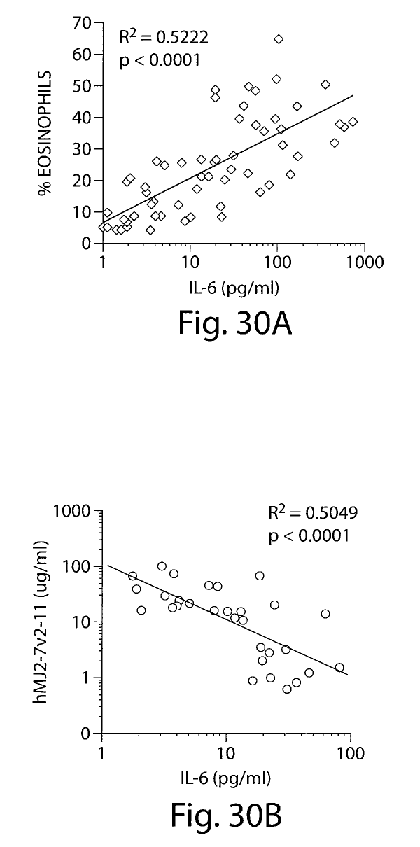

As shown in FIGS. 30A-30B, IL-6 BAL cytokine levels were related to the degree of inflammation. Furthermore, higher levels of humanized MJ2-7v.2-11 in BAL fluid inversely correlated with cytokine concentration, strongly implying a treatment effect.

The levels of antibody required to reduce IL-13 bioactivity in vivo in this model were high. The best efficacy was seen at a dose of 500 μg antibody, corresponding to approximately 25 mg/kg in the mouse. This high dose requirement for antibody is most likely a consequence of the high levels of IL-13 (5 μg/dose×3 doses) used to elicit lung responses. Interestingly, good neutralization of in vivo IL-13 bioactivity was seen only when humanized MJ2-7v.2-11 was administered intranasally, and not when the antibody was administered via intravenous or intraperitoneal. Distribution studies showed that following intravenous and intraperitoneal dosing, high levels of antibody were recovered in serum at the time of sacrifice, but very low levels were found in BAL. In contrast, following intranasal dosing, comparable levels of antibody were found in serum and in BAL. Thus, levels of humanized MJ2-7v.2-11 in BAL fluid were approximately 100-fold higher following intranasal dosing than intravenous and intraperitoneal dosing. The observation that intranasal dosing was efficacious but intravenous and intraperitoneal dosing was not indicates that in this model, the site of antibody action was the lung. This site of action is expected based on the intratracheal or intranasal delivery route of IL-13, and was confirmed by the observation that antibody trapped IL-13 in the BAL fluid, but very little antibody/IL-13 complex was seen in the serum.

In conclusion, these findings further support the IL-13 neutralization activity of humanized MJ2-7v.2-11 in vivo.

### Example 20

### Pharmacokinetics, Pharmacodynamics, and Interspecies Scaling of Humanized Anti-IL-13 Antibodies

Antibody 13.2 (IgG1, k) was cloned from BALB/c mice immunized with human IL-13, and the humanized version of this antibody is designated “humanized 13.2v.2.” Antibody MJ2-7 (IgG1, k) was cloned from BALB/c mice immunized with the N-terminal 19 amino acids of non-human primate IL-13, and the humanized version of this antibody is designated herein as “humanized MJ2-7v.2-11” or “hMJ2-7v.2-11.” Both antibodies were formulated in 10 mM L-histidine, pH 6, containing 5% sucrose. Both anti-IL-13 antibodies are cross reactive with monkey IL-13, and humanized 13.2v.2 is cross reactive with sheep IL-13. However, humanized 13.2v.2 and humanized MJ2-7v.2-II antibodies do not cross react with rodent (e.g., mouse and rat) IL-13.

To support pre-clinical testing of anti-IL-13 antibodies, single dose pharmacokinetic (PK) and pharmacodynamic (PD) studies were performed in mice, rats, sheep, and cynomolgus monkeys after IV and SC administration. In addition, PK studies were conducted using the Ascaris-challenged monkey model, described in Example 21a, and an Ascaris-challenged sheep model, described below. PK parameters were calculated using non-compartmental models and WinNonLin software (Model 201 and 200). Finally, PK animal profiles have been extrapolated using PK-PD modeling to predict the disposition of anti-IL-13 in humans.

Single dose PK studies were performed in mice (e.g., male A/J for humanized 13.2v.2 and female BALB/c for humanized MJ2-7v.2-11), male Spraugue-Dawley rats, naïve male cynomolgus monkeys, and the Ascaris-challenged cynomolgus monkey model, described in Example 21a. IV doses were administered, according to the most recent scheduled body weights, as a single bolus injection into the tail vein, jugular vein via catheter, or saphenous vein for mice, rats, and monkeys, respectively.

For the Ascaris-challenged cynomolgus monkey model, animals selected according to the protocol described in Example 21a, were treated with humanized MJ2-7v.2 administered via a short (e.g., approximately 10 minutes) IV infusion as described supra. 24 hours post IV infusion, animals were challenged with 0.75 μg Ascaris suum antigen reconstituted with PBS (Greer Diagnostics, Lenoir, N.C.) and administered by aerosol delivery.

For the Ascaris-challenged sheep model, female sheep, pre-screened for airway hypersensitivity to Ascaris suum antigen, were treated with an IV bolus injection of humanized 13.2v.2 (2 mg/kg) or IVIG (2 mg/kg). Ascaris-challenge was then administered 24 hours later using aerosol delivery.

Following the appropriate treatment, described above, blood samples were collected at pre-determined time points into serum separator tubes and allowed to clot at room temperature for 15 minutes, before processing by serum centrifugation (e.g., approximately 11,000 rpm for 10 minutes). Pre-determined time points were; pre-test and 5 minutes to 42 days in the humanized 13.2v.2 A/J mouse studies; 5 minutes to 21 days in the humanized MJ2-7v.2-11 BALB/c mouse studies, with 3-4 animals analyzed per time point; pre-test and 5 minutes to 35 days in both humanized 13.2v.2 and humanized MJ2-7v.2-11 rat studies; pre-test and 5 minutes to 42 days in the 1 mg/kg and 5 minutes to 55 days in the 100 mg/kg humanized 13.2v.2 and humanized MJ2-7v.2-11 naïve cynomolgus monkey studies; 5 minutes to 42 days in the humanized 13.2v.2 Ascaris-challenged sheep studies; and 24 hours to 113 days in the Ascaris-challenged cynomolgus monkey studies.

The concentrations of anti-IL-13 antibodies in mouse, rat, and cynomolgus monkey serum samples were determined using quantitative enzyme-linked immunosorbant assays (ELISA). In this assay, an IL-13 ligand, which contains a FLAG octapeptide fusion tag (Asp-Tyr-Lys-Asp-Asp-Asp-Asp-Lys), was captured onto a microtiter plate by an anti-FLAG monoclonal antibody. After blocking and washing, serum samples containing anti-IL-13 antibodies or anti-IL-13 standards were incubated on the plate to allow for binding to the FLAG tagged IL-13. Bound anti-IL-13 antibodies or anti-IL-13 standards were detected using a mouse anti-human IgG (Fc) antibody fused to horse radish peroxidase (HRP). Finally, bound antibodies were quantified using the HRP substrate 2,2′ azino-di (3-ethyl-benzthiazoline-6-sulfonate (ABTS) and optical densities were measured at 405 nm.

The ELISA to quantify humanized 13.2v.2 in sheep was performed as follows. Briefly, biotinylated humanized 13.2v.2 was pre-incubated with recombinant human IL-13-FLAG in the presence of either unlabeled humanized 13.2v.2 standards or humanized 13.2v.2-containing sheep serum. This mixture was transferred to a pre-washed and blocked anti-FLAG coated ELISA plate. Following a second incubation, the plate was washed and biotinylated humanized 13.2v.2 was detected with peroxidase-linked streptavidin. ELISA sample concentrations were determined by interpolation from a calibration curve fit using a 4-parameter equation (Softmax Pro).

Mouse PK parameters were based on mean concentrations for 3-4 animals at each time point, whereas rat and monkey PK parameters were determined for individual animals, as follows. All data was generated using a non-compartmental analysis module of the pharmacokinetic software package, WinNonlin (Pharsight). The area under the serum concentration versus time curve (AUC) was calculated using the linear trapezoidal model. The slope of the apparent terminal phase was estimated by log-linear regression using at least 3 data points and the terminal rate constant (Σ) was derived from the slope. AUC_{0-∞} was estimated as the sum of the AUC₀₋ₜ (t=time of last measurable concentration) and Cₜ/Σ. The apparent terminal half-life (t_{1/2}) was calculated as 0.693/Σ.

Human PK parameters were predicted for a subject with a body weight of 60 Kg using an allometric scaling approach, as follows. PK parameters calculated from each species were plotted on log-coordinates, and the allometric coefficient (a) and allometric exponent (b) were estimated from the linear regression: log Y=log(a)+log(w)*b (where log(a)=y intercept; b=slope of fit). PK parameters were then scaled using the equation: Y=a·W^{b} (where; Y═PK parameter of interest; W=body weight of species; a=allometric coefficient; b=allometric exponent), as shown in Table 7. PK data is presented in Tables 5A-5C.

TABLE 5A
Interspecies Comparison of Mean (±SD) Pharmacokinetic
Parameters for Humanized 13.2v.2 and Humanized MJ2-7v.2-11
Following Single IV Administration

| | Humanized 13.2v.2 | | | |
|---|---|---|---|---|
| | Dosage | Mean CL | Mean Vdₛₛ | Mean T_{1/2} |
| Species | (mg/kg) | (mL/hr/kg) | (mL/kg) | (hr) |
| Mouse | 1 | 0.813 | 60 | 78 |
| Rat | 2 | 0.418 ± 0.050 | 115 ± 18 | 207 ± 23 |
| (N = 4) | | | | |
| Monkey | 1 | 0.134 ± 0.034 | 54 ± 12 | 341 ± 47 |
| (N = 3) | | | | |
| Monkey | ND | ND | ND | ND |
| Ascaris | | | | |
| (N = 7) | | | | |
| Monkey | 100 | 0.172 ± 0.030 | 61 ± 12 | 245 ± 70 |
| (N = #3) | | | | |
| Sheep | 2 | 0.131 | 61 | 330 |
| Ascaris | | | | |
| (N = 2) | | | | |
| Predicted | N/A | 0.067 | 68 | 708 |
| Human | | | | |
| (60 kg) | | | | |

TABLE 5B
Interspecies Comparison of Mean Pharmacokinetic Parameters for
Humanized 13.2v.2 and Humanized MJ2-7v.2-11 Following Single IV
Administration

| | Humanized MJ2-7v.2-11 | | | |
|---|---|---|---|---|
| | Dosage | Mean CL | Mean Vdₛₛ | Mean T_{1/2} |
| Species | (mg/kg) | (mL/hr/kg) | (mL/kg) | (hr) |
| Mouse | 2 | 0.35 | 65 | 138.5 |
| Rat | 2 | 0.276 ± 0.090 | 86 ± 15 | 252 ± 87 |
| (N = 4) | | | | |
| Monkey | 1 | 0.134 ± 0.012 | 77 ± 7 | 396 ± 25 |
| (N = 3) | | | | |
| Monkey | 10 | 0.100 ± 0.033 | 45 ± 11 | 359 ± 115 |
| Ascaris | | | | |
| (N = 8) | | | | |
| Monkey | 100 | 0.171 ± 0.046 | 63 ± 4 | 299 ± 187 |
| (N = 3) | | | | |
| Predicted | N/A | 0.104 | 94 | 663 |
| Human | | | | |
| (60 kg) | | | | |

TABLE 5C
Dose-Normalized Exposure of Humanized 13.2v.2 and
Humanized MJ2-7v.2-11 Following Single IV Administration

| | h13.2v.2 | | hMJ2-7v.2-11 | |
|---|---|---|---|---|
| | Dosage | Mean | Dosage | Mean |
| | Level | AUC/Dose | Level | AUC/Dose |
| | (single dose | [(μg * hr/mL)/ | (single dose | [(μg * hr/mL)/ |
| Species | IV, mg/kg) | (mg/kg)] | IV, mg/kg) | (mg/kg)] |
| Mouse | 1 | 1231 | 2 | 3226 |
| Rat | 2 | 2418 | 2 | 3867 |
| Monkey | 1 | 7877 | 1 | 7410 |
| Predicted | 1 | 14886 | 1 | 9628 |
| Human | | | | |

PK profiles were determined for humanized 13.2v.2 and humanized MJ2-7v.2-11 in mice, rats, sheep, and monkeys as described above. As shown in Table 5A-5B, in general, PK parameters were comparable for all species analyzed. More specifically, PK data clearly demonstrates the elimination of both anti-IL-13 antibodies was slow, with serum clearances (CL) ranging from 0.13 mL/hr/kg in monkeys and sheep to 0.81 mL/hr/kg in mice. Steady state volume of distribution (Vdₛₛ) was also low for all species (<120 mL/kg), indicating that the anti-IL-13 antibodies were present mainly in the vascular circulation. Interestingly, the apparent terminal half life (T_{1/2}) was 3-6 days in mice (a non-binding species) compared to 14-17 days in monkeys and sheep (IL-13 binding species). In monkeys, PK parameters were determined at both 1 mg/kg and 100 mg/kg dosage levels. PK parameters for humanized 13.2v.2 and humanized MJ2-7v.2-11 antibodies were approximately dose-proportional in the 1-100 mg/kg range, as CL, t_{1/2}, Vdₛₛ, and dose-normalized exposure (AUC/dose) were not significantly different between the 1 and 100 mg/kg dosage levels. In general, PK parameters in naïve and Ascaris-challenged monkeys were not significantly different, suggesting that there is no apparent target-mediated clearance of the anti-IL-13 antibodies at the therapeutic dose level. However, the Vdₛₛ of humanized MJ2-7v.2-11 was lower in Ascaris-challenged monkeys, particularly when compared to 1 mg/kg of humanized MJ2-7v.2-11-treated naïve monkeys, possibly due to IL-13 redistribution caused by vascular re-modeling.

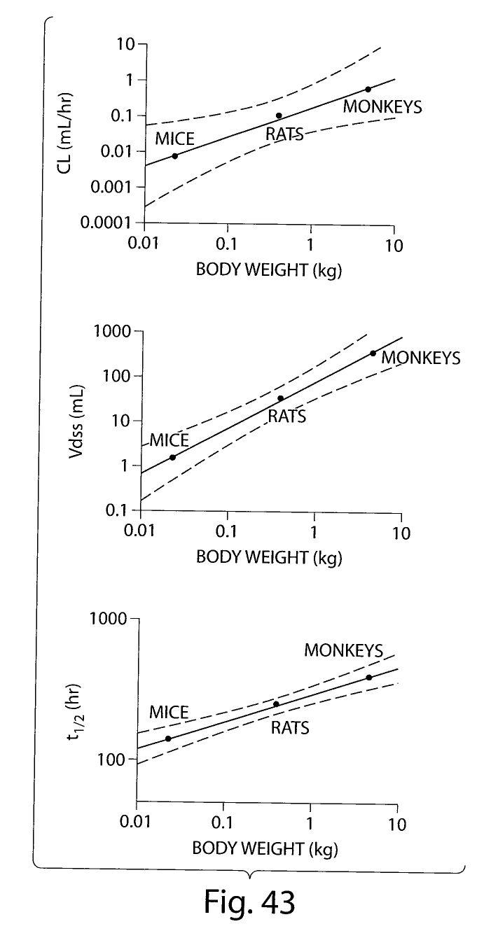

Allometric scaling was applied to predict PK of humanized 13.2v.2 and humanized MJ2-7v.2-11 antibodies in humans after IV administration. As shown in Tables 5A-5B and FIG. 43, both anti-IL-13 antibodies were predicted to have a highly favorable PK profile in humans with a low CL (e.g., approximately 0.07-0.1 mL/hr/kg), a low Vdₛₛ (e.g., approximately 68-90 mL/kg), and a long t_{1/2} (e.g., approximately 27-29 days).

Dose-normalized exposure data (AUC_{0-∞}/Dose) obtained from the above described IV studies were used to calculate bioavailability following 2 mg/kg subcutaneous (SC) administration of humanized 13.2v.2 and humanized MJ2-7v.2-11 antibodies.

TABLE 6
PK parameters of anti-IL-13 antibodies after SC administration

| | Humanized 13.2v.2 | | | | | Humanized MJ2-7v.2-11 | | | | |
|---|---|---|---|---|---|---|---|---|---|---|
| | F | Cₘₐₓ | Tₘₐₓ | AUC_{0-∞} | t_{1/2} | F | Cₘₐₓ | Tₘₐₓ | AUC_{0-∞} | t_{1/2} |
| Species | (%) | (μg/mL) | (h) | (μg h/mL) | (h) | (%) | (μg/mL) | (h) | (μg h/mL) | (h) |
| Mouse^{a} | 87 | 7.3 | 48 | 1065 | 45 | 86 | 24.2 | 24 | 5535 | 162 |
| Rat^{b} | 91 ± 16 | 11.3 ± 1.2 | 54 ± 12 | 4385 ± 766 | 206 ± 45 | ND | ND | ND | ND | ND |
| Monkey^{b} | 61 ± 8 | 22.6 ± 5.5 | 80 ± 14 | 9584 ± 1230 | 272 ± 59 | 74 ± 33 | 22.6 ± 6.4 | 40 ± 14 | 11,283 ± 5343 | 280 ± 147 |

F = bioavailability after SC dosage;
t_{1/2} = apparent terminal half-life;
Cₘₐₓ = maximum observed serum concentration;
Tₘₐₓ = time when Cₘₐₓ was reached;
AUC = area under the concentration-versus-time curve;
ND = not determined.
^{a}In mice, PK parameters were calculated based on the mean value from 3-4 animals per time-point. A/J and BALB/c mice were used for humanized 13.2v.2 (1 mg/kg) and MJ2-7v.2-11 (2 mg/kg), respectively.
^{b}In rats (Sprague-Dawley, N = 4) and monkeys (cynomolgus, N = 3), PK parameters were calculated for each individual animal.
Data show mean ± standard deviation. A single SC dosage of 2 mg/kg was used for both humanized 13.2v.2 and humanized MJ2-7v.2-11.

As shown in Table 6, the bioavailability of anti-IL-13 antibodies was 60-100% in all species tested. The maximum serum concentration (Cₘₐₓ) observed at 1-3 days post dosing ranged from 7.25 μg/mL in mice to 22.6 μg/kg in monkeys for humanized 13.2v.2, and 24.2 μg/mL in mice to 22.5 μg/mL in monkeys for humanized MJ2-7v.2-11. Absorption from the injection site of both anti-IL-13 antibodies was slow; however, slightly faster for humanized MJ2-7v.2-11. Based on the high levels of SC bioavailability in preclinical species, both anti-IL-13 antibodies were predicted to have ≧50% bioavailability in humans.

As described above, human PK parameters were predicted for a subject with a body weight of 60 kg using an allometric scaling approach. Briefly, PK parameters presented in Table 5 for mice, rats, and monkeys, were regressed against body weights (e.g., PK parameter=a·Weight^{b}) to obtain R². PK parameters for each species were then plotted on log coordinates and the allometric coefficient (a) and the allometric exponent (b) were estimated from the linear regression, as shown in Table 7.

TABLE 7
Allometric Scaling of Anti-IL-13 Antibody PK Parameters

| | Humanized 13.2v.2 | | | Humanized MJ2-7v.2-11 | | |
|---|---|---|---|---|---|---|
| | a | b | R² | a | b | R² |
| CL | 0.2524 | 0.6767 | 0.991 | 0.1931 | 0.8485 | 0.993 |
| Vdₛₛ | 71.051 | 0.9882 | 0.978 | 78.15 | 1.0327 | 0.999 |
| t_{1/2} | 235.69 | 0.2687 | 0.982 | 295.5 | 0.1974 | 0.999 |

Table 7 shows the allometric coefficients (a), allometric exponents (b), and R² values obtained from regression of PK parameters against body weight and the CL, t_{1/2}, and Vdₛₛ for both anti-IL-13 antibodies.

Humanized 13.2v.2 and humanized MJ2-7v.2-11 antibody biodistribution assays were performed in A/J mice and Sprague-Dawley rats, respectively, using radio labeled anti-IL-13 antibodies. Briefly, humanized 13.2v.2 was labeled using the Iodo-gen reagent (1,3,4,6-tetrachloro-3,6-diphenylglycoluril, supplied by Pierce). A 20 μL aliquot of Iodo-gen solution was combined with 1 mCi [¹²⁵I] dissolved in 100 TL PBS and 10 μL of humanized 13.2v.2 antibody and incubated for 15 minutes at room temperature. [¹²⁵I]-labeled humanized 13.2v.2 was purified using a NAP 5 column (Pharmacia, Uppsala, Sweden). Similarly, humanized MJ2-7v.2 was iodinated using the IODO-BEADS method (Pierce, Rockford, Ill.) in which 300 μg of humanized MJ2-7v.2-11 antibody was incubated for 25 minutes with 3 mCi of [¹²⁵I], IODO BEADS, and PBS. Unincorporated [¹²⁵I] was separated from the IODO BEADS by filtration (Centricon, 10 kD cut-off), and the resulting [¹²⁵I]-labeled humanized MJ2-7v.2-11 antibody was mixed with unlabeled HMJ2-7V2-11. The specific activities of [¹²⁵I]-labeled humanized 13.2v.2 and [¹²⁵I]-labeled HMJ2-7v.2-11 anti-IL-13 antibodies were 2.79×10⁸ cpm/mg (unincorporated iodine ≦5%) and 2.56×10⁷ cpm/mg (unincorporated iodine ≦1.1%), respectively. [¹²⁵I]-labeled humanized 13.2v.2 was then administered IV at a dose of 1 mg/kg and [¹²⁵I]-labeled humanized MJ2-7v.2-11 was administered at a dose of 2 mg/kg. Tissue samples were subsequently collected at 1, 24, 168, and 336 hours for the [¹²⁵I] labeled humanized 13.2v.2 mouse study and at 1, 48, 168, 336 and 840 hours for the [¹²⁵I]-labeled humanized MJ2-7v.2-11 rat study. Tissues including, for example, spleen, lung, heart, liver, kidney, skeletal muscle, stomach, small intestine, large intestine, lymph node, skin, and fat were collected immediately after blood sampling and whole body perfusion with heparinized PBS at 25 U/mL.

Anti-IL-13 antibody levels, defined as radioactive equivalent concentrations, in serum (μg eq./mL) and tissue (μg eq./g) were estimated by gamma-counting trichloroacetic acid (TCA)-precipitable or total radioactivity, respectively, and the following formulas: For serum; [average TCA precipitable cpm/EXP(0.693/60.2×(tₛ−t_{D}))]/[specific activity×sample volume]: For tissue; [average TCA precipitable cpm/EXP(0.693/60.2×(tₛ−t_{D}))]/[specific activity×sample weight], where tₛ is dates of sample and t_{D} is dosing solution measurement after correction for the half-life of [¹²⁵I].

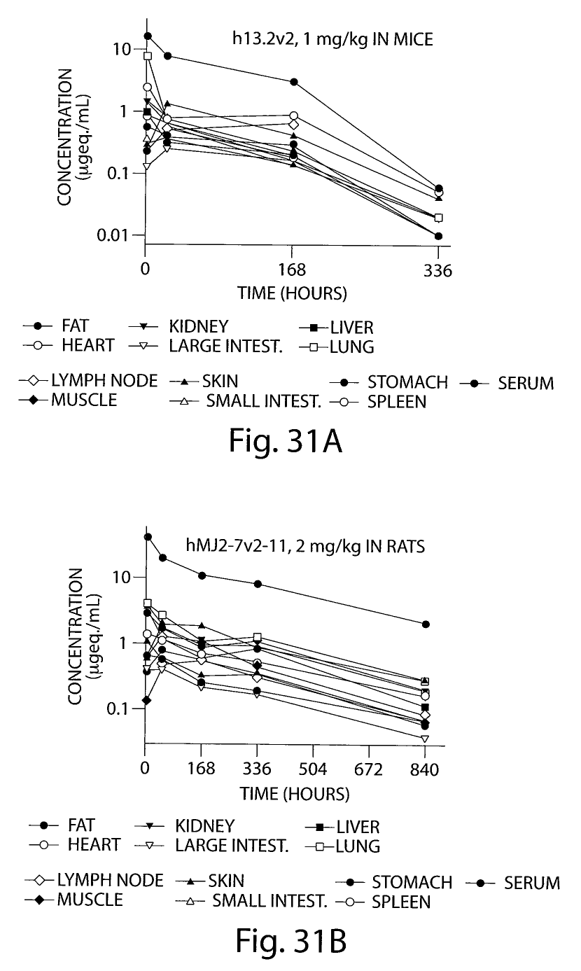

As shown in FIGS. 31A-31B, following IV administration of [¹²⁵I] labeled humanized 13.2v.2 and [¹²⁵I]-labeled humanized MJ2-7v.2-11 antibodies, the highest levels of both antibodies were detected in the serum, confirming that both anti-IL-13 antibodies are present predominantly in the vasculature. Other tissues with high levels of both anti-IL-13 antibodies include highly perfused tissues, for example, lung, kidney, liver, heart, and spleen. Of all the tissue compartments analyzed, humanized 13.2v.2 and humanized MJ2-7v.2-11 antibody levels were highest at the 1 hour time point in the lung, indicating that both anti-IL-13 antibodies are rapidly delivered to this tissue, which is also the desired target organ for future therapeutic application. Finally, both humanized 13.2v.2 and humanized MJ2-7v.2-11 antibody levels declined over the duration of this study, and only trace amounts were detected at the final time points.

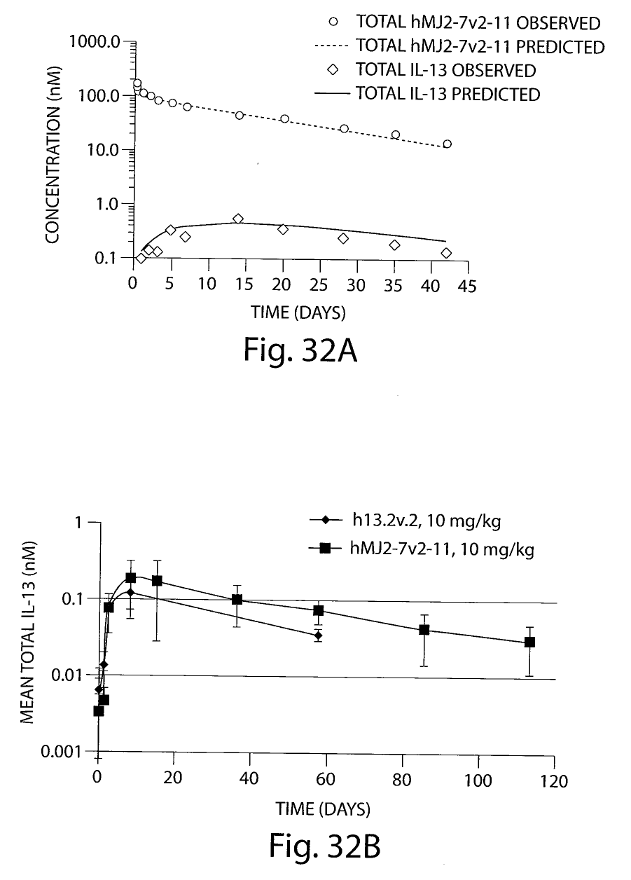

Humanized 13.2v.2 and humanized MJ2-7v.2-11 antibody pharmacodynamics (PD) were also analyzed using the ELISA described above. As shown in FIG. 32A-B, total IL-13 levels transiently increased following IV administration of both humanized 13.2v.2 and humanized MJ2-7v.2-11 antibodies in naive and Ascaris-challenged cynomolgus monkeys. Importantly, however, IL-13 in the serum of these animals had no biological activity when tested in a cell-based potency assay (data not shown). IL-13 was not detectable at all time points in sera from IVIG or saline-treated animals (data not shown).

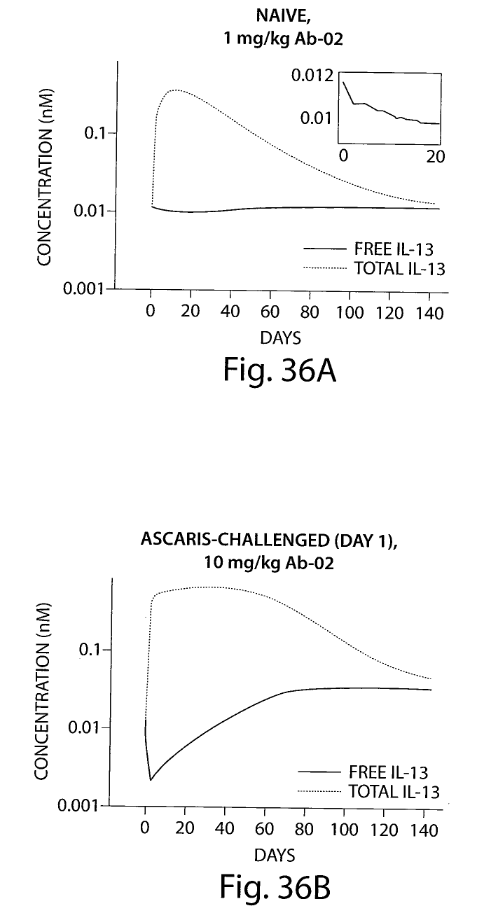

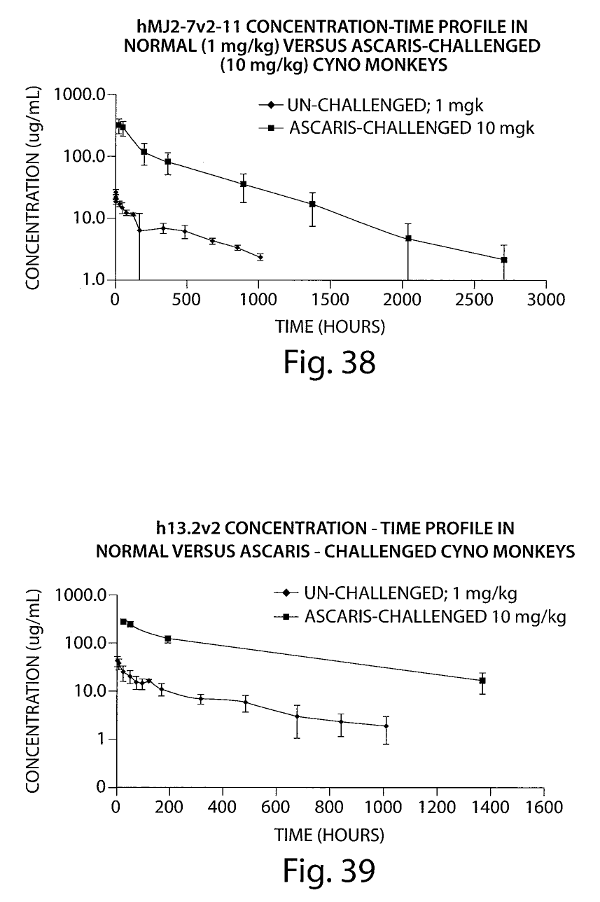

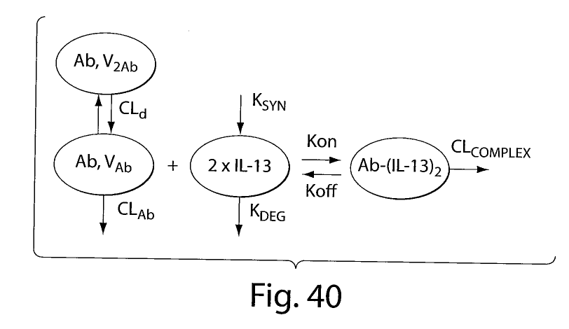

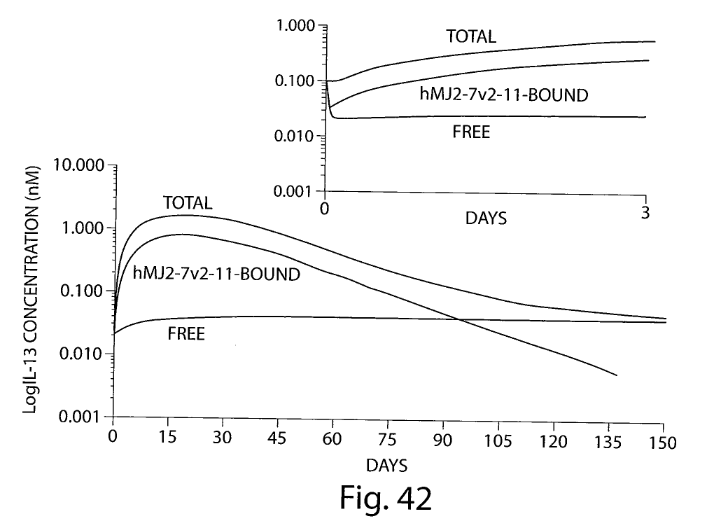

Further analysis of IL-13 levels following administration of anti-IL-13 antibodies was conducted using allometric scaling, as described above. Briefly, as shown in FIGS. 38 and 39, concentration-time profiles were calculated for humanized MJ2-7v.2-11 and humanized 13.2v.2, respectively, in naïve versus normal cynomolgus monkeys. This data was combined with PK data presented in Table 5 and applied to the model depicted in FIG. 40 and the equation described above. The resulting allometric scaling data for humanized MJ2-7v.2-11 in naïve cynomolgus monkeys is presented in FIG. 36 and Table 8. The resulting allometric data for humanized MJ2-7v.2-11 in Ascaris-challenged monkeys is presented in FIG. 42 and Table 10.

### Example 21a

### Pharmacokinetic and Pharmacodynamic Modeling of a Humanized Anti-IL-13 Antibody in Naive and Ascaris-Challenged Cynomolgus Monkeys (“Sequential Model”)

This example discusses an integrated model that describes pharmacokinetics and pharmacodynamics of an anti-IL-13 antibody in both naive animals and in the animal pharmacology study. The model is used to characterize the kinetics of IL-13 neutralization by an anti-IL-13 antibody in both naïve and pharmacology-study settings. The model exemplified herein with IL-13 can be extended to evaluate other drug-ligand interaction, particularly where free cytokine levels are difficult to assay directly.

Cytokine neutralization by monoclonal antibodies or cytokine receptor/Fc fusion proteins is being explored as a therapeutic approach for a variety of cytokine-mediated disorders, including autoimmune diseases, such as rheumatoid arthritis (RA), asthma, and systemic lupus erythematosus (SLE) (Ichinose et al., Curr Drug Targets Inflamm Allergy 2004; 3(3):263-9; Economides et al., Nat Med 2003; 9(1):47-52; Toussirot et al., Expert Opin Pharmacother 2007; 8(13):2089-107; and Anolik et al., Best Pract Res Clin Rheumatol 2005; 19(5):859-78). A common problem in the development of therapeutic proteins is that cytokine neutralization cannot be directly monitored in the presence of a drug, due to unavailability of an assay method of sufficient sensitivity to measure free cytokine levels. Instead, total (free plus drug-bound) cytokine levels are often used as a surrogate pharmacodynamic (PD) marker of drug activity. There are several examples of anti-cytokine proteins acting as “cytokine traps”, resulting in increased total circulating cytokine levels following drug administration, presumably due to slower elimination of a drug-bound circulating cytokine, compared to that of a free circulating cytokine (Margolin et al., J Clin Oncol 2001; 19(3):851-6; Charles et al., J Immunol 1999; 163(3):1521-8; Ito et al., Gastroenterology 2004; 126(4):989-96; discussion 947).

When free cytokine levels (in the presence and often in the absence of an anti-cytokine protein) are difficult to assay directly, PK-PD modeling can be a useful tool for delineating a relationship between the kinetics of ligand neutralization and the concentration-time profile of an anti-cytokine therapeutic, using total cytokine levels as a PD marker. These models can be especially useful when data from both healthy and disease subjects (animals or humans) subjects are available, so that the free cytokine levels can be estimated before and after therapy in both settings. Establishing a relationship between the kinetics of ligand neutralization and the concentration-time profile of potential therapeutic, combined with efficacy data, can be useful for design of an optimal dosing regimen in animal pharmacology or in clinical studies.

Neutralization of interleukin-13 (IL-13) is an attractive approach for therapeutic intervention in asthma, as this Th2 cytokine plays an important role in asthma pathogenesis in animal models of asthma (Andrews et al., J Biol Chem 2002; 277(48):46073-8; Corry et al., Am J Respir Med 2002; 1(3):185-93; Wills-Karp et al., Curr Allergy Asthma Rep 2004; 4(2):123-31; Grunig et al., Science 1998; 282(5397):2261-3; Padilla et al., J Immunol 2005; 174(12):8097-105; Taube et al., J Immunol 2002; 169(11):6482-9). In addition, there are consistent correlations between polymorphism in the IL-13 gene and asthma susceptibility in humans (Vercelli, Curr Opin Allergy Clin Immunol 2002; 2(5):389-93). Neutralization of IL-13 with anti-IL-13 antibodies or with IL-13 receptor α2/Fc fusion protein (IL-13Rα2-Fc) prevents airway hyperresponsiveness and other asthmatic changes in mice (Taube et al.; Grunig et al; Kumar, Am J Respir Crit Care Med 2004; 170(10):1043-8; Wills-Karp et al., Science 1998; 282(5397):2258-61; Yang et al., J Pharmacol Exp Ther 2005; 313(1):8-15), sheep (Kasaian et al., Am J Respir Cell Mol Biol 2007; 36(3):368-76), and cynomolgus monkeys (Bree et al., J Allergy Clin Immunol 2007; 119(5):1251-7).

IL-13 signals via a receptor complex consisting of IL-13 receptor α1 (IL13αR1) and interleukin-4 receptor alpha (IL-4Rα) subunits (Andrews et al., J Biol Chem 2002; 277(48):46073-8; Corry et al., Am J Respir Med 2002; 1(3): 185-93). IL-13 first undergoes a low affinity interaction with IL-13Rα1, which recruits IL-4Rα to form an active signaling complex with high affinity for IL-13, leading to phosphorylation of STAT6 and downstream cellular activation events.

hMJ2-7v.2-11, discussed herein is a humanized anti-IL-13 antibody that blocks binding of IL13Rα1 to human and non-human primate IL-13. hMJ2-7v.2-11 does not substantially cross-react with either rodent or sheep IL-13; thus non-human primates were used as pharmacological species. As discussed herein, hMJ2-7v.2-11 has been shown to be efficacious (at 10 mg/kg IV dose) in the model of acute airway inflammation induced by Ascaris challenge in cynomolgus monkeys. In this example, the PK and total IL-13 (PD) data following hMJ2-7v.2-11 administration to naive and Ascaris-challenged monkeys were used to establish an integrated PK-PD model and characterize the kinetics of IL-13 neutralization.

The study design is summarized in Table 8. Single dose pharmacokinetic studies in protein-free adult fed cynomolgus monkeys were conducted at Wyeth Research (Pearl River, N.Y. and Andover, Mass. for Study 1 and Study 2, respectively), as previously described. hMJ2-7v.2-11 was administered by IV injection into saphenous vein or by SC route. The dose was based on the most recent scheduled body weights, prior to dosing. Blood samples were collected into serum separator tubes at the designated time-points (Table 8), allowed to clot at room temperature for approximately 15 minutes, and processed for serum by centrifugation (approximately 11,000 rpm for 10 minutes).

TABLE 8
Study Design

| | | hMJ2- | | |
|---|---|---|---|---|
| | | 7v.2-11 | | |
| | | HMJ2- | | |
| | | 7v.2- | | PK and PD |
| | | 11Dose | Dosing volume | sampling |
| Study Number | N, sex | (mg/kg) | and buffer | time-points (days) |
| Study 1, naive | 3, males | 1(IV) | 1 mL/kg in | 0, 0.004, 0.042, |
| monkeys | | and 2 | Histidine-Sucrose | 0.125, 0.25, 1, |
| | | (SC) | Buffer^{b} | 2, 3, 5, 7, 14, 20, |
| | | | | 28, 35, 42 |
| Study 2, | 8, males | 10 (IV) | 2-3 mL/kg in PBS | 0, 1, 2, 8, 15, 36, |
| Ascaris- | | | | 57, 85, 113 |
| challenged | | | | |
| monkeys^{a} | | | | |

^{a}Animals were challenged with 0.75 mg Ascaris suum 24 hours post hMJ2-7v.2-administration.
^{b}10 mM histidine, 5% sucrose, pH 6.0

Ascaris-challenge study protocol was described previously (Bree et al., J Allergy Clin Immunol 2007; 119(5):1251-7). In brief, several months prior to the study untreated monkeys were given an initial screening challenge with Ascaris suum antigen. Monkeys that responded with at least a 2-fold increase in bronchoalveolar lavage (BAL) eosinophil content 24 hours post-challenge were selected for the study. Animals were administered either hMJ2-7v.2-11 (10 mg/kg) or a negative control (IVIG, 10 mg/kg) by IV route and were challenged with 0.75 μg Ascaris suum antigen (obtained from Greer Diagnostics, Lenoir, N.C. and reconstituted with PBS) 24 hours post administration of hMJ2-7v.2-11 or a negative control.

The concentrations of hMJ2-7v.2-11 in serum samples were determined using quantitative enzyme-linked immunosorbent assays (ELISA). In this assay, the recombinant human IL-13 ligand, which contains a FLAG ocatapeptide fusion tag (Asp-Tyr-Lys-Asp-Asp-Asp-Asp-Lys) was captured onto a microtiter plate by an anti-FLAG monoclonal antibody. After blocking and washing, the serum samples containing hMJ2-7v.2-11 or the hMJ2-7v.2-11 standards were incubated on the plate to allow for binding to the IL-13. Bound hMJ2-7v.2-11 were detected with a mouse anti-human IgG (Fc) antibody conjugated to horseradish peroxidase (HRP). The enzyme substrate 2,2′ azino-di (3-ethyl-benzothiazoline-6-sulfonate (ABTS) was added and optical densities were measured at 405 nm. The low limit of quantitation of the assay was approximately 10.5 ng/mL.

The concentrations of total IL-13 in serum samples obtained from hMJ2-7v.2-11-treated monkeys were determined using quantitative ELISA. In this assay, an anti-IL-13 antibody (humanized 13.2 antibody, 13.2v.2, Wyeth Research) that was able to bind IL-13 in the presence of hMJ2-7v.2-11 HMJ2-7v.2-11 was used as a capture. After blocking and washing, the serum samples containing IL-13 from in vivo studies or the non-human primate IL-13 standards were incubated on the plate to allow for binding to the anti-IL-13 capture antibody. Total IL-13 was detected with a biotinylated Jin2, an anti-IL-13 antibody that binds to an IL-13 epitope that is distinct from those of humanized 13.2 and hMJ2-7v.2-11. Streptavidin conjugated to HRP and the enzyme substrate 3,3′,5,5′-tetramethylbenzidine (TMB) peroxidase were added and optical densities were measured at 450 nm. The low limit of quantitation of the assay was approximately 0.15 ng/mL.

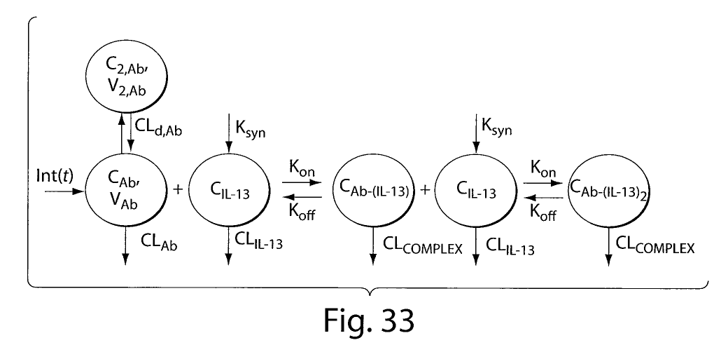

An integrated pharmacokinetic and pharmacodynamic model that described the relationship between observed serum concentrations of hMJ2-7v.2-11 and total IL-13, was developed using WinNonlin software V 5.1.1 (Pharsight, Mountain View, Calif.) (FIG. 33). The pharmacokinetics of hMJ2-7v.2-11 was evaluated with a two-compartmental model including a central compartment (C_{Ab}, V) and a peripheral compartment (C_{2, Ab}, V₂). CL_{d,Ab} represented the distribution clearance between these two compartments. Clearance (CL_{Ab}) of hMJ2-7v.2-11 was assumed only through the central compartment. The pharmacodynamics of hMJ2-7v.2-11 was characterized with the neutralization of endogenous IL-13. Based on the bivalent feature of IgG, the model assumed that each hMJ2-7v.2-11 molecule had two independent binding sites for IL-13 with identical association (Kₒₙ) and disassociation (K_{off}) rate constants. Kₒₙ was a 2^{nd} order rate constant governing the formation of hMJ2-7v.2-11/IL-13 (Ab-IL-13) complex and K_{off} was a 1^{st} order rate constant governing the disassociation of Ab-IL-13 complex. CL_{complex} represented the serum clearance of Ab-IL-13 complex. The homeostasis of IL-13 was assumed to be regulated by IL-13 production (zero order, K_{syn,}) and degradation (CL_{IL-13}). Differential equations derived from the model scheme in FIG. 33 are as follows:

dC_{Ab}/dt=[In(t)+CL_{d,Ab}·C_{2,Ab}−(CL_{d,Ab}+CL_{Ab})·C_{Ab}]/V−Kₒₙ·C_{Ab}*(C_{IL-13}−C_{Ab-(IL-13)}−C_{Ab-(IL-13)}_{₂})+K_{off}·C_{Ab-(IL-13)} when t=0,C_{Ab}⁰=In(0)/V (1)

dC_{2,Ab}/dt=(CL_{d,Ab}·C_{Ab}−CL_{d,Ab}·C_{2,Ab})/V₂ when t=0,C_{2,Ab}⁰=0 (2)

dC_{Ab-(IL-13)}/dt=Kₒₙ·C_{Ab}·(C_{IL-13}−C_{Ab-(IL-13)}−C_{Ab-(IL-13)}_{₂})−CL_{complex}·C_{Ab-(IL-13)}−K_{off}·C_{Ab-(IL-13)}+K_{off}·C_{Ab-(IL-13)}_{₂}−Kₒₙ·C_{Ab-(IL-13)}·(C_{IL-13}−C_{Ab-(IL-13)}−C_{Ab-(IL-13)}_{₂}) when t=0,C_{Ab-(IL-13)}⁰=0 (3)

dC_{Ab-(IL-13)}_{₂}/dt=Kₒₙ·C_{Ab-(IL-13)}·(C_{IL-13}−C_{Ab-(IL-13)}−C_{Ab-(IL-13)}_{₂})−CL_{complex}·C_{Ab-(IL-13})_{₂}−K_{off}·C_{Ab-(IL-3)}_{₂} when t=0,C_{Ab-(IL-13)}_{₂}⁰=0 (4)

dC_{IL-13}/dt=[K_{syn}−CL_{IL-13}·(C_{IL-13}−C_{Ab-(IL-13)}−C_{Ab-(IL-13)}_{₂})]/V−Kₒₙ·C_{Ab}·(C_{IL-13}−C_{Ab-(IL-13)}−C_{Ab-(IL-13)}_{₂})−Kₒₙ·C_{Ab-(IL-13)}·(C_{IL-13}−C_{Ab-(IL-13)}−C_{Ab-(IL-13)}_{₂})+k_{off}·C_{A-(IL-13)}+K_{off}·C_{Ab-(IL-13)}_{₂} when t=0,C_{Il-13}⁰=K_{syn}/CL_{IL-13} (5)

For iv bolus dose:

In(t)=Dose (6)

For sc dose:

In(t)=Kₐ·F·Dose (7)

Since preliminary modeling indicated that hMJ2-7v.2-11, IL-13 and Ab-IL-13 complex had similar estimates of volume of distribution in a central compartment (approximately 0.1 to −0.3 L), a single volume variable (V) was used in the final modeling for model parsimony. A 1^{st} order absorption rate constant (Kₐ) was used to describe the absorption process for a subcutaneous dose.

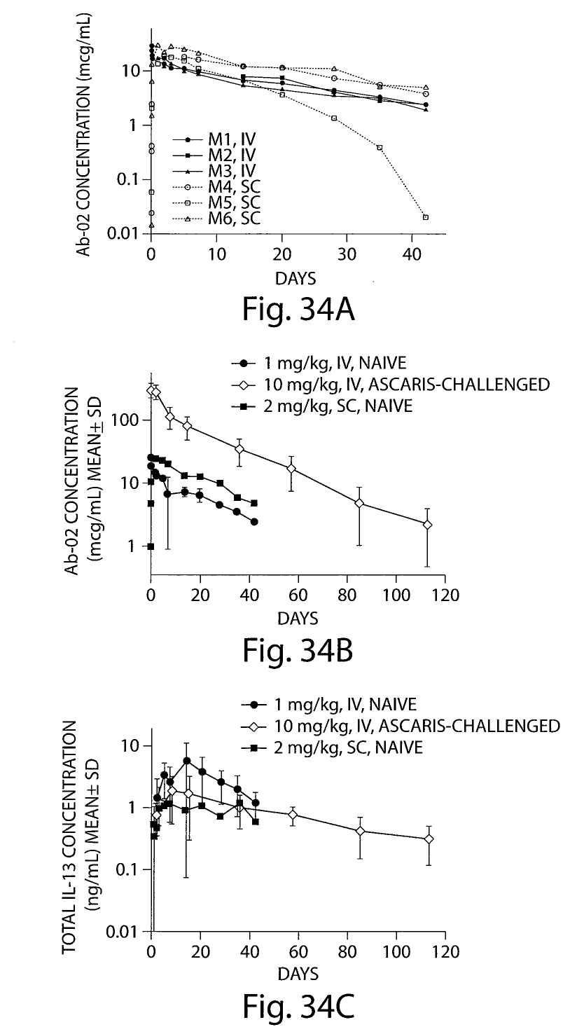

Except for estimate of bioavailability (F), PK/PD parameter estimates were obtained by simultaneously fitting the model to both serum hMJ2-7v.2-11 HMJ2-7v.2-11 and total IL-13 concentration-time profiles from either individual naive or Ascaris-challenged monkeys. The integrated PK/PD model was fitted first to data from naive monkeys with IV (n=3) and SC (n=3) doses. Bioavailability (F) of the anti-IL-13 antibody after the SC dose was estimated with non-compartmental analysis as shown in Example 21b. One naive monkey (Monkey #5) in the SC arm of the study, had a sharp decline in hMJ2-7v.2-11 HMJ2-7v.2-11 levels in the terminal phase (and a faster drop of total IL-13 levels), compared to other naive monkeys in both the IV and SC arms (FIG. 34A), likely due to formation of antibodies against hMJ2-7v.2-11. Therefore Monkey #5 was excluded from the calculation of the mean model parameters in the naive-model settings. It was assumed that Kₒₙ and K_{off} were not altered by Ascaris challenge. Therefore, mean Kₒₙ and K_{off} estimates obtained from naive monkeys were used in the model fitting for Ascaris-challenged monkeys. The onset of inflammation in Ascaris-challenged monkeys was assumed to occur instantaneously after the challenge at 24 hours post dose. Thus, naive condition was assumed for Ascaris-challenged monkeys in the pre-challenge period (0-24 hr) by fitting the data with mean parameters obtained from naive monkeys. All data were reported as mean±SD (n=5 for naive and n=8 for Ascaris-challenged monkeys). Statistical significance (p<0.05) was assessed with unpaired Student t-test.

Simulations for concentrations of hMJ2-7v.2-11, total IL-13, and free (unbound) IL-13 in naive or Ascaris-challenge settings after different dose regimens of hMJ2-7v.2-11, were conducted with the corresponding mean parameters obtained from PK/PD modeling. When Ascaris challenge was assumed at Day 1 (as used in the experiment design of Study 2), simulations for the 0-24 hours period were performed with mean parameter estimates from naive settings, while simulations for Day 1 onward were performed with mean parameter estimates from the Ascaris-challenge settings. When Ascaris challenge was assumed at Day 0 (for a hypothetic “established inflammation” situation), simulations for all time-points were performed with mean parameter estimates from the Ascaris-challenge settings.

Mean concentration-time profiles of hMJ2-7v.2-11 (1 mg/kg, IV and 2 mg/kg SC, Study 1) in naive cynomolgus monkeys were reported in Example 21b. Individual concentration-time profiles of hMJ2-7v.2-11 in Study 1 are shown in FIG. 34A. A sharp decline of hMJ2-7v.2-11 serum levels after approximately 14 days post-dose was observed in one animal (Monkey #5) in the SC arm of the study, relative to other five animals (three in the IV arm and two in the SC arm) in Study 1. Mean concentration-time profiles of hMJ2-7v.2-11 in Ascaris-challenged monkeys (10 mg/kg, IV, with Ascaris challenge 24 hours post-dose, Study 2) together with those in naive monkeys are summarized in FIG. 34B.

Quantitative ELISA were developed to measure total IL-13 levels in the absence or presence of hMJ2-7v.2-11. Serum IL-13 levels were undetectable by these assays in pre-dose samples or in all samples from control animals treated with IVIG (data not shown). After hMJ2-7v.2-11 administration, total IL-13 levels were transiently increased in both Study 1 (naive monkeys; 1 mg/kg IV or 2 mg/kg SC) and in Study 2 (10 mg/kg IV, with Ascaris challenge 24 hours post-dose) (FIG. 34C). There was high inter-animal variability in the concentration-time profiles of total IL-13. However, Monkey #5 in the arm of Study 1 had an apparent sharp decline in the total IL-13 levels, compared to other five naive monkeys on Study 1 that were treated with hMJ2-7v.2-11, likely due to formation of anti-hMJ2-7v.2-11 antibodies in this animal. The onset of decline in total IL-13 in Monkey #5 coincided with that in hMJ2-7v.2-11 levels in this monkey (data not shown).

Results of the previously reported cell-based assay performed with sera from Ascaris-challenged animals indicated that samples with detectable levels of total IL-13 had no IL-13-mediated biological activity (Kasaian et al., submitted), suggesting that the transient increase in total IL-13 levels in naive and Ascaris-challenged monkeys was due to the increase in hMJ2-7v.2-11-bound IL-13. However, the concentration-time profile of free, (biologically active) IL-13 following hMJ2-7v.2-11 administration to naive or Ascaris-challenged animals remained to be characterized.

An integrated drug-ligand binding PK-PD model depicted in FIG. 33 was developed to describe the relationship between the observed total serum concentrations of IL-13 and hMJ2-7v.2-11 in naive and Ascaris-challenged monkeys. In this model, the pharmacokinetics of hMJ2-7v.2-11 was described with a two-compartmental model and the pharmacodynamics of hMJ2-7v.2-11 was characterized with the neutralization of endogenous IL-13. Based on the bivalent feature of IgG, the models were developed under the assumption that hMJ2-7v.2-11 can bind either one or two IL-13 molecules, in a sequential manner. The homeostasis of IL-13 was assumed to be regulated by the zero-order synthesis (K_{syn}) and degradation (CL_{IL-13}) of IL-13.

For PK-PD modeling, raw concentration data (measured in ng/mL or μg/mL) was converted to nM units, using molecular weights of 150 kDa and 10 kDa for hMJ2-7v.2-II and IL-13 respectively.

TABLE 9
Summary of hMJ2-7v.2-11 Pharmacokinetic and Pharmacodynamic
Parameters from Individual Fittings of Data for Naive and
Ascaris-Challenged Cynomolgus Monkeys

| | | | Ascaris-challenged | |
|---|---|---|---|---|
| | Naive monkeys | | monkeys | |
| | Mean ± SD | | Mean ± SD | |
| | (N = 5)^{a} | % CV | (N = 8) | % CV |
| CL_{Ab} | 0.0148 ± 0.0022 | 15 | 0.0130 ± 0.0046 | 35 |
| (L/day) | | | | |
| V | 0.222 ± 0.045 | 20 | 0.145 ± 0.048* | 33 |
| (L) | | | | |
| CL_{d,Ab} | 0.1877 ± 0.1840 | 98 | 0.0238 ± 0.0192* | 81 |
| (L/day) | | | | |
| V₂ | 0.136 ± 0.071 | 53 | 0.111 ± 0.058 | 22 |
| (L) | | | | |
| Kₒₙ | 0.0896 ± 0.0917 | 102 | fixed | NA |
| nM⁻¹ day⁻¹ | | | | |
| K_{off} | 0.1630 ± 0.0959 | 59 | fixed | NA |
| (1/day) | | | | |
| CL_{complex} | 0.0024 ± 0.0006 | 23 | 0.0097 ± 0.0073* | 75 |
| (L/day) | | | | |
| K_{syn}/CL_{IL-13} | 0.0115 ± 0.0055 | 47 | 0.0346 ± 0.0101*** | 29 |
| (nM) | | | | |

^{a}For estimation of mean parameters in naive animals, three animals in the 1 mg/kg, IV group and 2 animals in the 2 mg/kg, SC group were used. One animal in the SC group was excluded from calculations of mean parameters due to a sharp decline in hMJ2-7v.2-11 levels (and total IL-13 levels) in the terminal phase, compared to other naive monkeys in the study.
Stars (* or ***) indicate that a mean parameter in the Ascaris-challenged animals was significantly different (p ≦ 0.05 or ≦0.001, respectively) from a corresponding value in naive monkeys, based on unpaired Student t test.

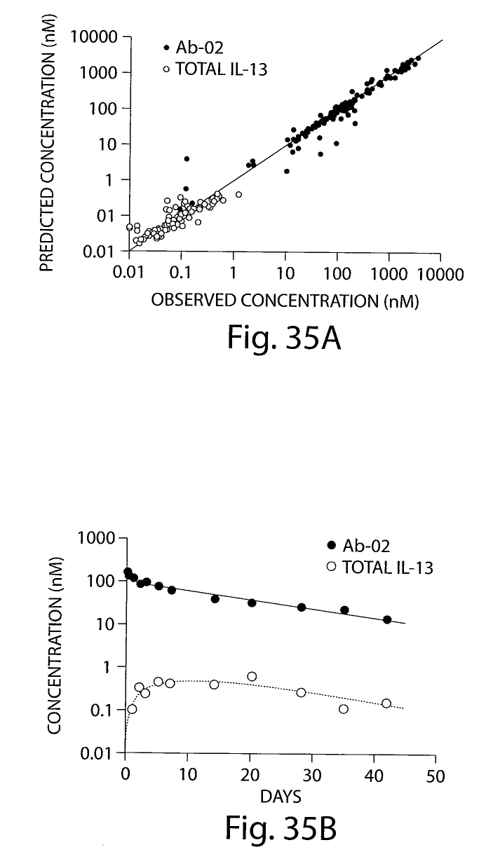

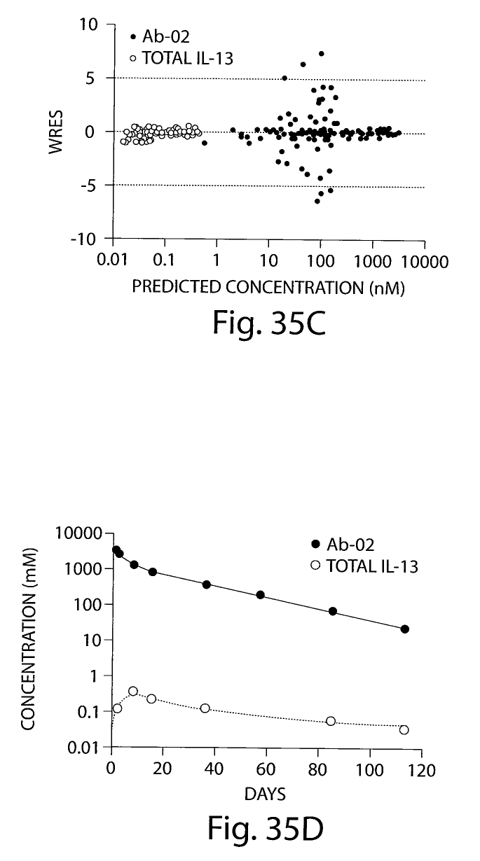

In general, this model adequately characterized the animal data (FIG. 35A and Table 9). The residuals were evenly distributed, without noticeable systematic bias (FIG. 35B). The representative fits for naïve (Study 1) and Ascaris-challenged (Study 2) monkeys are shown in FIGS. 35C and 35D, respectively. However, the sharp decline of both hMJ2-7v.2-11 and total IL-13 serum levels in Monkey #5 from the SC arm of Study 1 could not be described by this integrated PK/PD model. Therefore, the PK parameters from Monkey #5 were excluded from the calculation of the mean model parameters in the naive-animal settings.

PK and PD parameters generated from the model fitting for both naive and Ascaris-challenged monkeys are summarized in Table 9. The clearance of unbound hMJ2-7v.2-11 (CL_{Ab}) from the central compartment was low (approximately 0.013-0.015 L/day) and was similar between the naive and Ascaris-challenged monkeys. In naive animals, the clearance of hMJ2-7v.2-11/IL-13 complex from the central compartment (CL_{complex}) was approximately 5-6 fold lower, compared to CL_{Ab}. In Ascaris-challenged animals, CL_{complex} was similar to CL_{Ab}. Thus, CL_{complex} was approximately 5-fold higher in Ascaris-challenged animals, when compared to that in naive monkeys. The volume of hMJ2-7v.2-11 in the central compartment (V) was found to be similar to the average plasma volume in cynomolgus monkeys for both naive and Ascaris-challenged animals. However, V and the distribution clearance of hMJ2-7v.2-11 (CL_{d,Ab}) were significantly lower in the Ascaris-challenged monkeys, when compared to that in naive monkeys. This result is in accord with the lower estimate for the volume of distribution in Ascaris-challenged monkeys obtained with earlier non-compartment analysis (Vugmeyster et al., submitted).

The neutralization of IL-13 was governed by Kₒₙ and K_{off}, the rate constants of the coupling/uncoupling of hMJ2-7v.2-11 and free IL-13. The mean Kₒₙ and K_{off} estimates were 0.0896 nM⁻¹ day⁻¹ and 0.1630 day⁻¹, respectively. Baseline IL-13 levels were defined by the ratio of endogenous IL-13 synthesis rate (K_{syn}) and the clearance of IL-13 from the central compartment (CL_{IL-13}) (Benincosa et al., J Pharmacol Exp Ther 2000; 292(2):810-6; Ng et al., Pharm Res 2006; 23(1):95-103; Mager et al., J Pharmacokinet Pharmacodyn 2001; 28(6):507-32). The estimated baseline IL-13 level was approximately 0.0115 nM in naive monkeys and it was approximately 3-fold higher (approximately 0.0346 nM) in Ascaris-challenged monkeys (p<0.001).

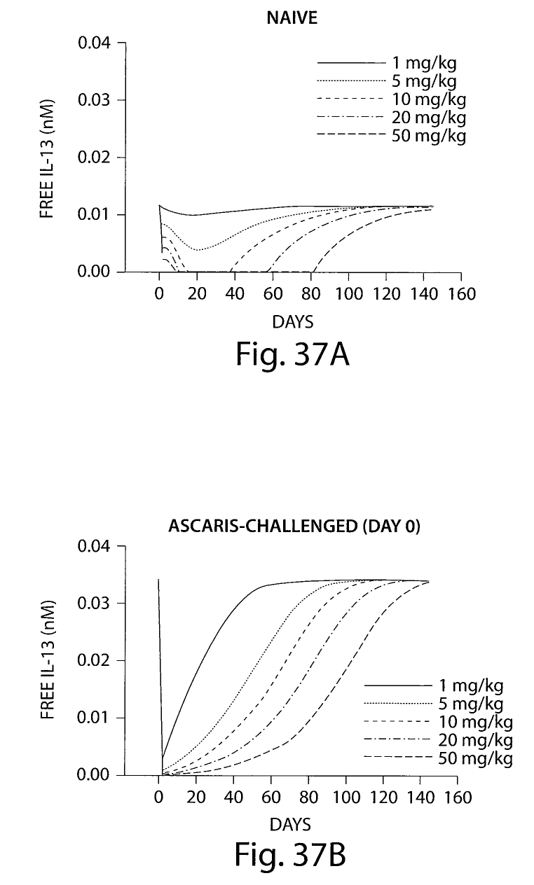

Model simulation with mean parameter estimates of the integrated PK-PD model were used to predict the levels of free and hMJ2-7v.2-11-bound IL-13 post hMJ2-7v.2-11 administration. These simulations predicted that the transient increase in total IL-13 levels in both Study 1 (naive) and Study 2 (Ascaris-challenged at Day 1) was due to the increase in hMJ2-7v.2-11-bound IL-13, while free IL-13 was decreased after the IV administration of hMJ2-7v.2-11 (FIGS. 36A and 36B). The decrease in free IL-13 appeared more dramatic in Ascaris-challenged monkeys, because of the higher hMJ2-7v.2-11 dose (10 mg/kg) used, relative to naive monkeys (1 mg/kg). In the Ascaris-challenge monkeys (Study 2), free IL-13 levels were predicted to remain at or below the estimate of free IL-13 levels in naive monkeys (i.e. below 0.0115 nM) for approximately 35 days post 10 mg/kg single IV administration of hMJ2-7v.2-11. Free IL-13 levels in Ascaris-challenged monkeys were predicted to rise above the naive baseline average when hMJ2-7v.2-11 concentration was approximately 160 nM. Along with the elimination of hMJ2-7v.2-11, free IL-13 levels in naive and Ascaris-challenged monkeys gradually rose to the corresponding baseline levels (defined by K_{syn}/CL_{IL-13}). The kinetics of IL-13 neutralization was also simulated with the different IV doses of hMJ2-7v.2-11 (1-50 mg/kg) in monkeys with a hypothetic established airway inflammation, i.e. assuming Ascaris challenge at Day 0. Predicted free IL-13 levels in naive monkeys and in monkeys with established airway inflammation after a single IV administration of hMJ2-7v.2-11 are shown in FIGS. 37A and 37B. In both naive monkeys and in monkeys with established airway inflammation, the time at which free IL-13 levels were below baseline IL-13 levels increased with hMJ2-7v.2-11 dosage used for the simulations. However, the extent and duration of IL-13 neutralization by hMJ2-7v.2-11 appeared to differ between the naïve monkeys and the monkeys with established airway inflammation. For example, after 10-mg/kg IV dosage of hMJ2-7v.2-11 to naive monkeys, most of IL-13 appeared to be hMJ2-7v.2-11-bound as late as Day 40 post-dose, with free IL-13 levels of <0.001 nM (or <7% of baseline). In contrast, after 10-mg/kg IV dosage of hMJ2-7v.2-11 to monkeys with established airway inflammation, there was an initial drop in free IL-13 to nearly-zero levels followed by a steady rise to approximately 0.008 nM or 21% of baseline at Day 40.

In this example, an integrated antibody-ligand binding PK-PD model was developed that described the relationship between the total serum concentrations of IL-13 and hMJ2-7v.2-11, an anti-IL-13 humanized IgG1 antibody, in naïve cynomolgus monkeys and in the disease model of acute airway inflammation induced by Ascaris challenge to cynomolgus monkeys. Due to lack of a bioanalytical method of sufficient sensitivity, free IL-13 levels could not be directly measured in either the presence or the absence of hMJ2-7v.2-11. Therefore, total IL-13 levels were used as a PD marker, as total IL-13 levels were transiently increased in both naive and Ascaris-challenged monkeys. The model presented in this report was developed under the assumption that hMJ2-7v.2-11 can bind either one or two IL-13 molecules, in a sequential manner. This assumption is based on the physiological mechanism of anti-IL-13/IL-13 interaction and is different from those used in the previously published integrated antibody-ligand binding PK-PD models for therapeutic antibodies, in which either 1:1 or 1:2 stoichiometry was assumed (Benincosa et al., J Pharmacol Exp Ther 2000; 292(2):810-6; Mager et al., J Pharmacokinet Pharmacodyn 2001; 28(6):507-32; Ng et al., Pharm Res 2006; 23(1):95-103; Hayashi et al., Br J Clin Pharmacol 2007; 63(5):548-61; Chow et al., Clin Pharmacol Ther 2002; 71(4):235-45).

The novel PK-PD model presented in this example described the data in both naive and Ascaris-challenge settings reasonable well and this model was used for analysis of the kinetics of neutralization of IL-13 by hMJ2-7v.2-11.

The hMJ2-7v.2-11 PK parameters estimated by the integrated PK/PD modeling were consistent with those estimated by non-compartmental analysis in Example 21b. hMJ2-7v.2-11 had a low clearance and a small volume of distribution in monkeys, typical of those seen for other humanized IgG1 therapeutic proteins (Adams et al., Cancer Immunol Immunother 2006; 55(6):717-27; Lin et al., J Pharmacol Exp Ther 1999; 288(1):371-8; Zia-Amirhosseini et al., J Pharmacol Exp Ther 1999; 291(3):1060-7). The integrated PK/PD modeling further confirmed that hMJ2-7v.2-11 volume of distribution was smaller in Ascaris-challenged monkeys, when compared to that in naive monkeys, in line with the results of non-compartmental analysis. Volume of distribution of hMJ2-7v.2-11 in the central (V) and, to some degree, the peripheral (V₂) compartments, as well as the distribution clearance (CL_{d,Ab}) of hMJ2-7v.2-11 between these two compartments were decreased in Ascaris-challenged monkeys when compared to those in naive monkeys. The difference of hMJ2-7v.2-11 volume of distribution between naive and Ascaris-challenged monkeys was unlikely due to the difference in hMJ2-7v.2-11 dosage used (1 or 2 mg/kg in naive monkey and 10 mg/kg in Ascaris-challenged monkeys), since the steady-state volume of distribution (Vdₛₛ) of hMJ2-7v.2-11 was similar among naive monkeys over a wide dose range (1-100 mg/kg) (Example 21b).

For both naive and Ascaris-challenged monkeys, the model also demonstrated that the transient increase in total IL-13 levels in Ascaris-challenged and naive animals was due to the increase in hMJ2-7v.2-11-bound IL-13, while free IL-13 was decreased. The neutralization of IL-13, leading to decrease in free IL-13 levels, is the intended biological effect of hMJ2-7v.2-11 and is consistent the observed efficacy of hMJ2-7v.2-11 in reducing airway inflammation in the Ascaris-challenged animals (Study 2), as well as with the lack of IL-13-mediated biological activity in the sera obtained from these animals.

Results of the PK-PD modeling and simulations indicated a number of differences in IL-13 neutralization between the naive and Ascaris-challenge settings. In the Ascaris-challenged animals, baseline IL-13 levels were estimated be approximately 3-fold higher, when compared to those in naive monkeys. This estimation was consistent with the notion that acute airway inflammation induced by Ascaris challenge in cynomolgus monkeys was mediated by IL-13. In human subjects, including normal human volunteers and subjects with a variety of disorders, there is a wide range of reported baseline IL-13 levels (from <10 pg/mL to >150 pg/mL), in part dependent on assay methodology employed for the measurements (Fiumara et al., Blood 2001; 98(9):2877-8; Wang et al., J Clin Virol 2006; 37(1):47-52). In general, baseline IL-13 levels in estimated for naive monkeys (approximately 100 pg/mL or approximately 0.01 nM) appeared to be higher, compared to those reported for healthy humans.

In Ascaris-challenged animals (Study 2), free circulating IL-13 levels were maintained below the average free IL-13 levels in naive monkeys for approximately one month after a 10 mg/kg IV administration of hMJ2-7v.2-11. Modeling indicated that for a given dose level of hMJ2-7v.2-11, extent and duration of hMJ2-7v.2-11-mediated IL-13 neutralization in the naïve- and Ascaris-challenged monkeys were different. Thus, caution should be used when applying PK-PD data from normal human volunteers to the design of clinical studies in subjects with airway inflammation.

It should be noted that the levels of free IL-13 in the target tissue (lung) may be a more direct indicator of effectiveness of IL-13 neutralization by a therapeutic protein. However, the level at which tissue (and circulating) IL-13 needs to be maintained to suppress Ascaris-induced airway inflammation in monkeys (and in asthmatic patients), as well as the required duration of the neutralization is not known. Total IL-13 levels were below the limit of detection in BAL (bronchoalveolar lavage) fluid of animals in Study 2 (data not shown), so that it was not possible to obtain a PD readout in the tissue compartment.

In summary, a novel PK-PD model was developed that described the relationship between the total serum concentrations of IL-13 and hMJ2-7v.2-11, in naive and Ascaris-challenged monkeys. The model prediction on IL-13 neutralization were the following: (1) The estimated circulating IL-13 levels were increased approximately 3-fold after the Ascaris-challenge, consistent with the notion that Ascaris-induced acute airway inflammation was IL-13-mediated; (2) the transient increase in total IL-13 levels observed in both naive and Ascaris-challenged monkeys, was due to the increase in hMJ2-7V.2-11-bound IL-13, while free IL-13 was decreased after IV administration of hMJ2-7V.2-11; and (3) when identical hMJ2-7v.2-11 dose regimens were used for simulations, the extent and duration of IL-13 neutralization in the circulation were different in naive and airway inflammation settings. However, this prediction needs to be interpreted with caution, as the model does not describe neutralization of IL-13 in the lung, the target organ. The PK-PD model presented in this Example can be applied to study drug-ligand interactions for other therapeutics proteins, in cases when free ligand (such as a cytokine or growth factor) cannot be readily assayed directly but total ligand levels change with drug administration. The differences in the ligand neutralization by a therapeutic protein between the healthy and pharmacology-model settings described in this report, illustrates the importance of conducting preclinical PK-PD studies in both settings, if practically feasible.

### Example 21b

### Pharmacokinetic and Pharmacodynamic Modeling of a Humanized Anti-IL-13 Antibody in Naive and Ascaris-Challenged Cynomolgus Monkeys (“Stoichiometric Model”)

Prior to conducting PK-PD modeling using the “sequential” integrated PK-PD model described in Example 21a, hMJ2-7v.2-11 PK-PD profile after 1 mg/kg IV administration of hMJ2-7v.2-11 to unchallenged monkeys (Table 8, Study 1), was analyzed using a “stoichiometrc” PK-PD model. The hMJ2-7v.2-11 PK concentration and total IL-13 concentration data-sets used for modeling was from Study 1, described in Table 8 and obtained using bioanalytical methods described in Example 21a.

The “stoichiometric” PK-PD model assumes two-to-one stoichiometry for the IL-13-hMJ2-7v.2-11 complex, i.e., one antibody molecule is bound to the two IL-13 molecules bound. The stoichiometric model is similar to previously published models in which either 1:1 or 2:1 stoichiometry was assumed. (Benincosa et al., J Pharmacol Exp Ther 2000; 292(2):810-6; Mager et al., J Pharmacokinet Pharmacodyn 2001; 28(6):507-32; Ng et al., Pharm Res 2006; 23(1):95-103; Hayashi et al., Br J Clin Pharmacol 2007; 63(5):548-61; Chow et al., Clin Pharmacol Ther 2002; 71(4):235-45).)

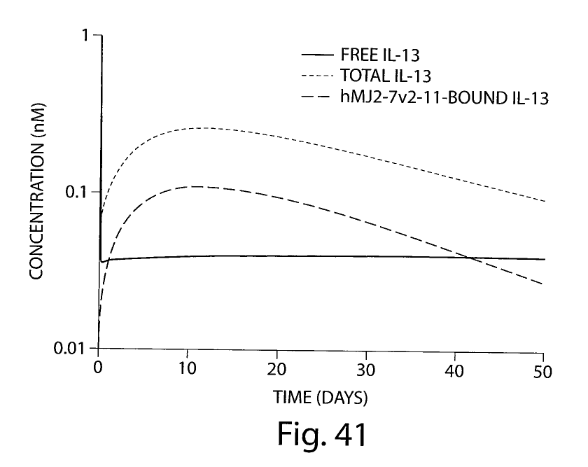

Specifically, an integrated “stoichiometric” pharmacokinetic and pharmacodynamic model that described the relationship between observed serum concentrations of hMJ2-7v.2-11 and total IL-13, was developed using WinNonlin software V 5.1.1 (Pharsight, Mountain View, Calif.) (FIG. 41). The pharmacokinetics of hMJ2-7v.2-11 was evaluated with a two-compartmental model including a central compartment (C_{Ab}, V) and a peripheral compartment (C_{2, Ab}, V₂). CL_{d,Ab} represented the distribution clearance between these two compartments. Clearance (CL_{Ab}) of hMJ2-7v.2-11 was assumed only through the central compartment. The pharmacodynamics of hMJ2-7v.2-11 was characterized with the neutralization of endogenous IL-13. Based on the bivalent femnature of IgG, the model assumed that each hMJ2-7v.2-11 molecule binds two IL-13 molecules simulatenousely with association (Kₒₙ) and disassociation (K_{off}) rate constants. Kₒₙ was a 3^{d} order rate constant governing the formation of hMJ2-7v.2-11/(IL-13)₂ (Ab-IL-13) complex and K_{off} was a 1^{st} order rate constant governing the disassociation of Ab-IL-13 complex. CL_{complex} represented the serum clearance of Ab-IL-13 complex. The homeostasis of IL-13 was assumed to be regulated by IL-13 production (zero order, K_{syn}) and degradation (CL_{I-13}). The following assumptions were also used (similar to that in Example 21a): V_{anti-IL-13}=V_{complex}=V_{IL-13}=V for model parsimony. The integrated PK/PD model was fitted to individual PK-PD data from 3 naive animals. The representative fit is shown in FIG. 32A.

The PK-PD parameters of hMJ2-7v.2-11 after 1 mg/kg IV administration to naive (unchallenged) cynomolgus monkeys, as derived from the “stoichiometric” PK-PD model are shown in Table 10.

TABLE 10
Mean Parameter Estimates from a Stoichiometric PK-PD Model of
Humanized MJ2-7v.2-11 and IL13 Disposition in Unchallenged
Cynomolgus Monkeys

| Parameters | Estimate | SD | CV % |
|---|---|---|---|
| CL_{Ab} (L day⁻¹) | 0.016 | 0.001 | 8.8 |
| V_{Ab} (L) | 0.196 | 0.026 | 13.1 |
| CLd_{Ab} (L day⁻¹) | 0.336 | 0.313 | 93.0 |
| V2_{Ab} (L) | 0.147 | 0.027 | 18.5 |
| K_{ON} (nM⁻² day⁻¹) | 0.202 | 0.157 | 77.8 |
| CL_{complex} (L day⁻¹) | 0.000 | 0.000 | 11.7 |
| K_{SYN} (nmol day⁻¹) | 0.097 | 0.025 | 26.1 |
| K_{deg} (L day⁻¹) | 2.405 | 1.028 | 42.8 |
| K_{OFF} (L day⁻¹) | 0.032 | 0.036 | 113.5 |

The mean model parameters described in Table 10 were used to simulate levels of free IL-13 and anti-IL-13-bound IL-13 using the WinNonlin software V 5.1.1 (Pharsight, Mountain View, Calif.).

In general, the results of the simulations of free and ant-IL-13 bound IL-13, after a single 1 mg/kg IV dosage to naïve monkeys, were similar for the “stoichiometric” (this example) and “sequential” models (Example 21a). As shown in FIG. 41, the “stoichiometric” model predicted a transient increase in total IL-13 following IV administration of 1 mg/kg of humanized MJ2-7v.2-11 to naïve monkeys. Following administration of anti-IL-13 antibody, the majority of IL-13 is in complex with humanized MJ2-7v.2-11 antibody. Thus, the results of stoichiometric model are consistent with those of sequential model (Example 21a) and suggest that the transient increase observed for total IL-13 due to increased levels of IL-13/anti-IL-13 antibody complex, while free IL-13 levels are decreased.

The stoichiometric model was also used to fit PK-PD from Ascaris-challenged monkeys (Study 2 in Table 8), obtain a set of PK-PD parameters and then simulate free, anti-IL-13-bound, and total IL-13 levels after 1 mg/kg IV dosage to Ascaris-challenged monkeys. The results of these simulations are shown in FIG. 41. Similar to simulations results for naïve monkeys, the “stoichiometirc” PK-PD model predicted that the transient increase observed for total IL-13 due to increased levels of IL-13/anti-IL-13 antibody complex, while free IL-13 levels are decreased (FIG. 42).

### Example 22

### Humanized 13.2v2 Antibody Effective in Allergen Challenge Study in Human Subjects

Study Design: Subjects with mild allergic asthma and dual airway responses to allergen challenge (AC) were randomized to receive two subcutaneous 2 mg/kg doses of a humanized anti-IL-13 antibody, 13.2v2, (n=14) or placebo (n=13) one week apart, in a multi-centre, double-blind, placebo controlled parallel-group study. AC was performed 2 weeks (Day 14) and 5 weeks (Day 35) after the first dose. Allergen-induced early (EAR) and late (LAR) asthmatic responses and airway hyperresponsiveness to methacholine were measured at each AC. Safety, tolerability and pharmacokinetics (PK) were evaluated throughout the study.

Results and Discussion: Humanized anti-IL-13 antibody, 13.2v2, was well tolerated, and was not associated with any serious adverse events, changes in blood hematology, chemistry, or vital signs. The frequency of adverse events was similar in the antibody 13.2v2 and placebo groups.

Human subjects with mild atopic asthma were selected for the study. Fourteen of the subjects were selected to receive anti-IL-13 antibody, and 13 subjects to receive placebo. The percent change in FEV1 for each subject was measured over 7 hours at various time points after allergen challenge. FEV1 (Forced Expiratory Volume in the first second) is the volume of air that can be forced out in one second after taking a deep breath, an important measure of lung function. A negative change in FEV1 indicates a decrease of lung function.

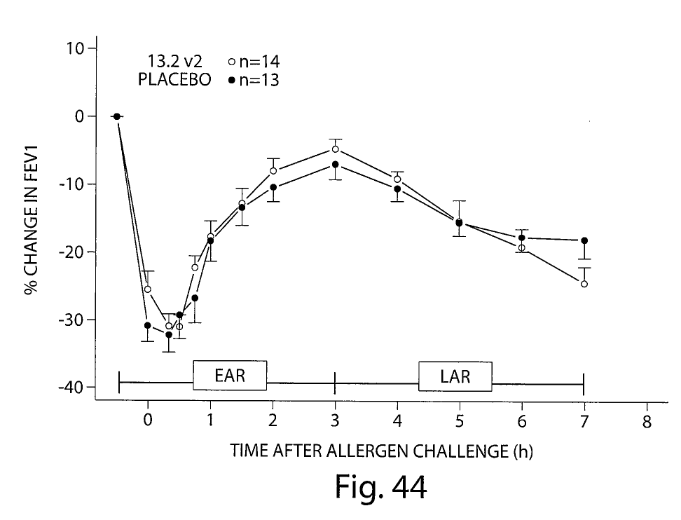

The subjects were challenged with allergen (Ag) on the screening visit (two weeks before the first administration of antibody). The allergen challenge was administered and the percent change in FEV1 was measured for each subject over 7 hours at various time points after allergen challenge. The results are shown in FIG. 44 as the mean (including standard error (STERR)) in FEV1 over time. Both groups of subjects responded similarly to the allergen challenge during the screening period.

Two weeks later, the subjects were administered 2 mg/kg of antibody (or a placebo control) subcutaneously. One week later, the subjects received another dose of 2 mg/kg of antibody (or a placebo control) subcutaneously.

Peak plasma concentrations were reached on ˜Day 14 of the study (two weeks after the initial dose of antibody was administered).

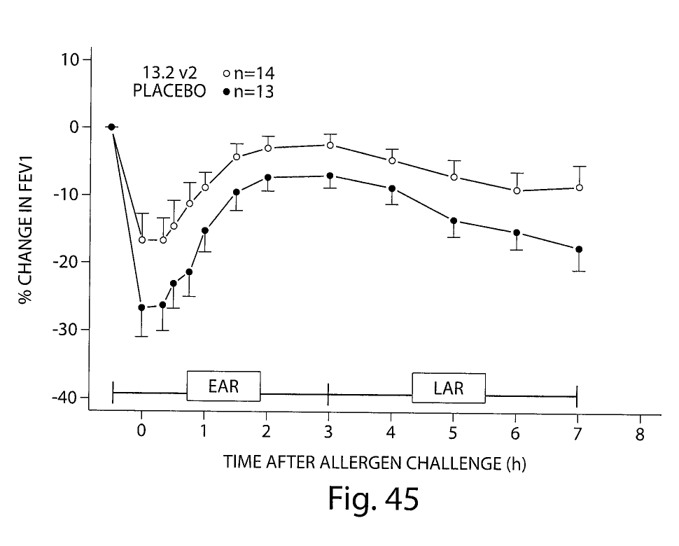

On Day 14, an allergen challenge was administered and the percent change in FEV1 was measured for each subject over 7 hours at various time points after allergen challenge. The results of the study are shown in FIG. 45 as the mean (including standard error (STERR)) in FEV1 over time. As indicated in the figure, subjects that received the 13.2v2 antibody had less of a percent change in FEV1 at all time points tested as compared to the placebo-treated control subjects. The differences in percent change in FEV1 were statistically significant for the early asthmatic response (EAR; 0-3 hours after challenge, p=0.042) and nearly reached significance for the late asthmatic response (LAR; 3-7 hours after challenge) time points (p=0.095). Also on Day 14, area under the curve (AUC) measurements were taken, and the area of the EAR and LAR were both significantly inhibited by the 13.2v2 antibody compared to placebo (EAR AUC₀₋₃ₕ: 46.3% inhibition versus placebo, p=0.030; LAR AUC₃₋₇ₕ: 49.0% inhibition versus placebo, p=0.039).

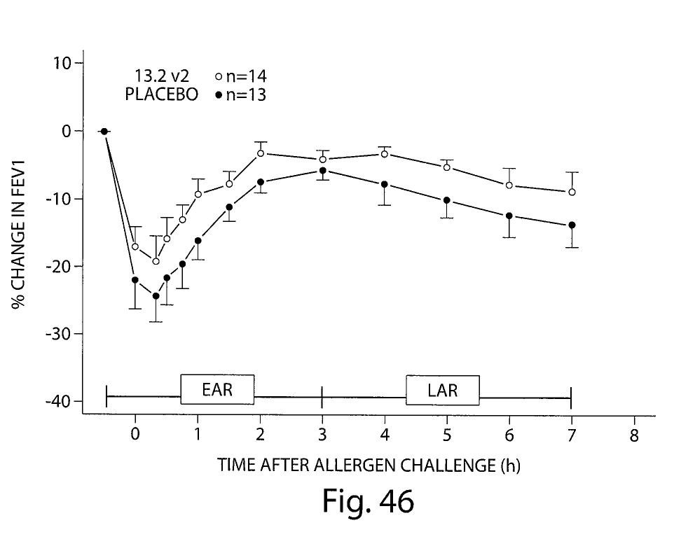

The percent change in FEV1 was also measured over 7 hours at various time points after allergen challenge on Day 35 (relative to the day of the first administration of antibody). The results of the study are shown in FIG. 46 as the mean (including standard error (STERR)) in FEV1over time. As indicated in the figure, subjects that received the antibody had less of a percent change in FEV1 at all time points tested as compared to the placebo-treated control subjects. The differences in percent change in FEV1 were seen at both the early asthmatic response (EAR; 0-3 hours after challenge) and late asthmatic response (LAR; 3-7 hours after challenge) time points, and continued the trend seen on Day 14. Also on Day 35, area under the curve (AUC) measurements were taken. There was a similar trend for inhibition of the area of the EAR and LAR at week 5 (Day 35), however this did not reach statistical significance (p=0.13 for both).

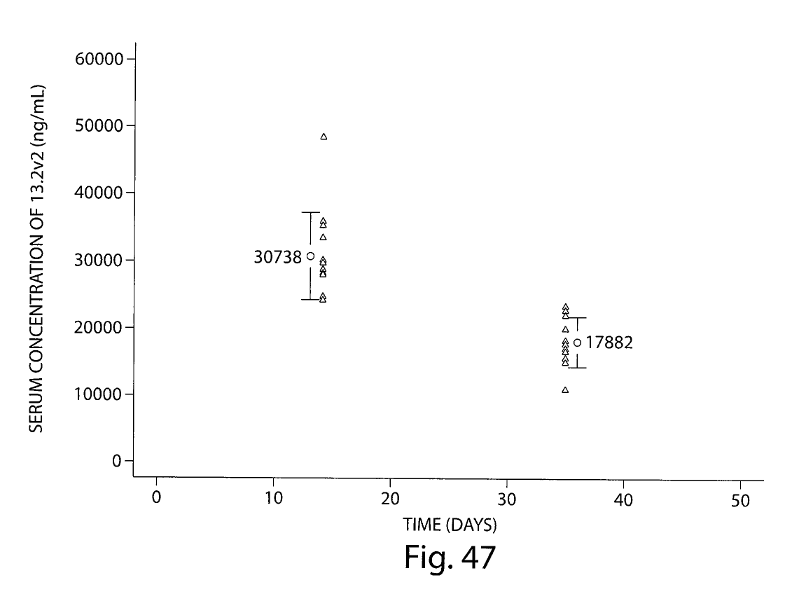

The serum concentration (ng/mL) of the 13.2v2 antibody on Day 14 and Day 35 are shown in FIG. 47.

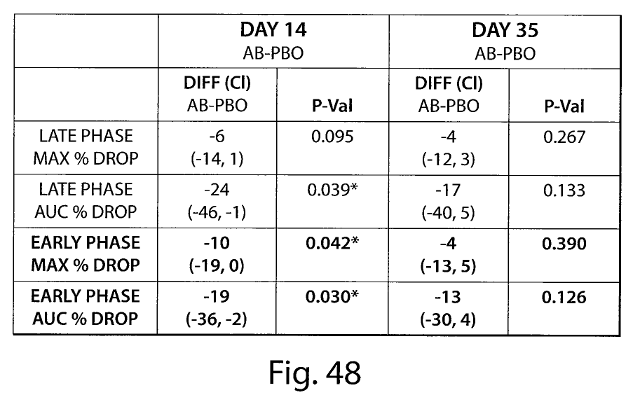

The results of repeated measures and statistical analysis for late phase (LAR) and early phase (EAR) maximum percent drop in FEV1 and AUC percent drop at Day 14 and Day 35 of this study are shown in FIG. 48. The differences (Diff) are shown as the value measured for the 13.2v2 antibody (AB) group minus the value measured for the placebo (PBO) group (AB-PBO). P values (P-Val) are also provided. Statistical significance is indicated by an asterisk (*). The statistical 95% confidence interval (CI) is also provided.

The ability of the antibody to affect allergen-induced hyperresponsiveness to methacholine was also measured at Days 14 and 35. No effect was seen on this parameter on either day.

Conclusions: Allergen-induced EAR and LAR at Day 14 were significantly inhibited by antibody 13.2v2, which also corresponded with peak plasma PK levels. These data demonstrate that IL-13 has a significant role in the early and late allergen-induced bronchoconstriction in humans.

### Example 23

### PK Profiles for Antibody 13.2v2 in Human Subjects

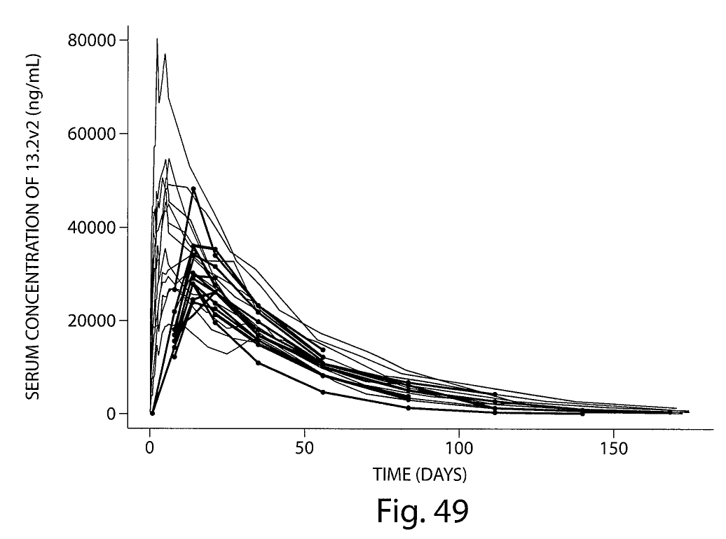

The PK profiles of 13.2v2 in human subjects were determined. Serum antibody concentration (ng/ml) was measured over a time course (days). The antibody was administered subcutaneously as a single ascending dose (SAD) of 4 mg/kg, or as two 2 mg/kg doses that were administered a week apart for the allergen challenge (AC) study. The results are shown in FIG. 49.

The half life of the antibody is approximately 23-29 days.

### Example 23

### Antibody 13.2v2 Pharmacokinetics and Product Metabolism in Humans

Pharmacokinetic data were obtained for non-Asian patients with mild asthma in SAD study A; and for healthy Japanese and non-Asian volunteers in SAD study B. Except for an additional IV cohort of 3 mg/kg dose in study A, both SAD studies were of similar design with 4 SC cohorts of 13.2v2 doses of 0.3, 1, 2, and 4 mg/kg. The mean (SD) serum concentration-time profiles of 13.2v2 in mild asthmatic non-Asian patients in study A and non-Asian volunteers in study B were determined. The pharmacokinetic profiles of 13.2v2 were consistent and parallel from 0.3 mg/kg to 4 mg/kg in both studies.

Non-Compartment Analysis of Serum 13.2v2 Data in Japanese and Foreign Subjects:

The serum 13.2v2 concentration time data in both study A and study B were analyzed using model independent noncompartment methods. The summary statistics on noncompartmental pharmacokinetic parameters of 13.2v2 are presented in Table 11 for study A and Table 12 for study B.

TABLE 11
Summary statistics of PK parameters in study A

| | | Cmax | Tmax | AUClast | AUCINF | Terminal | Vz_F | Cl_F |
|---|---|---|---|---|---|---|---|---|
| REGIMEN | | (ug/mL) | (Day) | (ug * hr/mL) | (ug * hr/mL) | T½ (Day) | (L) | (L/hr) |
| 0.3 mg/kg SC | NObs | 7 | 7 | 7 | 7 | 7 | 7 | 7 |
| | Mean | 2.962 | 4.290 | 2744.179 | 3079.906 | 25.473 | 6.613 | 0.00783 |
| | SD | 0.706 | 1.782 | 725.566 | 950.083 | 6.647 | 1.150 | 0.00179 |
| | Min | 2.19 | 2.47 | 1407.76 | 1443.88 | 14.91 | 5.62 | 0.00598 |
| | Median | 2.71 | 4.43 | 2984.78 | 3025.30 | 27.41 | 5.98 | 0.00797 |
| | Max | 3.85 | 6.08 | 3712.43 | 4637.12 | 33.22 | 8.18 | 0.01089 |
| 1 mg/kg SC | NObs | 8 | 8 | 8 | 8 | 8 | 8 | 8 |
| | Mean | 9.494 | 9.157 | 10601.482 | 11286.634 | 27.550 | 7.268 | 0.00773 |
| | SD | 2.679 | 8.250 | 2778.987 | 3047.617 | 4.642 | 1.546 | 0.00167 |
| | Min | 6.66 | 2.00 | 7199.59 | 7317.11 | 21.24 | 4.39 | 0.00421 |
| | Median | 8.92 | 5.95 | 9935.09 | 10445.92 | 26.95 | 8.00 | 0.00807 |
| | Max | 13.88 | 27.28 | 16604.72 | 17038.97 | 36.10 | 8.45 | 0.00932 |
| 2 mg/kg SC | NObs | 7 | 7 | 7 | 7 | 7 | 7 | 7 |
| | Mean | 23.144 | 5.985 | 26552.669 | 27105.914 | 29.014 | 6.541 | 0.00664 |
| | SD | 6.848 | 3.535 | 9100.027 | 9490.247 | 4.015 | 1.626 | 0.00190 |
| | Min | 17.20 | 2.42 | 17664.64 | 17829.23 | 24.25 | 4.07 | 0.00362 |
| | Median | 20.48 | 5.98 | 23940.64 | 24202.41 | 27.84 | 6.72 | 0.00599 |
| | Max | 34.90 | 13.06 | 45009.15 | 46251.10 | 35.97 | 9.09 | 0.00894 |
| 3 mg/kg IV | NObs | 8 | 8 | 8 | 8 | 8 | 8 | 8 |
| | Mean | 103.551 | 0.151 | 44420.523 | 44985.712 | 24.757 | 5.197 | 0.00621 |
| | SD | 22.486 | 0.188 | 11509.118 | 11959.224 | 4.040 | 1.414 | 0.00204 |
| | Min | 77.50 | 0.01 | 28144.17 | 28176.71 | 17.30 | 3.51 | 0.00434 |
| | Median | 99.80 | 0.07 | 44243.83 | 44849.68 | 25.48 | 5.09 | 0.00551 |
| | Max | 150.14 | 0.51 | 63902.13 | 65307.96 | 29.23 | 8.16 | 0.01029 |
| 4 mg/kg SC | NObs | 7 | 7 | 7 | 7 | 7 | 7 | 7 |
| | Mean | 52.399 | 5.759 | 48679.068 | 49617.936 | 26.110 | 6.195 | 0.00697 |
| | SD | 14.869 | 3.416 | 10865.726 | 11590.949 | 3.435 | 1.810 | 0.00217 |
| | Min | 30.73 | 2.34 | 36616.59 | 37058.24 | 21.37 | 4.68 | 0.00442 |
| | Median | 50.80 | 4.93 | 46964.71 | 47249.23 | 26.63 | 5.29 | 0.00650 |
| | Max | 80.45 | 13.05 | 64245.18 | 66774.68 | 30.57 | 9.45 | 0.01025 |

TABLE 12
Summary statistics of PK parameters in study B

| | | Cmax | Tmax | AUClast | AUCINF | Terminal | Vz_F | Cl_F |
|---|---|---|---|---|---|---|---|---|
| REGIMEN | | (ug/mL) | (Day) | (ug * hr/mL) | (ug * hr/mL) | T½ (Day) | (L) | (L/hr) |
| 0.3 mg/kg SC | NObs | 8 | 8 | 8 | 8 | 8 | 8 | 8 |
| Japanese | Mean | 3.119 | 6.627 | 2873.661 | 2933.902 | 24.578 | 6.235 | 0.00766 |
| | SD | 1.279 | 4.071 | 1009.677 | 1022.128 | 4.738 | 2.792 | 0.00408 |
| | Min | 0.97 | 3.00 | 1104.57 | 1138.54 | 18.88 | 3.45 | 0.00439 |
| | Median | 2.98 | 5.00 | 3032.36 | 3078.17 | 23.28 | 5.19 | 0.00593 |
| | Max | 5.62 | 13.06 | 4082.37 | 4133.51 | 34.49 | 12.53 | 0.01660 |
| Non-Japanese | NObs | 5 | 5 | 5 | 5 | 5 | 5 | 5 |
| | Mean | 3.379 | 5.617 | 3211.327 | 3256.416 | 24.029 | 6.437 | 0.00785 |
| | SD | 0.413 | 0.553 | 634.790 | 645.507 | 2.033 | 1.045 | 0.00193 |
| | Min | 2.95 | 5.00 | 2254.42 | 2284.98 | 20.63 | 5.02 | 0.00569 |
| | Median | 3.36 | 6.00 | 3380.78 | 3432.64 | 24.79 | 6.68 | 0.00756 |
| | Max | 3.83 | 6.04 | 3914.05 | 3972.80 | 25.52 | 7.84 | 0.01098 |
| 1 mg/kg SC | NObs | 7 | 7 | 7 | 7 | 7 | 7 | 7 |
| Japanese | Mean | 11.654 | 5.257 | 9698.563 | 10246.867 | 25.448 | 6.048 | 0.00679 |
| | SD | 3.463 | 3.624 | 1757.734 | 2055.584 | 2.430 | 1.969 | 0.00175 |
| | Min | 5.99 | 2.00 | 7512.15 | 7751.11 | 22.02 | 3.88 | 0.00489 |
| | Median | 11.92 | 3.96 | 9913.16 | 10563.57 | 25.65 | 5.32 | 0.00677 |
| | Max | 16.65 | 12.94 | 11923.06 | 12625.13 | 28.52 | 9.43 | 0.00955 |
| Non-Japanese | NObs | 6 | 6 | 6 | 6 | 6 | 6 | 6 |
| | Mean | 12.379 | 5.500 | 11188.010 | 11482.734 | 25.754 | 6.097 | 0.00691 |
| | SD | 2.084 | 4.059 | 1733.946 | 1832.206 | 5.624 | 1.320 | 0.00110 |
| | Min | 8.54 | 2.00 | 9561.01 | 9904.13 | 19.43 | 4.84 | 0.00553 |
| | Median | 12.80 | 4.96 | 11013.06 | 11298.43 | 23.79 | 5.70 | 0.00678 |
| | Max | 14.44 | 13.05 | 14411.62 | 14897.91 | 34.44 | 8.38 | 0.00880 |
| 2 mg/kg SC | NObs | 7 | 7 | 7 | 7 | 7 | 7 | 7 |
| Japanese | Mean | 20.144 | 3.856 | 18116.575 | 18344.994 | 24.858 | 6.086 | 0.00725 |
| | SD | 3.966 | 1.472 | 3786.057 | 3926.702 | 3.643 | 0.977 | 0.00186 |
| | Min | 15.88 | 2.00 | 13941.93 | 14000.69 | 20.15 | 5.00 | 0.00539 |
| | Median | 17.83 | 4.00 | 16655.30 | 16863.10 | 25.22 | 5.80 | 0.00687 |
| | Max | 26.67 | 6.06 | 23495.70 | 23840.76 | 29.80 | 7.46 | 0.01066 |
| Non-Japanese | NObs | 5 | 5 | 5 | 5 | 5 | 5 | 5 |
| | Mean | 34.530 | 4.420 | 24798.285 | 25067.707 | 23.373 | 4.798 | 0.00590 |
| | SD | 9.900 | 1.534 | 3388.686 | 3394.688 | 2.581 | 0.834 | 0.00046 |
| | Min | 22.21 | 3.00 | 21735.42 | 21880.10 | 20.58 | 3.73 | 0.00523 |
| | Median | 37.34 | 4.03 | 23290.71 | 23597.80 | 23.56 | 4.68 | 0.00589 |
| | Max | 46.73 | 6.04 | 30354.34 | 30646.46 | 27.22 | 5.88 | 0.00640 |
| 4 mg/kg SC | NObs | 6 | 6 | 6 | 6 | 6 | 6 | 6 |
| Japanese | Mean | 31.495 | 7.094 | 35990.823 | 36770.423 | 26.492 | 8.016 | 0.00897 |
| | SD | 8.777 | 2.937 | 8573.982 | 8846.049 | 2.938 | 1.753 | 0.00282 |
| | Min | 19.02 | 4.97 | 27405.02 | 28128.08 | 21.92 | 5.42 | 0.00515 |
| | Median | 31.52 | 6.09 | 35366.17 | 36121.76 | 26.16 | 8.40 | 0.00895 |
| | Max | 44.94 | 13.01 | 50408.70 | 51707.05 | 30.39 | 10.29 | 0.01355 |

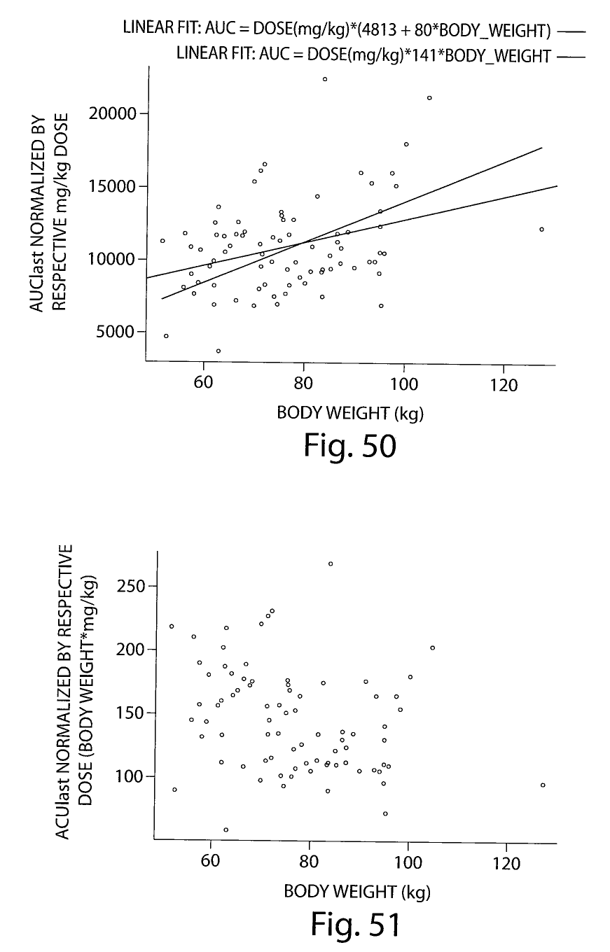

Since body weight normalized 13.2v2 dosing was employed for both studies, subject with larger body weight received a larger dose of 13.2v2. The effect of body weight on 13.2v2 exposures was graphically assessed in FIGS. 50 and 51.

In FIG. 50, AUC exposure normalized by respective mg/kg dose in all 81 subjects in both studies appeared to be positively correlated to body weight, suggesting the difference in exposure is related to body weight difference.

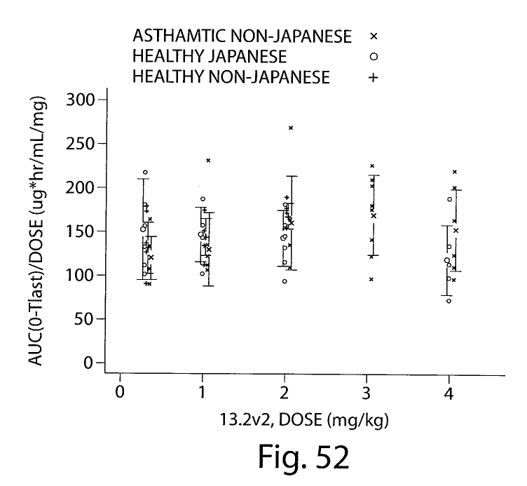

In FIG. 51, exposure normalized by actual doses appeared to be consistent across all doses in all 81 subjects, suggesting that body weight is not a significant factor affecting 13.2v2 exposure. Furthermore, when exposure normalized by actual doses were compared in mild asthmatic US subjects and healthy Japanese and US subjects in FIG. 52, the 13.2v2 AUC per unit of 13.2v2 dose were independent of mg/kg dose and consistent between study A and B. This suggests that 13.2v2 exposure increases approximately with the dose increment, and neither ethnicity nor presence of mild asthma remarkably affects 13.2v2 exposure. In addition, the 13.2v2 AUC per unit of 13.2v2 SC dose is close to the 13.2v2 AUC per unit of IV dose suggest that close to complete systemic absorption of 13.2v2 following SC administration.

### Population Pharmacokinetic Analyses of 13.2v2 Exposure Data in Japanese and Foreign Subjects:

In addition to the non-compartmental analysis, serum 13.2v2 concentration data in both study A and B were combined and analyzed using population pharmacokinetic methodology based on nonlinear mixed effect pharmacostatistical model implemented in NONMEM software package. While the PK exposure and parameters derived from distinct dose levels by non-compartmental analysis are based on a small number of subjects (5-8), the point estimate of the mean and variability is expected to vary from dose to dose and prone to chance findings. In comparison, the population analyses took advantage of the mixed effect model methodology, provides a systemic framework to examine 13.2v2 exposure and potential important covariate across all dose in both Japanese and Non-Asian populations. The population method is more sensitive than non-compartment method to detect significant covariate.

The population PK analyses employed NONMEM PREDD library routine ADVAN3 with TRANS3 in NONMEM version VI. The first order conditional estimation method with η-ε was used throughout the model building and covariate analysis process. The analysis identified an optimal base population PK model consisting of a two-compartmental structure PK model component and combined proportional and additive error model components. Covariate analyses were performed based on the base population PK model. Body weight, body surface area, ethnicity and presence of mild asthma/health status were evaluated as potential covariates, and none of these factors was found to affect 13.2v2 serum exposure in a statistically significant manner. The base and optimal population PK model parameters are listed in Table 13.

TABLE 13
Population PK parameters of 13.2v2 based
on the base and optimal model

| | | Typical Value ± |
|---|---|---|
| Parameter | Units | SE |
| CL | L/h | 0.0058 ± 0.00056 |
| V₁ | L | 2.82 ± 0.30 |
| Q | L/h | 0.0239 ± 0.0028 |
| V₂ | L | 2.00 ± 0.22 |
| F₁ | — | 0.805 ± 0.081 |
| Variance on CL | — | 0.076 ± 0.012 |
| Variance on V₁ | — | 0.146 ± 0.031 |
| Variance on Q | — | 0.345 ± 0.061 |
| Variance on V₂ | — | 0.084 ± 0.030 |
| Proportional error | — | 0.0238 ± 0.0030 |
| (Variance) | | |
| Additive error | ng/mL | 2390 ± 1240 |
| (Variance) | | |

Note: The population PK model was developed based on 13.2v2 exposure data from both study A and study B.

The final model adequately describes the serum 13.2v2 observations in both studies, as measured by Postier predictive checks of the base and optimal population PK model of 13.2v2. Furthermore, the PK parameters derived from the population analysis are consistent with those derived from the non-compartmental analyses.

Based on the optimal population PK model, a series of simulations were performed based on the optimal population PK model to compare 13.2v2 exposure and associated variability of 3 mg/kg dosing versus flat dosing of 225 mg (3 mg/kg in a 75 kg subject) in typical subjects with body weight of 50 kg, 75 kg and 130 kg, respectively. The 90% confidence interval of expected 13.2v2 exposure in these typical subjects was determined. Flat dosing produced consistent 13.2v2 exposure in these subjects of different body weight, while mg/kg dose resulted in higher 13.2v2 exposure in subjects with larger body weights, lower 13.2v2 exposure in subjects with lower body weights. When these subjects were pulled together, as expected in any clinical study enrolling subjects of various body weights, the mg/kg dosing resulted in larger variability than flat dosing.

Summary of Pharmacokinetic Findings in Study A and Study B:

13.2v2 exposure increases with dose increment from 0.3 mg to 4 mg/kg in both asthmatic US subjects and healthy Japanese and US subjects;

Ethnicity dose not affect 13.2v2 pharmacokinetics, 13.2v2 exposure in Japanese subjects was similar to that in non-Asian subjects receiving identical doses;

Body weight does not affect 13.2v2 pharmacokinetics, as a result, flat dosing is better than mg/kg dosing and results in less exposure variability;

Being healthy or having mild asthma does not affect 13.2v2 pharmacokinetics, the 13.2v2 exposure in healthy Japanese and non-Asian are similar to that in asthmatic US patients.

Those skilled in the art will recognize, or be able to ascertain using no more than routine experimentation, many equivalents of the specific embodiments described herein described herein. Other embodiments are within the following claims.

What is claimed is:

1. A method of evaluating an anti-IL13 antibody molecule, comprising:
providing a mean test value for at least one pharmacokinetic/pharmacodynamic (PK/PD) parameter of the anti-IL13 antibody molecule in a subject; and
comparing the mean test value provided with at least one mean reference value,
thereby evaluating the anti-IL13 antibody molecule,
wherein the mean reference value is selected from the group consisting of:
a mean CL value in the range of about 0.05 to 0.9 mL/hr/kg after intravenous administration of the anti-IL13 antibody molecule to the subject; a mean V_{dss} value of less than about 150 mL/kg after intravenous administration to the subject; a mean half-life (t_{1/2}) of about 500 to 800 hours after intravenous administration in a human; a dose normalized mean maximum serum or plasma concentration of about 2 to 40 μg/ml after intravenous administration to the subject, or about 0.1 to 30 μg/ml after subcutaneous administration to the subject; a mean dose normalized exposure of about 800 to 18,000 (μghr/mL)/(mg/kg) after intravenous administration to the subject, or 400 to 18000 (μghr/mL)/(mg/kg) after subcutaneous administration to the subject; a bioavailability of about 60 to 90% after subcutaneous administration to the subject; and a tissue-to-serum ratio of less than about 0.5, wherein the anti-IL13 antibody molecule comprises a a full-length antibody;
a mean half-life (t_{1/2}) of about 0.5 to 30 hours after subcutaneous or intravenous administration, to the subject, wherein the anti-IL-13 antibody molecule comprises an antigen-binding site of the antibody molecule; and
a mean clearance rate of less than 0.004 mL/hr/kg after administration to the subject, wherein the anti-IL-13 antibody molecule is complexed to IL-13.

2. A method of determining a treatment modality of an anti-IL13 antibody molecule for an IL-13-mediated disorder, in a subject, comprising:
providing a mean test value for at least one PK/PD parameter of the anti-IL13 antibody molecule in a subject;
comparing the mean test value provided with at least one mean reference value; and
selecting one or more of dosage, timing, or mode of administration based on the comparison of at least one mean test value to the mean reference value, wherein the mean reference value is selected from the group consisting of:
a mean CL value in the range of about 0.05 to 0.9 mL/hr/kg after intravenous administration of the anti-IL13 antibody molecule to the subject; a mean V_{dss} value of less than about 150 mL/kg after intravenous administration to the subject; a mean half-life (t_{1/2}) of about 500 to 800 hours after intravenous administration in a human; a dose normalized mean maximum serum or plasma concentration of about 2 to 40 μg/ml after intravenous administration to the subject, or about 0.1 to 30 μg/ml after subcutaneous administration to the subject; a mean dose normalized exposure of about 800 to 18,000 (μghr/mL)/(mg/kg) after intravenous administration to the subject, or 400 to 18000 (μghr/mL)/(mg/kg) after subcutaneous administration to the subject; a bioavailability of about 60 to 90% after subcutaneous administration to the subject; and a tissue-to-serum ratio of less than about 0.5, wherein the anti-IL13 antibody molecule comprises a full-length antibody;
a mean half-life (t_{1/2}) of about 0.5 to 30 hours after subcutaneous or intravenous administration, to the subject, wherein the anti-IL-13 antibody molecule comprises an antigen-binding site of the antibody molecule; and
a mean clearance rate of less than 0.004 mL/hr/kg after administration to the subject, wherein the anti-IL-13 antibody molecule is complexed to IL-13.

3. The method of claim 1 or 2, wherein the mean reference value comprises a mean serum clearance (CL) value in the range of about 0.065 to 0.3 mL/hr/kg after intravenous administration to the subject.

4. The method of claim 1 or 2, wherein the mean reference value comprises a mean steady state volume of distribution (V_{dss}) value of less than about 110 mL/kg after intravenous administration to the subject.

5. The method of claim 1 or 2, wherein the mean reference value comprises a mean half-life (t_{1/2}) of about 670 to 725.

6. The method of claim 1 or 2, wherein the mean test value, or an indication of whether the preselected relationship is met, is memorialized.

7. The method of claim 1, wherein the step of providing a mean test value comprises obtaining a sample of the antibody molecule and testing at least one of said PK/PD parameters.

8. The method of claim 1 or 2, wherein the subject is a rodent or a primate.

9. The method of claim 1 or 2, wherein the subject is a human.

10. The method of claim 9, wherein the human has a body weight of about 50-80 kg.

11. A method of treating an IL-13-associated disorder in a subject, comprising:
administering, to a subject having, or being at risk of having, the IL-13-associated disorder, an effective amount of an anti-IL-13 antibody molecule evaluated by the method of claim 1.

12. A method of treating an IL-13-associated disorder in a subject, comprising:
administering, to a subject having, or being at risk of having, the IL-13-associated disorder, an anti-IL-13 antibody molecule at a dosage, timing or mode of administration determined by the method of claim 2.

13. The method of claim 12 or 13, wherein the IL-13 associated disorder is selected from the group consisting of: asthmatic disorders, atopic disorders, chronic obstructive pulmonary disease (COPD), conditions involving airway inflammation, eosinophilia, fibrosis and excess mucus production, inflammatory conditions, autoimmune conditions, tumors or cancers, viral infection, and suppression of expression of protective type 1 immune responses.

14. A method of instructing a recipient on the use of an anti-IL13 antibody molecule to treat an IL-13-associated disorder, comprising:
instructing the recipient that the anti-IL13 antibody molecule has at least one mean test value for a PK/PD parameter selected from the group consisting of:
wherein the mean reference value is selected from the group consisting of:
a mean CL value in the range of about 0.05 to 0.9 mL/hr/kg after intravenous administration of the anti-IL13 antibody molecule to the subject; a mean V_{dss} value of less than about 150 mL/kg after intravenous administration to the subject; a mean half-life (t_{1/2}) of about 500 to 800 hours after intravenous administration in a human; a dose normalized mean maximum serum or plasma concentration of about 2 to 40 μg/ml after intravenous administration to the subject, or about 0.1 to 30 μg/ml after subcutaneous administration to the subject; a mean dose normalized exposure of about 800 to 18,000 (μghr/mL)/(mg/kg) after intravenous administration to the subject, or 400 to 18000 (μghr/mL)/(mg/kg) after subcutaneous administration to the subject; a bioavailability of about 60 to 90% after subcutaneous administration to the subject; and a tissue-to-serum ratio of less than about 0.5, wherein the anti-IL13 antibody molecule comprises a full-length antibody;
a mean half-life (t_{1/2}) of about 0.5 to 30 hours after subcutaneous or intravenous administration, to the subject, wherein the anti-IL-13 antibody molecule comprises an antigen-binding site of the antibody molecule; and
a mean clearance rate of less than 0.004 mL/hr/kg after administration to the subject, wherein the anti-IL-13 antibody molecule is complexed to IL-13.

15. The method of claim 14, wherein the recipient is a patient, a pharmacist, a caregiver, a clinician, a member of a medical staff, a manufacturer, or a distributor.

16. The method of claim 14, wherein the method further comprises recording or memorializing one of more of the test values of the antibody molecule.

17. A method of treating an IL-13-associated disorder in a subject having, or being at risk of having, the IL-13-associated disorder, comprising:
instructing a caregiver or a patient that an anti-IL13 antibody has at least one mean test value for a PK/PD parameter selected from the group consisting of:
wherein the mean reference value is selected from the group consisting of:
a mean CL value in the range of about 0.05 to 0.9 mL/hr/kg after intravenous administration of the anti-IL13 antibody molecule to the subject; a mean V_{dss} value of less than about 150 mL/kg after intravenous administration to the subject; a mean half-life (t_{1/2}) of about 500 to 800 hours after intravenous administration in a human; a dose normalized mean maximum serum or plasma concentration of about 2 to 40 μg/ml after intravenous administration to the subject, or about 0.1 to 30 μg/ml after subcutaneous administration to the subject; a mean dose normalized exposure of about 800 to 18,000 (μghr/mL)/(mg/kg) after intravenous administration to the subject, or 400 to 18000 (μghr/mL)/(mg/kg) after subcutaneous administration to the subject; a bioavailability of about 60 to 90% after subcutaneous administration to the subject; and a tissue-to-serum ratio of less than about 0.5, wherein the anti-IL13 antibody molecule comprises a full-length antibody;
a mean half-life (t_{1/2}) of about 0.5 to 30 hours after subcutaneous or intravenous administration, to the subject, wherein the anti-IL-13 antibody molecule comprises an antigen-binding site of the antibody molecule; and
a mean clearance rate of less than 0.004 mL/hr/kg after administration to the subject, wherein the anti-IL-13 antibody molecule is complexed to IL-13.

18. The method of any of claims 1, 2, 11, 12, 14 or 17, wherein the anti-IL-13 antibody molecule comprises a heavy chain immunoglobulin variable domain sequence and a light chain immunoglobulin variable domain sequence that form an antigen binding site that binds to IL-13 with a K_{D} of less than 10⁻⁷ M, wherein the antibody molecule has one or more of the following properties:
(a) the heavy chain immunoglobulin variable domain sequence comprises a heavy chain CDR3 that differs by fewer than 3 amino acid substitutions from a heavy chain CDR3 of mAb MJ2-7;
(b) the light chain immunoglobulin variable domain sequence comprises a light chain CDR that differs by fewer than 3 amino acid substitutions from a corresponding light chain CDR of mAb MJ2-7;
(c) the heavy chain immunoglobulin variable domain sequence comprises a sequence encoded by a nucleic acid that hybridizes under high stringency conditions to the complement of a nucleic acid encoding a heavy chain variable domain of V2.1, V2.3, V2.4, V2.5, V2.6, V2.7, or V2.11;
(d) the light chain immunoglobulin variable domain sequence comprises a sequence encoded by a nucleic acid that hybridizes under high stringency conditions to the complement of a nucleic acid encoding a light chain variable domain of V2.11;
(e) the heavy chain immunoglobulin variable domain sequence is at least 90% identical a heavy chain variable domain of V2.1, V2.3, V2.4, V2.5, V2.6, V2.7, or V2.11;
(f) the light chain immunoglobulin variable domain sequence is at least 90% identical a light chain variable domain of V2.11;
(g) the antibody molecule competes with mAb MJ2-7 for binding to human IL-13;
(h) the antibody molecule contacts one or more amino acid residues from IL-13 selected from the group consisting of residues 116, 117, 118, 122, 123, 124, 125, 126, 127, and 128 of SEQ ID NO:24 or SEQ ID NO:178;
(i) the heavy chain variable domain sequence has the same canonical structure as mAb MJ2-7 in hypervariable loops 1, 2 and/or 3;
(j) the light chain variable domain sequence has the same canonical structure as mAb MJ2-7 in hypervariable loops 1, 2 and/or 3; and
(k) the heavy chain variable domain sequence and/or the light chain variable domain sequence has FR1, FR2, and FR3 framework regions from VH segments encoded by germline genes DP-54 and DPK-9 respectively or a sequence at least 95% identical to VH segments encoded by germline genes DP-54 and DPK-9.

19. The method of claim 18, wherein the anti-IL-13 antibody molecule is a full length antibody or a fragment thereof.

20. The method of claim 18, wherein the anti-IL-13 antibody molecule reduces the ability of IL-13 to bind to IL-13RI1 or IL-13RI2.

21. The method of claim 18, wherein the anti-IL-13 antibody molecule comprises a heavy chain variable domain sequence having a sequence:
(SEQ ID NO:48)

| | |
|---|---|
| (i) | G-(YF)-(NT)-I-K-D-T-Y-(MI)-H, in CDR1, |

(SEQ ID NO:49)

| | |
|---|---|
| (ii) | (WR)-I-D-P-(GA)-N-D-N-I-K-Y-(SD)-(PQ)-K-F- |
| | Q-G, in CDR2, |
| and | |

(SEQ ID NO:17)

| | |
|---|---|
| (iii) | SEENWYDFFDY, in CDR3; |
| and | |

a light chain variable domain sequence having the sequence:
(SEQ ID NO:25)

| | |
|---|---|
| (i) | (RK)-S-S-Q-S-(LI)-(KV)-H-S-(ND)-G-N-(TN)-Y- |
| | L-(EDNQYAS), in CDR1, |

(SEQ ID NO:27)

| | |
|---|---|
| (ii) | K-(LVI)-S-(NY)-(RW)-(FD)-S, in CDR2, |
| and | |

(SEQ ID NO:28)

| | |
|---|---|
| (iii) | Q-(GSA)-(ST)-(HEQ)-I-P, in CDR3. |

22. The method of claim 18, wherein the anti-IL-13 antibody molecule comprises a heavy chain variable domain sequence having a sequence:

| | | |
|---|---|---|
| GFNIKDTYIH, | (SEQ ID NO:15) | in CDR1, |
| RIDPANDNIKYDPKFQG, | (SEQ ID NO:16) | in CDR2, |
| and | | |
| SEENWYDFFDY, | (SEQ ID NO:17) | in CDR3; |
| and | | |

a light chain variable domain sequence having the sequence:

| | | |
|---|---|---|
| RSSQSIVHSNGNTYLE, | (SEQ ID NO:18) | in CDR1 |
| KVSNRFS, | (SEQ ID NO:19) | in CDR2, |
| and | | |
| FQGSHIPYT, | (SEQ ID NO:20) | in CDR3. |

23. A method of evaluating the amount of a drug-ligand complex in a subject using a two-compartmental model that includes a central compartment (C_{Ab}, V) and a peripheral compartment (C_{2,Ab}, V₂), said method comprising:
providing at least one pharmacokinetic parameter value of the drug-ligand concentration in the subject at a predetermined time interval, said value chosen from one or more of: a clearance of the drug from the central compartment (CL_{Ab}); a distribution clearance between the central compartment and the peripheral compartment (CL_{d,Ab}); an association rate constant (Kₒₙ); a dissociation rate constant (K_{off}); a serum clearance of the drug-ligand complex (CL_{complex}); or an endogenous rate constant for ligand production divided by a serum clearance of the ligand (Ksyn/CL_{IL-13});
evaluating the at least one pharmacokinetic parameter in the subject using the two-compartmental model as represented in FIG. 39.

24. The method of claim 23, wherein the two-compartmental model is represented as follows:
dC_{Ab}/dt=[In(t)+CL_{d,Ab}·C_{2,Ab}−(CL_{d,Ab}+CL_{Ab})·C_{Ab}]/V−Kₒₙ·C_{Ab}*(C_{IL-13}−C_{Ab-(IL-13)}−C_{Ab-(IL-13)}_{₂})+K_{off}·C_{Ab-(IL-13)} when t=0,C_{Ab}⁰=In(0)/V (1)
dC_{2,Ab}/dt=(CL_{d,Ab}·C_{Ab}−CL_{d,Ab}·C_{2,Ab})/V₂ when t=0,C_{2,Ab}⁰=0 (2)
dC_{Ab-(IL-13)}/dt=Kₒₙ·C_{Ab}·(C_{IL-13}−C_{Ab-(IL-13)}−C_{Ab-(IL-13)}_{₂})−CL_{complex}·C_{Ab-(IL-13)}−K_{off}·C_{Ab-(IL-13)}+K_{off}·C_{Ab-(IL-13)}_{₂}−Kₒₙ·C_{Ab-(IL-13)}·(C_{IL-13}−C_{Ab-(IL-13)}−C_{Ab-(IL-13)}_{₂}) when t=0,C_{Ab-(IL-13)}⁰=0 (3)
dC_{Ab-(IL-13)}_{₂}/dt=Kₒₙ·C_{Ab-(IL-13)}·(C_{IL-13}−C_{Ab-(IL-13)}−C_{Ab-(IL-13)}_{₂})−CL_{complex}·C_{Ab-(IL-13})_{₂}−K_{off}·C_{Ab-(IL-3)}_{₂} when t=0,C_{Ab-(IL-13)}_{₂}⁰=0 (4)
dC_{IL-13}/dt=[K_{syn}−CL_{IL-13}·(C_{IL-13}−C_{Ab-(IL-13)}−C_{Ab-(IL-13)}_{₂})]/V−Kₒₙ·C_{Ab}·(C_{IL-13}−C_{Ab-(IL-13)}−C_{Ab-(IL-13)}_{₂})−Kₒₙ·C_{Ab-(IL-13)}·(C_{IL-13}−C_{Ab-(IL-13)}−C_{Ab-(IL-13)}_{₂})+k_{off}·C_{A-(IL-13)}+K_{off}·C_{Ab-(IL-13)}_{₂} when t=0,C_{Il-13}⁰=K_{syn}/CL_{IL-13} (5)
For iv bolus dose:
In(t)=Dose (6)
For sc dose:
In(t)=Kₐ·F·Dose (7)
wherein,
C_{Ab} is a concentration of antibody (binding agent);
In(t) is a dose administered (for a bolus dose), and In(t) is Kₐ*F*Dose for a subcutaneous does, wherein Kₐ is a first order rate constant and F is an estimate of bioavailability;
CL_{d,Ab} is a distribution clearance between the central compartment and the peripheral compartment;
C_{2,Ab} is a concentration of the ligand binding agent in the peripheral compartment;
V is a volume distribution in a central component;
Kₒₙ is a second order rate constant;
C_{ligand} (or C_{IL-13}) is a concentration of ligand;
C_{Ab-(ligand)} (or C_{Ab-(IL-13)}) is a concentration of ligand binding agent/ligand complex;
K_{off} is a first order disassociation rate constant, V₂ is a volume of distribution in a peripheral compartment;
CL_{complex} is the serum clearance of the ligand binding agent/ligand complex; and
K_{syn} is a zero order rate constant for endogenous ligand.

25. The method of claim 23 or 24, wherein drug-ligand complex is a ligand-antibody complex or a ligand-soluble receptor complex.

26. A method of treating or preventing an early asthmatic response (EAR) in a subject, the method comprising administering, to a subject having, or being at risk of having, an EAR, an anti-IL-13 antibody molecule.

27. The method of claim 26, wherein the anti-IL-13 antibody molecule decreases or prevents one or more one or more of: a release of at least one allergic mediator such as a leukotriene and/or histamine; an increase in the levels of at least one allergic mediator such as a leukotriene and/or histamine; bronchoconstriction; and/or airway edema.

28. A method of treating or preventing an early asthmatic response (EAR) in a subject, the method comprising:
administering, to a subject having, or being at risk of having, an EAR, an anti-IL-13 antibody molecule at a dosage, timing or mode of administration determined by the method of claim 2.

29. A method of treating or preventing a late asthmatic response (LAR) in a subject, the method comprising administering, to a subject having, or being at risk of having, an LAR, an anti-IL-13 antibody molecule.

30. A method of treating or preventing a late asthmatic response (LAR) in a subject, the method comprising:
administering, to a subject having, or being at risk of having, an LAR, an anti-IL-13 antibody molecule at a dosage, timing or mode of administration determined by the method of claim 2.

31. The method of any of claims 26 to 30, wherein the anti-IL-13 antibody molecule comprises a heavy chain immunoglobulin variable domain sequence and a light chain immunoglobulin variable domain sequence that form an antigen binding site that binds to IL-13 with a K_{D} of less than 10⁻⁷ M, wherein the antibody molecule has one or more of the following properties:
(a) the heavy chain immunoglobulin variable domain sequence comprises a heavy chain CDR3 that differs by fewer than 3 amino acid substitutions from a heavy chain CDR3 of mAb MJ2-7;
(b) the light chain immunoglobulin variable domain sequence comprises a light chain CDR that differs by fewer than 3 amino acid substitutions from a corresponding light chain CDR of mAb MJ2-7;
(c) the heavy chain immunoglobulin variable domain sequence comprises a sequence encoded by a nucleic acid that hybridizes under high stringency conditions to the complement of a nucleic acid encoding a heavy chain variable domain of V2.1, V2.3, V2.4, V2.5, V2.6, V2.7, or V2.11;
(d) the light chain immunoglobulin variable domain sequence comprises a sequence encoded by a nucleic acid that hybridizes under high stringency conditions to the complement of a nucleic acid encoding a light chain variable domain of V2.11;
(e) the heavy chain immunoglobulin variable domain sequence is at least 90% identical a heavy chain variable domain of V2.1, V2.3, V2.4, V2.5, V2.6, V2.7, or V2.11;
(f) the light chain immunoglobulin variable domain sequence is at least 90% identical a light chain variable domain of V2.11;
(g) the antibody molecule competes with mAb MJ2-7 for binding to human IL-13;
(h) the antibody molecule contacts one or more amino acid residues from IL-13 selected from the group consisting of residues 116, 117, 118, 122, 123, 124, 125, 126, 127, and 128 of SEQ ID NO:24 or SEQ ID NO:178;
(i) the heavy chain variable domain sequence has the same canonical structure as mAb MJ2-7 in hypervariable loops 1, 2 and/or 3;
(j) the light chain variable domain sequence has the same canonical structure as mAb MJ2-7 in hypervariable loops 1, 2 and/or 3; and
(k) the heavy chain variable domain sequence and/or the light chain variable domain sequence has FR1, FR2, and FR3 framework regions from VH segments encoded by germline genes DP-54 and DPK-9 respectively or a sequence at least 95% identical to VH segments encoded by germline genes DP-54 and DPK-9.

32. A method of treating an IL-13-associated disorder in a subject, the method comprising:
administering, to a subject having, or being at risk of having, the IL-13-associated disorder, one or more flat doses of an anti-IL-13 antibody molecule.

33. The method of claim 32, wherein the flat dose is between about 75 mg and about 500 mg.

34. The method of claim 33, wherein the flat dose is about 75 mg, 100 mg, 200 mg or 225 mg.

35. The method of any of claims 32-34, wherein the flat dose is administered to the subject approximately every week, approximately every 2 weeks, approximately every 3 weeks, approximately every 4 weeks, or approximately every month.
